SEQUENCE LISTING (124 sequences)

SEQ ID NO: 1 - protein, 319 aa, Artificial Sequence
  synthetic sequence
DCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTS

SEQ ID NO: 2 - protein, 251 aa, Artificial Sequence
  synthetic sequence
DCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRG

SEQ ID NO: 3 - protein, 251 aa, Artificial Sequence
  synthetic sequence
DCGLPPDVPNAQPALEGRTSFPEDTVITYKCEESFVKIPGEKDSVICLKGSQWSDIEEFCQRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRG

SEQ ID NO: 4 - protein, 315 aa, Artificial Sequence
  synthetic sequence
DCGPPPAVPNAQPALKGLTSFPENTVITYRCDENFMKIPGKQDSVMCLPDSQWSDIEEFCNRSCGAPTRLKFASLKQLYIPQSYFPVGTVVEYECRPGYRRDPSLLAKLTCLQNLKWSTAAEFCKKKSCPNPGEIPNGQIDTSNGILFGAAISFSCNTGYKLFGPTSSLCLVSGSGVQWSDTLPECREIYCPAPPQIDNGIIQGEREHYGYRQSITYLCNRGFTMIGEHSIYCTVNDDEGEWSGPPPTCRANSLVSKAPPTVQKPTTVNVRTTEVSPTSQKTTTPNAQATRSTPASRTTKHFHKTTPDKGSGTSS

SEQ ID NO: 5 - protein, 328 aa, Artificial Sequence
  synthetic sequence
DCGPPPDIPNARPILGRHSKFAEQSKVAYSCNNGFKQVPDKSNIVVCLENGQWSSHETFCEKSCVAPERLSFASLKKEYLNMNFFPVGTIVEYECRPGFRKQPPLPGKATCLEDLVWSPVAQFCKKKSCPNPKDLDNGHINIPTGILFGSEINFSCNPGYRLVGVSSTFCSVTGNTVDWDDEFPVCTEIHCPEPPKINNGIMRGESDSYTYSQVVTYSCDKGFILVGNASIYCTVSKSDVGQWSSPPPRCIEKSKVPTKKPTINVPSTGTPSTPQKPTTESVPNPGDQPTPQKPSTVKVSATQHVPVTKTTVRHPIRTSTDKGEPNTG

SEQ ID NO: 6 - protein, 252 aa, Artificial Sequence
  synthetic sequence
DCGPPPDIPNARPILGRHSKFAEQSKVAYSCNNGFKQVPDKSNIVVCLENGQWSSHETFCEKSCVAPERLSFASLKKEYLNMNFFPVGTIVEYECRPGFRKQPPLPGKATCLEDLVWSPVAQFCKKKSCPNPKDLDNGHINIPTGILFGSEINFSCNPGYRLVGVSSTFCSVTGNTVDWDDEFPVCTEIHCPEPPKINNGIMRGESDSYTYSQVVTYSCDKGFILVGNASIYCTVSKSDVGQWSSPPPRCIE

SEQ ID NO: 7 - protein, 77 aa, Artificial Sequence
  synthetic sequence
LQCYNCPNPTADCKTAVNCSSDFDACLITKAGLQVYNKCWKFEHCNFNDVTTRLRENELTYYCCKKDLCNFNEQLEN

SEQ ID NO: 8 - protein, 77 aa, Artificial Sequence
  synthetic sequence
LQCYNCPNPTTDCKTAINCSSGFDTCLIARAGLQVYNQCWKFANCNYNDISTLLKESELRYFCCKKDLCNFNEQLES

SEQ ID NO: 9 - protein, 73 aa, Artificial Sequence
  synthetic sequence
LTCYHCFQPVVSSCNMNSTCSPDQDSCLYAVAGMQVYQRCWKQSDCHGEIIMDQLEETKLKFRCCQFNLCNKS

SEQ ID NO: 10 - protein, 1930 aa, Artificial Sequence
  synthetic sequence
QCNAPEWLPFARPTNLTDEFEFPIGTYLNYECRPGYSGRPFSIICLKNSVWTGAKDRCRRKSCRNPPDPVNGMVHVIKGIQFGSQIKYSCTKGYRLIGSSSATCIISGDTVIWDNETPICDRIPCGLPPTITNGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKTVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPDVLHAERTQRDKDNFSPGQEVFYSCEPGYDLRGAASMRCTPQGDWSPAAPTCEVKSCDDFMGQLLNGRVLFPVNLQLGAKVDFVCDEGFQLKGSSASYCVLAGMESLWNSSVPVCEQIFCPSPPVIPNGRHTGKPLEVFPFGKAVNYTCDPHPDRGTSFDLIGESTIRCTSDPQGNGVWSSPAPRCGILGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCIIPNKCTPPNVENGILVSDNRSLFSLNEVVEFRCQPGFVMKGPRRVKCQALNKWEPELPSCSRVCQPPPEILHGEHTPSHQDNFSPGQEVFYSCEPGYDLRGAASLHCTPQGDWSPEAPRCAVKSCDDFLGQLPHGRVLFPLNLQLGAKVSFVCDEGFRLKGSSVSHCVLVGMRSLWNNSVPVCEHIFCPNPPAILNGRHTGTPSGDIPYGKEISYTCDPHPDRGMTFNLIGESTIRCTSDPHGNGVWSSPAPRCELSVRAGHCKTPEQFPFASPTIPINDFEFPVGTSLNYECRPGYFGKMFSISCLENLVWSSVEDNCRRKSCGPPPEPFNGMVHINTDTQFGSTVNYSCNEGFRLIGSPSTTCLVSGNNVTWDKKAPICEIISCEPPPTISNGDFYSNNRTSFHNGTVVTYQCHTGPDGEQLFELVGERSIYCTSKDDQVGVWSSPPPRCISTNKCTAPEVENAIRVPGNRSFFSLTEIIRFRCQPGFVMVGSHTVQCQTNGRWGPKLPHCSRVCQPPPEILHGEHTLSHQDNFSPGQEVFYSCEPSYDLRGAASLHCTPQGDWSPEAPRCTVKSCDDFLGQLPHGRVLLPLNLQLGAKVSFVCDEGFRLKGRSASHCVLAGMKALWNSSVPVCEQIFCPNPPAILNGRHTGTPFGDIPYGKEISYACDTHPDRGMTFNLIGESSIRCTSDPQGNGVWSSPAPRCELSVPAACPHPPKIQNGHYIGGHVSLYLPGMTISYICDPGYLLVGKGFIFCTDQGIWSQLDHYCKEVNCSFPLFMNGISKELEMKKVYHYGDYVTLKCEDGYTLEGSPWSQCQADDRWDPPLAKCTSRTHD

SEQ ID NO: 11 - protein, 365 aa, Artificial Sequence
  synthetic sequence
ELRGGLGKHGHTVHREPAVNRLCADSKRWSGLPVSAQRPFPMGHCPAPSQLPSAKPINLTDESMFPIGTYLLYECLPGYIKRQFSITCKQDSTWTSAEDKCIRKQCKTPSDPENGLVHVHTGIQFGSRINYTCNQGYRLIGSSSAVCVITDQSVDWDTEAPICEWIPCEIPPGIPNGDFFSSTREDFHYGMVVTYRCNTDARGKALFNLVGEPSLYCTSNDGEIGVWSGPPPQCIELNKCTPPPYVENAVMLSENRSLFSLRDIVEFRCHPGFIMKGASSVHCQSLNKWEPELPSCFKGVICRLPQEMSGFQKGLGMKKEYYYGENVTLECEDGYTLEGSSQSQCQSDGSWNPLLAKCVSRSISG

SEQ ID NO: 12 - protein, 253 aa, Artificial Sequence
  synthetic sequence
SCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLIGSYSSFCKISGSSVQWSDKLPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKV

SEQ ID NO: 13 - protein, 290 aa, Artificial Sequence
  synthetic sequence
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 14 - protein, 313 aa, Artificial Sequence
  synthetic sequence
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVA

SEQ ID NO: 15 - protein, 133 aa, Artificial Sequence [sequence omitted]

SEQ ID NO: 16 - protein, 153 aa, Artificial Sequence
  synthetic sequence
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 17 - protein, 153 aa, Artificial Sequence
  synthetic sequence
MYRMQLLSCIALSLALVTNSAPTSSSTKKTQLQLEHLLLDLQMILNGISNHKNPRLARMLTFKFYMPEKATEIKHLQCLEEELKPLEEALRLAPSKNFHLRPRDLISDINVIVLELKGSETTFMCEYADETATIVEFLNRWITFSQSIISTLT

SEQ ID NO: 18 - protein, 153 aa, Artificial Sequence
  synthetic sequence
MYRMQLLSCIALSLALVTNSAPTSSSTKKTQLQLEHLLLDLQMILNGISNHKNPRLARMLTFKFYMPEKATELKHIQCLEEELKPLEEALRLAPSKNFHLRPRDLISDINVIVLELKGSETTFMCEYADETATIVEFLNRWITFSQSIISTLT

SEQ ID NO: 19 - protein, 153 aa, Artificial Sequence
  synthetic sequence
MYRMQLLSCIALSLALVTNSAPTSSSTKKTQLQLEHLLLDLQMILNGISNHKNPRLARMLTFKFYMPEKATELKHLQCLEEELKPLEEALRLAPSKNFHIRPRDLISDINVIVLELKGSETTFMCEYADETATIVEFLNRWITFSQSIISTLT

SEQ ID NO: 20 - protein, 153 aa, Artificial Sequence
  synthetic sequence
MYRMQLLSCIALSLALVTNSAPTSSSTKKTQLQLEHLLLDLQMILNGISNHKNPRLARMLTFKFYMPEKATELKHLQCLEEELKPLEEALRLAPSKNFHLRPRDLISDINVIVLELKGSETTFMCEYADETATIVEFINRWITFSQSIISTLT

SEQ ID NO: 21 - protein, 20 aa, Artificial Sequence
  synthetic sequence
MYRMQLLSCIALSLALVTNS

SEQ ID NO: 22 - skipped

SEQ ID NO: 23 - protein, 226 aa, Artificial Sequence
  synthetic sequence
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - protein, 20 aa, Artificial Sequence
  synthetic sequence
GGGGSGGGGSGGGGSGGGGS

SEQ ID NO: 25 - protein, 15 aa, Artificial Sequence
  synthetic sequence
GGGGSGGGGSGGGGS

SEQ ID NO: 26 - protein, 5 aa, Artificial Sequence
  synthetic sequence
GGGGS

SEQ ID NO: 27 - protein, 5 aa, Artificial Sequence
  synthetic sequence
GGGGA

SEQ ID NO: 28 - protein, 399 aa, Artificial Sequence
  synthetic sequence
MYRMQLLSCIALSLALVTNSAPTSSSTKKTQLQLEHLLLDLQMILNGISNHKNPRLARMLTFKFYMPEKATEIKHLQCLEEELKPLEEALRLAPSKNFHLRPRDLISDINVIVLELKGSETTFMCEYADETATIVEFLNRWITFSQSIISTLTGGGGSGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 29 - protein, 399 aa, Artificial Sequence
  synthetic sequence
MYRMQLLSCIALSLALVTNSAPTSSSTKKTQLQLEHLLLDLQMILNGISNHKNPRLARMLTFKFYMPEKATELKHIQCLEEELKPLEEALRLAPSKNFHLRPRDLISDINVIVLELKGSETTFMCEYADETATIVEFLNRWITFSQSIISTLTGGGGSGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - protein, 399 aa, Artificial Sequence
  synthetic sequence
MYRMQLLSCIALSLALVTNSAPTSSSTKKTQLQLEHLLLDLQMILNGISNHKNPRLARMLTFKFYMPEKATELKHLQCLEEELKPLEEALRLAPSKNFHIRPRDLISDINVIVLELKGSETTFMCEYADETATIVEFLNRWITFSQSIISTLTGGGGSGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 399 aa, Artificial Sequence
  synthetic sequence
MYRMQLLSCIALSLALVTNSAPTSSSTKKTQLQLEHLLLDLQMILNGISNHKNPRLARMLTFKFYMPEKATELKHLQCLEEELKPLEEALRLAPSKNFHLRPRDLISDINVIVLELKGSETTFMCEYADETATIVEFINRWITFSQSIISTLTGGGGSGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 32 - protein, 369 aa, Artificial Sequence
  synthetic sequence
APTSSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFSQSIISTLTGGGGAGGGGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 356 aa, Artificial Sequence
  synthetic sequence
APTSSSTKKTQLQLEHLLLHLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 34 - protein, 359 aa, Artificial Sequence
  synthetic sequence
APTSSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFSQSIISTLTDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 35 - protein, 364 aa, Artificial Sequence
  synthetic sequence
APTSSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFSQSIISTLTGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 369 aa, Artificial Sequence
  synthetic sequence
APTSSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFSQSIISTLTGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 37 - protein, 374 aa, Artificial Sequence
  synthetic sequence
APTSSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFSQSIISTLTGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNYHTQKSLSLSPG

SEQ ID NO: 38 - protein, 379 aa, Artificial Sequence
  synthetic sequence
APTSSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFSQSIISTLTGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 39 - protein, 374 aa, Artificial Sequence
  synthetic sequence
APTSSSTKKTQLQLEHLLLHLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGGGGSGGGGSGGGGSDKTHTCPPCPAPELLGGPSVFLFPPKPKDTLMISRTPEVTCVVVDVSHEDPEVKFNWYVDGVEVHNAKTKPREEQYASTYPVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 40 - protein, 133 aa, Artificial Sequence
  synthetic sequence
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 41 - protein, 133 aa, Artificial Sequence
  synthetic sequence
APASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 42 - protein, 133 aa, Artificial Sequence
  synthetic sequence
APTSSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFSQSIISTLT

SEQ ID NO: 43 - protein, 133 aa, Artificial Sequence [sequence omitted]

SEQ ID NO: 44 - protein, 133 aa, Artificial Sequence
  synthetic sequence
APTSSSTKKTQLQLEHLLLDLQMILNGINNYKNPKLTRMLTFKFYMPKKATELKHLQCLEEELKPLEEALNLAPSKNFHLRPRDLISDINVIVLELKGSETTFMCEYADETATIVEFLNRWITFSQSIISTLT

SEQ ID NO: 45 - protein, 133 aa, Artificial Sequence
  synthetic sequence
APTSSSTKKTQLQLEHLLLDLQMILNGINNYKNPKLTRMLTFKFYMPKKATELKHLQCLEEELKPLEEALNLAPSKNFHLRPRDLISRINVIVLELKGSETTFMCEYADETATIVEFLNRWITFSQSIISTLT

SEQ ID NO: 46 - protein, 133 aa, Artificial Sequence
  synthetic sequence
APTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLT

SEQ ID NO: 47 - protein, 133 aa, Artificial Sequence
  synthetic sequence
APTSSSTKKTQLQLEHLLLDLQMILNGINNYKNPKLTRMLTFKFYMPKKATEIKHLQCLEEELKPLEEALNLAPSKNFHLRPRDLISDINVIVLELKGSETTFMCEYADETATIVEFLNRWITFSQSIISTLT

SEQ ID NO: 48 - protein, 133 aa, Artificial Sequence
  synthetic sequence
APTSSSTKKTQLQLEHLLLDLQMILNGINNYKNPKLTRMLTFKFYMPKKATELKHIQCLEEELKPLEEALNLAPSKNFHLRPRDLISDINVIVLELKGSETTFMCEYADETATIVEFLNRWITFSQSIISTLT

SEQ ID NO: 49 - protein, 133 aa, Artificial Sequence
  synthetic sequence
APTSSSTKKTQLQLEHLLLDLQMILNGINNYKNPKLTRMLTFKFYMPKKATELKHLQCLEEELKPLEEALNLAPSKNFHIRPRDLISDINVIVLELKGSETTFMCEYADETATIVEFLNRWITFSQSIISTLT

SEQ ID NO: 50 - protein, 133 aa, Artificial Sequence
  synthetic sequence
APTSSSTKKTQLQLEHLLLDLQMILNGINNYKNPKLTRMLTFKFYMPKKATELKHLQCLEEELKPLEEALNLAPSKNFHLRPRDLISDINVIVLELKGSETTFMCEYADETATIVEFINRWITFSQSIISTLT

SEQ ID NO: 51 - protein, 226 aa, Artificial Sequence
  synthetic sequence
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 52 - protein, 226 aa, Artificial Sequence
  synthetic sequence
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 53 - protein, 329 aa, Artificial Sequence
  synthetic sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 54 - protein, 107 aa, Artificial Sequence [sequence omitted]

SEQ ID NO: 55 - protein, 374 aa, Artificial Sequence
  synthetic sequence
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 56 - protein, 374 aa, Artificial Sequence
  synthetic sequence
APASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 57 - protein, 374 aa, Artificial Sequence
  synthetic sequence
APTSSSTKKTQLQLEHLLLDLQMILNGINNYKNPKLTRMLTFKFYMPKKATELKHLQCLEEELKPLEEVLNLAQSKNFHLRPRDLISRINVIVLELKGSETTFMCEYADETATIVEFLNRWITFSQSIISTLTGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 58 - protein, 374 aa, Artificial Sequence
  synthetic sequence
APTSSSTKKTQLQLEHLLLDLQMILNGINNYKNPKLTRMLTFKFYMPKKATELKHLQCLEEELKPLEEALNLAPSKNFHLRPRDLISNINVIVLELKGSETTFMCEYADETATIVEFLNRWITFSQSIISTLTGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 59 - protein, 374 aa, Artificial Sequence
  synthetic sequence
APTSSSTKKTQLQLEHLLLDLQMILNGINNYKNPKLTRMLTFKFYMPKKATELKHLQCLEEELKPLEEALNLAPSKNFHLRPRDLISDINVIVLELKGSETTFMCEYADETATIVEFLNRWITFSQSIISTLTGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 60 - protein, 374 aa, Artificial Sequence
  synthetic sequence
APTSSSTKKTQLQLEHLLLDLQMILNGINNYKNPKLTRMLTFKFYMPKKATELKHLQCLEEELKPLEEALNLAPSKNFHLRPRDLISRINVIVLELKGSETTFMCEYADETATIVEFLNRWITFSQSIISTLTGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 61 - protein, 374 aa, Artificial Sequence
  synthetic sequence
APTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFSQSIISTLTGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 62 - protein, 379 aa, Artificial Sequence
  synthetic sequence
APTSSSTKKTQLQLEHLLLDLQMILNGINNYKNPKLTRMLTFKFYMPKKATEIKHLQCLEEELKPLEEALNLAPSKNFHLRPRDLISDINVIVLELKGSETTFMCEYADETATIVEFLNRWITFSQSIISTLTGGGGSGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 63 - protein, 379 aa, Artificial Sequence
  synthetic sequence
APTSSSTKKTQLQLEHLLLDLQMILNGINNYKNPKLTRMLTFKFYMPKKATELKHIQCLEEELKPLEEALNLAPSKNFHLRPRDLISDINVIVLELKGSETTFMCEYADETATIVEFLNRWITFSQSIISTLTGGGGSGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 64 - protein, 379 aa, Artificial Sequence
  synthetic sequence
APTSSSTKKTQLQLEHLLLDLQMILNGINNYKNPKLTRMLTFKFYMPKKATELKHLQCLEEELKPLEEALNLAPSKNFHIRPRDLISDINVIVLELKGSETTFMCEYADETATIVEFLNRWITFSQSIISTLTGGGGSGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 65 - protein, 379 aa, Artificial Sequence
  synthetic sequence
APTSSSTKKTQLQLEHLLLDLQMILNGINNYKNPKLTRMLTFKFYMPKKATELKHLQCLEEELKPLEEALNLAPSKNFHLRPRDLISDINVIVLELKGSETTFMCEYADETATIVEFINRWITFSQSIISTLTGGGGSGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 66 - protein, 379 aa, Artificial Sequence
  synthetic sequence
APTSSSTKKTQLQLEHLLLDLQMILNGINNYKNPKLTRMLTFKFYMPKKATELKHLQCLEEELKPLEEALNLAPSKNFHLRPRDLISDINVIVLELKGSETTFMCEYADETATIVEFLNRWITFSQSIISTLTGGGGSGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 67 - protein, 364 aa, Artificial Sequence
  synthetic sequence
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSAPTSSSTKKTQLQLEHLLLDLQMILNGINNYKNPKLTRMLTFKFYMPKKATELKHLQCLEEELKPLEEALNLAPSKNFHLRPRDLISDINVIVLELKGSETTFMCEYADETATIVEFLNRWITFAQSIISTLT

SEQ ID NO: 68 - protein, 379 aa, Artificial Sequence
  synthetic sequence
  MISC_FEATURE (53)..(118): At least one of X is I and the remainder are L or I.
  MISC_FEATURE (53)..(53): At least one of X is I and the remainder are L or I.
  MISC_FEATURE (56)..(56): At least one of X is I and the remainder are L or I.
  MISC_FEATURE (80)..(80): At least one of X is I and the remainder are L or I.
  MISC_FEATURE (118)..(118): At least one of X is I and the remainder are L or I.
APTSSSTKKTQLQLEHLLLDLQMILNGINNYKNPKLTRMLTFKFYMPKKATEXKHXQCLEEELKPLEEALNLAPSKNFHXRPRDLISDINVIVLELKGSETTFMCEYADETATIVEFXNRWITFSQSIISTLTGGGGSGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 69 - protein, 133 aa, Artificial Sequence
  synthetic sequence
  MISC_FEATURE (53)..(118): At least one of X1, X2, X3, and X4 is I and the remainder are L or I.
  MISC_FEATURE (53)..(53): At least one of X1, X2, X3, and X4 is I and the remainder are L or I.
  MISC_FEATURE (56)..(56): At least one of X1, X2, X3, and X4 is I and the remainder are L or I.
  MISC_FEATURE (80)..(80): At least one of X1, X2, X3, and X4 is I and the remainder are L or I.
  MISC_FEATURE (118)..(118): At least one of X1, X2, X3, and X4 is I and the remainder are L or I.
APTSSSTKKTQLQLEHLLLDLQMILNGINNYKNPKLTRMLTFKFYMPKKATEXKHXQCLEEELKPLEEALNLAPSKNFHXRPRDLISDINVIVLELKGSETTFMCEYADETATIVEFXNRWITFSQSIISTLT

SEQ ID NO: 70 - protein, 838 aa, Artificial Sequence
  synthetic sequence
SRLRQEDFPPRIVEHPSDVIVSKGEPTTLNCKAEGRPTPTIEWYKDGERVETDKDDPRSHRMLLPSGSLFFLRIVHGRRSKPDEGSYVCVARNYLGEAVSRNASLEVALLRDDFRQNPTDVVVAAGEPAILECQPPRGHPEPTIYWKKDKVRIDDKEERISIRGGKLMISNTRKSDAGMYTCVGTNMVGERDSDPAELTVFERPTFLRRPINQVVLEEEAVEFRCQVQGDPQPTVRWKKDDADLPRGRYDIKDDYTLRIKKTMSTDEGTYMCIAENRVGKMEASATLTVRAPPQFVVRPRDQIVAQGRTVTFPCETKGNPQPAVFWQKEGSQNLLFPNQPQQPNSRCSVSPTGDLTITNIQRSDAGYYICQALTVAGSILAKAQLEVTDVLTDRPPPIILQGPANQTLAVDGTALLKCKATGDPLPVISWLKEGFTFPGRDPRATIQEQGTLQIKNLRISDTGTYTCVATSSSGETSWSAVLDVTESGATISKNYDLSDLPGPPSKPQVTDVTKNSVTLSWQPGTPGTLPASAYIIEAFSQSVSNSWQTVANHVKTTLYTVRGLRPNTIYLFMVRAINPQGLSDPSPMSDPVRTQDISPPAQGVDHRQVQKELGDVLVRLHNPVVLTPTTVQVTWTVDRQPQFIQGYRVMYRQTSGLQATSSWQNLDAKVPTERSAVLVNLKKGVTYEIKVRPYFNEFQGMDSESKTVRTTEEAPSAPPQSVTVLTVGSYNSTSISVSWDPPPPDHQNGIIQEYKIWCLGNETRFHINKTVDAAIRSVIIGGLFPGIQYRVEVAASTSAGVGVKSEPQPIIIGRRNEVVITENNNSITEQITDVVKQP

SEQ ID NO: 71 - protein, 838 aa, Artificial Sequence
  synthetic sequence
SRLRQEDFPPRIVEHPSDVIVSKGEPTTLNCKAEGRPTPTIEWYKDGERVETDKDDPRSHRMLLPSGSLFFLRIVHGRRSKPDEGSYVCVARNYLGEAVSRNASLEVALLRDDFRQNPTDVVVAAGEPAILECQPPRGHPEPTIYWKKDKVRIDDKEERISIRGGKLMISNTRKSDAGMYTCVGTNMVGERDSDPAELTVFERPTFLRRPINQVVLEEEAVEFRCQVQGDPQPTVRWKKDDADLPRGRYDIKDDYTLRIKKTMSTDEGTYMCIAENRVGKMEASATLTVRAPPQFVVRPRDQIVAQGRTVTFPCETKGNPQPAVFWQKEGSQNLLFPNQPQQPNSRCSVSPTGDLTITNIQRSDAGYYICQALTVAGSILAKAQLEVTDVLTDRPPPIILQGPANQTLAVDGTALLKCKATGDPLPVISWLKEGFTFLSRDPRATIQEQGTLQIKNLRISDTGTYTCVATSSSGETSWSAVLDVTESGATISKNYDLNDLPGPPSKPQVTDVTKNSVTLSWQPGTPGTLPASAYIIEAFSQSVSNSWQTVANHVKTTLYTVRGLRPNTIYLFMVRAINPQGLSDPSPMSDPVRTQDISPPAQGVDHRQVQKELGDVLVRLHNPVVLTPTTVQVTWTVDRQPQFIQGYRVMYRQTSGLQAASSWQNLDAKVPNERSAVLVNLKKGVTYEIKVRPYFNEFQGMDSESKTVRTTEEAPSAPPQSVTVLTVGSYNSTSISVSWDPPPPDHQNGILQEYKIWCLGNETRFHINKTVDAAIRSVIIGGLFPGIQYRVEVAASTSAGVGVKSEPQPIIIGRRNEVVITENNNSITEQITDVVKQP

SEQ ID NO: 72 - protein, 842 aa, Artificial Sequence
  synthetic sequence
SRLRQEDFPPRIVEHPSDVIVSKGEPTTLNCKAEGRPTPTIEWYKDGERVETDKDDPRSHRMLLPSGSLFFLRIVHGRRSKPDEGSYVCVARNYLGEAVSRNASLEVALLRDDFRQNPTDVVVAAGEPAILECQPPRGHPEPTIYWKKDKVRIDDKEERISIRGGKLMISNTRKSDAGMYTCVGTNMVGERDSDPAELTVFERPTFLRRPINQVVLEEEAVEFRCQVQGDPQPTVRWKKDDADLPRGRYDIKDDYTLRIKKAMSTDEGTYVCIAENRVGKVEASATLTVRVRPVAPPQFVVRPRDQIVAQGRTVTFPCETKGNPQPAVFWQKEGSQNLLFPNQPQQPNSRCSVSPTGDLTITNIQRSDAGYYICQALTVAGSILAKAQLEVTDVLTDRPPPIILQGPINQTLAVDGTALLKCKATGEPLPVISWLKEGFTFLGRDPRATIQDQGTLQIKNLRISDTGTYTCVATSSSGETSWSAVLDVTESGATISKNYDMNDLPGPPSKPQVTDVSKNSVTLSWQPGTPGVLPASAYIIEAFSQSVSNSWQTVANHVKTTLYTVRGLRPNTIYLFMVRAINPQGLSDPSPMSDPVRTQDISPPAQGVDHRQVQKELGDVVVRLHNPVVLTPTTVQVTWTVDRQPQFIQGYRVMYRQTSGLQASTVWQNLDAKVPTERSAVLVNLKKGVTYEIKVRPYFNEFQGMDSESKTVRTTEEAPSAPPQSVTVLTVGSHNSTSISVSWDPPPADHQNGIIQEYKIWCLGNETRFHINKTVDAAIRSVVIGGLFPGIQYRVEVAASTSAGVGVKSEPQPIIIGGRNEVVITENNNSITEQITDVVKQP

SEQ ID NO: 73 - protein, 842 aa, Artificial Sequence
  synthetic sequence
SRLRQEDFPPRIVEHPSDVIVSKGEPTTLNCKAEGRPTPTIEWYKDGERVETDKDDPRSHRMLLPSGSLFFLRIVHGRRSKPDEGTYVCVARNYLGEAVSRNASLEVALLRDDFRQNPTDVVVAAGEPAILECQPPRGHPEPTIYWKKDKVRIDEKEERISIRGGKLMISNTRKSDAGMYTCVGTNMVGERDSDPAELTVFERPTFLRRPINQVVLEDEPAEFRCQVQGDPQPTVRWKKDDADLPRGRYDIKDDYTLRIKKAISADEGTYVCIAENRVGKVEASATLTVRVRPVAPPQFVVRPRDQIVAQGRTVTFPCETKGNPQPAVFWQKEGSQNLLFPNQPQQPNSRCSVSPTGDLTITNIQRSDAGYYICQALTVAGSILAKAQLEVTDVLTDRPPPIILQGPINQTLAVDGTALLKCKATGEPLPVISWLKEGFTFLGRDPRATIQDQGTLQIKNLRISDTGTYTCVATSSSGETSWSAVLDVTESGATISKNYDTNDLPGPPSKPQVTDVTKNSVTLSWQPGTPGVLPASAYIIEAFSQSVSNSWQTVANHVKTTLYTVRGLRPNTIYLFMVRAINPQGLSDPSPMSDPVRTQDISPPAQGVDHRQVQKELGDVTVRLHNPVVLTPTTVQVTWTVDRQPQFIQGYRVMYRQTSGLQASTVWQNLDAKVPTERSAVLVNLKKGVTYEIKVRPYFNEFQGMDSESKTIRTTEEAPSAPPQSVTVLTVGSHNSTSISVSWDPPPADHQNGIIQEYKIWCLGNETRFHINKTVDATIRSVVIGGLFPGIQYRVEVAASTSAGVGVKSEPQPIIIGGRNEVVITENNNSITEQITDVVKQP

SEQ ID NO: 74 - protein, 872 aa, Artificial Sequence
  synthetic sequence
QLIPDPEDVERGNDHGTPIPTSDNDDNSLGYTGSRLRQEDFPPRIVEHPSDLIVSKGEPATLNCKAEGRPTPTIEWYKGGERVETDKDDPRSHRMLLPSGSLFFLRIVHGRKSRPDEGVYVCVARNYLGEAVSHNASLEVAILRDDFRQNPSDVMVAVGEPAVMECQPPRGHPEPTISWKKDGSPLDDKDERITIRGGKLMITYTRKSDAGKYVCVGTNMVGERESEVAELTVLERPSFVKRPSNLAVTVDDSAEFKCEARGDPVPTVRWRKDDGELPKSRYEIRDDHTLKIRKVTAGDMGSYTCVAENMVGKAEASATLTVQEPPHFVVKPRDQVVALGRTVTFQCEATGNPQPAIFWRREGSQNLLFSYQPPQSSSRFSVSQTGDLTITNVQRSDVGYYICQTLNVAGSIITKAYLEVTDVIADRPPPVIRQGPVNQTVAVDGTFVLSCVATGSPVPTILWRKDGVLVSTQDSRIKQLENGVLQIRYAKLGDTGRYTCIASTPSGEATWSAYIEVQEFGVPVQPPRPTDPNLIPSAPSKPEVTDVSRNTVTLSWQPNLNSGATPTSYIIEAFSHASGSSWQTVAENVKTETSAIKGLKPNAIYLFLVRAANAYGISDPSQISDPVKTQDVLPTSQGVDHKQVQRELGNAVLHLHNPTVLSSSSIEVHWTVDQQSQYIQGYKILYRPSGANHGESDWLVFEVRTPAKNSVVIPDLRKGVNYEIKARPFFNEFQGADSEIKFAKTLEEAPSAPPQGVTVSKNDGNGTAILVSWQPPPEDTQNGMVQEYKVWCLGNETRYHINKTVDGSTFSVVIPFLVPGIRYSVEVAASTGAGSGVKSEPQFIQLDAHGNPVSPEDQVSLAQQISDVVKQP

SEQ ID NO: 75 - protein, 230 aa, Artificial Sequence
  synthetic sequence
WTTRGFVFTRHSQTTAIPSCPEGTVPLYSGFSFLFVQGNQRAHGQDLGTLGSCLQRFTTMPFLFCNVNDVCNFASRNDYSYWLSTPALMPMNMAPITGRALEPYISRCTVCEGPAIAIAVHSQTTDIPPCPHGWISLWKGFSFIMFTSAGSEGTGQALASPGSCLEEFRASPFLECHGRGTCNYYSNSYSFWLASLNPERMFRKPIPSTVKAGELEKIISRCQVCMKKRH

SEQ ID NO: 76 - protein, 230 aa, Artificial Sequence
  synthetic sequence
WTTRGFVFTRHSQTTAIPSCPEGTAPLYSGFSFLFVQGNERAHGQDLGTLGSCLQRFTTMPFLFCNVNDVCNFASRNDYSYWLSTPALMPMNMAPITGRALEPYISRCTVCEAPAIAIAVHSQTTDIPPCPHGWISLWKGFSFIMFTSAGSEGTGQALASPGSCLEEFRASPFLECHGRGTCNYYSNSYSFWLASLNPERMFRKPIPSTVKAGELEKIISRCQVCMKKRH

SEQ ID NO: 77 - protein, 231 aa, Artificial Sequence
  synthetic sequence
GTRMRGFIFTRHSQTTAIPSCPEGTQPLYSGFSLLFVQGNKRAHGQDLGTLGSCLQRFTTMPFLFCNINNVCNFASRNDYSYWLSTPALMPMDMAPISGRALEPYISRCTVCEGPAMAIAVHSQTTAIPPCPQDWVSLWKGFSFIMFTSAGSEGAGQALASPGSCLEEFRASPFIECHGRGTCNYYSNSYSFWLASLNPERMFRKPIPSTVKAGDLEKIISRCQVCMKKRH

SEQ ID NO: 78 - protein, 231 aa, Artificial Sequence
  synthetic sequence
GTRMRGFIFTRHSQTTANPSCPEGTQPLYSGFSLLFVQGNEHAHGQDLGTLGSCLQRFTTMPFLFCNVDNVCNFASRNDYSYWLSTPAPMPMDMAPITGRALEPYVSRCTVCEGPAMAIAVHSQTTAIPPCPQGWVSLWKGFSFVMFTSAGSEGAGQALASPGSCLEEFRASPFIECHGRGTCNYYSNSYSFWLASLNPERMFRKPIPSTVKAGDLEKIISRCQVCMKKRH

SEQ ID NO: 79 - protein, 230 aa, Artificial Sequence
  synthetic sequence
GYLGGFLLVLHSQTDQEPTCPLGMPRLWTGYSLLYLEGQEKAHNQDLGLAGSCLPVFSTLPFAYCNIHQVCHYAQRNDRSYWLASAAPLPMMPLSEEAIRPYVSRCAVCEAPAQAVAVHSQDQSIPPCPQTWRSLWIGYSFLMHTGAGDQGGGQALMSPGSCLEDFRAAPFLECQGRQGTCHFFANKYSFWLTTVKADLQFSSAPAPDTLKESQAQRQKISRCQVCVKYS

SEQ ID NO: 80 - protein, 230 aa, Artificial Sequence
  synthetic sequence
SSVAHGFLITRHSQTTDAPQCPQGTLQVYEGFSLLYVQGNKRAHGQDLGTAGSCLRRFSTMPFMFCNINNVCNFASRNDYSYWLSTPEPMPMSMQPLKGQSIQPFISRCAVCEAPAVVIAVHSQTIQIPHCPQGWDSLWIGYSFMMHTSAGAEGSGQALASPGSCLEEFRSAPFIECHGRGTCNYYANSYSFWLATVDVSDMFSKPQSETLKAGDLRTRISRCQVCMKRT

SEQ ID NO: 81 - protein, 780 aa, Artificial Sequence
  synthetic sequence
EVQLVQSGAEVKKPGASVKVSCKASGYTFTGYYMHWVRQAPGQGLEWMGWINPKNGDTEFPQKFQGRVTMTRDTSITTAYMDLSRLRSDDTAVYYCARESGDDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSDCGPPPDIPNARPILGRHSKFAEQSKVAYSCNNGFKQVPDKSNIVVCLENGQWSSHETFCEKSCVAPERLSFASLKKEYLNMNFFPVGTIVEYECRPGFRKQPPLPGKATCLEDLVWSPVAQFCKKKSCPNPKDLDNGHINIPTGILFGSEINFSCNPGYRLVGVSSTFCSVTGNTVDWDDEFPVCTEIHCPEPPKINNGIMRGESDSYTYSQVVTYSCDKGFILVGNASIYCTVSKSDVGQWSSPPPRCIEKSKVPTKKPTINVPSTGTPSTPQKPTTESVPNPGDQPTPQKPSTVKVSATQHVPVTKTTVRHPIRTSTDKGEPNTG

SEQ ID NO: 82 - protein, 118 aa, Artificial Sequence
  synthetic sequence
EVQLVQSGAEVKKPGASVKVSCKASGYTFTGYYMHWVRQAPGQGLEWMGWINPKNGDTEFPQKFQGRVTMTRDTSITTAYMDLSRLRSDDTAVYYCARESGDDAFDIWGQGTMVTVSS

SEQ ID NO: 83 - protein, 212 aa, Artificial Sequence
  synthetic sequence
DIVMTQSPSSLSASVGDRVTITCRASQSISSYLNWYQQKPGKAPKLLIYAASSLQSGVPSRFSGSGSGTDFTLTISSLQPEDFATYYCQQSVGLFFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 105 aa, Artificial Sequence
  synthetic sequence
DIVMTQSPSSLSASVGDRVTITCRASQSISSYLNWYQQKPGKAPKLLIYAASSLQSGVPSRFSGSGSGTDFTLTISSLQPEDFATYYCQQSVGLFFGGGTKVEIK

SEQ ID NO: 85 - protein, 783 aa, Artificial Sequence
  synthetic sequence
EVQLVESGGGLVQPGRSLKLSCAASGFTFSDYYMAWVRQAPKKGLEWVASISYEGSSTNYGDSVKGRFTISRDNAKSTLYLQMNSLRSEDTATYYCARQYHYYSGDGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSDCGPPPDIPNARPILGRHSKFAEQSKVAYSCNNGFKQVPDKSNIVVCLENGQWSSHETFCEKSCVAPERLSFASLKKEYLNMNFFPVGTIVEYECRPGFRKQPPLPGKATCLEDLVWSPVAQFCKKKSCPNPKDLDNGHINIPTGILFGSEINFSCNPGYRLVGVSSTFCSVTGNTVDWDDEFPVCTEIHCPEPPKINNGIMRGESDSYTYSQVVTYSCDKGFILVGNASIYCTVSKSDVGQWSSPPPRCIEKSKVPTKKPTINVPSTGTPSTPQKPTTESVPNPGDQPTPQKPSTVKVSATQHVPVTKTTVRHPIRTSTDKGEPNTG

SEQ ID NO: 86 - protein, 121 aa, Artificial Sequence
  synthetic sequence
EVQLVESGGGLVQPGRSLKLSCAASGFTFSDYYMAWVRQAPKKGLEWVASISYEGSSTNYGDSVKGRFTISRDNAKSTLYLQMNSLRSEDTATYYCARQYHYYSGDGFAYWGQGTLVTVSS

SEQ ID NO: 87 - protein, 214 aa, Artificial Sequence
  synthetic sequence
DIQMTQSPSSLSASLGDRVTITCRASQDFGDYFAWFQQTPGKSPRLLIYGATDLEDGVPSRFSGSRSGSDYSLTISSLESEDTAIYYCLQYDKYPLTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - protein, 107 aa, Artificial Sequence
  synthetic sequence
DIQMTQSPSSLSASLGDRVTITCRASQDFGDYFAWFQQTPGKSPRLLIYGATDLEDGVPSRFSGSRSGSDYSLTISSLESEDTAIYYCLQYDKYPLTFGSGTKLEIK

SEQ ID NO: 89 - protein, 593 aa, Artificial Sequence
  synthetic sequence
DCGPPPDIPNARPILGRHSKFAEQSKVAYSCNNGFKQVPDKSNIVVCLENGQWSSHETFCEKSCVAPERLSFASLKKEYLNMNFFPVGTIVEYECRPGFRKQPPLPGKATCLEDLVWSPVAQFCKKKSCPNPKDLDNGHINIPTGILFGSEINFSCNPGYRLVGVSSTFCSVTGNTVDWDDEFPVCTEIHCPEPPKINNGIMRGESDSYTYSQVVTYSCDKGFILVGNASIYCTVSKSDVGQWSSPPPRCIEKSKVPTKKPTINVPSTGTPSTPQKPTTESVPNPGDQPTPQKPSTVKVSATQHVPVTKTTVRHPIRTSTDKGEPNTGGGGGSGGGGSGGGGSGGGGSEVQLVQSGAEVKKPGASVKVSCKASGYTFTGYYMHWVRQAPGQGLEWMGWINPKNGDTEFPQKFQGRVTMTRDTSITTAYMDLSRLRSDDTAVYYCARESGDDAFDIWGQGTMVTVSSGGGGGSGGGGSGGGGSDIVMTQSPSSLSASVGDRVTITCRASQSISSYLNWYQQKPGKAPKLLIYAASSLQSGVPSRFSGSGSGTDFTLTISSLQPEDFATYYCQQSVGLFFGGGTKVEIKHHHHHH

SEQ ID NO: 90 - protein, 602 aa, Artificial Sequence
  synthetic sequence
DCGPPPDIPNARPILGRHSKFAEQSKVAYSCNNGFKQVPDKSNIVVCLENGQWSSHETFCEKSCVAPERLSFASLKKEYLNMNFFPVGTIVEYECRPGFRKQPPLPGKATCLEDLVWSPVAQFCKKKSCPNPKDLDNGHINIPTGILFGSEINFSCNPGYRLVGVSSTFCSVTGNTVDWDDEFPVCTEIHCPEPPKINNGIMRGESDSYTYSQVVTYSCDKGFILVGNASIYCTVSKSDVGQWSSPPPRCIEKSKVPTKKPTINVPSTGTPSTPQKPTTESVPNPGDQPTPQKPSTVKVSATQHVPVTKTTVRHPIRTSTDKGEPNTGGGGGSGGGGSGGGGSGGGGSEVQLVESGGGLVQPGRSLKLSCAASGFTFSDYYMAWVRQAPKKGLEWVASISYEGSSTNYGDSVKGRFTISRDNAKSTLYLQMNSLRSEDTATYYCARQYHYYSGDGFAYWGQGTLVTVSSGGGGSGGGGSGGGGSGGGGSDIQMTQSPSSLSASLGDRVTITCRASQDFGDYFAWFQQTPGKSPRLLIYGATDLEDGVPSRFSGSRSGSDYSLTISSLESEDTAIYYCLQYDKYPLTFGSGTKLEIKHHHHHH

SEQ ID NO: 91 - protein, 338 aa, Artificial Sequence
  synthetic sequence
LTCYHCFQPVVSSCNMNSTCSPDQDSCLYAVAGMQVYQRCWKQSDCHGEIIMDQLEETKLKFRCCQFNLCNKSGGGGSGGGGSGGGGSGGGGSEVQLVQSGAEVKKPGASVKVSCKASGYTFTGYYMHWVRQAPGQGLEWMGWINPKNGDTEFPQKFQGRVTMTRDTSITTAYMDLSRLRSDDTAVYYCARESGDDAFDIWGQGTMVTVSSGGGGGSGGGGSGGGGSDIVMTQSPSSLSASVGDRVTITCRASQSISSYLNWYQQKPGKAPKLLIYAASSLQSGVPSRFSGSGSGTDFTLTISSLQPEDFATYYCQQSVGLFFGGGTKVEIKHHHHHH

SEQ ID NO: 92 - protein, 833 aa, Artificial Sequence
  synthetic sequence
DCGPPPDIPNARPILGRHSKFAEQSKVAYSCNNGFKQVPDKSNIVVCLENGQWSSHETFCEKSCVAPERLSFASLKKEYLNMNFFPVGTIVEYECRPGFRKQPPLPGKATCLEDLVWSPVAQFCKKKSCPNPKDLDNGHINIPTGILFGSEINFSCNPGYRLVGVSSTFCSVTGNTVDWDDEFPVCTEIHCPEPPKINNGIMRGESDSYTYSQVVTYSCDKGFILVGNASIYCTVSKSDVGQWSSPPPRCIEKSKVPTKKPTINVPSTGTPSTPQKPTTESVPNPGDQPTPQKPSTVKVSATQHVPVTKTTVRHPIRTSTDKGEPNTGGGGGSGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLVQSGAEVKKPGASVKVSCKASGYTFTGYYMHWVRQAPGQGLEWMGWINPKNGDTEFPQKFQGRVTMTRDTSITTAYMDLSRLRSDDTAVYYCARESGDDAFDIWGQGTMVTVSSGGGGGSGGGGSGGGGSDIVMTQSPSSLSASVGDRVTITCRASQSISSYLNWYQQKPGKAPKLLIYAASSLQSGVPSRFSGSGSGTDFTLTISSLQPEDFATYYCQQSVGLFFGGGTKVEIK

SEQ ID NO: 93 - protein, 578 aa, Artificial Sequence
  synthetic sequence
LTCYHCFQPVVSSCNMNSTCSPDQDSCLYAVAGMQVYQRCWKQSDCHGEIIMDQLEETKLKFRCCQFNLCNKSGGGGSGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSGGGGSEVQLVQSGAEVKKPGASVKVSCKASGYTFTGYYMHWVRQAPGQGLEWMGWINPKNGDTEFPQKFQGRVTMTRDTSITTAYMDLSRLRSDDTAVYYCARESGDDAFDIWGQGTMVTVSSGGGGGSGGGGSGGGGSDIVMTQSPSSLSASVGDRVTITCRASQSISSYLNWYQQKPGKAPKLLIYAASSLQSGVPSRFSGSGSGTDFTLTISSLQPEDFATYYCQQSVGLFFGGGTKVEIK

SEQ ID NO: 94 - protein, 771 aa, Artificial Sequence
  synthetic sequence
EVQLVQSGAEVKKPGASVKVSCKASGYTFTGYYMHWVRQAPGQGLEWMGWINPKNGDTEFPQKFQGRVTMTRDTSITTAYMDLSRLRSDDTAVYYCARESGDDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTS

SEQ ID NO: 95 - protein, 703 aa, Artificial Sequence
  synthetic sequence
EVQLVQSGAEVKKPGASVKVSCKASGYTFTGYYMHWVRQAPGQGLEWMGWINPKNGDTEFPQKFQGRVTMTRDTSITTAYMDLSRLRSDDTAVYYCARESGDDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRG

SEQ ID NO: 96 - protein, 703 aa, Artificial Sequence
  synthetic sequence
EVQLVQSGAEVKKPGASVKVSCKASGYTFTGYYMHWVRQAPGQGLEWMGWINPKNGDTEFPQKFQGRVTMTRDTSITTAYMDLSRLRSDDTAVYYCARESGDDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSDCGLPPDVPNAQPALEGRTSFPEDTVITYKCEESFVKIPGEKDSVICLKGSQWSDIEEFCQRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRG

SEQ ID NO: 97 - protein, 453 aa, Artificial Sequence
  synthetic sequence
EVQLVESGGGWQPGGSLRLSCAASISIFDIYAMHWYRQAPGKQRELVATSFRDGSTYYADSVKGRFTISRDNSKNTLYLQMNSLRAEDTAVYLCHVSLYRDPLGVAGGMGVYWGKGALVTVSSGGGGSDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSHHHHHH

SEQ ID NO: 98 - protein, 123 aa, Artificial Sequence
  synthetic sequence
EVQLVESGGGWQPGGSLRLSCAASISIFDIYAMHWYRQAPGKQRELVATSFRDGSTYYADSVKGRFTISRDNSKNTLYLQMNSLRAEDTAVYLCHVSLYRDPLGVAGGMGVYWGKGALVTVSS

SEQ ID NO: 99 - protein, 705 aa, Artificial Sequence
  synthetic sequence
EVQLVQSGAEVKKPGASVKVSCKASGYTFTGYYMHWVRQAPGQGLEWMGWINPKNGDTEFPQKFQGRVTMTRDTSITTAYMDLSRLRSDDTAVYYCARESGDDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLIGSYSSFCKISGSSVQWSDKLPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKV

SEQ ID NO: 100 - protein, 782 aa, Artificial Sequence
  synthetic sequence
QVQLLESGPGLVKPSGTLSLTCTVSGGSISSTNWWTWVRQSPGTGLEWIGHIYHSGSTDYNPSLKSRVTISIDKSKNQFSLKMTSVTAADTAVYYCACAAQYHWKGLDPWGHGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSDCGPPPDIPNARPILGRHSKFAEQSKVAYSCNNGFKQVPDKSNIVVCLENGQWSSHETFCEKSCVAPERLSFASLKKEYLNMNFFPVGTIVEYECRPGFRKQPPLPGKATCLEDLVWSPVAQFCKKKSCPNPKDLDNGHINIPTGILFGSEINFSCNPGYRLVGVSSTFCSVTGNTVDWDDEFPVCTEIHCPEPPKINNGIMRGESDSYTYSQVVTYSCDKGFILVGNASIYCTVSKSDVGQWSSPPPRCIEKSKVPTKKPTINVPSTGTPSTPQKPTTESVPNPGDQPTPQKPSTVKVSATQHVPVTKTTVRHPIRTSTDKGEPNTG

SEQ ID NO: 101 - protein, 120 aa, Artificial Sequence
  synthetic sequence
QVQLLESGPGLVKPSGTLSLTCTVSGGSISSTNWWTWVRQSPGTGLEWIGHIYHSGSTDYNPSLKSRVTISIDKSKNQFSLKMTSVTAADTAVYYCACAAQYHWKGLDPWGHGTLVTVSS

SEQ ID NO: 102 - protein, 219 aa, Artificial Sequence
  synthetic sequence
DIVMTQTPLSLSVTPGQPASISCMSSQSLLHSDGKTYLYWYLQKPGQPPQLLIYEVSNRFSGVPDRFSGSGSGTDFTLKISRVEAEDVGIYYCMQSIQLPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 112 aa, Artificial Sequence
  synthetic sequence
DIVMTQTPLSLSVTPGQPASISCMSSQSLLHSDGKTYLYWYLQKPGQPPQLLIYEVSNRFSGVPDRFSGSGSGTDFTLKISRVEAEDVGIYYCMQSIQLPITFGQGTRLEIK

SEQ ID NO: 104 - protein, 602 aa, Artificial Sequence
  synthetic sequence
DCGPPPDIPNARPILGRHSKFAEQSKVAYSCNNGFKQVPDKSNIVVCLENGQWSSHETFCEKSCVAPERLSFASLKKEYLNMNFFPVGTIVEYECRPGFRKQPPLPGKATCLEDLVWSPVAQFCKKKSCPNPKDLDNGHINIPTGILFGSEINFSCNPGYRLVGVSSTFCSVTGNTVDWDDEFPVCTEIHCPEPPKINNGIMRGESDSYTYSQVVTYSCDKGFILVGNASIYCTVSKSDVGQWSSPPPRCIEKSKVPTKKPTINVPSTGTPSTPQKPTTESVPNPGDQPTPQKPSTVKVSATQHVPVTKTTVRHPIRTSTDKGEPNTGGGGGSGGGGSGGGGSGGGGSQVQLLESGPGLVKPSGTLSLTCTVSGGSISSTNWWTWVRQSPGTGLEWIGHIYHSGSTDYNPSLKSRVTISIDKSKNQFSLKMTSVTAADTAVYYCACAAQYHWKGLDPWGHGTLVTVSSGGGGGSGGGGSGGGGSDIVMTQTPLSLSVTPGQPASISCMSSQSLLHSDGKTYLYWYLQKPGQPPQLLIYEVSNRFSGVPDRFSGSGSGTDFTLKISRVEAEDVGIYYCMQSIQLPITFGQGTRLEIKHHHHHH

SEQ ID NO: 105 - protein, 347 aa, Artificial Sequence
  synthetic sequence
LTCYHCFQPVVSSCNMNSTCSPDQDSCLYAVAGMQVYQRCWKQSDCHGEIIMDQLEETKLKFRCCQFNLCNKSGGGGSGGGGSGGGGSGGGGSQVQLLESGPGLVKPSGTLSLTCTVSGGSISSTNWWTWVRQSPGTGLEWIGHIYHSGSTDYNPSLKSRVTISIDKSKNQFSLKMTSVTAADTAVYYCACAAQYHWKGLDPWGHGTLVTVSSGGGGGSGGGGSGGGGSDIVMTQTPLSLSVTPGQPASISCMSSQSLLHSDGKTYLYWYLQKPGQPPQLLIYEVSNRFSGVPDRFSGSGSGTDFTLKISRVEAEDVGIYYCMQSIQLPITFGQGTRLEIKHHHHHH

SEQ ID NO: 106 - protein, 841 aa, Artificial Sequence
  synthetic sequence
DCGPPPDIPNARPILGRHSKFAEQSKVAYSCNNGFKQVPDKSNIVVCLENGQWSSHETFCEKSCVAPERLSFASLKKEYLNMNFFPVGTIVEYECRPGFRKQPPLPGKATCLEDLVWSPVAQFCKKKSCPNPKDLDNGHINIPTGILFGSEINFSCNPGYRLVGVSSTFCSVTGNTVDWDDEFPVCTEIHCPEPPKINNGIMRGESDSYTYSQVVTYSCDKGFILVGNASIYCTVSKSDVGQWSSPPPRCIEKSKVPTKKPTINVPSTGTPSTPQKPTTESVPNPGDQPTPQKPSTVKVSATQHVPVTKTTVRHPIRTSTDKGEPNTGGGGGSGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSQVQLLESGPGLVKPSGTLSLTCTVSGGSISSTNWWTWVRQSPGTGLEWIGHIYHSGSTDYNPSLKSRVTISIDKSKNQFSLKMTSVTAADTAVYYCACAAQYHWKGLDPWGHGTLVTVSSGGGGGSGGGGSGGGGSDIVMTQTPLSLSVTPGQPASISCMSSQSLLHSDGKTYLYWYLQKPGQPPQLLIYEVSNRFSGVPDRFSGSGSGTDFTLKISRVEAEDVGIYYCMQSIQLPITFGQGTRLEIK

SEQ ID NO: 107 - protein, 586 aa, Artificial Sequence
  synthetic sequence
LTCYHCFQPVVSSCNMNSTCSPDQDSCLYAVAGMQVYQRCWKQSDCHGEIIMDQLEETKLKFRCCQFNLCNKSGGGGSGGGGSGGGGSGGGGSDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSQVQLLESGPGLVKPSGTLSLTCTVSGGSISSTNWWTWVRQSPGTGLEWIGHIYHSGSTDYNPSLKSRVTISIDKSKNQFSLKMTSVTAADTAVYYCACAAQYHWKGLDPWGHGTLVTVSSGGGGGSGGGGSGGGGSDIVMTQTPLSLSVTPGQPASISCMSSQSLLHSDGKTYLYWYLQKPGQPPQLLIYEVSNRFSGVPDRFSGSGSGTDFTLKISRVEAEDVGIYYCMQSIQLPITFGQGTRLEIK

SEQ ID NO: 108 - protein, 791 aa, Artificial Sequence
  synthetic sequence
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDWYYDFWSGYPGPDYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSDCGPPPDIPNARPILGRHSKFAEQSKVAYSCNNGFKQVPDKSNIVVCLENGQWSSHETFCEKSCVAPERLSFASLKKEYLNMNFFPVGTIVEYECRPGFRKQPPLPGKATCLEDLVWSPVAQFCKKKSCPNPKDLDNGHINIPTGILFGSEINFSCNPGYRLVGVSSTFCSVTGNTVDWDDEFPVCTEIHCPEPPKINNGIMRGESDSYTYSQVVTYSCDKGFILVGNASIYCTVSKSDVGQWSSPPPRCIEKSKVPTKKPTINVPSTGTPSTPQKPTTESVPNPGDQPTPQKPSTVKVSATQHVPVTKTTVRHPIRTSTDKGEPNTG

SEQ ID NO: 109 - protein, 129 aa, Artificial Sequence
  synthetic sequence
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDWYYDFWSGYPGPDYYGMDVWGQGTTVTVSS

SEQ ID NO: 110 - protein, 214 aa, Artificial Sequence
  synthetic sequence
AIRMTQSPSSLSASTGDRVTITCRASQGISSYLAWYQQKPGKAPKLLIYAASTLQSGVPSRFSGSGSGTDFTLTISCLQSEDFATYYCQQYYSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 111 - protein, 107 aa, Artificial Sequence
  synthetic sequence
AIRMTQSPSSLSASTGDRVTITCRASQGISSYLAWYQQKPGKAPKLLIYAASTLQSGVPSRFSGSGSGTDFTLTISCLQSEDFATYYCQQYYSYPWTFGQGTKVEIK

SEQ ID NO: 112 - protein, 24 aa, Artificial Sequence
  synthetic sequence
MKWVTFISLLFLFSSAYSRGVFRR

SEQ ID NO: 113 - protein, 585 aa, Artificial Sequence [sequence omitted]

SEQ ID NO: 114 - skipped

SEQ ID NO: 115 - protein, 329 aa, Artificial Sequence
  synthetic sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 116 - protein, 329 aa, Artificial Sequence
  synthetic sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 117 - protein, 329 aa, Artificial Sequence
  synthetic sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 118 - protein, 329 aa, Artificial Sequence
  synthetic sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 119 - protein, 21 aa, Artificial Sequence
  synthetic sequence
EPKSCCVECPPCPAPPAAAGA

SEQ ID NO: 120 - protein, 226 aa, Artificial Sequence
  synthetic sequence
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 121 - protein, 226 aa, Artificial Sequence
  synthetic sequence
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 122 - protein, 226 aa, Artificial Sequence
  synthetic sequence
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 123 - protein, 226 aa, Artificial Sequence
  synthetic sequence
DKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 124 - protein, 16 aa, Artificial Sequence
  synthetic sequence
GGGGGSGGGGSGGGGS

(19) United States
(12) Patent Application Publication
(10) Pub. No.: US 2025/0320292 A1
(43) Pub. Date: Oct. 16, 2025

(54) KIDNEY-GLOMERULAR TARGETED IMMUNOTHERAPY

(71) Applicant: Pandion Operations, Inc., Rahway, NJ (US)

(72) Inventors: Hussam Shaheen, Auburn, NH (US); Bridget Larkin, Somerville, MA (US); Jiwon Jung, San Mateo, CA (US); Ivan Mascanfroni, Watertown, MA (US); Joanne L. Viney, Belmont, MA (US); Nathan Higginson-Scott, Hingham, MA (US)

(73) Assignee: Pandion Operations, Inc., Rahway, NJ (US)

(21) Appl. No.: 18/556,216

(22) PCT Filed: Apr. 22, 2022
(86) PCT No.: PCT/US2022/026013

§ 371 Date: Oct. 19, 2023

Related U.S. Application Data
(60) Provisional application No. 63/178,866, filed on Apr. 23, 2021.

(51) Int. Cl. C07K 16/28; A61K 39/00; A61P 13/12; C07K 16/18

(52) U.S. Cl. CPC C07K 16/2803; A61P 13/12; C07K 16/18; A61K 2039/505; C07K 2317/31; C07K 2317/569; C07K 2317/622; C07K 2317/76; C07K 2317/92

(57) ABSTRACT

Methods and compounds for conferring kidney-glomerular-specific immunotherapy.

### CROSS-REFERENCE TO RELATED APPLICATIONS

This application claims priority to U.S. Provisional Application No. 63/178,866, filed Apr. 23, 2021, which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Mar. 21, 2022, is named 145256_002002_ST25.txt and is 364,774 bytes in size.

### FIELD

The embodiments provided herein relate to, for example, methods and compositions for kidney-glomerular-targeted immunotherapy.

### BACKGROUND

Glomerular diseases are chronic diseases that cause damage to the filters, glomeruli, in the kidneys. Damaged glomeruli allow red blood cells and protein to leak into the urine, cause waste products to build up in the blood, and can lead to kidney failure. Glomerular diseases affect individuals of all ages and tend to progress slowly in many patients. Despite years of studies and developments relating to kidney diseases, glomerular diseases remain a major health problem. There is, therefore, a need for new methods and compositions for preventing and treating glomerular diseases.

### SUMMARY

Disclosed herein are methods, compositions, proteins, and compounds that provide kidney-glomerular specific immunotherapy. Embodiments disclosed herein are incorporated by reference into this section.

In some embodiments, a protein comprising a glomerular targeting moiety and an effector moiety, is provided, wherein the glomerular targeting moiety is an antibody that binds to a Robo2 protein, an antibody that binds to a COL4A3 protein, an antibody that binds to a COL4A4 protein, or an antibody that binds to a COL4A5 protein; and the effector moiety is a complement modulator selected from the group consisting of a CD55 protein, a CD59 protein, a CR1 protein, and a DCP protein.

In some embodiments, an antibody or antigen binding fragment thereof is provided, wherein the antibody or antigen binding fragment thereof comprises: a light chain comprising an amino acid sequence as set forth in SEQ ID NO:83 or an amino acid sequence having at least 90% identity to SEQ ID NO: 83, and a heavy chain comprising an amino acid sequence as set forth in SEQ ID NO:81 or an amino acid sequence having at least 90% identity to SEQ ID NO: 81; a light chain comprising an amino acid sequence as set forth in SEQ ID NO:87 or an amino acid sequence having at least 90% identity to SEQ ID NO: 87, and a heavy chain comprising an amino acid sequence as set forth in SEQ ID NO:85 or an amino acid sequence having at least 90% identity to SEQ ID NO: 85; a light chain comprising an amino acid sequence as set forth in SEQ ID NO:102 or an amino acid sequence having at least 90% identity to SEQ ID NO: 102, and a heavy chain comprising an amino acid sequence as set forth in SEQ ID NO:100 or an amino acid sequence having at least 90% identity to SEQ ID NO: 100; or a light chain comprising an amino acid sequence as set forth in SEQ ID NO:108 or an amino acid sequence having at least 90% identity to SEQ ID NO:108, and a heavy chain comprising an amino acid sequence as set forth in SEQ ID NO:110 or an amino acid sequence having at least 90% identity SEQ ID NO: 110.

In some embodiments, an antibody or antigen binding fragment thereof is provided, wherein the antibody or antigen binding fragment thereof comprises: a light chain comprising an amino acid sequence as set forth in SEQ ID NO: 83, and a heavy chain comprising an amino acid sequence as set forth in SEQ ID NO: 81; a light chain comprising an amino acid sequence as set forth in SEQ ID NO: 87, and a heavy chain comprising an amino acid sequence as set forth in SEQ ID NO: 85; a light chain comprising an amino acid sequence as set forth in SEQ ID NO: 102, and a heavy chain comprising an amino acid sequence as set forth in SEQ ID NO: 100; or a light chain comprising an amino acid sequence as set forth in SEQ ID NO: 108, and a heavy chain comprising an amino acid sequence as set forth in SEQ ID NO: 110.

In some embodiments, a protein is provided, wherein the protein comprises: an amino acid sequence as set forth in SEQ ID NO: 81 or an amino acid sequence having at least 90% identity to SEQ ID NO: 81; an amino acid sequence as set forth in SEQ ID NO: 83 or an amino acid sequence having at least 90% identity to SEQ ID NO: 83; an amino acid sequence as set forth in SEQ ID NO: 85 or an amino acid sequence having at least 90% identity to SEQ ID NO: 85; an amino acid sequence as set forth in SEQ ID NO: 87 or an amino acid sequence having at least 90% identity to SEQ ID NO: 87; an amino acid sequence as set forth in SEQ ID NO: 89 or an amino acid sequence having at least 90% identity to SEQ ID NO: 89; an amino acid sequence as set forth in SEQ ID NO: 90 or an amino acid sequence having at least 90% identity to SEQ ID NO: 90; an amino acid sequence as set forth in SEQ ID NO:91 or an amino acid sequence having at least 90% identity to SEQ ID NO: 91; an amino acid sequence as set forth in SEQ ID NO: 92 or an amino acid sequence having at least 90% identity to SEQ ID NO: 92; an amino acid sequence as set forth in SEQ ID NO: 93 or an amino acid sequence having at least 90% identity to SEQ ID NO: 93; an amino acid sequence as set forth in SEQ ID NO: 94 or an amino acid sequence having at least 90% identity to SEQ ID NO: 94; an amino acid sequence as set forth in SEQ ID NO: 95 or an amino acid sequence having at least 90% identity to SEQ ID NO: 95; an amino acid sequence as set forth in SEQ ID NO: 96 or an amino acid sequence having at least 90% identity to SEQ ID NO: 96; an amino acid sequence as set forth in SEQ ID NO: 97 or an amino acid sequence having at least 90% identity to SEQ ID NO: 97; an amino acid sequence as set forth in SEQ ID NO: 99 or an amino acid sequence having at least 90% identity to SEQ ID NO: 99; an amino acid sequence as set forth in SEQ ID NO: 100 or an amino acid sequence having at least 90% identity to SEQ ID NO: 100; an amino acid sequence as set forth in SEQ ID NO: 102 or an amino acid sequence having at least 90% identity to SEQ ID NO: 102; an amino acid sequence as set forth in SEQ ID NO: 104 or an amino acid sequence having at least 90% identity to SEQ ID NO: 104; an amino acid sequence as set forth in SEQ ID NO: 105 or an amino acid sequence having at least 90% identity to SEQ ID NO: 105; an amino acid sequence as set forth in SEQ ID NO: 106 or an amino acid sequence having at least 90% identity to SEQ ID NO: 106; an amino acid sequence as set forth in SEQ ID NO: 107 or an amino acid sequence having at least 90% identity to SEQ ID NO: 107; an amino acid sequence as set forth in SEQ ID NO: 108 or an amino acid sequence having at least 90% identity to SEQ ID NO: 108; or an amino acid sequence as set forth in SEQ ID NO: 110 or an amino acid sequence having at least 90% identity to SEQ ID NO: 110.

In some embodiments, a protein is provided, wherein the protein comprises: an amino acid sequence as set forth in SEQ ID NO: 81; an amino acid sequence as set forth in SEQ ID NO: 83; an amino acid sequence as set forth in SEQ ID NO: 85; an amino acid sequence as set forth in SEQ ID NO: 87; an amino acid sequence as set forth in SEQ ID NO: 89; an amino acid sequence as set forth in SEQ ID NO: 90; an amino acid sequence as set forth in SEQ ID NO: 91; an amino acid sequence as set forth in SEQ ID NO: 92; an amino acid sequence as set forth in SEQ ID NO: 93; an amino acid sequence as set forth in SEQ ID NO: 94; an amino acid sequence as set forth in SEQ ID NO: 95; an amino acid sequence as set forth in SEQ ID NO: 96; an amino acid sequence as set forth in SEQ ID NO: 97; an amino acid sequence as set forth in SEQ ID NO: 99; an amino acid sequence as set forth in SEQ ID NO: 100; an amino acid sequence as set forth in SEQ ID NO: 102; an amino acid sequence as set forth in SEQ ID NO: 104; an amino acid sequence as set forth in SEQ ID NO: 105; an amino acid sequence as set forth in SEQ ID NO: 106; an amino acid sequence as set forth in SEQ ID NO: 107; an amino acid sequence as set forth in SEQ ID NO: 108; or an amino acid sequence as set forth in SEQ ID NO: 110.

### BRIEF DESCRIPTION OF THE DRAWINGS

FIG. 1 depicts ex vivo staining of bi-functional molecules comprising an anti-COL4A3 antibody and CD55, wherein the staining was in the kidney.

FIG. 2A depicts in vivo staining of bi-functional molecules comprising an anti-Robo2 antibody and CD55, wherein the staining was in the kidney and was positive.

FIG. 2B depicts in vivo staining of isotype control, wherein the staining was in the kidney and was negative.

FIG. 3 depicts a non-limiting illustration of the therapeutic compounds provided herein.

FIG. 3A depicts a non-limiting illustration of the therapeutic compounds provided herein, wherein the therapeutic molecule comprises an anti-PD1 moiety at the N-terminus or the C-terminus, and an anti-COL4A3 moiety at the C-terminus or the N-terminus, respectively.

FIG. 3B depicts a non-limiting illustration of the therapeutic compounds provided herein, wherein the therapeutic molecule comprises a CD55 moiety at the C-terminus or the N-terminus, and an anti-COL4A3 moiety at the N-terminus or the C-terminus, respectively.

FIG. 3C depicts a non-limiting illustration of the therapeutic compounds provided herein, wherein the therapeutic molecule comprises a CD55 moiety directly linked to an anti-COL4A3 scFv moiety, or an anti-COL3A3 VHH moiety directly linked to a CD55 moiety.

FIG. 4 depicts a non-limiting illustration of the therapeutic compounds provided herein, wherein the therapeutic compound comprises two identical Fab domains.

FIG. 5 depicts a non-limiting illustration of the therapeutic compounds provided herein, wherein the therapeutic compound comprises two identical Fab domains and two identical scFv domains.

FIG. 6 depicts a non-limiting illustration of the therapeutic compounds provided herein, wherein the therapeutic compound is asymmetric and bispecific.

FIG. 7 depicts a non-limiting illustration of the therapeutic compounds provided herein, wherein the therapeutic compound is asymmetric and bispecific.

FIG. 8 depicts a non-limiting illustration of the therapeutic compounds provided herein, wherein the therapeutic compound is asymmetric and bispecific.

FIG. 9 depicts a non-limiting illustration of the therapeutic compounds provided herein.

FIG. 10 depicts a non-limiting illustration of the therapeutic compounds provided herein, wherein the therapeutic compound has a mixed format.

FIG. 11 depicts a non-limiting illustration of the therapeutic compounds provided herein.

FIG. 12 depicts a non-limiting illustration of the therapeutic compounds provided herein, wherein the therapeutic compound is constructed to have shorter systemic PK and increased tissue penetration.

FIG. 13 depicts a non-limiting illustration of the therapeutic compounds provided herein, wherein the therapeutic compound is constructed to have shorter systemic PK and increased tissue penetration.

FIG. 14 depicts a non-limiting illustration of the therapeutic compounds provided herein, wherein the therapeutic compound is constructed to have shorter systemic PK and increased tissue penetration.

FIG. 15 depicts a non-limiting illustration of the therapeutic compounds provided herein.

FIG. 16 depicts a non-limiting illustration of the therapeutic compounds provided herein.

FIG. 17 depicts a non-limiting illustration of the therapeutic compounds provided herein, wherein the therapeutic compounds is a tandem scFv.

FIG. 18 depicts a non-limiting illustration of the therapeutic compounds provided herein, wherein the therapeutic compounds is a F(ab′)2 scFv fusion.

FIG. 19 depicts a non-limiting illustration of the therapeutic compounds provided herein.

### DETAILED DESCRIPTION

As used herein and in the appended claims, the singular forms “a”, “an” and “the” include plural reference unless the context clearly dictates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by +5% and remain within the scope of the disclosed embodiments. Thus, about 100 means 95 to 105.

As used herein, the term “animal” includes, but is not limited to, humans and non-human vertebrates such as wild, domestic, and farm animals. As used herein, the term “mammal” includes a rodent (i.e., a mouse, a rat, or a guinea pig), a monkey, a cat, a dog, a cow, a horse, a pig, or a human. In some embodiments, the mammal is a human.

As used herein, the term “contacting” means bringing together of two elements in an in vitro system or an in vivo system. For example, “contacting” a therapeutic compound with an individual or patient or cell includes the administration of the compound to an individual or patient, such as a human, as well as, for example, introducing a compound into a sample containing a cellular or purified preparation containing target.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps. Any composition or method that recites the term “comprising” should also be understood to also describe such compositions as consisting, consisting of, or consisting essentially of the recited components or elements.

As used herein, the term “fused” or “linked” when used in reference to a protein having different domains or heterologous sequences means that the protein domains are part of the same peptide chain that are connected to one another with either peptide bonds or other covalent bonding. The domains or section can be linked or fused directly to one another or another domain or peptide sequence can be between the two domains or sequences and such sequences would still be considered to be fused or linked to one another. In some embodiments, the various domains or proteins provided for herein are linked or fused directly to one another or via a linker sequence, such as the glycine/serine sequences described herein to link the two domains together.

As used herein, the term “individual,” “subject,” or “patient,” used interchangeably, means any animal, including mammals, such as mice, rats, other rodents, rabbits, dogs, cats, swine, cattle, sheep, horses, or primates, such as humans.

As used herein, the term “inhibit” refers to a result, symptom, or activity being reduced as compared to the activity or result in the absence of the compound that is inhibiting the result, symptom, or activity. In some embodiments, the result, symptom, or activity, is inhibited by about, or, at least, 10%, 20%, 30%, 40%, 50%, 60%, 70%, 80%, 90%, 95%, or 99%. A result, symptom, or activity can also be inhibited if it is completely eliminated or extinguished.

As used herein, the phrase “in need thereof” means that the subject has been identified as having a need for the particular method or treatment. In some embodiments, the identification can be by any means of diagnosis. In any of the methods and treatments described herein, the subject can be in need thereof. In some embodiments, the subject is in an environment or will be traveling to an environment in which a particular disease, disorder, or condition is prevalent. In some embodiments, the subject is at risk of developing a particular disease or disorder that a treatment is intended to treat and/or prevent. Those “in need of treatment” include those patients that may benefit form treatment with the methods of the inventions, e.g. a patient suffering from or at risk of developing an autoimmune disorder or diabetes.

As used herein, the phrase “integer from X to Y” means any integer that includes the endpoints. For example, the phrase “integer from 1 to 5” means 1, 2, 3, 4, or 5.

In some embodiments, therapeutic compounds are provided herein. In some embodiments, the therapeutic compound is a protein or a polypeptide, that has multiple peptide chains that interact with one another. The polypeptides can interact with one another through non-covalent interactions or covalent interactions, such as through disulfide bonds or other covalent bonds. Therefore, if an embodiment refers to a therapeutic compound it can also be said to refer to a protein or polypeptide as provided for herein and vice versa as the context dictates.

In some embodiments, the term “therapeutic molecule” can be used interchangeably with “therapeutic compound,” “molecule,” or “therapeutic,” and refers to any polypeptide, or protein described herein.

“Specific binding” or “specifically binds to” or is “specific for” a particular antigen, target, or an epitope means binding that is measurably different from a non-specific interaction.

Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen, target, or an epitope can be exhibited, for example, by an antibody having a K_{D} for an antigen or epitope of at least about 10^{-4M}, at least about 10^{-5M}, at least about 10^{-6M}, at least about 10^{-7M}, at least about 10^{-8M}, at least about 10^{-9M} alternatively at least about 10^{-10M}, at least about 10^{-11M} at least about 10−^{12M}, or greater, where K_{D} refers to a dissociation rate of a particular antibody-target interaction. Typically, an antibody that specifically binds an antigen or target will have a K_{D} that is, or at least, 2-, 4-, 5-, 10-, 20-, 50-, 100-, 500-, 1000-, 5,000-, 10,000-, or more times greater for a control molecule relative to the antigen or epitope.

In some embodiments, specific binding for a particular antigen, target, or an epitope can be exhibited, for example, by an antibody having a K_{A} or Kₐ for a target, antigen, or epitope of at least 2-, 4-, 5-, 20-, 50-, 100-, 500-, 1000-, 5,000-, 10,000- or more times greater for the target, antigen, or epitope relative to a control, where K_{A} or Kₐ refers to an association rate of a particular antibody-antigen interaction.

Provided herein are therapeutic compounds, e.g., therapeutic protein molecules, e.g., fusion proteins, including a targeting moiety and an effector binding/modulating moiety, typically as separate domains. Also provided are methods of using and making the therapeutic compounds. The targeting moiety serves to localize the therapeutic compound, and thus the effector binding/modulating moiety, to a site at which immune-privilege is desired. As used herein, “immune privilege” means lack of, or suppression of an inflammatory response. As a non-limiting example, immune privilege includes situations where a tissue or site in the body is able to tolerate the introduction of antigens without eliciting an inflammatory immune response (Forester J. V., Lambe H. Xu, Cornall R. Immune Privilege or privileged immunity? Mucosal Immunology, 1, 372-381 (2008)).

The present disclosure provides, for example, molecules that can act as complement inhibitors. An inhibitor of the complement system for use in the methods and/or medicaments of the present invention may be an antagonist, polypeptide, peptide, antibody, anti-sense oligonucleotide, aptamer, miRNA, ribozyme, siRNA, or small molecule. Complement inhibitors, as provided herein, prevent the activation of a complement system, and include: (i) DAF (decay accelerating factor or CD55), which accelerates decay of the C3 convertases C4b2a (classical pathway) and C3bBb (alternative pathway) by interacting with C3b or C4b and Bb or C2a; (ii), complement receptor 1 (CR1 or CD35) which similarly accelerates convertase decay but additionally has cofactor activity for factor I cleavage; (iii) factor I, a plasma protease that cleaves C3b and C4b into their inactive forms to block formation of the convertases; and (ivii) factor H, a soluble protein which can compete with factor B, displace Bb from the convertase, act as a cofactor for factor I, and bind C3b that is already bound to cells. CD59 is a complement regulatory protein that inhibits MAC (C5b-9). The complement inhibitors, as provided herein, can be linked to a targeting, moiety, such as one that binds to COL4A3 or Robo2.

In some embodiments, the tethers provided for herein are linked to a PD-1 agonist. Examples of PD-1 agonists, such as antibodies, are provided for herein. In some embodiments, the PD-1 agonist can be linked to a targeting moiety, such as one that binds to COL4A3 or Robo2.

As used herein, the term “COL4A3” refers to the protein collagen type IV alpha 3, which can also be referred to as collagen type IV alpha 3 chain, collagen alpha-3(IV) chain, tumstatin, collagen IV alpha-3 polypeptide, goodpasture antigen, COL4A3, ATS2, or ATS3.

As used herein, the term “COL4A4” refers to the protein collagen type IV alpha 4, which can also be referred to as collagen type IV alpha 4 chain, collagen alpha-4(IV) chain, CA44, collagen IV alpha-4 polypeptide, C04A4, collagen of basement membrane alpha-4 chain, or COL4A4.

As used herein, the term “COL4A5” refers to the protein collagen type IV alpha 5, which can also be referred to as collagen type IV alpha 5 chain, collagen alpha-5(IV) chain, collagen IV alpha-5 polypeptide, C04A4, collagen of basement membrane alpha-5 chain, ASLN, ATS, CA54, C04A5, or Alport syndrome.

As used herein, the term “Robo2” refers to the protein roundabout guidance receptor 2, which can also be referred to as roundabout homolog 2, KIAA1568, roundabout axon guidance receptor homolog 2, ROBO2, or SAX3.

In some embodiments, the targeting moiety (i.e. that binds to COL4A3, COL4A4, COL4A5, or Robo2) and effector binding/modulating moiety (e.g. complement modulator, PD-1 agonist, and/or IL-2 mutein) are physically tethered, covalently or non-covalently, directly or through a linker entity, to one another, e.g., as a member of the same protein molecule in a therapeutic protein molecule. In some embodiments, the targeting and effector moieties are provided in a therapeutic protein molecule, e.g., a fusion protein, typically as separate domains. In some embodiments, the targeting moiety, the effector binding/modulating moiety, or both each comprises a single domain antibody molecule, e.g., a camelid antibody VHH molecule or human soluble VH domain. It may also contain a single-chain fragment variable (scFv) or a Fab domain. In some embodiments, the therapeutic protein molecule, or a nucleic acid, e.g., an mRNA or DNA, encoding the therapeutic protein molecule, can be administered to a subject. In some embodiments, the targeting and effector molecule binding/modulating moieties are linked to a third entity, e.g., a carrier, e.g., a polymeric carrier, a dendrimer, or a particle, e.g., a nanoparticle. The therapeutic compounds can be used to down regulate an immune response at or in a tissue at a selected target or site while having no or substantially less immunosuppressive function systemically. The target or site can comprise donor tissue or autologous tissue.

In some embodiments, the subject that is treated with the proteins provided for herein are characterized as having end renal stage disease.

Provided herein are methods of treating, e.g., therapeutically treating or prophylactically treating (or preventing), or ameliorating symptoms of glomerulonephropathies and glomerulonephritides. Examples of glomerulonephropathies that can be treated include, not are not limited to, immune-complex glomerulonephritis (GN), pauci-immune GN, anti-glomerular basement membrane GN, monoclonal immunoglobulin GN, C3 glomerulopathy, nephrotic syndrome (NS), primary congenital NS (CNS), renal tubular acidosis (RTA), inherited renal tubulopathies, Faconi syndrome, primary nephrogenic diabetes insipidus. In some embodiments, the glomerulonephritis is a primary glomerulonephritis. In some embodiments, the primary glomerulonephritis can be, but is not limited to, minimal change disease, focal segmental glomerular sclerosis, membranous nephropathy, immunoglobulin A nephropathy, C3 glomerulopathy (DDD, C3 GN) and idiopathic immune complex membranoproliferative GN, C4 glomerulopathy, infection-related and renal-limited GN, renal limited vasculitis, collagenofibrotic glomerulopathy, thin basement membranes nephropathy, lipoprotein glomerulopathy, ‘Pure’ mesangial proliferative GN, IgM nephropathy, C1q nephropathy, and idiopathic nodular glomerulosclerosis (diabetic nephropathy without diabetes).

Also provided herein are methods of treating, e.g., therapeutically treating or prophylactically treating (or preventing), an autoimmune disorder or autoimmune response in a subject by administration of a therapeutic compound disclosed herein, e.g., to provide site or tissue specific modulation of the immune system. Examples of autoimmune diseases or inflammation that impact the kidney that can be treated include, but are not limited to Goodpasture's Syndrome (anti-GBM disease), inflammatory renal disease, Alport syndrome, glomerulonephritis, nephritis, lupus, lupus nephritis, IgA nephritis, membranous nephropathy, membranoproliferative glomerulonephritis, acute kidney injury, and chronic kidney disease as well as any other autoimmune or inflammation disorders that can affect the kidneys.

The therapeutic compounds and compositions of the invention can be used in methods of treatment as provided herein. As used herein, the terms “treat,” “treated,” or “treating” in regards to therapeutic treatment refer to methods of treatment wherein the object is to slow down (lessen) an undesired physiological condition, disorder or disease, or obtain beneficial or desired clinical results. For example, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response without excessive levels of side effects. Treatment also includes prolonging survival as compared to expected survival if not receiving treatment. Thus, “treatment of an autoimmune disease/disorder” means an activity that alleviates or ameliorates any of the primary phenomena or secondary symptoms associated with the autoimmune disease/disorder or other condition described herein. Methods for the treatment of various diseases or conditions are provided herein. The therapeutic treatment can also be administered prophylactically to prevent or reduce the disease or condition before the onset.

In some embodiments, administration of the therapeutic compound, which can also be referred to as a protein throughout the present specification, begins after the disorder is apparent. In some embodiments, administration of the therapeutic compound, begins prior to onset, or full onset, of the disorder. In some embodiments, administration of the therapeutic compound, begins prior to onset, or full onset, of the disorder, e.g., in a subject having the disorder, a high-risk subject, a subject having a biomarker for risk or presence of the disorder, a subject having a family history of the disorder, or other indicator of risk of, or asymptomatic presence of the disorder.

While not wishing to be bound by theory, it is believed that the targeting moiety functions to bind and accumulate the therapeutic compound to a target selectively or preferentially expressed at the anatomical site where immune privilege is desired. For treatment of autoimmune disorders, the targeting moiety binds a target preferentially expressed at the anatomical site where immune privilege is desired, e.g., in the kidney.

Again, while not wishing to be bound by theory, it is believed that the effector binding/modulating moiety serves to deliver an immunosuppressive signal or otherwise create an immune privileged environment.

Unless otherwise defined, all technical and scientific terms used herein have the same meaning as commonly understood by one of ordinary skill in the art to which these embodiments belong. Although methods and materials similar or equivalent to those described herein can be used in the practice or testing of the present embodiments, suitable methods and materials are described below. All publications, patent applications, patents, and other references mentioned herein are incorporated by reference in their entirety. In addition, the materials, methods, and examples are illustrative only and not intended to be limiting. Headings, sub-headings or numbered or lettered elements, e.g., (a), (b), (i) etc., are presented merely for ease of reading. The use of headings or numbered or lettered elements in this document does not require the steps or elements be performed in alphabetical order or that the steps or elements are necessarily discrete from one another. Other features, objects, and advantages of the embodiments will be apparent from the description and drawings, and from the claims.

### Additional Definitions

Unless defined otherwise, all technical and scientific terms used herein have the same meaning as commonly understood by one of ordinary skill in the art to which the embodiments pertains. In describing and claiming the present embodiments, the following terminology and terminology otherwise referenced throughout the present application will be used according to how it is defined, where a definition is provided.

It is also to be understood that the terminology used herein is for the purpose of describing particular embodiments only, and is not intended to be limiting.

Antibody molecule, as that term is used herein, refers to a protein, e.g., an immunoglobulin chain or fragment thereof, comprising at least one functional immunoglobulin variable domain sequence. An antibody molecule encompasses antibodies (e.g., full-length antibodies) and antibody fragments. In some embodiments, an antibody molecule comprises an antigen binding or functional fragment of a full-length antibody, or a full-length immunoglobulin chain. For example, a full-length antibody is an immunoglobulin (Ig) molecule (e.g., an IgG antibody) that is naturally occurring or formed by normal immunoglobulin gene fragment recombinatorial processes. In embodiments, an antibody molecule refers to an immunologically active, antigen binding portion of an immunoglobulin molecule, such as an antibody fragment. An antibody fragment, e.g., functional fragment, comprises a portion of an antibody, e.g., Fab, Fab′, F(ab′)2, F(ab)2, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules include full-length antibodies and antibody fragments, e.g., dAb (domain antibody), single chain, Fab, Fab′, and F(ab′)2 fragments, and single chain variable fragments (scFvs).

The term “antibody molecule” also encompasses whole or antigen binding fragments of domain, or single domain, antibodies, which can also be referred to as “sdAb” or “VHH.” Domain antibodies comprise either V_{H} or V_{L} that can act as stand-alone, antibody fragments. Additionally, domain antibodies include heavy-chain-only antibodies (HCAbs). Domain antibodies also include a CH2 domain of an IgG as the base scaffold into which CDR loops are grafted. It can also be generally defined as a polypeptide or protein comprising an amino acid sequence that is comprised of four framework regions interrupted by three complementarity determining regions. This is represented as FR1-CDR1-FR2-CDR2-FR3-CDR3-FR4. sdAbs can be produced in camelids such as llamas, but can also be synthetically generated using techniques that are well known in the art. The numbering of the amino acid residues of a sdAb or polypeptide is according to the general numbering for VH domains given by Kabat et al. (“Sequence of proteins of immunological interest,” US Public Health Services, NIH Bethesda, MD, Publication No. 91, which is hereby incorporated by reference). According to this numbering, FR1 of a sdAb comprises the amino acid residues at positions 1-30, CDR1 of a sdAb comprises the amino acid residues at positions 31-36, FR2 of a sdAb comprises the amino acids at positions 36-49, CDR2 of a sdAb comprises the amino acid residues at positions 50-65, FR3 of a sdAb comprises the amino acid residues at positions 66-94, CDR3 of a sdAb comprises the amino acid residues at positions 95-102, and FR4 of a sdAb comprises the amino acid residues at positions 103-113. Domain antibodies are also described in WO2004041862 and WO2016065323, each of which is hereby incorporated by reference. The domain antibodies can be a targeting moiety as described herein.

Antibody molecules can be monospecific (e.g., monovalent or bivalent), bispecific (e.g., bivalent, trivalent, tetravalent, pentavalent, or hexavalent), trispecific (e.g., trivalent, tetravalent, pentavalent, or hexavalent), or with higher orders of specificity (e.g., tetraspecific) and/or higher orders of valency beyond hexavalency. An antibody molecule can comprise a functional fragment of a light chain variable region and a functional fragment of a heavy chain variable region, or heavy and light chains may be fused together into a single protein.

Examples of formats for multispecific therapeutic compounds, e.g., bispecific antibody molecules are shown in the following non-limiting examples. Although illustrated with antibody molecules, they can be used as platforms for therapeutic molecules that include other non-antibody moieties as specific binding or effector moieties. In some embodiments, these non-limiting examples are based upon either a symmetrical or asymmetrical Fc formats.

For example, the figures illustrate non-limiting and varied symmetric homodimer approaches. In some embodiments, the dimerization interface centers around human IgG1 CH2-CH3 domains, which dimerize via a contact interface spanning both CH2/CH2 and CH3/CH3. The resulting bispecific antibodies shown have a total valence comprised of four binding units with two identical binding units at the N-terminus on each side of the dimer and two identical units at the C-terminus on each side of the dimer. In each case the binding units at the N-terminus of the homodimer are different from those at the C-terminus of the homodimer. Using this type of bivalency for both an inhibitory T cell receptor at either terminus of the molecule and bivalency for a tissue tethering antigen can be achieved at either end of the molecule.

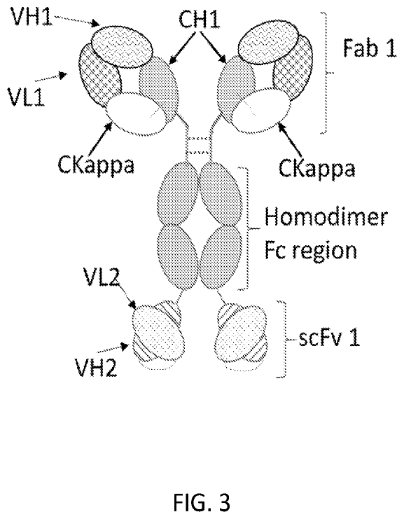

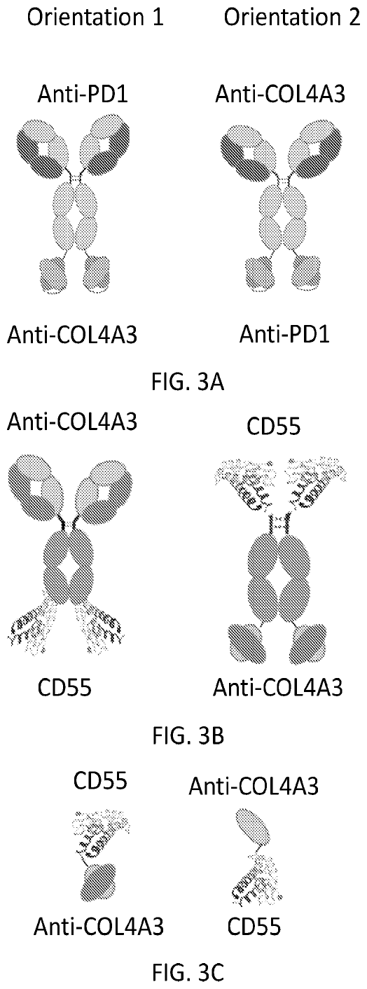

For example, in FIG. 3, a non-limiting embodiment is illustrated. The N-terminus of the homodimer contains two identical Fab domains comprised of two identical light chains, which are separate polypeptides, interfaced with the N-terminal VH-CH1 domains of each heavy chain via the VH/VL interaction and Ckappa or Clambda interaction with CH1. The native disulfide bond between the Ckappa or Clambda with CH1 is present providing a covalent anchor between the light and heavy chains. At the C-terminus of this design are two identical scFv units where by (in this example) the C-terminus of the CH3 domain of the Fc, is followed by a flexible, hydrophilic linker typically comprised of (but not limited to) serine, glycine, alanine, and/or threonine residues, which is followed by the VH domain of each scFv unit, which is followed by a glycine/serine rich linker, followed by a VL domain. These tandem VH and VL domains associate to form a single chain fragment variable (scFv) appended at the C-terminus of the Fc. Two such units exist at the C-terminus of this molecule owing to the homodimeric nature centered at the Fc. The domain order of scFvs may be configured to be from N- to C-terminus either VH-Linker-VL or VL-Linker-VH.

A non-limiting example of a molecule that has different binding regions on the different ends is a protein comprising a complement inhibitor on one end and an antibody that provides target specificity such as an anti-COL4A3 antibody, anti-COL4A4 antibody, anti-COL4A5 antibody, or anti-Robo2 antibody antibody on the other end. This can be illustrated as shown, for example, in FIG. 3A, which illustrates the molecules in different orientations.

In some embodiments, the complement inhibitor is replaced with a PD-1 agonist, or an IL-2 mutein, such as, but not limited to, the ones described herein.

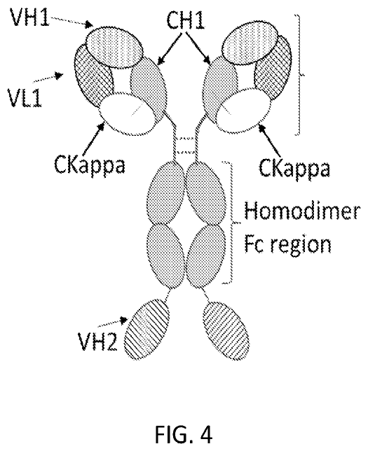

In another example, and as depicted in FIG. 4, the N-terminus of the homodimer contains two identical Fab domains comprised of two identical light chains, which are separate polypeptides, interfaced with the N-terminal VH-CH1 domains of each heavy chain via the VH/VL interaction and Ckappa or Clambda interaction with CH1. The native disulfide bond between the Ckappa or Clambda with CH1 is present providing a covalent anchor between the light and heavy chains. At the C-terminus of this design are two identical VH units (though non-antibody moieties could also be substituted here or at any of the four terminal attachment/fusion points) where by (in this example) the C-terminus of the CH3 domain of the Fc, is followed by a flexible, hydrophilic linker typically comprised of (but not limited to) serine, glycine, alanine, and/or threonine residues, which is followed by a soluble independent VH3 germline family based VH domain. Two such units exist at the C-terminus of this molecule owing to the homodimeric nature centered at the Fc.

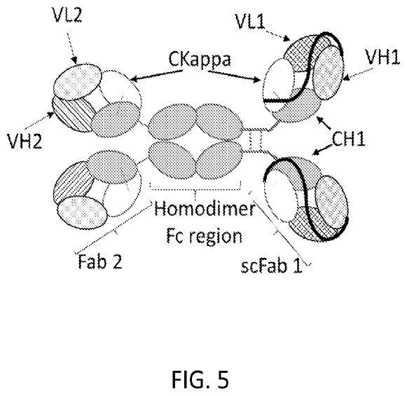

In another non-limiting example, as depicted in FIG. 5, the N-terminus of the homodimer contains two identical Fab domains comprised of two identical light chains, which, unlike FIG. 3 and FIG. 4, are physically conjoined with the heavy chain at the N-terminus via a linker between the C-terminus of Ckappa or Clambda and the N-terminus of the VH. The linker may be 36-80 amino acids in length and comprised of serine, glycine, alanine and threonine residues. The physically conjoined N-terminal light chains interface with the N-terminal VH-CH1 domains of each heavy chain via the VH/VL interaction and Ckappa or Clambda interaction with CH1. The native disulfide bond between the Ckappa or Clambda with CH1 is present providing additional stability between the light and heavy chains. At the C-terminus of this design are two identical Fab units where by (in this example) the C-terminus of the CH3 domain of the Fc, is followed by a flexible, hydrophilic linker typically comprised of (but not limited to) serine, glycine, alanine, and/or threonine residues, which is followed by a CH1 domain, followed by a VH domain at the C-terminus. The light chain that is designed to pair with the C-terminal CH1/VH domains is expressed as a separate polypeptide, unlike the N-terminal light chain which is conjoined to the N-terminal VH/CH1 domains as described. The C-terminal light chains form an interface at between VH/VL and Ckappa or Clambda with CH1. The native disulfide anchors this light chain to the heavy chain. Again, any of the antibody moieties at any of the four attachment/fusion points can be substituted with a non-antibody moiety, e.g., an effector binding/modulating moiety that does not comprise an antibody molecule.

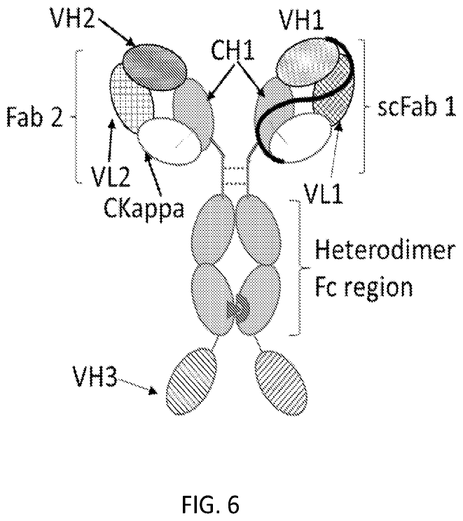

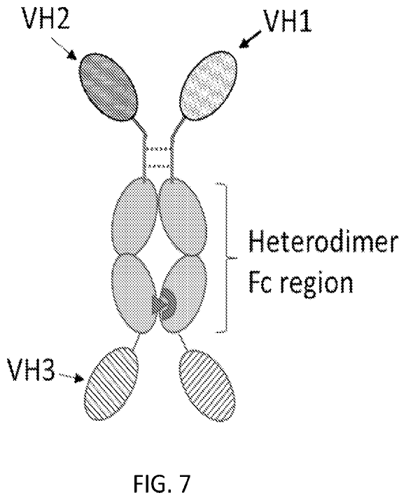

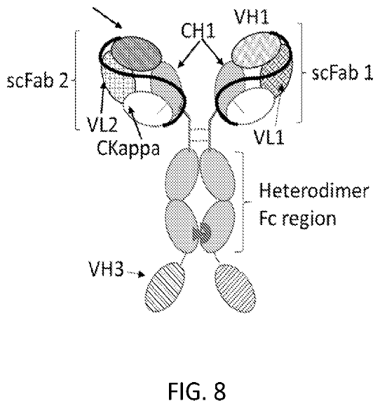

The bispecific antibodies can also be asymmetric as shown in the non-limiting examples depicted in FIG. 6, FIG. 7, and FIG. 8, which illustrate an asymmetric/heterodimer approach. Again, in any of these formats, any of the antibody moieties at any of the four attachment/fusion points can be substituted with a non-antibody moiety, e.g., an effector binding/modulating moiety that does not comprise an antibody molecule. In some embodiments, the dimerization interface centers around the human IgG1 CH2-CH3 domains, which dimerize via a contact interface spanning both CH2/CH2 and CH3/CH3. However, in order to achieve heterodimerization instead of homodimerization of each heavy chain, mutations are introduced in each CH3 domain. The heterodimerizing mutations include T366W mutation (Kabat) in one CH3 domain and T366S, L368A, and Y407V (Kabat) mutations in the other CH3 domain. The heterodimerizing interface may be further stabilized with de novo disulfide bonds via mutation of native residues to cysteine residues such as S354 and Y349 on opposite sides of the CH3/CH3 interface. The resulting bispecific antibodies shown have a total valence comprised of four binding units. With this approach, the overall molecule can be designed to have bispecificity at just one terminus and monospecificity at the other terminus (trispecificity overall) or bispecificity at either terminus with an overall molecular specificity of 2 or 4. In the illustrative examples below, the C-terminus comprises two identical binding domains which could, for example, provide bivalent monospecificity for a tissue tethering target. At the N-terminus of all three of the illustrative examples, both binding domains comprise different recognition elements/paratopes and which could achieve recognition of two different epitopes on the same effector moiety target, or could recognize for example a T cell inhibitory receptor and CD3. In some embodiments, the N-terminal binding moieties may be interchanged with other single polypeptide formats such as scFv, single chain Fab, tandem scFv, VH or VHH domain antibody configurations for example. Other types of recognition element may be used also, such as linear or cyclic peptides.

An example of an asymmetric molecule is depicted in FIG. 6. Referring to FIG. 6, the N-terminus of the molecule is comprised of a first light chain paired with a first heavy chain via VH/VL and Ckappa or Clambda/CH1 interactions and a covalent tether comprised of the native heavy/light chain disulfide bond. On the opposite side of this heterodimeric molecule at the N-terminus is a second light chain and a second heavy chain which are physically conjoined via a linker between the C-terminus of Ckappa or Clambda and the N-terminus of the VH. The linker may be 36-80 amino acids in length and comprised of serine, glycine, alanine and threonine residues. The physically conjoined N-terminal light chains interface with the N-terminal VH-CH1 domains of each heavy chain via the VH/VL interaction and Ckappa or Clambda interaction with CH1. The native disulfide bond between the Ckappa or Clambda with CH1 is present providing additional stability between the light and heavy chains. At the C-terminus of the molecule are two identical soluble VH3 germline family VH domains joined via an N-terminal glycine/serine/alanine/threonine based linker to the C-terminus of the CH3 domain of both heavy chain 1 and heavy chain 2.

In some embodiments, an asymmetric molecule can be as depicted in FIG. 7. For example, the N-terminus of the molecule is comprised of two different VH3 germlined based soluble VH domains linked to the human IgG1 hinge region via a glycine/serine/alanine/threonine based linker. The VH domain connected to the first heavy chain is different to the VH domain connected to the second heavy chain. At the C-terminus of each heavy chain is an additional soluble VH3 germline based VH domain, which is identical on each of the two heavy chains. The heavy chain heterodimerizes via the previously described knobs into holes mutations present at the CH3 interface of the Fc module.

In some embodiments, an asymmetric molecule can be as illustrated in FIG. 8. This example is similar to the molecule shown in FIG. 7, except both N-terminal Fab units are configured in a way that light chain 1 and light chain 2 are physically conjoined with heavy chain 1 and heavy chain 2 via a linker between the C-terminus of Ckappa or Clambda and the N-terminus of each respective VH. The linker in each case may be 36-80 amino acids in length and comprised of serine, glycine, alanine and threonine residues. The physically conjoined N-terminal light chains interface with the N-terminal VH-CH1 domains of each heavy chain via the VH/VL interaction and Ckappa or Clambda interaction with CH1. The native disulfide bond between the Ckappa or Clambda with CH1 is present providing additional stability between the light and heavy chains.

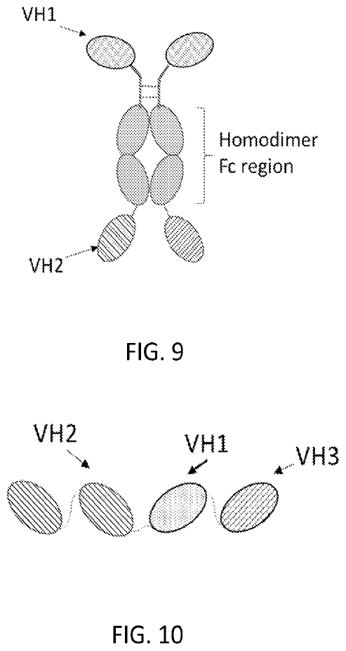

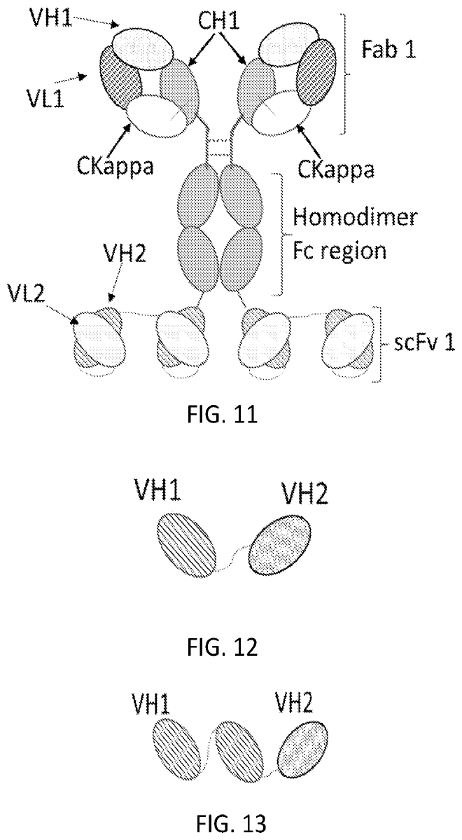

Bispecific molecules can also have a mixed format. This is illustrated, for example, in FIG. 9, FIG. 10, and FIG. 11.

For example, FIG. 9 illustrates a homodimer Fc based approach (see FIGS. 3, 4, and 5), combined with the moiety format selection of FIG. 7, whereby the total molecular valency is four, but specificity is restricted to two specificities. The N-terminus is comprised of two identical soluble VH3 germline based VH domains and the C-terminus is comprised of two identical soluble VH3 germlined based VH domains of different specificity to the N-terminal domains. Therefore, each specificity has a valence of two. Again, in this format, any of the antibody moieties at any of the four attachment/fusion points can be substituted with a non-antibody moiety, e.g., an effector binding/modulating moiety that does not comprise an antibody molecule.

FIG. 10 illustrates another example of a bispecific molecule having a mixed format. In this example, the molecule is comprised of four VH3 germline based soluble VH domains. The first two domains have the same specificity (for example an inhibitory receptor), the 3rd domain from the N-terminus may have specificity for a tissue antigen and the fourth domain from the N-terminus may have specificity for human serum albumin (HSA), thereby granting the molecule extended half-life in the absence of an Ig Fc domain. Three glycine, serine, alanine and/or threonine rich linkers exists between domains 1 and 2, domains 2 and 3, and domains 3 and 4. This format may be configured with up to tetraspecificity, but monovalent in each case, or to have bispecificity with bivalency in each case. The order of domains can be changed. Again, in this format, any of the antibody moieties can be substituted with a non-antibody moiety, e.g., an effector binding/modulating moiety that does not comprise an antibody molecule.

FIG. 11 illustrates yet another approach. This example is similar to FIGS. 3 and 4, in that it is Fc homodimer based with two identical Fab units (bivalent monospecificity) at the N-terminus of the molecule. This example differs from FIGS. 3 and 4 in that the C-terminus of each heavy chain is appended with a tandem-scFv. Thus, in each case the C-terminus of the CH3 domain of the Fc is linked via a glycine/serine/alanine/threonine based linker to the N-terminus of a first VH domain, which is linked via the C-terminus by a 12-15 amino acid glycine/serine rich linker to the N-terminus of a first VL domain, which linked via a 25-35 amino acid glycine/serine/alanine/threonine based linker at the C-terminus to the N-terminus of a second VH domain, which is linked via the C-terminus with a 12-15 amino acid glycine/serine based linker to the N-terminus of a 2nd VL domain. In this Fc homodimer based molecule there are therefore two identical tandem scFvs at the C-terminus of the molecule offering either tetravalency for a single tissue antigen for example or bivalency to two different molecules. This format could also be adapted with a heterodimer Fc core allowing two different tandem-scFvs at the C-terminus of the Fc allowing for monovalent tetraspecificity at the C-terminus while retaining either bivalent monospecificity at the N-terminus or monovalent bispecificity at the N-terminal via usage of single chain Fab configurations as in FIGS. 5, 6, and 7. This molecule can therefore be configured to have 2, 3, 4, 5, or 6 specificities. The domain order of scFvs within the tandem scFv units may be configured to be from N- to C-terminus either VH-Linker-VL or VL-Linker-VH. Again, in this format, any of the antibody moieties at any of the four attachment/fusion points can be substituted with a non-antibody moiety, e.g., an effector binding/modulating moiety that does not comprise an antibody molecule.

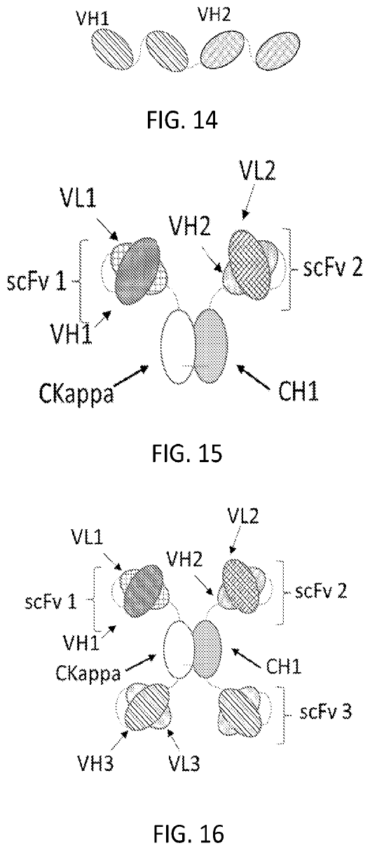

Bispecific antibodies can also be constructed to have, for example, shorter systemic PK while having increased tissue penetration. These types of antibodies can be based upon, for example, a human VH3 based domain antibody format. These are illustrated, for example, in FIGS. 12, 13, and 14. FIGS. 12, 13, and 14 each comprised a soluble VH3 germline family based VH domain modules. Each domain is approximately 12.5 kDa allowing for a small overall MW, which, without being bound to any particular theory, could be beneficial for enhanced tissue penetration. In these examples, none of the VH domains recognize any half-life extending targets such as FcRn or HSA. As illustrated in FIG. 12, the molecule is comprised of two VH domains joined with a flexible hydrophilic glycine/serine based linker between the C-terminus of the first domain and N-terminus of the second domain. In this example one domain may recognize a T cell costimulatory receptor and the second may recognize a tissue tethering antigen. As illustrated in FIG. 13, the molecule is comprised of three VH domains with N—C-terminal linkages of hydrophilic glycine/serine based linkers. The molecule may be configured to be trispecific but monovalent for each target. It may be bispecific with bivalency for one target and monovalency for another. As illustrated in FIG. 14, the molecule is comprised of four V_{H} domains with N—C-terminal glycine/serine rich linkers between each domain. This molecule may be configured to be tetraspecific, trispecific, or bispecific with varying antigenic valencies in each case. Again, in this format, any of the antibody moieties at can be substituted with a non-antibody moiety, e.g., an effector binding/modulating moiety that does not comprise an antibody molecule.

Other embodiments of bispecific antibodies are illustrated in FIGS. 15 and 16. FIGS. 15 and 16 are comprised of the naturally heterodimerizing core of the human IgG CH1/Ckappa interface, including the C-terminal heavy/light disulfide bond which covalently anchors the interaction. This format does not contain an Fc or any moieties for half-life extension. As illustrated in FIG. 15, the molecule, at the N-terminus of the Ckappa domain is appended with an scFv fragment consisting of an N-terminal VH domain, linked at its C-terminus to the N-terminus of a VL domain via a 12-15 amino acid glycine/serine based linker, which is linked by its C-terminus to the N-terminus of the Ckappa domain via the native VL-Ckappa elbow sequence. The CH1 domain is appended at the N-terminus with an scFv fragment consisting of an N-terminal VL domain linked at its C-terminus via a 12-15 amino acid glycine/serine linker to the N-terminus of a VH domain, which is linked at its C-terminus to the N-terminus of the CH1 domains via the natural VH-CH1 elbow sequence. As illustrated in FIG. 16, the molecule has the same N-terminal configuration to Example 13. However the C-terminus of the Ckappa and CH1 domains are appended with scFv modules which may be in either the VH-VL or VL-VH configuration and may be either specific for the same antigen or specific for two different antigens. The VH/VL inter-domain linkers may be 12-15 amino acids in length and consisting of glycine/serine residues. The scFv binding sub-units may be swapped for soluble VH domains, or peptide recognition elements, or even tandem-scFv elements. This approach can also be configured to use Vlambda and/or Clambda domains. Again, in this format, any of the antibody moieties at any of the attachment/fusion points can be substituted with a non-antibody moiety, e.g., an effector binding/modulating moiety that does not comprise an antibody molecule.

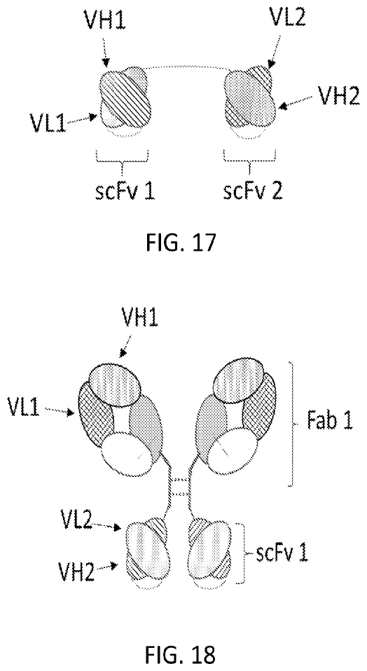

FIG. 17 illustrates another embodiment. FIG. 17 represents a tandem scFv format consisting of a first N-terminal VL domain linked at its C-terminus to the N-terminus of a first VH domain with a 12-15 amino acid glycine/serine rich linker, followed at the first VH C-terminus by a 25-30 amino acid glycine/serine/alanine/threonine based linker to the N-terminus of a second VL domain. The second VL domain is linked at the C-terminus to the N-terminus of a 2nd VH domain by a 12-15 amino acid glycine/serine linker. Each scFv recognizes a different target antigen such as a costimulatory T cell molecule and a tissue tethering target. Again, in this format, any of the antibody moieties can be substituted with a non-antibody moiety, e.g., an effector binding/modulating moiety that does not comprise an antibody molecule.

FIG. 18 illustrates another embodiment. FIG. 18 is a F(ab′)2 scFv fusion. This consists of two identical Fab components joined via two disulfide bonds in the native human IgG1 hinge region C-terminal of the human IgG CH1 domain. The human IgG1 CH2 and CH3 domains are absent. At the C-terminus of heavy chains 1 and 2 are two identical scFv fragments linked via a glycine/serine/alanine/threonine rich linker to the C-terminus of the huIgG1 hinge region. In the configuration shown, the VH is N-terminal in each scFv unit and linked via a 12-15 amino acid glycine/serine rich linker to the N-terminus of a VL domain. An alternative configuration would be N-term-VL-Linker-VH—C-term. In this design, the construct is bispecific with bivalency for reach target. Again, in this format, any of the antibody moieties at any of the four attachment/fusion points can be substituted with a non-antibody moiety, e.g., an effector binding/modulating moiety that does not comprise an antibody molecule.

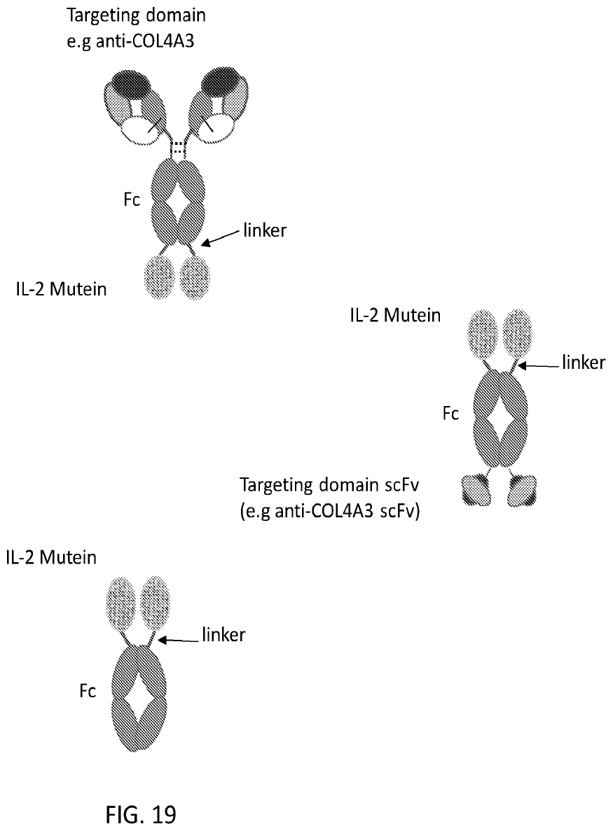

FIG. 19 illustrates another non-limiting embodiment. The N-terminus of the homodimer contains two targeting domains comprised of two targeting moieties, such as those provide herein. In another embodiments, the N-terminus of the homodimer comprises two IL-2 muteins, such as those provided herein. At the C-terminus of this design are two identical scFv units of the targeting domain. The scFv can comprise any two targeting moieties provided herein. In another embodiments, the N-terminus of the homodimer does not comprise a targeting moiety or an effector molecule.

### Complement System

Complement activation occurs via three main pathways, known as the classical, alternative, and lectin pathways (Kuby, Immunology, 2000). The classical pathway is usually triggered by binding of a complex of antigen and IgM or IgG antibody to C1 (though certain other activators can also initiate the pathway). Activated C1 cleaves C4 and C2 to produce C4a and C4b, in addition to C2a and C2b. C4b and C2a combine to form C3 convertase, which cleaves C3 to form C3a and C3b. Binding of C3b to C3 convertase produces C5 convertase, which cleaves C5 into C5a and C5b. C3a, C4a, and C5a are anaphylatoxins and mediate multiple reactions in the acute inflammatory response. C3a and C5a are also chemotactic factors that attract immune system cells such as neutrophils.

The alternative pathway is initiated by microbial surfaces and various complex polysaccharides. In this pathway, C3b, resulting from cleavage of C3, which occurs spontaneously at a low level, binds to targets, e.g., on cell surfaces and forms a complex with factor B, which is later cleaved by factor D, resulting in a C3 convertase. Cleavage of C3 and binding of another molecule of C3b to the C3 convertase gives rise to a C5 convertase.

The C5 convertases produced in both pathways cleave C5 to produce C5a and C5b. C5b then binds to C6, C7, and C8 to form C5b-8, which catalyzes polymerization of C9 to form the C5b-9 membrane attack complex (MAC). The MAC inserts itself into target cell membranes and causes cell lysis. Small amounts of MAC on the membrane of cells may have a variety of consequences other than cell death.

A third complement pathway, the lectin complement pathway is initiated by binding of mannose-binding lectin (MBL) and MBL-associated serine protease (MASP) to carbohydrates. In the human lectin pathway, MASP-1 and MASP-2 are involved in the proteolysis of C4, C2 and C3, leading to a C3 convertase described above.

Complement activity is regulated by members of the endogenous “regulators of complement activation” (RCA) family, also called “complement control proteins” (CCPs), or “complement regulatory proteins” (CRPs), which include complement receptor type 1 (CR1; C3b:C4b receptor), complement receptor type 2 (CR2), membrane cofactor protein (MCP; CD46), decay-accelerating factor (DAF), complement factor H (fH), complement receptor-related protein y (Crry), C4b-binding protein (C4 bp), and decay-cofactor protein (DCP) (Panwar et al., 2019, PNAS). CCPs are characterized by multiple (typically 4-56) homologous motifs known as short consensus repeats (SCR), complement control protein (CCP) modules, or SUSHI domains (Reid, K B M and Day, A J, Immunol Today, 10:177-80, 1989). Complement control proteins negatively regulate the complement system, e.g., by accelerating the normal decay of convertases and/or functioning as cofactors for factor I to enzymatically cleave C3b and/or C4b into smaller fragments.

As provided herein, the effector moiety that is linked or associated with can be a PD-1 agonist, IL-2 mutein, or complement modulator molecule. The term “complement modulator molecule,” as used herein, refers to a polypeptide having sufficient complement modulatory sequence that, as part of a therapeutic compound, inhibits the complement system. In some embodiments, a complement modulator molecule is a CD55 protein, a CD59 protein, a CR1 protein, or a DCP protein. In some embodiments, a CD55, CD59, CR1, or DCP molecule has at least 60, 70, 80, 90, 95, 99, or 100% sequence identity, or substantial sequence identity, with a naturally occurring CD55, CD59, CR1, or DCP.

In some embodiments, the complement modulator is CD55, CD59, CR1, or DCP. In some embodiments, the CD55 protein has the sequence as provided below. In some embodiments, the CD55 sequence is any functional isoform having at least 60, 70, 80, 90, 95, 99, or 100% sequence identity, or substantial sequence identity, with a naturally occurring CD55. In some embodiments, the CD59 protein has the sequence as provided below. In some embodiments, the CD59 sequence is any functional isoform having at least 60, 70, 80, 90, 95, 99, or 100% sequence identity, or substantial sequence identity, with a naturally occurring CD59. In some embodiments, the CR1 protein has the sequence as provided below. In some embodiments, the CR1 sequence is any functional isoform having at least 60, 70, 80, 90, 95, 99, or 100% sequence identity, or substantial sequence identity, with a naturally occurring CR1. In some embodiments, the DCP protein has the sequence as provided below. In some embodiments, the DCP sequence is any functional isoform having at least 60, 70, 80, 90, 95, 99, or 100% sequence identity, or substantial sequence identity, with a naturally occurring DCP. In some embodiments, the CD55 sequence or a mature form from the following sequence is provided:

```
  (SEQ ID NO: 1)
DCGLPPDVPNAQPALEGRTSFPEDTVITYKCEESFVKIPGEKDS
VICLKGSQWSDIEEFCNRSCEVPTRLNSASLKQPYITQNYFPVG
TVVEYECRPGYRREPSLSPKLTCLQNLKWSTAVEFCKKKSCPNP
GEIRNGQIDVPGGILFGATISFSCNTGYKLFGSTSSFCLISGSS
VQWSDPLPECREIYCPAPPQIDNGIIQGERDHYGYRQSVTYACN
KGFTMIGEHSIYCTVNNDEGEWSGPPPECRGKSLTSKVPPTVQK
PTTVNVPTTEVSPTSQKTTTKTTTPNAQATRSTPVSRTTKHFHE
TTPNKGSGTTS huCD55 ECD;
accession# P08174, amino acids 35-353,
extracellular domain (ECD);
  (SEQ ID NO: 2)
DCGLPPDVPNAQPALEGRTSFPEDTVITYKCEESFVKIPGEKD
SVICLKGSQWSDIEEFCNRSCEVPTRLNSASLKQPYITQNYFP
VGTVVEYECRPGYRREPSLSPKLTCLQNLKWSTAVEFCKKKSC
PNPGEIRNGQIDVPGGILFGATISFSCNTGYKLFGSTSSFCLI
SGSSVQWSDPLPECREIYCPAPPQIDNGIIQGERDHYGYRQSV
TYACNKGFTMIGEHSIYCTVNNDEGEWSGPPPECRG
huCD55-trunc; accession# P08174,
amino acids 35-285, functional sushi domain
1-4;
  (SEQ ID NO: 3)
DCGLPPDVPNAQPALEGRTSFPEDTVITYKCEESFVKIPGEKD
SVICLKGSQWSDIEEFCQRSCEVPTRLNSASLKQPYITQNYFP
VGTVVEYECRPGYRREPSLSPKLTCLQNLKWSTAVEFCKKKSC
PNPGEIRNGQIDVPGGILFGATISFSCNTGYKLFGSTSSFCLI
SGSSVQWSDPLPECREIYCPAPPQIDNGIIQGERDHYGYRQSV
TYACNKGFTMIGEHSIYCTVNNDEGEWSGPPPECRG
huCD55-trunc-N61Q; accession#
P08174, amino acids 35-285, N-glycan
removed by N61Q mutation;
  (SEQ ID NO: 4)
DCGPPPAVPNAQPALKGLTSFPENTVITYRCDENEMKIPGKQD
SVMCLPDSQWSDIEEFCNRSCGAPTRLKFASLKQLYIPQSYFP
VGTVVEYECRPGYRRDPSLLAKLTCLQNLKWSTAAEFCKKKSC
PNPGEIPNGQIDTSNGILFGAAISFSCNTGYKLFGPTSSLCLV
SGSGVQWSDTLPECREIYCPAPPQIDNGIIQGEREHYGYRQSI
TYLCNRGFTMIGEHSIYCTVNDDEGEWSGPPPTCRANSLVSKA
PPTVQKPTTVNVRTTEVSPTSQKTTTPNAQATRSTPASRTTKH
FHKTTPDKGSGTSS cyno CD55
ECD; accession# A0A2K5X1Z6;
  (SEQ ID NO: 5)
DCGPPPDIPNARPILGRHSKFAEQSKVAYSCNNGFKQVPDKSN
IVVCLENGQWSSHETFCEKSCVAPERLSFASLKKEYLNMNFFP
VGTIVEYECRPGFRKQPPLPGKATCLEDLVWSPVAQFCKKKSC
PNPKDLDNGHINIPTGILFGSEINFSCNPGYRLVGVSSTFCSV
TGNTVDWDDEFPVCTEIHCPEPPKINNGIMRGESDSYTYSQVV
TYSCDKGFILVGNASIYCTVSKSDVGQWSSPPPRCIEKSKVPT
KKPTINVPSTGTPSTPQKPTTESVPNPGDQPTPQKPSTVKVSA
TQHVPVTKTTVRHPIRTSTDKGEPNTG
mouse CD55 ECD; accession# Q61475;
  (SEQ ID NO: 6)
DCGPPPDIPNARPILGRHSKFAEQSKVAYSCNNGFKQVPDKSN
IVVCLENGQWSSHETFCEKSCVAPERLSFASLKKEYLNMNFFP
VGTIVEYECRPGFRKQPPLPGKATCLEDLVWSPVAQFCKKKSC
PNPKDLDNGHINIPTGILFGSEINESCNPGYRLVGVSSTFCSV
TGNTVDWDDEFPVCTEIHCPEPPKINNGIMRGESDSYTYSQVV
TYSCDKGFILVGNASIYCTVSKSDVGQWSSPPPRCIE
mouse CD55-trunc; accession#
Q61475, functional sushi domain 1-4.
```

In some embodiments, the CD59 sequence or a mature form from the following sequence is provided:

```
  (SEQ ID NO: 7)
LQCYNCPNPTADCKTAVNCSSDFDACLITKAGLQVYNKCWKFE
HCNFNDVTTRLRENELTYYCCKKDLCNFNEQLEN
huCD59 ECD; accession# P13987;
  (SEQ ID NO: 8)
LQCYNCPNPTTDCKTAINCSSGFDTCLIARAGLQVYNQCWKFA
NCNYNDISTLLKESELRYFCCKKDLCNFNEQLES
cyno CD59 ECD; accession# Q8SQ46;
  (SEQ ID NO: 9)
LTCYHCFQPVVSSCNMNSTCSPDQDSCLYAVAGMQVYQRCWKQ
SDCHGEIIMDQLEETKLKERCCQFNLCNKS
mouse CD59 ECD; accession# O55186.
```

In some embodiments, the CR1 or Crry sequence or a mature form from the following sequence is provided:

```
  (SEQ ID NO: 10)
QCNAPEWLPFARPTNLTDEFEFPIGTYLNYECRPGYSGRPFSI
ICLKNSVWTGAKDRCRRKSCRNPPDPVNGMVHVIKGIQFGSQI
KYSCTKGYRLIGSSSATCIISGDTVIWDNETPICDRIPCGLPP
TITNGDFISTNRENFHYGSVVTYRCNPGSGGRKVFELVGEPSI
YCTSNDDQVGIWSGPAPQCIIPNKCTPPNVENGILVSDNRSLF
SLNEVVEFRCQPGFVMKGPRRVKCQALNKWEPELPSCSRVCQP
PPDVLHAERTQRDKDNFSPGQEVFYSCEPGYDLRGAASMRCTP
QGDWSPAAPTCEVKSCDDFMGQLLNGRVLFPVNLQLGAKVDFV
CDEGFQLKGSSASYCVLAGMESLWNSSVPVCEQIFCPSPPVIP
NGRHTGKPLEVFPFGKTVNYTCDPHPDRGTSFDLIGESTIRCT
SDPQGNGVWSSPAPRCGILGHCQAPDHFLFAKLKTQTNASDEP
IGTSLKYECRPEYYGRPFSITCLDNLVWSSPKDVCKRKSCKTP
PDPVNGMVHVITDIQVGSRINYSCTTGHRLIGHSSAECILSGN
AAHWSTKPPICQRIPCGLPPTIANGDFISTNRENFHYGSVVTY
RCNPGSGGRKVFELVGEPSIYCTSNDDQVGIWSGPAPQCIIPN
KCTPPNVENGILVSDNRSLFSLNEVVEFRCQPGFVMKGPRRVK
CQALNKWEPELPSCSRVCQPPPDVLHAERTQRDKDNESPGQEV
FYSCEPGYDLRGAASMRCTPQGDWSPAAPTCEVKSCDDEMGQL
LNGRVLFPVNLQLGAKVDFVCDEGFQLKGSSASYCVLAGMESL
WNSSVPVCEQIFCPSPPVIPNGRHTGKPLEVFPFGKAVNYTCD
PHPDRGTSFDLIGESTIRCTSDPQGNGVWSSPAPRCGILGHCQ
APDHELFAKLKTQTNASDFPIGTSLKYECRPEYYGRPFSITCL
DNLVWSSPKDVCKRKSCKTPPDPVNGMVHVITDIQVGSRINYS
CTTGHRLIGHSSAECILSGNTAHWSTKPPICQRIPCGLPPTIA
NGDFISTNRENFHYGSVVTYRCNLGSRGRKVFELVGEPSIYCT
SNDDQVGIWSGPAPQCIIPNKCTPPNVENGILVSDNRSLFSLN
EVVEFRCQPGFVMKGPRRVKCQALNKWEPELPSCSRVCQPPPE
ILHGEHTPSHQDNFSPGQEVFYSCEPGYDLRGAASLHCTPQGD
WSPEAPRCAVKSCDDFLGQLPHGRVLFPLNLQLGAKVSFVCDE
GFRLKGSSVSHCVLVGMRSLWNNSVPVCEHIFCPNPPAILNGR
HTGTPSGDIPYGKEISYTCDPHPDRGMTFNLIGESTIRCTSDP
HGNGVWSSPAPRCELSVRAGHCKTPEQFPFASPTIPINDFEFP
VGTSLNYECRPGYFGKMFSISCLENLVWSSVEDNCRRKSCGPP
PEPFNGMVHINTDTQFGSTVNYSCNEGERLIGSPSTTCLVSGN
NVTWDKKAPICEIISCEPPPTISNGDFYSNNRTSFHNGTVVTY
QCHTGPDGEQLFELVGERSIYCTSKDDQVGVWSSPPPRCISTN
KCTAPEVENAIRVPGNRSFFSLTEIIRFRCQPGFVMVGSHTVQ
CQTNGRWGPKLPHCSRVCQPPPEILHGEHTLSHQDNESPGQEV
FYSCEPSYDLRGAASLHCTPQGDWSPEAPRCTVKSCDDELGQL
PHGRVLLPLNLQLGAKVSFVCDEGFRLKGRSASHCVLAGMKAL
WNSSVPVCEQIFCPNPPAILNGRHTGTPFGDIPYGKEISYACD
THPDRGMTFNLIGESSIRCTSDPQGNGVWSSPAPRCELSVPAA
CPHPPKIQNGHYIGGHVSLYLPGMTISYICDPGYLLVGKGFIF
CTDQGIWSQLDHYCKEVNCSFPLEMNGISKELEMKKVYHYGDY
VTLKCEDGYTLEGSPWSQCQADDRWDPPLAKCTSRTHD
huCR1 ECD; accession# P17927;
  (SEQ ID NO: 11)
ELRGGLGKHGHTVHREPAVNRLCADSKRWSGLPVSAQRPFPMG
HCPAPSQLPSAKPINLTDESMFPIGTYLLYECLPGYIKRQFSI
TCKQDSTWTSAEDKCIRKQCKTPSDPENGLVHVHTGIQFGSRI
NYTCNQGYRLIGSSSAVCVITDQSVDWDTEAPICEWIPCEIPP
GIPNGDFFSSTREDFHYGMVVTYRCNTDARGKALENLVGEPSL
YCTSNDGEIGVWSGPPPQCIELNKCTPPPYVENAVMLSENRSL
FSLRDIVEFRCHPGFIMKGASSVHCQSLNKWEPELPSCFKGVI
CRLPQEMSGFQKGLGMKKEYYYGENVTLECEDGYTLEGSSQSQ
CQSDGSWNPLLAKCVSRSISG mouse
Crry ECD; accession# Q64735.
```

In some embodiments, the DCP sequence or a mature form from the following sequence is provided:

(SEQ ID NO: 12)
SCEVPTRLNSASLKQPYITQNYFPVGTVVEYECRPGYRREPSL
SPKLTCLQNLKWSTAVEFCKKKSCPNPGEIRNGQIDVPGGILF
GATISFSCNTGYKLIGSYSSFCKISGSSVQWSDKLPICEKVLC
TPPPKIKNGKHTFSEVEVFEYLDAVTYSCDPAPGPDPFSLIGE
STIYCGDNSVWSRAAPECKVVKCRFPVVENGKQISGFGKKFYY
KATVMFECDKGFYLDGSDTIVCDSNSTWDPPVPKCLKV
DCP (Panwar et al., Molecular
engineering of an efficient four-domain
DAF-MCP chimera reveals the presence of
functional modularity in RCA proteins.
PNAS, 116 (20):9953-9958 (2019)).

Effector, as that term is used herein, refers to an entity, e.g., a cell or molecule, e.g., a soluble or cell surface molecule, which mediates an immune response. Non-limiting examples of effectors are PD-1 agonists, IL-2 muteins, and the complement modulator domains and polypeptides, such as those provided for herein.

Effector ligand binding molecule, as used herein, refers to a polypeptide that has sufficient sequence from a naturally occurring counter ligand of an effector, that it can bind the effector with sufficient specificity that it can serve as an effector binding/modulating molecule. In some embodiments, an effector ligand binding molecule binds to an effector with at least 10, 20, 30, 40, 50, 60, 70, 80, 90, or 95% of the affinity of the naturally occurring counter ligand. In some embodiments, it has at least 60, 70, 80, 90, 95, 99, or 100% sequence identity, or substantial sequence identity, with a naturally occurring counter ligand for the effector.

Effector specific binding polypeptide, as used herein, refers to a polypeptide that can bind with sufficient specificity that it can serve as an effector binding/modulating moiety. In some embodiments, a specific binding polypeptide comprises an effector ligand binding molecule.

Elevated risk, as used herein, refers to the risk of a disorder in a subject, wherein the subject has one or more of a medical history of the disorder or a symptom of the disorder, a biomarker associated with the disorder or a symptom of the disorder, or a family history of the disorder or a symptom of the disorder.

In some embodiments, the anti-effector, modulatory, or inhibitory immune checkpoint antibody molecule, when binding as a monomer (or binding when the therapeutic compound is not multimerized), to the effector, modulator, or inhibitory immune checkpoint molecule, does not antagonize, substantially antagonize, prevent binding, or prevent substantial binding, of an endogenous counter ligand of the modulatory, or inhibitory immune checkpoint molecule to modulatory, or inhibitory immune checkpoint molecule. In some embodiments, the anti-effector, modulator, or inhibitory immune checkpoint molecule antibody molecule when binding as a monomer (or binding when the therapeutic compound is not multimerized), to the modulatory, or inhibitory immune checkpoint molecule, does not agonize or substantially agonize, the effector, modulator, or inhibitory molecule.

IL-2 mutein molecule, as that term is used herein, refers to an IL-2 variant that binds with high affinity to the CD25 (IL-2R alpha chain) and with low affinity to the other IL-2R signaling components CD122 (IL-2R beta) and CD132 (IL-2R gamma). Such an IL-2 mutein molecule preferentially activates Treg cells. In embodiments, either alone, or as a component of a therapeutic compound, an IL-2 mutein activates Tregs at least 2, 5, 10, or 100 fold more than cytotoxic or effector T cells. Exemplary IL-2 mutein molecules are described in U.S. Pat. Nos. 10,174,091, 10,676,516, WO2010085495, WO2016/164937, US2014/0286898A1, WO2014153111A2, WO2010/085495, WO2016014428A2, WO2016025385A1, and US20060269515. Muteins disclosed in these references that include additional domains, e.g., an Fc domain, or other domain for extension of half-life can be used in the therapeutic compounds and methods described herein without such additional domains.

In some embodiments, the effector domain is an “inhibitory immune checkpoint molecule ligand molecule,” which refers to a polypeptide having sufficient inhibitory immune checkpoint molecule ligand sequence, e.g., in the case of a PD-L1 molecule, sufficient PD-L1 sequence, that when present as an ICIM binding/modulating moiety of a multimerized therapeutic compound, can bind and agonize its cognate inhibitory immune checkpoint molecule, e.g., again in the case of a PD-L1 molecule, PD-1.

In some embodiments, the inhibitory immune checkpoint molecule ligand molecule, e.g., a PD-L1 molecule, when binding as a monomer (or binding when the therapeutic compound is not multimerized), to its cognate ligand, e.g., PD-1, does not antagonize or substantially antagonize, or prevent binding, or prevent substantial binding, of an endogenous inhibitory immune checkpoint molecule ligand to the inhibitory immune checkpoint molecule. For example, in the case of a PD-L1 molecule, the PD-L1 molecule does not antagonize binding of endogenous PD-L1 to PD-1.

In some embodiments, the inhibitory immune checkpoint molecule ligand, when binding as a monomer to its cognate inhibitory immune checkpoint molecule does not agonize or substantially agonize the inhibitory immune checkpoint molecule. By way of example, a PD-L1 molecule when binding to PD-1, does not agonize or substantially agonize PD-1.

In some embodiments, an inhibitory immune checkpoint molecule ligand molecule has at least 60, 70, 80, 90, 95, 96, 97, 98, 99, or 100% sequence identity, or substantial sequence identity, with a naturally occurring inhibitory immune checkpoint molecule ligand.

Exemplary inhibitory immune checkpoint molecule ligand molecules include: a PD-L1 molecule, which binds to inhibitory immune checkpoint molecule PD-1, and in embodiments has at least 60, 70, 80, 90, 95, 96, 97, 98, 99, or 100% sequence identity, or substantial sequence identity, with a naturally occurring PD-L1, e.g., the PD-L1 molecule comprising the sequence of

(SEQ ID NO: 13)
MRIFAVFIFMTYWHLLNAFTVTVPKDLYVVEYGSNMTIECKFPVEKQLDL
AALIVYWEMEDKNIIQFVHGEEDLKVQHSSYRQRARLLKDQLSLGNAALQ
ITDVKLQDAGVYRCMISYGGADYKRITVKVNAPYNKINQRILVVDPVTSE
HELTCQAEGYPKAEVIWTSSDHQVLSGKTTTTNSKREEKLENVISTLRIN
TTTNEIFYCTFRRLDPEENHTAELVIPELPLAHPPNERTHLVILGAILLC
LGVALTFIFRLRKGRMMDVKKCGIQDTNSKKQSDTHLEET,

or an active fragment thereof, in some embodiments, the active fragment comprises residues 19 to 290 of the PD-L1 sequence; an HLA-G molecule, which binds to any of inhibitory immune checkpoint molecules KIR2DL4, LILRB1, and LILRB2, and in embodiments has at least 60, 70, 80, 90, 95, 99, or 100% sequence identity, or substantial sequence identity, with a naturally occurring HLA-G. Exemplary HLA-G sequences include, e.g., a mature form found in the sequence at GenBank P17693.1 RecName: Full=HLA class I histocompatibility antigen, alpha chain G; AltName: Full=HLA G antigen; AltName: Full=MHC class I antigen G; Flags: Precursor, or in the sequence

(SEQ ID NO: 14)
MVVMAPRTLFLLLSGALTLTETWAGSHSMRYFSAAVSRPGRGEPRFIAMG
YVDDTQFVREDSDSACPRMEPRAPWVEQEGPEYWEEETRNTKAHAQTDRM
NLQTLRGYYNQSEASSHTLQWMIGCDLGSDGRLLRGYEQYAYDGKDYLAL
NEDLRSWTAADTAAQISKRKCEAANVAEQRRAYLEGTCVEWLHRYLENGK
EMLQRADPPKTHVTHHPVFDYEATLRCWALGFYPAEIILTWQRDGEDQTQ
DVELVETRPAGDGTFQKWAAVVVPSGEEQRYTCHVQHEGLPEPLMLRWKQ
SSLPTIPIMGIVA.

Sequence identity, percentage identity, and related terms, as those terms are used herein, refer to the relatedness of two sequences, e.g., two nucleic acid sequences or two amino acid or polypeptide sequences. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity, for example, amino acid sequences that contain a common structural domain having at least about 85%, 90%, 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity, for example, nucleotide sequences having at least about 85%, 90%, 91%, 92%, 93%, 94%, 95%, 96%, 97%, 98% or 99% identity to a reference sequence, e.g., a sequence provided herein.

The term “functional variant” refers to polypeptides that have a substantially identical amino acid sequence to the naturally occurring sequence, or are encoded by a substantially identical nucleotide sequence, and are capable of having one or more activities of the naturally occurring sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 95%, 96%, 97%, 98%, 99%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at the GCG website at gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to for example any a nucleic acid sequence provided herein. BLAST protein searches can be performed with the XBLAST program, score=50, wordlength=3 to obtain amino acid sequences homologous to protein molecules provided herein. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See the NCBI website at ncbi.nlm.nih.gov.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified.

It is understood that the molecules and compounds of the present embodiments may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally occurring amino acids. Exemplary amino acids include naturally occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

In some embodiments, the molecule comprises a complement modulator molecule, an IL-2 mutein, or PD-1 agonist, such as an antibody.

Specific targeting moiety, as that term is used herein, refers to kidney-glomerular specific targeting moiety.

Target ligand binding molecule, as used herein, refers to a polypeptide that has sufficient sequence from a naturally occurring counter ligand of a target ligand that it can bind the target ligand on a target tissue (e.g., donor tissue or subject target tissue) with sufficient specificity that it can serve as a specific targeting moiety. In some embodiments, a target ligand binding molecule binds to target tissue or cells with at least 10, 20, 30, 40, 50, 60, 70, 80, 90, 95, 96, 97, 98, or 99% of the affinity of the naturally occurring counter ligand. In some embodiments, a target ligand binding molecule has at least 60, 70, 80, 90, 95, 96, 97, 98, 99, or 100% sequence identity, or substantial sequence identity, with a naturally occurring counter ligand for the target ligand.

Target site, as that term is used herein, refers to a site which contains the entity, e.g., epitope, bound by a targeting moiety. In some embodiments, the target site is the site at which immune privilege is established, such as the kidney or the structures within the kidney. In some embodiments, the target site is the kidney glomerulus.

Tissue specific targeting moiety, as that term is used herein, refers to a moiety, e.g., an antibody molecule, that as a component of a therapeutic molecule, localizes the therapeutic molecule preferentially to a target tissue, as opposed to other tissue of a subject. As a component of a therapeutic compound, the tissue specific targeting moiety provides site-specific immune privilege for a target tissue, e.g., an organ or tissue undergoing or at risk for autoimmune attack.

In some embodiments, a tissue specific targeting moiety binds to a product, e.g., a polypeptide product, which is not present outside the target tissue, or is present at sufficiently low levels that, at therapeutic concentrations of therapeutic molecule, unacceptable levels of immune suppression are absent or substantially absent. In some embodiments, a tissue specific targeting moiety binds to an epitope, which epitope is not present outside, or not substantially present outside, the target site.

In some embodiments, the targeting moiety binds to ROBO2 in kidney glomerulus. In some embodiments, the targeting moiety binds to the extracellular domain of ROBO2. In some embodiments, the molecule that binds to ROBO2 in kidney glomerulus is an anti-Robo2 antibody. In some embodiments, the Robo2 has the sequence of.

```
  (SEQ ID NO: 70)
SRLRQEDFPPRIVEHPSDVIVSKGEPTTLNCKAEGRPTPTIEW
YKDGERVETDKDDPRSHRMLLPSGSLFFLRIVHGRRSKPDEGS
YVCVARNYLGEAVSRNASLEVALLRDDFRQNPTDVVVAAGEPA
ILECQPPRGHPEPTIYWKKDKVRIDDKEERISIRGGKLMISNT
RKSDAGMYTCVGTNMVGERDSDPAELTVFERPTFLRRPINQVV
LEEEAVEFRCQVQGDPQPTVRWKKDDADLPRGRYDIKDDYTLR
IKKTMSTDEGTYMCIAENRVGKMEASATLTVRAPPQFVVRPRD
QIVAQGRTVTFPCETKGNPQPAVFWQKEGSQNLLFPNQPQQPN
SRCSVSPTGDLTITNIQRSDAGYYICQALTVAGSILAKAQLEV
TDVLTDRPPPIILQGPANQTLAVDGTALLKCKATGDPLPVISW
LKEGFTFPGRDPRATIQEQGTLQIKNLRISDTGTYTCVATSSS
GETSWSAVLDVTESGATISKNYDLSDLPGPPSKPQVTDVTKNS
VTLSWQPGTPGTLPASAYIIEAFSQSVSNSWQTVANHVKTTLY
TVRGLRPNTIYLEMVRAINPQGLSDPSPMSDPVRTQDISPPAQ
GVDHRQVQKELGDVLVRLHNPVVLTPTTVQVTWTVDRQPQFIQ
GYRVMYRQTSGLQATSSWQNLDAKVPTERSAVLVNLKKGVTYE
IKVRPYFNEFQGMDSESKTVRTTEEAPSAPPQSVTVLTVGSYN
STSISVSWDPPPPDHQNGIIQEYKIWCLGNETRFHINKTVDAA
IRSVIIGGLFPGIQYRVEVAASTSAGVGVKSEPQPIIIGRRNE
VVITENNNSITEQITDVVKQP
huRobo2; accession# Q9HCK4;
  (SEQ ID NO: 71)
SRLRQEDFPPRIVEHPSDVIVSKGEPTTLNCKAEGRPTPTIEW
YKDGERVETDKDDPRSHRMLLPSGSLFFLRIVHGRRSKPDEGS
YVCVARNYLGEAVSRNASLEVALLRDDFRQNPTDVVVAAGEPA
ILECQPPRGHPEPTIYWKKDKVRIDDKEERISIRGGKLMISNT
RKSDAGMYTCVGTNMVGERDSDPAELTVFERPTFLRRPINQVV
LEEEAVEFRCQVQGDPQPTVRWKKDDADLPRGRYDIKDDYTLR
IKKTMSTDEGTYMCIAENRVGKMEASATLTVRAPPQFVVRPRD
QIVAQGRTVTFPCETKGNPQPAVFWQKEGSQNLLFPNQPQQPN
SRCSVSPTGDLTITNIQRSDAGYYICQALTVAGSILAKAQLEV
TDVLTDRPPPIILQGPANQTLAVDGTALLKCKATGDPLPVISW
LKEGFTFLSRDPRATIQEQGTLQIKNLRISDTGTYTCVATSSS
GETSWSAVLDVTESGATISKNYDLNDLPGPPSKPQVTDVTKNS
VTLSWQPGTPGTLPASAYIIEAFSQSVSNSWQTVANHVKTTLY
TVRGLRPNTIYLFMVRAINPQGLSDPSPMSDPVRTQDISPPAQ
GVDHRQVQKELGDVLVRLHNPVVLTPTTVQVTWTVDRQPQFIQ
GYRVMYRQTSGLQAASSWQNLDAKVPNERSAVLVNLKKGVTYE
IKVRPYFNEFQGMDSESKTVRTTEEAPSAPPQSVTVLTVGSYN
STSISVSWDPPPPDHQNGILQEYKIWCLGNETRFHINKTVDAA
IRSVIIGGLFPGIQYRVEVAASTSAGVGVKSEPQPIIIGRRNE
VVITENNNSITEQITDVVKQP cyno
Robo2; accession# A0A2K5TJN4;
  (SEQ ID NO: 72)
SRLRQEDFPPRIVEHPSDVIVSKGEPTTLNCKAEGRPTPTIEW
YKDGERVETDKDDPRSHRMLLPSGSLFFLRIVHGRRSKPDEGS
YVCVARNYLGEAVSRNASLEVALLRDDFRQNPTDVVVAAGEPA
ILECQPPRGHPEPTIYWKKDKVRIDDKEERISIRGGKLMISNT
RKSDAGMYTCVGTNMVGERDSDPAELTVFERPTFLRRPINQVV
LEEEAVEFRCQVQGDPQPTVRWKKDDADLPRGRYDIKDDYTLR
IKKAMSTDEGTYVCIAENRVGKVEASATLTVRVRPVAPPQFVV
RPRDQIVAQGRTVTFPCETKGNPQPAVFWQKEGSQNLLFPNQP
QQPNSRCSVSPTGDLTITNIQRSDAGYYICQALTVAGSILAKA
QLEVTDVLTDRPPPIILQGPINQTLAVDGTALLKCKATGEPLP
VISWLKEGFTFLGRDPRATIQDQGTLQIKNLRISDTGTYTCVA
TSSSGETSWSAVLDVTESGATISKNYDMNDLPGPPSKPQVTDV
SKNSVTLSWQPGTPGVLPASAYIIEAFSQSVSNSWQTVANHVK
TTLYTVRGLRPNTIYLFMVRAINPQGLSDPSPMSDPVRTQDIS
PPAQGVDHRQVQKELGDVVVRLHNPVVLTPTTVQVTWTVDRQP
QFIQGYRVMYRQTSGLQASTVWQNLDAKVPTERSAVLVNLKKG
VTYEIKVRPYFNEFQGMDSESKTVRTTEEAPSAPPQSVTVLTV
GSHNSTSISVSWDPPPADHQNGIIQEYKIWCLGNETRFHINKT
VDAAIRSVVIGGLFPGIQYRVEVAASTSAGVGVKSEPQPIIIG
GRNEVVITENNNSITEQITDVVKQP
mouse Robo2; accession# Q7TPD3;
  (SEQ ID NO: 73)
SRLRQEDFPPRIVEHPSDVIVSKGEPTTLNCKAEGRPTPTIEW
YKDGERVETDKDDPRSHRMLLPSGSLFFLRIVHGRRSKPDEGT
YVCVARNYLGEAVSRNASLEVALLRDDFRQNPTDVVVAAGEPA
ILECQPPRGHPEPTIYWKKDKVRIDEKEERISIRGGKLMISNT
RKSDAGMYTCVGTNMVGERDSDPAELTVFERPTFLRRPINQVV
LEDEPAEFRCQVQGDPQPTVRWKKDDADLPRGRYDIKDDYTLR
IKKAISADEGTYVCIAENRVGKVEASATLTVRVRPVAPPQFVV
RPRDQIVAQGRTVTFPCETKGNPQPAVFWQKEGSQNLLFPNQP
QQPNSRCSVSPTGDLTITNIQRSDAGYYICQALTVAGSILAKA
QLEVTDVLTDRPPPIILQGPINQTLAVDGTALLKCKATGEPLP
VISWLKEGFTFLGRDPRATIQDQGTLQIKNLRISDTGTYTCVA
TSSSGETSWSAVLDVTESGATISKNYDTNDLPGPPSKPQVTDV
TKNSVTLSWQPGTPGVLPASAYIIEAFSQSVSNSWQTVANHVK
TTLYTVRGLRPNTIYLEMVRAINPQGLSDPSPMSDPVRTQDIS
PPAQGVDHRQVQKELGDVTVRLHNPVVLTPTTVQVTWTVDRQP
QFIQGYRVMYRQTSGLQASTVWQNLDAKVPTERSAVLVNLKKG
VTYEIKVRPYFNEFQGMDSESKTIRTTEEAPSAPPQSVTVLTV
GSHNSTSISVSWDPPPADHQNGIIQEYKIWCLGNETRFHINKT
VDATIRSVVIGGLFPGIQYRVEVAASTSAGVGVKSEPQPIIIG
GRNEVVITENNNSITEQITDVVKQP
rat Robo2; accession# A0A0G2JZA1.
```

ROBO proteins are a class of transmembrane receptor proteins with 1000 to 1600 amino acids and have highly conserved intracellular domains with no autocatalytic or intrinsic enzymatic activity. ROBO proteins with no autocatalytic and intrinsic enzymatic activity in the intracellular region mediate downstream signaling through the recruitment of different adaptors or proteins. The extracellular domains of the ROBO1, ROBO2, and ROBO3 proteins have the same structure, which includes 5 immunoglobulin domains, 3 fibronectin domains and one transmembrane domain. (Tong M., The Role of the Slit/Robo Signaling Pathway. J Cancer, 2019; 10(12): 2694-2705) ROBO1, ROBO2, ROBO3, and ROBO4 are single-pass transmembrane receptors for SLIT1, SLIT2, and SLIT3. (Fan X., et al., SLIT2/ROBO2 signaling pathway inhibits non-muscle myosin IIA activity and destabilized kidney podocyte adhesion. JCI Insight, 2016; 1(19): e86934) Without wishing to be bound to a particular theory, the repulsive guidance cue SLIT2 and its receptor ROBO2 are required for kidney development and podocyte foot process structure. ROBO2 is a podocyte protein expressed at the basal surface of kidney podocytes and co-localizes with nephrin and podocin. (Fan X., et al., Inhibitory Effects of Robo2 and Nephrin: A Crosstalk between Positive and Negative Signals Regulating Podocyte Structure. Cell Rep., 2012; 2(1): 52-61) Mouse mutants that lack Slit2 or Robo2 develop supernumerary ureteric buds, which leads to a broad-spectrum of urinary tract anomalies (Grieshammer U, et al., SLIT2-mediated ROBO2 signaling restricts kidney induction to a single site. Dev. Cell, 2004; 6:709-717; Lu W, et al., Disruption of ROBO2 is associated with urinary tract anomalies and confers risk of vesicoureteral reflux. Am J Hum Genet. 2007; 80:616-632). Disruption of ROBO2 in humans causes congenital anomalies of the kidneys and urinary tracts (CAKUT), and point mutations of ROBO2 have been identified in patients with vesicoureteral reflux (VUR) (Lu W, et al., Disruption of ROBO2 is associated with urinary tract anomalies and confers risk of vesicoureteral reflux. Am J Hum Genet. 2007; 80:616-632).

In some embodiments, the anti-Robo2 antibody blocks binding of ROBO2 to SLIT1, SLIT2, or SLIT3. In some embodiments, the anti-Robo2 antibody blocks binding of ROBO2 to SLIT1. In some embodiments, the anti-Robo2 antibody blocks binding of ROBO2 to SLIT2. In some embodiments, the anti-Robo2 antibody blocks binding of ROBO2 to SLIT3. In some embodiments, the anti-Robo2 antibody does not block binding of ROBO2 to SLIT1, SLIT2, or SLIT3. In some embodiments, the anti-Robo2 antibody does not block binding of ROBO2 to SLIT1. In some embodiments, the anti-Robo2 antibody does not block binding of ROBO2 to SLIT2. In some embodiments, the anti-Robo2 antibody does not block binding of ROBO2 to SLIT3.

In some embodiments, the targeting moiety binds to ROBO1 in the kidney glomerulus. In some embodiments, the targeting moiety binds to the extracellular domain of ROBO1. In some embodiments, the molecule that binds to ROBO1 in kidney glomerulus is an anti-Robo1 antibody. In some embodiments, the Robo1 has the sequence of:

(SEQ ID NO: 74)
QLIPDPEDVERGNDHGTPIPTSDNDDNSLGYTGSRLRQEDFPP
RIVEHPSDLIVSKGEPATLNCKAEGRPTPTIEWYKGGERVETD
KDDPRSHRMLLPSGSLFFLRIVHGRKSRPDEGVYVCVARNYLG
EAVSHNASLEVAILRDDFRQNPSDVMVAVGEPAVMECQPPRGH
PEPTISWKKDGSPLDDKDERITIRGGKLMITYTRKSDAGKYVC
VGTNMVGERESEVAELTVLERPSFVKRPSNLAVTVDDSAEFKC
EARGDPVPTVRWRKDDGELPKSRYEIRDDHTLKIRKVTAGDMG
SYTCVAENMVGKAEASATLTVQEPPHFVVKPRDQVVALGRTVT
FQCEATGNPQPAIFWRREGSQNLLFSYQPPQSSSRFSVSQTGD
LTITNVQRSDVGYYICQTLNVAGSIITKAYLEVTDVIADRPPP
VIRQGPVNQTVAVDGTFVLSCVATGSPVPTILWRKDGVLVSTQ
DSRIKQLENGVLQIRYAKLGDTGRYTCIASTPSGEATWSAYIE
VQEFGVPVQPPRPTDPNLIPSAPSKPEVTDVSRNTVTLSWQPN
LNSGATPTSYIIEAFSHASGSSWQTVAENVKTETSAIKGLKPN
AIYLFLVRAANAYGISDPSQISDPVKTQDVLPTSQGVDHKQVQ
RELGNAVLHLHNPTVLSSSSIEVHWTVDQQSQYIQGYKILYRP
SGANHGESDWLVFEVRTPAKNSVVIPDLRKGVNYEIKARPFEN
EFQGADSEIKFAKTLEEAPSAPPQGVTVSKNDGNGTAILVSWQ
PPPEDTQNGMVQEYKVWCLGNETRYHINKTVDGSTFSVVIPFL
VPGIRYSVEVAASTGAGSGVKSEPQFIQLDAHGNPVSPEDQVS
LAQQISDVVKQP huRobo1;
accession# Q9Y6N7.

In some embodiments, the anti-Robo1 antibody blocks binding of ROBO1 to SLIT1, SLIT2, and/or SLIT3. In some embodiments, the anti-Robo1 antibody blocks binding of ROBO1 to SLIT1. In some embodiments, the anti-Robo1 antibody blocks binding of ROBO1 to SLIT2. In some embodiments, the anti-Robo1 antibody blocks binding of ROBO1 to SLIT3. In some embodiments, the anti-Robo1 antibody does not block binding of ROBO1 to SLIT1, SLIT2, and/or SLIT3. In some embodiments, the anti-Robo1 antibody does not block binding of ROBO1 to SLIT1. In some embodiments, the anti-Robo1 antibody does not block binding of ROBO1 to SLIT2. In some embodiments, the anti-Robo1 antibody does not block binding of ROBO1 to SLIT3.

In some embodiments, the targeting moiety binds to COL4A3 in the kidney glomerulus. In some embodiments, the targeting moiety binds to the non-collagenous domain (NC1) of COL4A3. In some embodiments, the molecule that binds to COL4A3 in kidney glomerulus is an anti-COL4A3 antibody. In some embodiments, the COL4A3 has the sequence of:

```
  (SEQ ID NO: 75)
WTTRGFVFTRHSQTTAIPSCPEGTVPLYSGFSFLFVQGNQRAH
GQDLGTLGSCLQRFTTMPFLFCNVNDVCNFASRNDYSYWLSTP
ALMPMNMAPITGRALEPYISRCTVCEGPAIAIAVHSQTTDIPP
CPHGWISLWKGFSFIMFTSAGSEGTGQALASPGSCLEEFRASP
FLECHGRGTCNYYSNSYSFWLASLNPERMFRKPIPSTVKAGEL
EKIISRCQVCMKKRH
huCOL4A3-NC1; accession# Q01955;
  (SEQ ID NO: 76)
WTTRGFVFTRHSQTTAIPSCPEGTAPLYSGFSFLFVQGNERAH
GQDLGTLGSCLQRFTTMPFLFCNVNDVCNFASRNDYSYWLSTP
ALMPMNMAPITGRALEPYISRCTVCEAPAIAIAVHSQTTDIPP
CPHGWISLWKGFSFIMFTSAGSEGTGQALASPGSCLEEFRASP
FLECHGRGTCNYYSNSYSFWLASLNPERMERKPIPSTVKAGEL
EKIISRCQVCMKKRH cyno
COL4A3-NC1; accession# A0A2K5U207;
  (SEQ ID NO: 77)
GTRMRGFIFTRHSQTTAIPSCPEGTQPLYSGESLLFVQGNKRA
HGQDLGTLGSCLQRFTTMPFLFCNINNVCNFASRNDYSYWLST
PALMPMDMAPISGRALEPYISRCTVCEGPAMAIAVHSQTTAIP
PCPQDWVSLWKGFSFIMFTSAGSEGAGQALASPGSCLEEFRAS
PFIECHGRGTCNYYSNSYSFWLASLNPERMERKPIPSTVKAGD
LEKIISRCQVCMKKRH
mouse
COL4A3-NC1; accession# Q9QZS0;
  (SEQ ID NO: 78)
GTRMRGFIFTRHSQTTANPSCPEGTQPLYSGFSLLFVQGNEHA
HGQDLGTLGSCLQRFTTMPFLFCNVDNVCNFASRNDYSYWLST
PAPMPMDMAPITGRALEPYVSRCTVCEGPAMAIAVHSQTTAIP
PCPQGWVSLWKGFSFVMFTSAGSEGAGQALASPGSCLEEFRAS
PFIECHGRGTCNYYSNSYSFWLASLNPERMERKPIPSTVKAGD
LEKIISRCQVCMKKRH rat
COL4A3-NC1; accession# F1LRJ1.
```

In some embodiments, the targeting moiety binds to COL4A4 in the kidney glomerulus In some embodiments, the targeting moiety binds to the non-collagenous domain (NC1) of COL4A4. In some embodiments, the molecule that binds to COL4A4 in kidney glomerulus is an anti-COL4A4 antibody. In some embodiments, the COL4A4 has the sequence of.

(SEQ ID NO: 79)
GYLGGFLLVLHSQTDQEPTCPLGMPRLWTGYSLLYLEGQEKAH
NQDLGLAGSCLPVESTLPFAYCNIHQVCHYAQRNDRSYWLASA
APLPMMPLSEEAIRPYVSRCAVCEAPAQAVAVHSQDQSIPPCP
QTWRSLWIGYSFLMHTGAGDQGGGQALMSPGSCLEDFRAAPEL
ECQGRQGTCHFFANKYSFWLTTVKADLQFSSAPAPDTLKESQA
QRQKISRCQVCVKYS
huCOL4A4-NC1; accession# P53420.

In some embodiments, the targeting moiety binds to COL4A5 in the kidney glomerulus. In some embodiments, the targeting moiety binds to the non-collagenous domain (NC1) of COL4A5. In some embodiments, the molecule that binds to COL4A5 in kidney glomerulus is an anti-COL4A5 antibody. In some embodiments, the COL4A5 has the sequence of.

(SEQ ID NO: 80)
SSVAHGFLITRHSQTTDAPQCPQGTLQVYEGFSLLYVQGNKRA
HGQDLGTAGSCLRRESTMPFMFCNINNVCNFASRNDYSYWLST
PEPMPMSMQPLKGQSIQPFISRCAVCEAPAVVIAVHSQTIQIP
HCPQGWDSLWIGYSEMMHTSAGAEGSGQALASPGSCLEEFRSA
PFIECHGRGTCNYYANSYSFWLATVDVSDMFSKPQSETLKAGD
LRTRISRCQVCMKRT
huCOL4A5-NC1; accession# P29400.

In some embodiments, the complement modulator fusion protein is selected from the following tables: Complement Modulator Antibody Table (Full and Domain Sequences) and Complement Modulator Antibody Table (Linker Sequences).

Complement Modulator Antibody Table (Full and Domain Sequences)

| Clone | | | |
|---|---|---|---|
| (scFv) | Full Sequence | V Domain Seq | C Domain Seq |
| CDAB1- | EVQLVQSGAEVKKPGASVKVSCKA | EVQLVQSGAEVKKPGASVK | ASTKGPSVFPLAPSSKSTSGGT |
| HC | SGYTFTGYYMHWVRQAPGQGLEWM | VSCKASGYTFTGYYMHWVR | AALGCLVKDYFPEPVTVSWNSG |
| | GWINPKNGDTEFPQKFQGRVTMTR | QAPGQGLEWMGWINPKNGD | ALTSGVHTFPAVLQSSGLYSLS |
| | DTSITTAYMDLSRLRSDDTAVYYC | TEFPQKFQGRVTMTRDTSI | SVVTVPSSSLGTQTYICNVNHK |
| | ARESGDDAFDIWGQGTMVTVSSAS | TTAYMDLSRLRSDDTAVYY | PSNTKVDKKVEPKSCDKTHTCP |
| | TKGPSVFPLAPSSKSTSGGTAALG | CARESGDDAFDIWGQGTMV | PCPAPEAAGAPSVFLFPPKPKD |
| | CLVKDYFPEPVTVSWNSGALTSGV | TVSS (SEQ ID NO: | TLMISRTPEVTCVVVDVSHEDP |
| | HTFPAVLQSSGLYSLSSVVTVPSS | 82) | EVKFNWYVDGVEVHNAKTKPRE |
| | SLGTQTYICNVNHKPSNTKVDKKV | | EQYNSTYRVVSVLTVLHQDWLN |
| | EPKSCDKTHTCPPCPAPEAAGAPS | | GKEYKCKVSNKALPAPIEKTIS |
| | VFLFPPKPKDTLMISRTPEVTCVV | | KAKGQPREPQVYTLPPSREEMT |
| | VDVSHEDPEVKFNWYVDGVEVHNA | | KNQVSLTCLVKGFYPSDIAVEW |
| | KTKPREEQYNSTYRVVSVLTVLHQ | | ESNGQPENNYKTTPPVLDSDGS |
| | DWLNGKEYKCKVSNKALPAPIEKT | | FFLYSKLTVDKSRWQQGNVFSC |
| | ISKAKGQPREPQVYTLPPSREEMT | | SVMHEALHNHYTQKSLSLSPG |
| | KNQVSLTCLVKGFYPSDIAVEWES | | (SEQ ID NO: 53) |
| | NGQPENNYKTTPPVLDSDGSFFLY | | |
| | SKLTVDKSRWQQGNVFSCSVMHEA | | |
| | LHNHYTQKSLSLSPGGGGGSDCGP | | |
| | PPDIPNARPILGRHSKFAEQSKVA | | |
| | YSCNNGFKQVPDKSNIVVCLENGQ | | |
| | WSSHETFCEKSCVAPERLSFASLK | | |
| | KEYLNMNFFPVGTIVEYECRPGER | | |
| | KQPPLPGKATCLEDLVWSPVAQFC | | |
| | KKKSCPNPKDLDNGHINIPTGILF | | |
| | GSEINFSCNPGYRLVGVSSTFCSV | | |
| | TGNTVDWDDEFPVCTEIHCPEPPK | | |
| | INNGIMRGESDSYTYSQVVTYSCD | | |
| | KGFILVGNASIYCTVSKSDVGQWS | | |
| | SPPPRCIEKSKVPTKKPTINVPST | | |
| | GTPSTPQKPTTESVPNPGDQPTPQ | | |
| | KPSTVKVSATQHVPVTKTTVRHPI | | |
| | RTSTDKGEPNTG (SEQ ID NO: | | |
| | 81) | | |
| CDAB1- | DIVMTQSPSSLSASVGDRVTITCR | DIVMTQSPSSLSASVGDRV | RTVAAPSVFIFPPSDEQLKSGT |
| LC | ASQSISSYLNWYQQKPGKAPKLLI | TITCRASQSISSYLNWYQQ | ASVVCLLNNFYPREAKVQWKVD |
| | YAASSLQSGVPSRFSGSGSGTDFT | KPGKAPKLLIYAASSLQSG | NALQSGNSQESVTEQDSKDSTY |
| | LTISSLQPEDFATYYCQQSVGLFF | VPSRFSGSGSGTDFTLTIS | SLSSTLTLSKADYEKHKVYACE |
| | GGGTKVEIKRTVAAPSVFIFPPSD | SLQPEDFATYYCQQSVGLF | VTHQGLSSPVTKSENRGEC |
| | EQLKSGTASVVCLLNNFYPREAKV | FGGGTKVEIK (SEQ ID | (SEQ ID NO: 54) |
| | QWKVDNALQSGNSQESVTEQDSKD | NO: 84) | |
| | STYSLSSTLTLSKADYEKHKVYAC | | |
| | EVTHQGLSSPVTKSENRGEC | | |
| | (SEQ ID NO: 83) | | |
| CDAB2- | EVQLVESGGGLVQPGRSLKLSCAA | EVQLVESGGGLVQPGRSLK | ASTKGPSVFPLAPSSKSTSGGT |
| HC | SGFTFSDYYMAWVRQAPKKGLEWV | LSCAASGFTFSDYYMAWVR | AALGCLVKDYFPEPVTVSWNSG |
| | ASISYEGSSTNYGDSVKGRFTISR | QAPKKGLEWVASISYEGSS | ALTSGVHTFPAVLQSSGLYSLS |
| | DNAKSTLYLQMNSLRSEDTATYYC | TNYGDSVKGRFTISRDNAK | SVVTVPSSSLGTQTYICNVNHK |
| | ARQYHYYSGDGFAYWGQGTLVTVS | STLYLQMNSLRSEDTATYY | PSNTKVDKKVEPKSCDKTHTCP |
| | SASTKGPSVFPLAPSSKSTSGGTA | CARQYHYYSGDGFAYWGQG | PCPAPEAAGAPSVFLFPPKPKD |
| | ALGCLVKDYFPEPVTVSWNSGALT | TLVTVSS (SEQ ID NO: | TLMISRTPEVTCVVVDVSHEDP |
| | SGVHTFPAVLQSSGLYSLSSVVTV | 86) | EVKFNWYVDGVEVHNAKTKPRE |
| | PSSSLGTQTYICNVNHKPSNTKVD | | EQYNSTYRVVSVLTVLHQDWLN |
| | KKVEPKSCDKTHTCPPCPAPEAAG | | GKEYKCKVSNKALPAPIEKTIS |
| | APSVFLFPPKPKDTLMISRTPEVT | | KAKGQPREPQVYTLPPSREEMT |
| | CVVVDVSHEDPEVKENWYVDGVEV | | KNQVSLTCLVKGFYPSDIAVEW |
| | HNAKTKPREEQYNSTYRVVSVLTV | | ESNGQPENNYKTTPPVLDSDGS |
| | LHQDWLNGKEYKCKVSNKALPAPI | | FFLYSKLTVDKSRWQQGNVFSC |
| | EKTISKAKGQPREPQVYTLPPSRE | | SVMHEALHNHYTQKSLSLSPG |
| | EMTKNQVSLTCLVKGFYPSDIAVE | | (SEQ ID NO: 53) |
| | WESNGQPENNYKTTPPVLDSDGSF | | |
| | FLYSKLTVDKSRWQQGNVFSCSVM | | |
| | HEALHNHYTQKSLSLSPGGGGGSD | | |
| | CGPPPDIPNARPILGRHSKFAEQS | | |
| | KVAYSCNNGFKQVPDKSNIVVCLE | | |
| | NGQWSSHETFCEKSCVAPERLSFA | | |
| | SLKKEYLNMNFFPVGTIVEYECRP | | |
| | GFRKQPPLPGKATCLEDLVWSPVA | | |
| | QFCKKKSCPNPKDLDNGHINIPTG | | |
| | ILFGSEINFSCNPGYRLVGVSSTF | | |
| | CSVTGNTVDWDDEFPVCTEIHCPE | | |
| | PPKINNGIMRGESDSYTYSQVVTY | | |
| | SCDKGFILVGNASIYCTVSKSDVG | | |
| | QWSSPPPRCIEKSKVPTKKPTINV | | |
| | PSTGTPSTPQKPTTESVPNPGDQP | | |
| | TPQKPSTVKVSATQHVPVTKTTVR | | |
| | HPIRTSTDKGEPNTG (SEQ ID | | |
| | NO: 85) | | |
| CDAB2- | DIQMTQSPSSLSASLGDRVTITCR | DIQMTQSPSSLSASLGDRV | RTVAAPSVFIFPPSDEQLKSGT |
| LC | ASQDFGDYFAWFQQTPGKSPRLLI | TITCRASQDFGDYFAWFQQ | ASVVCLLNNFYPREAKVQWKVD |
| | YGATDLEDGVPSRFSGSRSGSDYS | TPGKSPRLLIYGATDLEDG | NALQSGNSQESVTEQDSKDSTY |
| | LTISSLESEDTAIYYCLQYDKYPL | VPSRFSGSRSGSDYSLTIS | SLSSTLTLSKADYEKHKVYACE |
| | TFGSGTKLEIKRTVAAPSVFIFPP | SLESEDTAIYYCLQYDKYP | VTHQGLSSPVTKSENRGEC |
| | SDEQLKSGTASVVCLLNNFYPREA | LTFGSGTKLEIK (SEQ | (SEQ ID NO: 54) |
| | KVQWKVDNALQSGNSQESVTEQDS | ID NO: 88) | |
| | KDSTYSLSSTLTLSKADYEKHKVY | | |
| | ACEVTHQGLSSPVTKSENRGEC | | |
| | (SEQ ID NO: 87) | | |
| CDAB3 | DCGPPPDIPNARPILGRHSKFAEQ | EVQLVQSGAEVKKPGASVK | (Absent) |
| | SKVAYSCNNGFKQVPDKSNIVVCL | VSCKASGYTFTGYYMHWVR | |
| | ENGQWSSHETFCEKSCVAPERLSF | QAPGQGLEWMGWINPKNGD | |
| | ASLKKEYLNMNFFPVGTIVEYECR | TEFPQKFQGRVTMTRDTSI | |
| | PGFRKQPPLPGKATCLEDLVWSPV | TTAYMDLSRLRSDDTAVYY | |
| | AQFCKKKSCPNPKDLDNGHINIPT | CARESGDDAFDIWGQGTMV | |
| | GILFGSEINFSCNPGYRLVGVSST | TVSS (SEQ ID NO: | |
| | FCSVTGNTVDWDDEFPVCTEIHCP | 82); | |
| | EPPKINNGIMRGESDSYTYSQVVT | DIVMTQSPSSLSASVGDRV | |
| | YSCDKGFILVGNASIYCTVSKSDV | TITCRASQSISSYLNWYQQ | |
| | GQWSSPPPRCIEKSKVPTKKPTIN | KPGKAPKLLIYAASSLQSG | |
| | VPSTGTPSTPQKPTTESVPNPGDQ | VPSRFSGSGSGTDFTLTIS | |
| | PTPQKPSTVKVSATQHVPVTKTTV | SLQPEDFATYYCQQSVGLF | |
| | RHPIRTSTDKGEPNTGGGGGSGGG | FGGGTKVEIK (SEQ ID | |
| | GSGGGGSGGGGSEVQLVQSGAEVK | NO: 84) | |
| | KPGASVKVSCKASGYTFTGYYMHW | | |
| | VRQAPGQGLEWMGWINPKNGDTEF | | |
| | PQKFQGRVTMTRDTSITTAYMDLS | | |
| | RLRSDDTAVYYCARESGDDAFDIW | | |
| | GQGTMVTVSSGGGGGSGGGGSGGG | | |
| | GSDIVMTQSPSSLSASVGDRVTIT | | |
| | CRASQSISSYLNWYQQKPGKAPKL | | |
| | LIYAASSLQSGVPSRFSGSGSGTD | | |
| | FTLTISSLQPEDFATYYCQQSVGL | | |
| | FFGGGTKVEIKHHHHHH (SEQ | | |
| | ID NO: 89) | | |
| CDAB4 | DCGPPPDIPNARPILGRHSKFAEQ | EVQLVESGGGLVQPGRSLK | (Absent) |
| | SKVAYSCNNGFKQVPDKSNIVVCL | LSCAASGFTFSDYYMAWVR | |
| | ENGQWSSHETFCEKSCVAPERLSF | QAPKKGLEWVASISYEGSS | |
| | ASLKKEYLNMNFFPVGTIVEYECR | TNYGDSVKGRFTISRDNAK | |
| | PGFRKQPPLPGKATCLEDLVWSPV | STLYLQMNSLRSEDTATYY | |
| | AQFCKKKSCPNPKDLDNGHINIPT | CARQYHYYSGDGFAYWGQG | |
| | GILFGSEINFSCNPGYRLVGVSST | TLVTVSS (SEQ ID NO: | |
| | FCSVTGNTVDWDDEFPVCTEIHCP | 86); | |
| | EPPKINNGIMRGESDSYTYSQVVT | DIQMTQSPSSLSASLGDRV | |
| | YSCDKGFILVGNASIYCTVSKSDV | TITCRASQDFGDYFAWFQQ | |
| | GQWSSPPPRCIEKSKVPTKKPTIN | TPGKSPRLLIYGATDLEDG | |
| | VPSTGTPSTPQKPTTESVPNPGDQ | VPSRESGSRSGSDYSLTIS | |
| | PTPQKPSTVKVSATQHVPVTKTTV | SLESEDTAIYYCLQYDKYP | |
| | RHPIRTSTDKGEPNTGGGGGSGGG | LTFGSGTKLEIK (SEQ | |
| | GSGGGGSGGGGSEVQLVESGGGLV | ID NO: 88) | |
| | QPGRSLKLSCAASGFTFSDYYMAW | | |
| | VRQAPKKGLEWVASISYEGSSTNY | | |
| | GDSVKGRFTISRDNAKSTLYLQMN | | |
| | SLRSEDTATYYCARQYHYYSGDGF | | |
| | AYWGQGTLVTVSSGGGGSGGGGSG | | |
| | GGGSGGGGSDIQMTQSPSSLSASL | | |
| | GDRVTITCRASQDFGDYFAWFQQT | | |
| | PGKSPRLLIYGATDLEDGVPSRES | | |
| | GSRSGSDYSLTISSLESEDTAIYY | | |
| | CLQYDKYPLTFGSGTKLEIKHHHH | | |
| | HH (SEQ ID NO: 90) | | |
| CDAB5 | LTCYHCFQPVVSSCNMNSTCSPDQ | EVQLVQSGAEVKKPGASVK | (Absent) |
| | DSCLYAVAGMQVYQRCWKQSDCHG | VSCKASGYTFTGYYMHWVR | |
| | EIIMDQLEETKLKFRCCQFNLCNK | QAPGQGLEWMGWINPKNGD | |
| | SGGGGSGGGGSGGGGSGGGGSEVQ | TEFPQKFQGRVTMTRDTSI | |
| | LVQSGAEVKKPGASVKVSCKASGY | TTAYMDLSRLRSDDTAVYY | |
| | TFTGYYMHWVRQAPGQGLEWMGWI | CARESGDDAFDIWGQGTMV | |
| | NPKNGDTEFPQKFQGRVTMTRDTS | TVSS (SEQ ID NO: | |
| | ITTAYMDLSRLRSDDTAVYYCARE | 82); | |
| | SGDDAFDIWGQGTMVTVSSGGGGG | DIVMTQSPSSLSASVGDRV | |
| | SGGGGSGGGGSDIVMTQSPSSLSA | TITCRASQSISSYLNWYQQ | |
| | SVGDRVTITCRASQSISSYLNWYQ | KPGKAPKLLIYAASSLQSG | |
| | QKPGKAPKLLIYAASSLQSGVPSR | VPSRFSGSGSGTDFTLTIS | |
| | FSGSGSGTDFTLTISSLQPEDFAT | SLQPEDFATYYCQQSVGLF | |
| | YYCQQSVGLFFGGGTKVEIKHHHH | FGGGTKVEIK (SEQ ID | |
| | HH (SEQ ID NO: 91) | NO: 84) | |
| CDAB6 | DCGPPPDIPNARPILGRHSKFAEQ | EVQLVQSGAEVKKPGASVK | DKTHTCPPCPAPEAAGAPSVEL |
| | SKVAYSCNNGFKQVPDKSNIVVCL | VSCKASGYTFTGYYMHWVR | FPPKPKDTLMISRTPEVTCVVV |
| | ENGQWSSHETFCEKSCVAPERLSF | QAPGQGLEWMGWINPKNGD | DVSHEDPEVKFNWYVDGVEVHN |
| | ASLKKEYLNMNFFPVGTIVEYECR | TEFPQKFQGRVTMTRDTSI | AKTKPREEQYNSTYRVVSVLTV |
| | PGFRKQPPLPGKATCLEDLVWSPV | TTAYMDLSRLRSDDTAVYY | LHQDWLNGKEYKCKVSNKALPA |
| | AQFCKKKSCPNPKDLDNGHINIPT | CARESGDDAFDIWGQGTMV | PIEKTISKAKGQPREPQVYTLP |
| | GILFGSEINFSCNPGYRLVGVSST | TVSS (SEQ ID NO: | PSREEMTKNQVSLTCLVKGFYP |
| | FCSVTGNTVDWDDEFPVCTEIHCP | 82); | SDIAVEWESNGQPENNYKTTPP |
| | EPPKINNGIMRGESDSYTYSQVVT | DIVMTQSPSSLSASVGDRV | VLDSDGSFFLYSKLTVDKSRWQ |
| | YSCDKGFILVGNASIYCTVSKSDV | TITCRASQSISSYLNWYQQ | QGNVFSCSVMHEALHNHYTOKS |
| | GQWSSPPPRCIEKSKVPTKKPTIN | KPGKAPKLLIYAASSLQSG | LSLSPG (SEQ ID NO: 51) |
| | VPSTGTPSTPQKPTTESVPNPGDQ | VPSRFSGSGSGTDFTLTIS | |
| | PTPQKPSTVKVSATQHVPVTKTTV | SLQPEDFATYYCQQSVGLF | |
| | RHPIRTSTDKGEPNTGGGGGSGGG | FGGGTKVEIK (SEQ ID | |
| | GSGGGGSGGGGSDKTHTCPPCPAP | NO: 84) | |
| | EAAGAPSVFLFPPKPKDTLMISRT | | |
| | PEVTCVVVDVSHEDPEVKFNWYVD | | |
| | GVEVHNAKTKPREEQYNSTYRVVS | | |
| | VLTVLHQDWLNGKEYKCKVSNKAL | | |
| | PAPIEKTISKAKGQPREPQVYTLP | | |
| | PSREEMTKNQVSLTCLVKGFYPSD | | |
| | IAVEWESNGQPENNYKTTPPVLDS | | |
| | DGSFFLYSKLTVDKSRWQQGNVES | | |
| | CSVMHEALHNHYTQKSLSLSPGGG | | |
| | GGSGGGGSGGGGSGGGGSEVQLVQ | | |
| | SGAEVKKPGASVKVSCKASGYTFT | | |
| | GYYMHWVRQAPGQGLEWMGWINPK | | |
| | NGDTEFPQKFQGRVTMTRDTSITT | | |
| | AYMDLSRLRSDDTAVYYCARESGD | | |
| | DAFDIWGQGTMVTVSSGGGGGSGG | | |
| | GGSGGGGSDIVMTQSPSSLSASVG | | |
| | DRVTITCRASQSISSYLNWYQQKP | | |
| | GKAPKLLIYAASSLQSGVPSRFSG | | |
| | SGSGTDFTLTISSLQPEDFATYYC | | |
| | QQSVGLFFGGGTKVEIK (SEQ | | |
| | ID NO: 92) | | |
| CDAB7 | LTCYHCFQPVVSSCNMNSTCSPDQ | EVQLVQSGAEVKKPGASVK | DKTHTCPPCPAPEAAGAPSVFL |
| | DSCLYAVAGMQVYQRCWKQSDCHG | VSCKASGYTFTGYYMHWVR | FPPKPKDTLMISRTPEVTCVVV |
| | EIIMDQLEETKLKFRCCQFNLCNK | QAPGQGLEWMGWINPKNGD | DVSHEDPEVKFNWYVDGVEVHN |
| | SGGGGSGGGGSGGGGSGGGGSDKT | TEFPQKFQGRVTMTRDTSI | AKTKPREEQYNSTYRVVSVLTV |
| | HTCPPCPAPEAAGAPSVELFPPKP | TTAYMDLSRLRSDDTAVYY | LHQDWLNGKEYKCKVSNKALPA |
| | KDTLMISRTPEVTCVVVDVSHEDP | CARESGDDAFDIWGQGTMV | PIEKTISKAKGQPREPQVYTLP |
| | EVKFNWYVDGVEVHNAKTKPREEQ | TVSS (SEQ ID NO: | PSREEMTKNQVSLTCLVKGFYP |
| | YNSTYRVVSVLTVLHQDWLNGKEY | 82); | SDIAVEWESNGQPENNYKTTPP |
| | KCKVSNKALPAPIEKTISKAKGQP | DIVMTQSPSSLSASVGDRV | VLDSDGSFFLYSKLTVDKSRWQ |
| | REPQVYTLPPSREEMTKNQVSLTC | TITCRASQSISSYLNWYQQ | QGNVFSCSVMHEALHNHYTQKS |
| | LVKGFYPSDIAVEWESNGQPENNY | KPGKAPKLLIYAASSLQSG | LSLSPG (SEQ ID NO: 51) |
| | KTTPPVLDSDGSFFLYSKLTVDKS | VPSRFSGSGSGTDFTLTIS | |
| | RWQQGNVFSCSVMHEALHNHYTQK | SLQPEDFATYYCQQSVGLF | |
| | SLSLSPGGGGGSGGGGSGGGGSGG | FGGGTKVEIK (SEQ ID | |
| | GGSEVQLVQSGAEVKKPGASVKVS | NO: 84) | |
| | CKASGYTFTGYYMHWVRQAPGQGL | | |
| | EWMGWINPKNGDTEFPQKFQGRVT | | |
| | MTRDTSITTAYMDLSRLRSDDTAV | | |
| | YYCARESGDDAFDIWGQGTMVTVS | | |
| | SGGGGGSGGGGSGGGGSDIVMTQS | | |
| | PSSLSASVGDRVTITCRASQSISS | | |
| | YLNWYQQKPGKAPKLLIYAASSLQ | | |
| | SGVPSRFSGSGSGTDFTLTISSLQ | | |
| | PEDFATYYCQQSVGLFFGGGTKVE | | |
| | IK (SEQ ID NO: 93) | | |
| CDAB8- | EVQLVQSGAEVKKPGASVKVSCKA | EVQLVQSGAEVKKPGASVK | ASTKGPSVFPLAPSSKSTSGGT |
| HC | SGYTFTGYYMHWVRQAPGQGLEWM | VSCKASGYTFTGYYMHWVR | AALGCLVKDYFPEPVTVSWNSG |
| | GWINPKNGDTEFPQKFQGRVTMTR | QAPGQGLEWMGWINPKNGD | ALTSGVHTFPAVLQSSGLYSLS |
| | DTSITTAYMDLSRLRSDDTAVYYC | TEFPQKFQGRVTMTRDTSI | SVVTVPSSSLGTQTYICNVNHK |
| | ARESGDDAFDIWGQGTMVTVSSAS | TTAYMDLSRLRSDDTAVYY | PSNTKVDKKVEPKSCDKTHTCP |
| | TKGPSVFPLAPSSKSTSGGTAALG | CARESGDDAFDIWGQGTMV | PCPAPEAAGAPSVFLFPPKPKD |
| | CLVKDYFPEPVTVSWNSGALTSGV | TVSS (SEQ ID NO: | TLMISRTPEVTCVVVDVSHEDP |
| | HTFPAVLQSSGLYSLSSVVTVPSS | 82) | EVKFNWYVDGVEVHNAKTKPRE |
| | SLGTQTYICNVNHKPSNTKVDKKV | | EQYNSTYRVVSVLTVLHQDWLN |
| | EPKSCDKTHTCPPCPAPEAAGAPS | | GKEYKCKVSNKALPAPIEKTIS |
| | VFLFPPKPKDTLMISRTPEVTCVV | | KAKGQPREPQVYTLPPSREEMT |
| | VDVSHEDPEVKFNWYVDGVEVHNA | | KNQVSLTCLVKGFYPSDIAVEW |
| | KTKPREEQYNSTYRVVSVLTVLHQ | | ESNGQPENNYKTTPPVLDSDGS |
| | DWLNGKEYKCKVSNKALPAPIEKT | | FFLYSKLTVDKSRWQQGNVFSC |
| | ISKAKGQPREPQVYTLPPSREEMT | | SVMHEALHNHYTQKSLSLSPG |
| | KNQVSLTCLVKGFYPSDIAVEWES | | (SEQ ID NO: 53) |
| | NGQPENNYKTTPPVLDSDGSFFLY | | |
| | SKLTVDKSRWQQGNVFSCSVMHEA | | |
| | LHNHYTQKSLSLSPGGGGGSDCGL | | |
| | PPDVPNAQPALEGRTSFPEDTVIT | | |
| | YKCEESFVKIPGEKDSVICLKGSQ | | |
| | WSDIEEFCNRSCEVPTRLNSASLK | | |
| | QPYITQNYFPVGTVVEYECRPGYR | | |
| | REPSLSPKLTCLQNLKWSTAVEFC | | |
| | KKKSCPNPGEIRNGQIDVPGGILF | | |
| | GATISFSCNTGYKLFGSTSSFCLI | | |
| | SGSSVQWSDPLPECREIYCPAPPQ | | |
| | IDNGIIQGERDHYGYRQSVTYACN | | |
| | KGFTMIGEHSIYCTVNNDEGEWSG | | |
| | PPPECRGKSLTSKVPPTVQKPTTV | | |
| | NVPTTEVSPTSQKTTTKTTTPNAQ | | |
| | ATRSTPVSRTTKHFHETTPNKGSG | | |
| | TTS (SEQ ID NO: 94) | | |
| CDAB9- | EVQLVQSGAEVKKPGASVKVSCKA | EVQLVQSGAEVKKPGASVK | ASTKGPSVFPLAPSSKSTSGGT |
| HC | SGYTFTGYYMHWVRQAPGQGLEWM | VSCKASGYTFTGYYMHWVR | AALGCLVKDYFPEPVTVSWNSG |
| | GWINPKNGDTEFPQKFQGRVTMTR | QAPGQGLEWMGWINPKNGD | ALTSGVHTFPAVLQSSGLYSLS |
| | DTSITTAYMDLSRLRSDDTAVYYC | TEFPQKFQGRVTMTRDTSI | SVVTVPSSSLGTQTYICNVNHK |
| | ARESGDDAFDIWGQGTMVTVSSAS | TTAYMDLSRLRSDDTAVYY | PSNTKVDKKVEPKSCDKTHTCP |
| | TKGPSVFPLAPSSKSTSGGTAALG | CARESGDDAFDIWGQGTMV | PCPAPEAAGAPSVFLFPPKPKD |
| | CLVKDYFPEPVTVSWNSGALTSGV | TVSS (SEQ ID NO: | TLMISRTPEVTCVVVDVSHEDP |
| | HTFPAVLQSSGLYSLSSVVTVPSS | 82) | EVKFNWYVDGVEVHNAKTKPRE |
| | SLGTQTYICNVNHKPSNTKVDKKV | | EQYNSTYRVVSVLTVLHQDWLN |
| | EPKSCDKTHTCPPCPAPEAAGAPS | | GKEYKCKVSNKALPAPIEKTIS |
| | VFLFPPKPKDTLMISRTPEVTCVV | | KAKGQPREPQVYTLPPSREEMT |
| | VDVSHEDPEVKFNWYVDGVEVHNA | | KNQVSLTCLVKGFYPSDIAVEW |
| | KTKPREEQYNSTYRVVSVLTVLHQ | | ESNGQPENNYKTTPPVLDSDGS |
| | DWLNGKEYKCKVSNKALPAPIEKT | | FFLYSKLTVDKSRWQQGNVFSC |
| | ISKAKGQPREPQVYTLPPSREEMT | | SVMHEALHNHYTQKSLSLSPG |
| | KNQVSLTCLVKGFYPSDIAVEWES | | (SEQ ID NO: 53) |
| | NGQPENNYKTTPPVLDSDGSFFLY | | |
| | SKLTVDKSRWQQGNVFSCSVMHEA | | |
| | LHNHYTQKSLSLSPGGGGGSDCGL | | |
| | PPDVPNAQPALEGRTSFPEDTVIT | | |
| | YKCEESFVKIPGEKDSVICLKGSQ | | |
| | WSDIEEFCNRSCEVPTRLNSASLK | | |
| | QPYITQNYFPVGTVVEYECRPGYR | | |
| | REPSLSPKLTCLQNLKWSTAVEFC | | |
| | KKKSCPNPGEIRNGQIDVPGGILF | | |
| | GATISFSCNTGYKLFGSTSSFCLI | | |
| | SGSSVQWSDPLPECREIYCPAPPQ | | |
| | IDNGIIQGERDHYGYRQSVTYACN | | |
| | KGFTMIGEHSIYCTVNNDEGEWSG | | |
| | PPPECRG (SEQ ID NO: 95) | | |
| CDAB10- | EVQLVQSGAEVKKPGASVKVSCKA | EVQLVQSGAEVKKPGASVK | ASTKGPSVFPLAPSSKSTSGGT |
| HC | SGYTFTGYYMHWVRQAPGQGLEWM | VSCKASGYTFTGYYMHWVR | AALGCLVKDYFPEPVTVSWNSG |
| | GWINPKNGDTEFPQKFQGRVTMTR | QAPGQGLEWMGWINPKNGD | ALTSGVHTFPAVLQSSGLYSLS |
| | DTSITTAYMDLSRLRSDDTAVYYC | TEFPQKFQGRVTMTRDTSI | SVVTVPSSSLGTQTYICNVNHK |
| | ARESGDDAFDIWGQGTMVTVSSAS | TTAYMDLSRLRSDDTAVYY | PSNTKVDKKVEPKSCDKTHTCP |
| | TKGPSVFPLAPSSKSTSGGTAALG | CARESGDDAFDIWGQGTMV | PCPAPEAAGAPSVFLFPPKPKD |
| | CLVKDYFPEPVTVSWNSGALTSGV | TVSS (SEQ ID NO: | TLMISRTPEVTCVVVDVSHEDP |
| | HTFPAVLQSSGLYSLSSVVTVPSS | 82) | EVKFNWYVDGVEVHNAKTKPRE |
| | SLGTQTYICNVNHKPSNTKVDKKV | | EQYNSTYRVVSVLTVLHQDWLN |
| | EPKSCDKTHTCPPCPAPEAAGAPS | | GKEYKCKVSNKALPAPIEKTIS |
| | VFLFPPKPKDTLMISRTPEVTCVV | | KAKGQPREPQVYTLPPSREEMT |
| | VDVSHEDPEVKFNWYVDGVEVHNA | | KNQVSLTCLVKGFYPSDIAVEW |
| | KTKPREEQYNSTYRVVSVLTVLHQ | | ESNGQPENNYKTTPPVLDSDGS |
| | DWLNGKEYKCKVSNKALPAPIEKT | | FFLYSKLTVDKSRWQQGNVFSC |
| | ISKAKGQPREPQVYTLPPSREEMT | | SVMHEALHNHYTQKSLSLSPG |
| | KNQVSLTCLVKGFYPSDIAVEWES | | (SEQ ID NO: 53) |
| | NGQPENNYKTTPPVLDSDGSFFLY | | |
| | SKLTVDKSRWQQGNVFSCSVMHEA | | |
| | LHNHYTQKSLSLSPGGGGGSDCGL | | |
| | PPDVPNAQPALEGRTSFPEDTVIT | | |
| | YKCEESFVKIPGEKDSVICLKGSQ | | |
| | WSDIEEFCQRSCEVPTRLNSASLK | | |
| | QPYITQNYFPVGTVVEYECRPGYR | | |
| | REPSLSPKLTCLQNLKWSTAVEFC | | |
| | KKKSCPNPGEIRNGQIDVPGGILF | | |
| | GATISFSCNTGYKLFGSTSSFCLI | | |
| | SGSSVQWSDPLPECREIYCPAPPQ | | |
| | IDNGIIQGERDHYGYRQSVTYACN | | |
| | KGFTMIGEHSIYCTVNNDEGEWSG | | |
| | PPPECRG (SEQ ID NO: 96) | | |
| CDAB11 | EVQLVESGGGWQPGGSLRLSCAAS | EVQLVESGGGWQPGGSLRL | (Absent) |
| | ISIFDIYAMHWYRQAPGKQRELVA | SCAASISIFDIYAMHWYRQ | |
| | TSFRDGSTYYADSVKGRFTISRDN | APGKQRELVATSFRDGSTY | |
| | SKNTLYLQMNSLRAEDTAVYLCHV | YADSVKGRFTISRDNSKNT | |
| | SLYRDPLGVAGGMGVYWGKGALVT | LYLQMNSLRAEDTAVYLCH | |
| | VSSGGGGSDCGLPPDVPNAQPALE | VSLYRDPLGVAGGMGVYWG | |
| | GRTSFPEDTVITYKCEESFVKIPG | KGALVTVSS (SEQ ID | |
| | EKDSVICLKGSQWSDIEEFCNRSC | NO: 98) | |
| | EVPTRLNSASLKQPYITQNYFPVG | | |
| | TVVEYECRPGYRREPSLSPKLTCL | | |
| | QNLKWSTAVEFCKKKSCPNPGEIR | | |
| | NGQIDVPGGILFGATISFSCNTGY | | |
| | KLFGSTSSFCLISGSSVQWSDPLP | | |
| | ECREIYCPAPPQIDNGIIQGERDH | | |
| | YGYRQSVTYACNKGFTMIGEHSIY | | |
| | CTVNNDEGEWSGPPPECRGKSLTS | | |
| | KVPPTVQKPTTVNVPTTEVSPTSQ | | |
| | KTTTKTTTPNAQATRSTPVSRTTK | | |
| | HFHETTPNKGSGTTSHHHHHH | | |
| | (SEQ ID NO: 97) | | |
| CDAB12 | EVQLVQSGAEVKKPGASVKVSCKA | EVQLVQSGAEVKKPGASVK | ASTKGPSVFPLAPSSKSTSGGT |
| | SGYTFTGYYMHWVRQAPGQGLEWM | VSCKASGYTFTGYYMHWVR | AALGCLVKDYFPEPVTVSWNSG |
| | GWINPKNGDTEFPQKFQGRVTMTR | QAPGQGLEWMGWINPKNGD | ALTSGVHTFPAVLQSSGLYSLS |
| | DTSITTAYMDLSRLRSDDTAVYYC | TEFPQKFQGRVTMTRDTSI | SVVTVPSSSLGTQTYICNVNHK |
| | ARESGDDAFDIWGQGTMVTVSSAS | TTAYMDLSRLRSDDTAVYY | PSNTKVDKKVEPKSCDKTHTCP |
| | TKGPSVFPLAPSSKSTSGGTAALG | CARESGDDAFDIWGQGTMV | PCPAPEAAGAPSVFLFPPKPKD |
| | CLVKDYFPEPVTVSWNSGALTSGV | TVSS (SEQ ID NO: | TLMISRTPEVTCVVVDVSHEDP |
| | HTFPAVLQSSGLYSLSSVVTVPSS | 82) | EVKFNWYVDGVEVHNAKTKPRE |
| | SLGTQTYICNVNHKPSNTKVDKKV | | EQYNSTYRVVSVLTVLHQDWLN |
| | EPKSCDKTHTCPPCPAPEAAGAPS | | GKEYKCKVSNKALPAPIEKTIS |
| | VFLFPPKPKDTLMISRTPEVTCVV | | KAKGQPREPQVYTLPPSREEMT |
| | VDVSHEDPEVKFNWYVDGVEVHNA | | KNQVSLTCLVKGFYPSDIAVEW |
| | KTKPREEQYNSTYRVVSVLTVLHQ | | ESNGQPENNYKTTPPVLDSDGS |
| | DWLNGKEYKCKVSNKALPAPIEKT | | FFLYSKLTVDKSRWQQGNVFSC |
| | ISKAKGQPREPQVYTLPPSREEMT | | SVMHEALHNHYTQKSLSLSPG |
| | KNQVSLTCLVKGFYPSDIAVEWES | | (SEQ ID NO: 53) |
| | NGQPENNYKTTPPVLDSDGSFFLY | | |
| | SKLTVDKSRWQQGNVFSCSVMHEA | | |
| | LHNHYTQKSLSLSPGGGGGSSCEV | | |
| | PTRLNSASLKQPYITQNYFPVGTV | | |
| | VEYECRPGYRREPSLSPKLTCLQN | | |
| | LKWSTAVEFCKKKSCPNPGEIRNG | | |
| | QIDVPGGILFGATISFSCNTGYKL | | |
| | IGSYSSFCKISGSSVQWSDKLPIC | | |
| | EKVLCTPPPKIKNGKHTFSEVEVF | | |
| | EYLDAVTYSCDPAPGPDPFSLIGE | | |
| | STIYCGDNSVWSRAAPECKVVKCR | | |
| | FPVVENGKQISGFGKKFYYKATVM | | |
| | FECDKGFYLDGSDTIVCDSNSTWD | | |
| | PPVPKCLKV (SEQ ID NO: | | |
| | 99) | | |
| CDAB13- | QVQLLESGPGLVKPSGTLSLTCTV | QVQLLESGPGLVKPSGTLS | ASTKGPSVFPLAPSSKSTSGGT |
| HC | SGGSISSTNWWTWVRQSPGTGLEW | LTCTVSGGSISSTNWWTWV | AALGCLVKDYFPEPVTVSWNSG |
| | IGHIYHSGSTDYNPSLKSRVTISI | RQSPGTGLEWIGHIYHSGS | ALTSGVHTFPAVLQSSGLYSLS |
| | DKSKNQFSLKMTSVTAADTAVYYC | TDYNPSLKSRVTISIDKSK | SVVTVPSSSLGTQTYICNVNHK |
| | ACAAQYHWKGLDPWGHGTLVTVSS | NQFSLKMTSVTAADTAVYY | PSNTKVDKKVEPKSCDKTHTCP |
| | ASTKGPSVFPLAPSSKSTSGGTAA | CACAAQYHWKGLDPWGHGT | PCPAPEAAGAPSVELFPPKPKD |
| | LGCLVKDYFPEPVTVSWNSGALTS | LVTVSS (SEQ ID NO: | TLMISRTPEVTCVVVDVSHEDP |
| | GVHTFPAVLQSSGLYSLSSVVTVP | 101) | EVKFNWYVDGVEVHNAKTKPRE |
| | SSSLGTQTYICNVNHKPSNTKVDK | | EQYNSTYRVVSVLTVLHQDWLN |
| | KVEPKSCDKTHTCPPCPAPEAAGA | | GKEYKCKVSNKALPAPIEKTIS |
| | PSVFLFPPKPKDTLMISRTPEVTC | | KAKGQPREPQVYTLPPSREEMT |
| | VVVDVSHEDPEVKFNWYVDGVEVH | | KNQVSLTCLVKGFYPSDIAVEW |
| | NAKTKPREEQYNSTYRVVSVLTVL | | ESNGQPENNYKTTPPVLDSDGS |
| | HQDWLNGKEYKCKVSNKALPAPIE | | FFLYSKLTVDKSRWQQGNVFSC |
| | KTISKAKGQPREPQVYTLPPSREE | | SVMHEALHNHYTQKSLSLSPG |
| | MTKNQVSLTCLVKGFYPSDIAVEW | | (SEQ ID NO: 53) |
| | ESNGQPENNYKTTPPVLDSDGSFF | | |
| | LYSKLTVDKSRWQQGNVFSCSVMH | | |
| | EALHNHYTQKSLSLSPGGGGGSDC | | |
| | GPPPDIPNARPILGRHSKFAEQSK | | |
| | VAYSCNNGFKQVPDKSNIVVCLEN | | |
| | GQWSSHETFCEKSCVAPERLSFAS | | |
| | LKKEYLNMNFFPVGTIVEYECRPG | | |
| | FRKQPPLPGKATCLEDLVWSPVAQ | | |
| | FCKKKSCPNPKDLDNGHINIPTGI | | |
| | LFGSEINFSCNPGYRLVGVSSTFC | | |
| | SVTGNTVDWDDEFPVCTEIHCPEP | | |
| | PKINNGIMRGESDSYTYSQVVTYS | | |
| | CDKGFILVGNASIYCTVSKSDVGQ | | |
| | WSSPPPRCIEKSKVPTKKPTINVP | | |
| | STGTPSTPQKPTTESVPNPGDQPT | | |
| | PQKPSTVKVSATQHVPVTKTTVRH | | |
| | PIRTSTDKGEPNTG (SEQ ID | | |
| | NO: 100) | | |
| CDAB13- | DIVMTQTPLSLSVTPGQPASISCM | DIVMTQTPLSLSVTPGQPA | RTVAAPSVFIFPPSDEQLKSGT |
| LC | SSQSLLHSDGKTYLYWYLQKPGQP | SISCMSSQSLLHSDGKTYL | ASVVCLLNNFYPREAKVQWKVD |
| | PQLLIYEVSNRFSGVPDRESGSGS | YWYLQKPGQPPQLLIYEVS | NALQSGNSQESVTEQDSKDSTY |
| | GTDFTLKISRVEAEDVGIYYCMQS | NRFSGVPDRFSGSGSGTDF | SLSSTLTLSKADYEKHKVYACE |
| | IQLPITFGQGTRLEIKRTVAAPSV | TLKISRVEAEDVGIYYCMQ | VTHQGLSSPVTKSENRGEC |
| | FIFPPSDEQLKSGTASVVCLLNNE | SIQLPITFGQGTRLEIK | (SEQ ID NO: 54) |
| | YPREAKVQWKVDNALQSGNSQESV | (SEQ ID NO: 103) | |
| | TEQDSKDSTYSLSSTLTLSKADYE | | |
| | KHKVYACEVTHQGLSSPVTKSENR | | |
| | GEC (SEQ ID NO: 102) | | |
| CDAB14 | DCGPPPDIPNARPILGRHSKFAEQ | QVQLLESGPGLVKPSGTLS | (Absent) |
| | SKVAYSCNNGFKQVPDKSNIVVCL | LTCTVSGGSISSTNWWTWV | |
| | ENGQWSSHETFCEKSCVAPERLSF | RQSPGTGLEWIGHIYHSGS | |
| | ASLKKEYLNMNFFPVGTIVEYECR | TDYNPSLKSRVTISIDKSK | |
| | PGFRKQPPLPGKATCLEDLVWSPV | NQFSLKMTSVTAADTAVYY | |
| | AQFCKKKSCPNPKDLDNGHINIPT | CACAAQYHWKGLDPWGHGT | |
| | GILFGSEINFSCNPGYRLVGVSST | LVTVSS (SEQ ID NO: | |
| | FCSVTGNTVDWDDEFPVCTEIHCP | 101); | |
| | EPPKINNGIMRGESDSYTYSQVVT | DIVMTQTPLSLSVTPGQPA | |
| | YSCDKGFILVGNASIYCTVSKSDV | SISCMSSQSLLHSDGKTYL | |
| | GQWSSPPPRCIEKSKVPTKKPTIN | YWYLQKPGQPPQLLIYEVS | |
| | VPSTGTPSTPQKPTTESVPNPGDQ | NRFSGVPDRFSGSGSGTDF | |
| | PTPQKPSTVKVSATQHVPVTKTTV | TLKISRVEAEDVGIYYCMQ | |
| | RHPIRTSTDKGEPNTGGGGGSGGG | SIQLPITFGQGTRLEIK | |
| | GSGGGGSGGGGSQVQLLESGPGLV | (SEQ ID NO: 103) | |
| | KPSGTLSLTCTVSGGSISSTNWWT | | |
| | WVRQSPGTGLEWIGHIYHSGSTDY | | |
| | NPSLKSRVTISIDKSKNQFSLKMT | | |
| | SVTAADTAVYYCACAAQYHWKGLD | | |
| | PWGHGTLVTVSSGGGGGSGGGGSG | | |
| | GGGSDIVMTQTPLSLSVTPGQPAS | | |
| | ISCMSSQSLLHSDGKTYLYWYLQK | | |
| | PGQPPQLLIYEVSNRFSGVPDRES | | |
| | GSGSGTDFTLKISRVEAEDVGIYY | | |
| | CMQSIQLPITFGQGTRLEIKHHHH | | |
| | HH (SEQ ID NO: 104) | | |
| CDAB15 | LTCYHCFQPVVSSCNMNSTCSPDQ | QVQLLESGPGLVKPSGTLS | (Absent) |
| | DSCLYAVAGMQVYQRCWKQSDCHG | LTCTVSGGSISSTNWWTWV | |
| | EIIMDQLEETKLKERCCQFNLCNK | RQSPGTGLEWIGHIYHSGS | |
| | SGGGGSGGGGSGGGGSGGGGSQVQ | TDYNPSLKSRVTISIDKSK | |
| | LLESGPGLVKPSGTLSLTCTVSGG | NQFSLKMTSVTAADTAVYY | |
| | SISSTNWWTWVRQSPGTGLEWIGH | CACAAQYHWKGLDPWGHGT | |
| | IYHSGSTDYNPSLKSRVTISIDKS | LVTVSS (SEQ ID NO: | |
| | KNQFSLKMTSVTAADTAVYYCACA | 101); | |
| | AQYHWKGLDPWGHGTLVTVSSGGG | DIVMTQTPLSLSVTPGQPA | |
| | GGSGGGGSGGGGSDIVMTQTPLSL | SISCMSSQSLLHSDGKTYL | |
| | SVTPGQPASISCMSSQSLLHSDGK | YWYLQKPGQPPQLLIYEVS | |
| | TYLYWYLQKPGQPPQLLIYEVSNR | NRFSGVPDRFSGSGSGTDF | |
| | FSGVPDRFSGSGSGTDFTLKISRV | TLKISRVEAEDVGIYYCMQ | |
| | EAEDVGIYYCMQSIQLPITFGQGT | SIQLPITFGQGTRLEIK | |
| | RLEIKHHHHHH (SEQ ID NO: | (SEQ ID NO: 103) | |
| | 105) | | |
| CDAB16 | DCGPPPDIPNARPILGRHSKFAEQ | QVQLLESGPGLVKPSGTLS | DKTHTCPPCPAPEAAGAPSVEL |
| | SKVAYSCNNGFKQVPDKSNIVVCL | LTCTVSGGSISSTNWWTWV | FPPKPKDTLMISRTPEVTCVVV |
| | ENGQWSSHETFCEKSCVAPERLSF | RQSPGTGLEWIGHIYHSGS | DVSHEDPEVKFNWYVDGVEVHN |
| | ASLKKEYLNMNFFPVGTIVEYECR | TDYNPSLKSRVTISIDKSK | AKTKPREEQYNSTYRVVSVLTV |
| | PGFRKQPPLPGKATCLEDLVWSPV | NQFSLKMTSVTAADTAVYY | LHQDWLNGKEYKCKVSNKALPA |
| | AQFCKKKSCPNPKDLDNGHINIPT | CACAAQYHWKGLDPWGHGT | PIEKTISKAKGQPREPQVYTLP |
| | GILFGSEINFSCNPGYRLVGVSST | LVTVSS (SEQ ID NO: | PSREEMTKNQVSLTCLVKGFYP |
| | FCSVTGNTVDWDDEFPVCTEIHCP | 101); | SDIAVEWESNGQPENNYKTTPP |
| | EPPKINNGIMRGESDSYTYSQVVT | DIVMTQTPLSLSVTPGQPA | VLDSDGSFFLYSKLTVDKSRWQ |
| | YSCDKGFILVGNASIYCTVSKSDV | SISCMSSQSLLHSDGKTYL | QGNVFSCSVMHEALHNHYTOKS |
| | GQWSSPPPRCIEKSKVPTKKPTIN | YWYLQKPGQPPQLLIYEVS | LSLSPG (SEQ ID NO: 51) |
| | VPSTGTPSTPQKPTTESVPNPGDQ | NRFSGVPDRFSGSGSGTDF | |
| | PTPQKPSTVKVSATQHVPVTKTTV | TLKISRVEAEDVGIYYCMQ | |
| | RHPIRTSTDKGEPNTGGGGGSGGG | SIQLPITFGQGTRLEIK | |
| | GSGGGGSGGGGSDKTHTCPPCPAP | (SEQ ID NO: 103) | |
| | EAAGAPSVFLFPPKPKDTLMISRT | | |
| | PEVTCVVVDVSHEDPEVKENWYVD | | |
| | GVEVHNAKTKPREEQYNSTYRVVS | | |
| | VLTVLHQDWLNGKEYKCKVSNKAL | | |
| | PAPIEKTISKAKGQPREPQVYTLP | | |
| | PSREEMTKNQVSLTCLVKGFYPSD | | |
| | IAVEWESNGQPENNYKTTPPVLDS | | |
| | DGSFFLYSKLTVDKSRWQQGNVFS | | |
| | CSVMHEALHNHYTQKSLSLSPGGG | | |
| | GSGGGGSGGGGSGGGGSQVQLLES | | |
| | GPGLVKPSGTLSLTCTVSGGSISS | | |
| | TNWWTWVRQSPGTGLEWIGHIYHS | | |
| | GSTDYNPSLKSRVTISIDKSKNQF | | |
| | SLKMTSVTAADTAVYYCACAAQYH | | |
| | WKGLDPWGHGTLVTVSSGGGGGSG | | |
| | GGGSGGGGSDIVMTQTPLSLSVTP | | |
| | GQPASISCMSSQSLLHSDGKTYLY | | |
| | WYLQKPGQPPQLLIYEVSNRFSGV | | |
| | PDRFSGSGSGTDFTLKISRVEAED | | |
| | VGIYYCMQSIQLPITFGQGTRLEI | | |
| | K (SEQ ID NO: 106) | | |
| CDAB17 | LTCYHCFQPVVSSCNMNSTCSPDQ | QVQLLESGPGLVKPSGTLS | DKTHTCPPCPAPEAAGAPSVEL |
| | DSCLYAVAGMQVYQRCWKQSDCHG | LTCTVSGGSISSTNWWTWV | FPPKPKDTLMISRTPEVTCVVV |
| | EIIMDQLEETKLKFRCCQFNLCNK | RQSPGTGLEWIGHIYHSGS | DVSHEDPEVKFNWYVDGVEVHN |
| | SGGGGSGGGGSGGGGSGGGGSDKT | TDYNPSLKSRVTISIDKSK | AKTKPREEQYNSTYRVVSVLTV |
| | HTCPPCPAPEAAGAPSVFLFPPKP | NQFSLKMTSVTAADTAVYY | LHQDWLNGKEYKCKVSNKALPA |
| | KDTLMISRTPEVTCVVVDVSHEDP | CACAAQYHWKGLDPWGHGT | PIEKTISKAKGQPREPQVYTLP |
| | EVKFNWYVDGVEVHNAKTKPREEQ | LVTVSS (SEQ ID NO: | PSREEMTKNQVSLTCLVKGFYP |
| | YNSTYRVVSVLTVLHQDWLNGKEY | 101); | SDIAVEWESNGQPENNYKTTPP |
| | KCKVSNKALPAPIEKTISKAKGQP | DIVMTQTPLSLSVTPGQPA | VLDSDGSFFLYSKLTVDKSRWQ |
| | REPQVYTLPPSREEMTKNQVSLTC | SISCMSSQSLLHSDGKTYL | QGNVFSCSVMHEALHNHYTQKS |
| | LVKGFYPSDIAVEWESNGQPENNY | YWYLQKPGQPPQLLIYEVS | LSLSPG (SEQ ID NO: 51) |
| | KTTPPVLDSDGSFFLYSKLTVDKS | NRFSGVPDRFSGSGSGTDF | |
| | RWQQGNVFSCSVMHEALHNHYTQK | TLKISRVEAEDVGIYYCMQ | |
| | SLSLSPGGGGSGGGGSGGGGSGGG | SIQLPITFGQGTRLEIK | |
| | GSQVQLLESGPGLVKPSGTLSLTC | (SEQ ID NO: 103) | |
| | TVSGGSISSTNWWTWVRQSPGTGL | | |
| | EWIGHIYHSGSTDYNPSLKSRVTI | | |
| | SIDKSKNQFSLKMTSVTAADTAVY | | |
| | YCACAAQYHWKGLDPWGHGTLVTV | | |
| | SSGGGGGSGGGGSGGGGSDIVMTQ | | |
| | TPLSLSVTPGQPASISCMSSQSLL | | |
| | HSDGKTYLYWYLQKPGQPPQLLIY | | |
| | EVSNRFSGVPDRFSGSGSGTDFTL | | |
| | KISRVEAEDVGIYYCMQSIQLPIT | | |
| | FGQGTRLEIK (SEQ ID NO: | | |
| | 107) | | |
| CDAB18- | EVQLLESGGGLVQPGGSLRLSCAA | EVQLLESGGGLVQPGGSLR | ASTKGPSVFPLAPSSKSTSGGT |
| HC | SGFTFSSYAMSWVRQAPGKGLEWV | LSCAASGFTFSSYAMSWVR | AALGCLVKDYFPEPVTVSWNSG |
| | SAISGSGGSTYYADSVKGRFTISR | QAPGKGLEWVSAISGSGGS | ALTSGVHTFPAVLQSSGLYSLS |
| | DNSKNTLYLQMNSLRAEDTAVYYC | TYYADSVKGRFTISRDNSK | SVVTVPSSSLGTQTYICNVNHK |
| | AKDWYYDFWSGYPGPDYYGMDVWG | NTLYLQMNSLRAEDTAVYY | PSNTKVDKKVEPKSCDKTHTCP |
| | QGTTVTVSSASTKGPSVFPLAPSS | CAKDWYYDFWSGYPGPDYY | PCPAPEAAGAPSVFLFPPKPKD |
| | KSTSGGTAALGCLVKDYFPEPVTV | GMDVWGQGTTVTVSS | TLMISRTPEVTCVVVDVSHEDP |
| | SWNSGALTSGVHTFPAVLQSSGLY | (SEQ ID NO: 109) | EVKFNWYVDGVEVHNAKTKPRE |
| | SLSSVVTVPSSSLGTQTYICNVNH | | EQYNSTYRVVSVLTVLHQDWLN |
| | KPSNTKVDKKVEPKSCDKTHTCPP | | GKEYKCKVSNKALPAPIEKTIS |
| | CPAPEAAGAPSVFLFPPKPKDTLM | | KAKGQPREPQVYTLPPSREEMT |
| | ISRTPEVTCVVVDVSHEDPEVKEN | | KNQVSLTCLVKGFYPSDIAVEW |
| | WYVDGVEVHNAKTKPREEQYNSTY | | ESNGQPENNYKTTPPVLDSDGS |
| | RVVSVLTVLHQDWLNGKEYKCKVS | | FFLYSKLTVDKSRWQQGNVFSC |
| | NKALPAPIEKTISKAKGQPREPQV | | SVMHEALHNHYTQKSLSLSPG |
| | YTLPPSREEMTKNQVSLTCLVKGF | | (SEQ ID NO: 53) |
| | YPSDIAVEWESNGQPENNYKTTPP | | |
| | VLDSDGSFFLYSKLTVDKSRWQQG | | |
| | NVFSCSVMHEALHNHYTQKSLSLS | | |
| | PGGGGGSDCGPPPDIPNARPILGR | | |
| | HSKFAEQSKVAYSCNNGFKQVPDK | | |
| | SNIVVCLENGQWSSHETFCEKSCV | | |
| | APERLSFASLKKEYLNMNFFPVGT | | |
| | IVEYECRPGFRKQPPLPGKATCLE | | |
| | DLVWSPVAQFCKKKSCPNPKDLDN | | |
| | GHINIPTGILFGSEINFSCNPGYR | | |
| | LVGVSSTFCSVTGNTVDWDDEFPV | | |
| | CTEIHCPEPPKINNGIMRGESDSY | | |
| | TYSQVVTYSCDKGFILVGNASIYC | | |
| | TVSKSDVGQWSSPPPRCIEKSKVP | | |
| | TKKPTINVPSTGTPSTPQKPTTES | | |
| | VPNPGDQPTPQKPSTVKVSATQHV | | |
| | PVTKTTVRHPIRTSTDKGEPNTG | | |
| | (SEQ ID NO: 108) | | |
| CDAB18- | AIRMTQSPSSLSASTGDRVTITCR | AIRMTQSPSSLSASTGDRV | RTVAAPSVFIFPPSDEQLKSGT |
| LC | ASQGISSYLAWYQQKPGKAPKLLI | TITCRASQGISSYLAWYQQ | ASVVCLLNNFYPREAKVQWKVD |
| | YAASTLQSGVPSRFSGSGSGTDFT | KPGKAPKLLIYAASTLQSG | NALQSGNSQESVTEQDSKDSTY |
| | LTISCLQSEDFATYYCQQYYSYPW | VPSRFSGSGSGTDFTLTIS | SLSSTLTLSKADYEKHKVYACE |
| | TFGQGTKVEIKRTVAAPSVFIFPP | CLQSEDFATYYCQQYYSYP | VTHQGLSSPVTKSENRGEC |
| | SDEQLKSGTASVVCLLNNFYPREA | WTFGQGTKVEIK (SEQ | (SEQ ID NO: 54) |
| | KVQWKVDNALQSGNSQESVTEQDS | ID NO: 111) | |
| | KDSTYSLSSTLTLSKADYEKHKVY | | |
| | ACEVTHQGLSSPVTKSENRGEC | | |
| | (SEQ ID NO: 110) | | |

Complement Modulator Antibody Table (Linker Sequences).

| Clone | | | scFv Heavy-Light chain |
|---|---|---|---|
| (scFv) | Tether-Effector Linker | C domain-scFv Linker | Linker |
| CDAB1-HC | GGGGS (SEQ ID NO: 26) | (Absent) | (Absent) |
| CDAB1-LC | (Absent) | (Absent) | (Absent) |
| CDAB2-HC | GGGGS (SEQ ID NO: 26) | (Absent) | (Absent) |
| CDAB2-LC | (Absent) | (Absent) | (Absent) |
| CDAB3 | GGGGSGGGGSGGGGSGGGGS | (Absent) | GGGGGSGGGGSGGGGS (SEQ ID |
| | (SEQ ID NO: 24) | | NO: 124) |
| CDAB4 | GGGGSGGGGSGGGGSGGGGS | (Absent) | GGGGSGGGGSGGGGSGGGGS (SEQ |
| | (SEQ ID NO: 24) | | ID NO: 24) |
| CDAB5 | GGGGSGGGGSGGGGSGGGGS | (Absent) | GGGGGSGGGGSGGGGS (SEQ ID |
| | (SEQ ID NO: 24) | | NO: 124) |
| CDAB6 | GGGGSGGGGSGGGGSGGGGS | GGGGSGGGGSGGGGSGGG | GGGGGSGGGGSGGGGS (SEQ ID |
| | (SEQ ID NO: 24) | GS (SEQ ID NO: 24) | NO: 124) |
| CDAB7 | GGGGSGGGGSGGGGSGGGGS | GGGGSGGGGSGGGGSGGG | GGGGGSGGGGSGGGGS (SEQ ID |
| | (SEQ ID NO: 24) | GS (SEQ ID NO: 24) | NO: 124) |
| CDAB8-HC | GGGGS (SEQ ID NO: 26) | (Absent) | (Absent) |
| CDAB9-HC | GGGGS (SEQ ID NO: 26) | (Absent) | (Absent) |
| CDAB10-HC | GGGGS (SEQ ID NO: 26) | (Absent) | (Absent) |
| CDAB11 | GGGGS (SEQ ID NO: 26) | (Absent) | (Absent) |
| CDAB12 | GGGGS (SEQ ID NO: 26) | (Absent) | (Absent) |
| CDAB13-HC | GGGGS (SEQ ID NO: 26) | (Absent) | (Absent) |
| CDAB13-LC | (Absent) | (Absent) | (Absent) |
| CDAB14 | GGGGSGGGGSGGGGSGGGGS | (Absent) | GGGGGSGGGGSGGGGS (SEQ ID |
| | (SEQ ID NO: 24) | | NO: 124) |
| CDAB15 | GGGGSGGGGSGGGGSGGGGS | (Absent) | GGGGGSGGGGSGGGGS (SEQ ID |
| | (SEQ ID NO: 24) | | NO: 124) |
| CDAB16 | GGGGSGGGGSGGGGSGGGGS | (Absent) | GGGGGSGGGGSGGGGS (SEQ ID |
| | (SEQ ID NO: 24) | | NO: 124) |
| CDAB17 | GGGGSGGGGSGGGGSGGGGS | (Absent) | GGGGGSGGGGSGGGGS (SEQ ID |
| | (SEQ ID NO: 24) | | NO: 124) |
| CDAB18-HC | GGGGS (SEQ ID NO: 26) | (Absent) | (Absent) |
| CDAB18-LC | (Absent) | (Absent) | (Absent) |

In some embodiments, the complement modulator fusion molecule comprises a complement modulator molecule linked via a serine/glycine linker to a constant domain (C domain), linked via a serine/glycine linker to a single variable domain (V Domain). In some embodiments, the complement modulator fusion protein comprises a complement modulator molecule linked via a serine/glycine linker to a constant domain (C domain), linked via a serine/glycine linker to a variable domain (V Domain), further linked via a serine/glycine linker to another variable domain (V domain). In some embodiments, the V domain is as described in US20200157212; WO2016/124768; US20050107596; Sado et al. (1995), Histochem Cell Biol., 104(4):267-75; or Foster et al. (2016). Molecular Immunology; each of which is incorporated herein by reference in its entirety.

In some embodiments, methods of inhibiting the complement system in the kidney glomerulus are provided herein. In some embodiments, the methods of inhibiting the complement system in the kidney glomerulus comprise tethering the polypeptide, as provided herein, to the glomerulus. In some embodiments, tethering of the polypeptide, as provided herein, to the glomerulus allows localized inhibition of the complement system within the glomerulus.

In some embodiments, methods of inhibiting the complement system in the kidney glomerulus of a subject in need thereof are provided. In some embodiments, methods of inhibiting the complement system in the kidney glomerulus of a subject in need thereof comprise tethering of the polypeptide, as provided herein, to the glomerulus of a subject in need thereof. In some embodiments, tethering of the polypeptide of, as provided herein, to the glomerulus allows localized inhibition of the complement system within the glomerulus of a subject in need thereof.

In some embodiments, methods of inhibiting the complement system in the kidney glomerulus of a subject suffering from a kidney disease are provided. In some embodiments, methods of inhibiting the complement system in the kidney glomerulus of a subject suffering from a kidney disease comprise tethering of the polypeptide, as provided herein, to the glomerulus of a subject suffering from a kidney disease. In some embodiments, tethering of the polypeptide, as provided herein, to the glomerulus allows localized inhibition of the complement system within the glomerulus of a subject suffering from a kidney disease.

In some embodiments, a protein comprising a glomerular targeting moiety and an effector moiety is provided. In some embodiments, the glomerular targeting moiety is an antibody that binds to a Robo2 protein, an antibody that binds to a COL4A3 protein, an antibody that binds to a COL4A4 protein, or an antibody that binds to a COL4A5 protein; and the effector moiety is a complement modulator selected from a CD55 protein, a CD59 protein, a CR1 protein, and a DCP protein.

In some embodiments, the glomerular targeting moiety comprises an amino acid sequence having at least 90%, at least 91%, at least 92%, at least 93%, at least 94%, at least 95%, at least 96%, at least 97%, at least 98%, or at least 99% identity to an amino acid sequence of SEQ ID NO: 70-80. In some embodiments, the glomerular targeting moiety comprises an amino acid sequence having at least 90% identity to an amino acid sequence of SEQ ID NO: 70. In some embodiments, the glomerular targeting moiety comprises an amino acid sequence having at least 90% identity to an amino acid sequence of SEQ ID NO: 71. In some embodiments, the glomerular targeting moiety comprises an amino acid sequence having at least 90% identity to an amino acid sequence of SEQ ID NO: 72. In some embodiments, the glomerular targeting moiety comprises an amino acid sequence having at least 90% identity to an amino acid sequence of SEQ ID NO: 73. In some embodiments, the glomerular targeting moiety comprises an amino acid sequence having at least 90% identity to an amino acid sequence of SEQ ID NO: 74. In some embodiments, glomerular the targeting moiety comprises an amino acid sequence having at least 90% identity to an amino acid sequence of SEQ ID NO: 75. In some embodiments, the glomerular targeting moiety comprises an amino acid sequence having at least 90% identity to an amino acid sequence of SEQ ID NO: 76. In some embodiments, the glomerular targeting moiety comprises an amino acid sequence having at least 90% identity to an amino acid sequence of SEQ ID NO: 77. In some embodiments, the glomerular targeting moiety comprises an amino acid sequence having at least 90% identity to an amino acid sequence of SEQ ID NO: 78. In some embodiments, the glomerular targeting moiety comprises an amino acid sequence having at least 90% identity to an amino acid sequence of SEQ ID NO: 79. In some embodiments, the glomerular targeting moiety comprises an amino acid sequence having at least 90% identity to an amino acid sequence of SEQ ID NO: 80.

In some embodiments, the glomerular targeting moiety comprises an amino acid sequence of SEQ ID NO: 70-80. In some embodiments, the glomerular targeting moiety comprises an amino acid sequence of SEQ ID NO: 70. In some embodiments, the glomerular targeting moiety comprises an amino acid sequence of SEQ ID NO: 71. In some embodiments, the glomerular targeting moiety comprises an amino acid sequence of SEQ ID NO: 72. In some embodiments, the glomerular targeting moiety comprises an amino acid sequence of SEQ ID NO: 73. In some embodiments, the glomerular targeting moiety comprises an amino acid sequence of SEQ ID NO: 74. In some embodiments, the glomerular targeting moiety comprises an amino acid sequence of SEQ ID NO: 75. In some embodiments, the glomerular targeting moiety comprises an amino acid sequence of SEQ ID NO: 76. In some embodiments, the glomerular targeting moiety comprises an amino acid sequence of SEQ ID NO: 77. In some embodiments, the glomerular targeting moiety comprises an amino acid sequence of SEQ ID NO: 78. In some embodiments, the glomerular targeting moiety comprises an amino acid sequence of SEQ ID NO: 79. In some embodiments, the glomerular targeting moiety comprises an amino acid sequence of SEQ ID NO: 80.

In some embodiments, the protein comprises the glomerular targeting moiety comprising an amino acid sequence as set forth in any of SEQ ID NO: 70-80; and the effector moiety comprises an amino acid sequence as set forth in any of SEQ ID NO: 1-12.

In some embodiments, the polypeptide comprises a light chain comprising an amino acid sequence as set forth in SEQ ID NO:83 or an amino acid sequence having at least 90%, at least 95%, at least 96%, at least 97%, at least 98% or at least 99% identity to the amino acid sequence of SEQ ID NO: 83, and a heavy chain comprising an amino acid sequence as set forth in SEQ ID NO:81 or an amino acid sequence having at least 90%, at least 95%, at least 96%, at least 97%, at least 98% or at least 99% identity to the amino acid sequence of SEQ ID NO: 81. In some embodiments, the polypeptide comprises a light chain comprising an amino acid sequence as set forth in SEQ ID NO:87 or an amino acid sequence having at least 90%, at least 95%, at least 96%, at least 97%, at least 98% or at least 99% identity to the amino acid sequence of SEQ ID NO: 87, and a heavy chain comprising an amino acid sequence as set forth in SEQ ID NO:85 or an amino acid sequence having at least 90%, at least 95%, at least 96%, at least 97%, at least 98% or at least 99% identity to the amino acid sequence of SEQ ID NO: 85. In some embodiments, the polypeptide comprises a light chain comprising an amino acid sequence as set forth in SEQ ID NO:102 or an amino acid sequence having at least 90%, at least 95%, at least 96%, at least 97%, at least 98% or at least 99% identity to the amino acid sequence of SEQ ID NO: 102, and a heavy chain comprising an amino acid sequence as set forth in SEQ ID NO:100 or an amino acid sequence having at least 90%, at least 95%, at least 96%, at least 97%, at least 98% or at least 99% identity to the amino acid sequence of SEQ ID NO: 100. In some embodiments, the polypeptide comprises a light chain comprising an amino acid sequence as set forth in SEQ ID NO:108 or an amino acid sequence having at least 90%, at least 95%, at least 96%, at least 97%, at least 98% or at least 99% identity to the amino acid sequence of SEQ ID NO: 108, and a heavy chain comprising an amino acid sequence as set forth in SEQ ID NO:110 or an amino acid sequence having at least 90%, at least 95%, at least 96%, at least 97%, at least 98% or at least 99% identity to the amino acid sequence of SEQ ID NO: 110.

In some embodiments, the polypeptide comprises a light chain comprising an amino acid sequence as set forth in SEQ ID NO: 83, and a heavy chain comprising an amino acid sequence as set forth in SEQ ID NO: 81. In some embodiments, the polypeptide comprises a light chain comprising an amino acid sequence as set forth in SEQ ID NO: 87, and a heavy chain comprising an amino acid sequence as set forth in SEQ ID NO: 85. In some embodiments, the polypeptide comprises a light chain comprising an amino acid sequence as set forth in SEQ ID NO: 102, and a heavy chain comprising an amino acid sequence as set forth in SEQ ID NO: 100. In some embodiments, the polypeptide comprises a light chain comprising an amino acid sequence as set forth in SEQ ID NO: 108, and a heavy chain comprising an amino acid sequence as set forth in SEQ ID NO: 110.

In some embodiments, the protein comprises an amino acid sequence as set forth in SEQ ID NO: 81, or an amino acid sequence having at least 90%, at least 95%, at least 96%, at least 97%, at least 98%, or at least 99% identity to an amino acid sequence of SEQ ID NO: 81. In some embodiments, the protein comprises an amino acid sequence as set forth in SEQ ID NO:83, or an amino acid sequence having at least 90%, at least 95%, at least 96%, at least 97%, at least 98%, or at least 99% identity to an amino acid sequence of SEQ ID NO: 83. In some embodiments, the protein comprises an amino acid sequence as set forth in SEQ ID NO: 85, or an amino acid sequence having at least 90%, at least 95%, at least 96%, at least 97%, at least 98%, or at least 99% identity to an amino acid sequence of SEQ ID NO: 85. In some embodiments, the protein comprises an amino acid sequence as set forth in SEQ ID NO: 87, or an amino acid sequence having at least 90%, at least 95%, at least 96%, at least 97%, at least 98%, or at least 99% identity to an amino acid sequence of SEQ ID NO: 87. In some embodiments, the protein comprises an amino acid sequence as set forth in SEQ ID NO: 89, or an amino acid sequence having at least 90%, at least 95%, at least 96%, at least 97%, at least 98%, or at least 99% identity to an amino acid sequence of SEQ ID NO: 89. In some embodiments, the protein comprises an amino acid sequence as set forth in SEQ ID NO: 90, or an amino acid sequence having at least 90%, at least 95%, at least 96%, at least 97%, at least 98%, or at least 99% identity to an amino acid sequence of SEQ ID NO: 90. In some embodiments, the protein comprises an amino acid sequence as set forth in SEQ ID NO: 91, or an amino acid sequence having at least 90%, at least 95%, at least 96%, at least 97%, at least 98%, or at least 99% identity to an amino acid sequence of SEQ ID NO: 91. In some embodiments, the protein comprises an amino acid sequence as set forth in SEQ ID NO: 92, or an amino acid sequence having at least 90%, at least 95%, at least 96%, at least 97%, at least 98%, or at least 99% identity to an amino acid sequence of SEQ ID NO: 92. In some embodiments, the protein comprises an amino acid sequence as set forth in SEQ ID NO: 93, or an amino acid sequence having at least 90%, at least 95%, at least 96%, at least 97%, at least 98%, or at least 99% identity to an amino acid sequence of SEQ ID NO: 93. In some embodiments, the protein comprises an amino acid sequence as set forth in SEQ ID NO: 94, or an amino acid sequence having at least 90%, at least 95%, at least 96%, at least 97%, at least 98%, or at least 99% identity to an amino acid sequence of SEQ ID NO: 94. In some embodiments, the protein comprises an amino acid sequence as set forth in SEQ ID NO: 95, or an amino acid sequence having at least 90%, at least 95%, at least 96%, at least 97%, at least 98%, or at least 99% identity to an amino acid sequence of SEQ ID NO: 95. In some embodiments, the protein comprises an amino acid sequence as set forth in SEQ ID NO: 96, or an amino acid sequence having at least 90%, at least 95%, at least 96%, at least 97%, at least 98%, or at least 99% identity to an amino acid sequence of SEQ ID NO: 96. In some embodiments, the protein comprises an amino acid sequence as set forth in SEQ ID NO: 97, or an amino acid sequence having at least 90%, at least 95%, at least 96%, at least 97%, at least 98%, or at least 99% identity to an amino acid sequence of SEQ ID NO: 97. In some embodiments, the protein comprises an amino acid sequence as set forth in SEQ ID NO: 99, or an amino acid sequence having at least 90%, at least 95%, at least 96%, at least 97%, at least 98%, or at least 99% identity to an amino acid sequence of SEQ ID NO: 99. In some embodiments, the protein comprises an amino acid sequence as set forth in SEQ ID NO: 100, or an amino acid sequence having at least 90%, at least 95%, at least 96%, at least 97%, at least 98%, or at least 99% identity to an amino acid sequence of SEQ ID NO: 100. In some embodiments, the protein comprises an amino acid sequence as set forth in SEQ ID NO: 102, or an amino acid sequence having at least 90%, at least 95%, at least 96%, at least 97%, at least 98%, or at least 99% identity to an amino acid sequence of SEQ ID NO: 102. In some embodiments, the protein comprises an amino acid sequence as set forth in SEQ ID NO: 104, or an amino acid sequence having at least 90%, at least 95%, at least 96%, at least 97%, at least 98%, or at least 99% identity to an amino acid sequence of SEQ ID NO: 104. In some embodiments, the protein comprises an amino acid sequence as set forth in SEQ ID NO: 105, or an amino acid sequence having at least 90%, at least 95%, at least 96%, at least 97%, at least 98%, or at least 99% identity to an amino acid sequence of SEQ ID NO: 105. In some embodiments, the protein comprises an amino acid sequence as set forth in SEQ ID NO: 106, or an amino acid sequence having at least 90%, at least 95%, at least 96%, at least 97%, at least 98%, or at least 99% identity to an amino acid sequence of SEQ ID NO: 106. In some embodiments, the protein comprises an amino acid sequence as set forth in SEQ ID NO: 107, or an amino acid sequence having at least 90%, at least 95%, at least 96%, at least 97%, at least 98%, or at least 99% identity to an amino acid sequence of SEQ ID NO: 107. In some embodiments, the protein comprises an amino acid sequence as set forth in SEQ ID NO: 108, or an amino acid sequence having at least 90%, at least 95%, at least 96%, at least 97%, at least 98%, or at least 99% identity to an amino acid sequence of SEQ ID NO: 108. In some embodiments, the protein comprises an amino acid sequence as set forth in SEQ ID NO: 110, or an amino acid sequence having at least 90%, at least 95%, at least 96%, at least 97%, at least 98%, or at least 99% identity to an amino acid sequence of SEQ ID NO: 110.

In some embodiments, the polypeptide comprises an amino acid sequence comprising the sequence as set forth in SEQ ID NO: 81. In some embodiments, the polypeptide comprises an amino acid sequence comprising the sequence as set forth in SEQ ID NO: 83. In some embodiments, the polypeptide comprises an amino acid sequence comprising the sequence as set forth in SEQ ID NO: 85. In some embodiments, the polypeptide comprises an amino acid sequence comprising the sequence as set forth in SEQ ID NO: 87. In some embodiments, the polypeptide comprises an amino acid sequence comprising the sequence as set forth in SEQ ID NO: 89. In some embodiments, the polypeptide comprises an amino acid sequence comprising the sequence as set forth in SEQ ID NO: 90. In some embodiments, the polypeptide comprises an amino acid sequence comprising the sequence as set forth in SEQ ID NO: 91. In some embodiments, the polypeptide comprises an amino acid sequence comprising the sequence as set forth in SEQ ID NO: 92. In some embodiments, the polypeptide comprises an amino acid sequence comprising the sequence as set forth in SEQ ID NO: 93. In some embodiments, the polypeptide comprises an amino acid sequence comprising the sequence as set forth in SEQ ID NO: 94. In some embodiments, the polypeptide comprises an amino acid sequence comprising the sequence as set forth in SEQ ID NO: 95. In some embodiments, the polypeptide comprises an amino acid sequence comprising the sequence as set forth in SEQ ID NO: 96. In some embodiments, the polypeptide comprises an amino acid sequence comprising the sequence as set forth in SEQ ID NO: 97. In some embodiments, the polypeptide comprises an amino acid sequence comprising the sequence as set forth in SEQ ID NO: 99. In some embodiments, the polypeptide comprises an amino acid sequence comprising the sequence of SEQ ID NO: 100. In some embodiments, the polypeptide comprises an amino acid sequence comprising the sequence of SEQ ID NO: 102. In some embodiments, the polypeptide comprises an amino acid sequence comprising the sequence as set forth in SEQ ID NO: 104. In some embodiments, the polypeptide comprises an amino acid sequence comprising the sequence as set forth in SEQ ID NO: 105. In some embodiments, the polypeptide comprises an amino acid sequence comprising the sequence as set forth in SEQ ID NO: 106. In some embodiments, the polypeptide comprises an amino acid sequence comprising the sequence as set forth in SEQ ID NO: 107. In some embodiments, the polypeptide comprises an amino acid sequence comprising the sequence as set forth in SEQ ID NO: 108. In some embodiments, the polypeptide comprises an amino acid sequence comprising the sequence as set forth in SEQ ID NO: 110.

In some embodiments, a tissue specific targeting moiety, as a component of a therapeutic compound, preferentially binds to a target tissue or target tissue antigen, e.g., has a binding affinity for the target tissue or antigen that is greater for target antigen or tissue, e.g., at least 2, 4, 5, 10, 50, 100, 500, 1,000, 5,000, or 10,000 fold greater, than its affinity for non-target tissue or antigen present outside the target tissue. Affinity of a therapeutic compound of which the tissue specific moiety is a component, can be measured in a cell suspension, e.g., the affinity for suspended cells having the target antigen is compared with its affinity for suspended cells not having the target antigen. In some embodiments, the binding affinity for the target antigen bearing cells is below 10 nM.

In some embodiments, the binding affinity for the target antigen bearing cells is below 100 pM, 50 pM, or 10 pM. In some embodiments, the specificity for a target antigen is sufficient, that when the tissue specific targeting moiety is coupled to an immune down regulating effector: i) immune attack of the target tissue, e.g., as measured by histological inflammatory response, infiltrating T effector cells, or organ function, in the clinical setting, e.g., creatinine for kidney, is substantially reduced, e.g., as compared to what would be seen in an otherwise similar implant but lacking the tissue specific targeting moiety is coupled to an immune down regulating effector; and/or ii) immune function in the recipient, outside or away from the target tissue, is substantially maintained.

In some embodiments, one or more of the following is seen: at therapeutic levels of therapeutic compound, peripheral blood lymphocyte counts are not substantially impacted, e.g., the level of T cells is within 25, 50, 75, 85, 90, or 95% of normal, the level of B cells is within 25, 50, 75, 85, 90, or 95% of normal, and/or the level of granulocytes (PMN cells) is within 25, 50, 75, 85, 90, or 95% of normal, or the level of monocytes is within 25, 50, 75, 85, 90, or 95% of normal; at therapeutic levels of therapeutic compound, the ex vivo proliferative function of PBMCs against non-disease relevant antigens is substantially normal or is within 70, 80, or 90% of normal; at therapeutic levels of therapeutic compound, the incidence or risk of opportunistic infections and cancers associated with immunosuppression is not substantially increased over normal; or at therapeutic levels of therapeutic compound, the incidence or risk of opportunistic infections and cancers associated with immunosuppression is substantially less than would be seen with standard of care, or non-targeted, immunosuppression. In some embodiments, the tissue specific targeting moiety comprises an antibody molecule. In some embodiments, the donor specific targeting moiety comprises an antibody molecule, a target specific binding polypeptide, or a target ligand binding molecule. In some embodiments, the tissue specific targeting moiety binds a product, or a site on a product, that is present or expressed exclusively, or substantially exclusively, on target tissue. In some embodiments, the target tissue is the kidney tissue. In some embodiments, the target tissue is the kidney glomerular tissue.

### Exemplary Inhibitory Immune Checkpoint Molecules

As provided for herein, the targeting moiety that binds to Robo2, COL4A3, COL4A4, or COL4A5 can be linked or associated with an inhibitory immune checkpoint molecule. Exemplary inhibitory molecules (e.g., an inhibitory immune checkpoint molecule) (together with their counter ligands) can be found in Table 1. This table lists molecules to which exemplary ICIM binding moieties can bind.

TABLE 1
Cell surface inhibitory molecules, e.g., inhibitory immune
checkpoint molecules (column A), counter ligands
(column B) and cell types affected (column C).

| A | B | C |
|---|---|---|
| PD-1 | PD-L1, PD-L2 | T cells, B cells |
| Alkaline phosphatase | | |
| B7-H3 | Unknown | T cells |
| B7-H4 | Neuropilin 1, | T cells |
| | Neuropilin 2, | |
| | Plexin4A | |
| BTLA | HVEM | T cells, B cells |
| CTLA-4 | CD80, CD86 | T cells |
| IDO1 | Tryptophan | Lymphocytes |
| IDO2 | Tryptophan | Lymphocytes |
| KIR2DL1, | HLA MHC class I | NK cells |
| KIR2DL2/3, | | |
| KIR3DL1, KIR3DL2 | | |
| LAG3 | HLA MHC class II | T cells |
| TIM-3 | Galectin-9 | T cells |
| VISTA | Unknown | T cells, myeloid cells |
| TIGIT | CD155 | T cells |
| KIR2DL4 | HLA-G | NK cells |
| LILRB1 | HLA-G | T cells, NK cells, |
| | | B cells, monocytes, |
| | | dendritic cells |
| LILRB2 | HLA-G | Monocytes, dendritic |
| | | cells, neutrophils, some |
| | | tumor cells |
| NKG2A | Nonclassical MHC | T cells, NK cells |
| | Glycoproteins class I | |
| FCRL1-6 | FCRL1-2 not | B cells |
| | known | |
| | FCRL4 = IgA | |
| | FCRL5 = IgG | |
| | FCRL6 = MHC Class | |
| | II | |
| | BUTYROPHILINS, | Modulation of |
| | for example | immune cells |
| | BTN1A1, BTN2A2, | |
| | BTNL2, BTNL1, | |
| | BTNL8 | |

IL-2 Mutein Molecules: IL-2 Receptor Binders that Activate Tregs

IL-2 mutein molecules that preferentially expand or stimulate Treg cells (over cytotoxic T cells) can be used as a IIC binding/modulating moiety and be linked to, for example, the targeting moiety that binds to Robo2, COL4A3, COL4A4, or COL4A5. In some embodiments, cytotoxic T cell is an effector T cell. In some embodiments, effector T cell is a cytotoxic T cell.

In some embodiments, a IIC binding/modulating moiety comprises an IL-2 mutein molecule. As used herein, the term “IL-2 mutein molecule” or “IL-2 mutein” refers to an IL-2 variant that preferentially activates Treg cells. In some embodiments, either alone, or as a component of a therapeutic compound, an IL-2 mutein molecule activates Tregs at least 2, 5, 10, or 100 fold more than cytotoxic T cells. A suitable assay for evaluating preferential activation of Treg cells can be found in U.S. Pat. No. 9,580,486 at, for example, Examples 2 and 3, or in WO2016014428 at, for example, Examples 3, 4, and 5, each of which is incorporated by reference in its entirety. The sequence of mature IL-2 is

(SEQ ID NO: 15)
APTSSSTKKTQLQLEHLLLDLQMILNGINNYKNPKLTRMLT
FKFYMPKKATELKHLQCLEEELKPLEEVLNLAQSKNFHLRP
RDLISNINVIVLELKGSETTFMCEYADETATIVEFLNRWITFC
QSIISTLT (mature IL-2 sequence)

The immature sequence of IL-2 can be represented by

(SEQ ID NO: 16)
MYRMQLLSCIALSLALVTNSAPTSSSTKKTQLQLEHLLLDL
QMILNGINNYKNPKLTRMLTFKFYMPKKATELKHLQCLEE
ELKPLEEVLNLAQSKNFHLRPRDLISNINVIVLELKGSETTF
MCEYADETATIVEFLNRWITFCQSIISTLT.

In some embodiments, a IIC binding/modulating moiety comprises an IL-2 mutein, or active fragment thereof, coupled, e.g., fused, to another polypeptide, e.g., a polypeptide that extends in vivo half-life, e.g., an immunoglobulin constant region, or a multimer or dimer thereof.

An IL-2 mutein molecule can be prepared by mutating one or more of the residues of IL-2. Non-limiting examples of IL-2-muteins can be found in U.S. Pat. Nos. 10,174,091, 10,676,516 WO2016/164937, U.S. Pat. Nos. 9,580,486, 7,105,653, 9,616,105, 9,428,567, US2017/0051029, US2014/0286898, WO2014153111, WO2010/085495, WO2016014428, WO2016025385, and US20060269515, each of which are incorporated by reference in its entirety.

In some embodiments, the alanine at position 1 of the sequence above is deleted (i.e., the alanine at position 1 of SEQ ID NO: 15). In some embodiments, the IL-2 mutein molecule comprises a serine substituted for cysteine at position 125 of the mature IL-2 sequence. Other combinations of mutations and substitutions that are IL-2 mutein molecules are described in US20060269515, which is incorporated by reference in its entirety. In some embodiments, the cysteine at position 125 is also substituted with a valine or alanine. In some embodiments, the IL-2 mutein molecule comprises a V91K substitution. In some embodiments, the IL-2 mutein molecule comprises a N88D substitution. In some embodiments, the IL-2 mutein molecule comprises a N88R substitution. In some embodiments, the IL-2 mutein molecule comprises a substitution of H16E, D84K, V91N, N88D, V91K, or V91R, any combinations thereof. In some embodiments, these IL-2 mutein molecules also comprise a substitution at position 125 as described herein. In some embodiments, the IL-2 mutein molecule comprises one or more substitutions selected from the group consisting of: T3N, T3A, L12G, L12K, L12Q, L12S, Q13G, E15A, E15G, E15S, H16A, H16D, H16G, H16K, H16M, H16N, H16R, H16S, H16T, H16V, H16Y, L19A, L19D, L19E, L19G, L19N, L19R, L19S, L19T, L19V, D20A, D20E, D20H, D20I, D20Y, D20F, D20G, D20T, D20W, M23R, R81A, R81G, R81S, R81T, D84A, D84E, D84G, D84I, D84M, D84Q D84R, D84S, D84T, S87R, N88A, N88D, N88E, N88I, N88F, N88G, N88M, N88R, N88S, N88V, N88W, V91D, V91E, V91G, V91S, I92K, I92R, E95G, and Q126. In some embodiments, the amino acid sequence of the IL-2 mutein molecule differs from the amino acid sequence set forth in mature IL-2 sequence with a C125A or C125S substitution and with one substitution selected from T3N, T3A, L12G, L12K, L12Q L12S, Q13G, E15A, E15G, E15S, H16A, H16D, H16G, H16K, H16M, H16N, H16R, H16S, H16T, H16V, H16Y, L19A, L19D, L19E, L19G, L19N, L19R, L19S, L19T, L19V, D20A, D20E, D20F, D20G, D20T, D20W, M23R, R81A, R81G, R81S, R81T, D84A, D84E, D84G, D84I, D84M, D84Q, D84R, D84S, D84T, S87R, N88A, N88D, N88E, N88F, N88I, N88G, N88M, N88R, N88S, N88V, N88W, V91D, V91E, V91G, V91S, I92K, I92R, E95G, Q1261, Q126L, and Q126F. In some embodiments, the IL-2 mutein molecule differs from the amino acid sequence set forth in mature IL-2 sequence with a C125A or C125S substitution and with one substitution selected from D20H, D20I, D20Y, D20E, D20G, D20W, D84A, D84S, H16D, H16G, H16K, H16R, H16T, H16V, I92K, I92R, L12K, L19D, L19N, L19T, N88D, N88R, N88S, V91D, V91G, V91K, and V91S. In some embodiments, the IL-2 mutein comprises N88R and/or D20H mutations.

In some embodiments, the IL-2 mutein molecule comprises a mutation in the polypeptide sequence at a position selected from the group consisting of amino acid 30, amino acid 31, amino acid 35, amino acid 69, and amino acid 74. In some embodiments, the mutation at position 30 is N30S. In some embodiments, the mutation at position 31 is Y31H. In some embodiments, the mutation at position 35 is K35R. In some embodiments, the mutation at position 69 is V69A. In some embodiments, the mutation at position 74 is Q74P. In some embodiments, the mutein comprises a V69A mutation, a Q74P mutation, a N88D or N88R mutation, and one or more of L53I, L56I, L80I, or L118I mutations. In some embodiments, the mutein comprises a V69A mutation, a Q74P mutation, a N88D or N88R mutation, and a L to I mutation selected from the group consisting of: L53I, L56I, L80I, and L118I mutation. In some embodiments, the IL-2 mutein comprises a V69A, a Q74P, a N88D or N88R mutation, and a L53I mutation. In some embodiments, the IL-2 mutein comprises a V69A, a Q74P, a N88D or N88R mutation, and a L56I mutation. In some embodiments, the IL-2 mutein comprises a V69A, a Q74P, a N88D or N88R mutation, and a L80I mutation. In some embodiments, the IL-2 mutein comprises a V69A, a Q74P, a N88D or N88R mutation, and a L 118I mutation. As provided for herein, the muteins can also comprise a C125A or C125S mutation.

In some embodiments, the IL-2 mutein molecule comprises a substitution selected from the group consisting of: N88R, N88I, N88G, D20H, D109C, Q126L, Q126F, D84G, and D84I relative to mature human IL-2 sequence provided above. In some embodiments, the IL-2 mutein molecule comprises a substitution of D109C and one or both of a N88R substitution and a C125S substitution. In some embodiments, the cysteine that is in the IL-2 mutein molecule at position 109 is linked to a polyethylene glycol moiety, wherein the polyethylene glycol moiety has a molecular weight of between 5 and 40 kDa.

In some embodiments, any of the substitutions described herein are combined with a substitution at position 125. The substitution can be a C125S, C125A, or C125V substitution.

In addition to the substitutions or mutations described herein, in some embodiments, the IL-2 mutein has a substitution/mutation at one or more of positions 73, 76, 100, or 138 that correspond to SEQ ID NO: 15 or positions at one or more of positions 53, 56, 80, or 118 that correspond to SEQ ID NO: 6. In some embodiments, the IL-2 mutein comprises a mutation at positions 73 and 76; 73 and 100; 73 and 138; 76 and 100; 76 and 138; 100 and 138; 73, 76, and 100; 73, 76, and 138; 73, 100, and 138; 76, 100 and 138; or at each of 73, 76, 100, and 138 that correspond to SEQ ID NO: 15. In some embodiments, the IL-2 mutein comprises a mutation at positions 53 and 56; 53 and 80; 53 and 118; 56 and 80; 56 and 118; 80 and 118; 53, 56, and 80; 53, 56, and 118; 53, 80, and 118; 56, 80 and 118; or at each of 53, 56, 80, and 118 that correspond to SEQ ID NO: 15. As the IL-2 can be fused or tethered to other proteins, as used herein, the term “corresponds to” with reference to SEQ ID NOs: 15 or 16 refers to how the sequences would align with default settings for alignment software, such as the alignment tool available at the NCBI website. In some embodiments, the mutation is leucine to isoleucine.

Thus, the IL-2 mutein can comprise one more isoleucines at positions 73, 76, 100, or 138 that correspond to SEQ ID NO: 16 or positions at one or more of positions 53, 56, 80, or 118 that correspond to SEQ ID NO: 15. In some embodiments, the mutein comprises a mutation at L53 that corresponds to SEQ ID NO: 15. In some embodiments, the mutein comprises a mutation at L56 that corresponds to SEQ ID NO: 15. In some embodiments, the mutein comprises a mutation at L80 that corresponds to SEQ ID NO: 15. In some embodiments, the mutein comprises a mutation at L118 that corresponds to SEQ ID NO: 15. In some embodiments, the mutation is leucine to isoleucine. In some embodiments, the mutein also comprises a mutation as position 69, 74, 88, 125, or any combination thereof in these muteins that correspond to SEQ ID NO: 6. In some embodiments, the mutation is a V69A mutation. In some embodiments, the mutation is a Q74P mutation. In some embodiments, the mutation is a N88D or N88R mutation. In some embodiments, the mutation is a C125A or C125S mutation.

In some embodiments, the IL-2 mutein comprises a mutation at one or more of positions 49, 51, 55, 57, 68, 89, 91, 94, 108, and 145 that correspond to SEQ ID NO: 16 or one or more positions 29, 31, 35, 37, 48, 69, 71, 74, 88, and 125 that correspond to SEQ ID NO: 15. The substitutions can be used alone or in combination with one another. In some embodiments, the IL-2 mutein comprises 2, 3, 4, 5, 6, 7, 8, 9 or all substitutions at positions 49, 51, 55, 57, 68, 89, 91, 94, 108, and 145. Non-limiting examples such combinations include, but are not limited to, a mutation at positions 49, 51, 55, 57, 68, 89, 91, 94, 108, and 145; 49, 51, 55, 57, 68, 89, 91, 94, and 108; 49, 51, 55, 57, 68, 89, 91, and 94; 49, 51, 55, 57, 68, 89, and 91; 49, 51, 55, 57, 68, and 89; 49, 51, 55, 57, and 68; 49, 51, 55, and 57; 49, 51, and 55; 49 and 51; 51, 55, 57, 68, 89, 91, 94, 108, and 145; 51, 55, 57, 68, 89, 91, 94, and 108; 51, 55, 57, 68, 89, 91, and 94; 51, 55, 57, 68, 89, and 91; 51, 55, 57, 68, and 89; 55, 57, and 68; 55 and 57; 55, 57, 68, 89, 91, 94, 108, and 145; 55, 57, 68, 89, 91, 94, and 108; 55, 57, 68, 89, 91, and 94; 55, 57, 68, 89, 91, and 94; 55, 57, 68, 89, and 91; 55, 57, 68, and 89; 55, 57, and 68; 55 and 57; 57, 68, 89, 91, 94, 108, and 145; 57, 68, 89, 91, 94, and 108; 57, 68, 89, 91, and 94; 57, 68, 89, and 91; 57, 68, and 89; 57 and 68; 68, 89, 91, 94, 108, and 145; 68, 89, 91, 94, and 108; 68, 89, 91, and 94; 68, 89, and 91; 68 and 89; 89, 91, 94, 108, and 145; 89, 91, 94, and 108; 89, 91, and 94; 89 and 91; 91, 94, 108, and 145; 91, 94, and 108; 91, and 94; or 94 and 108. Each mutation can be combined with one another. The same substitutions can be made in SEQ ID NO: 15, but the numbering would adjust appropriately as is clear from the present disclosure (20 less than the numbering for SEQ ID NO: 16 corresponds to the positions in SEQ ID NO: 15).

In some embodiments, the IL-2 mutein comprises a mutation at one or more positions of 35, 36, 42, 104, 115, or 146 that correspond to SEQ ID NO: 16 or the equivalent positions at SEQ ID NO: 15 (i.e., positions 15, 16, 22, 84, 95, or 126). These mutations can be combined with the other leucine to isoleucine mutations described herein or the mutation at positions 73, 76, 100, or 138 that correspond to SEQ ID NO: 16 or at one or more of positions 53, 56, 80, or 118 that correspond to SEQ ID NO: 15. In some embodiments, the mutation is a E35Q, H36N, Q42E, D104N, E115Q, or Q146E substitution, or any combination thereof. In some embodiments, one or more of these substitutions is wild-type. In some embodiments, the mutein comprises a wild-type residue at one or more of positions 35, 36, 42, 104, 115, or 146 that correspond to SEQ ID NO: 16 or the equivalent positions at SEQ ID NO: 15 (i.e., positions 15, 16, 22, 84, 95, and 126).

The mutations at these positions can be combined with any of the other mutations described herein, including, but not limited to substitutions at positions 73, 76, 100, or 138 that correspond to SEQ ID NO: 16 or positions at one or more of positions 53, 56, 80, or 118 that correspond to SEQ ID NO: 15 described herein and above. In some embodiments, the IL-2 mutein comprises a N49S mutation that corresponds to SEQ ID NO: 16. In some embodiments, the IL-2 mutein comprises a Y51S or a Y51H mutation that corresponds to SEQ ID NO: 16. In some embodiments, the IL-2 mutein comprises a K55R mutation that corresponds to SEQ ID NO: 16. In some embodiments, the IL-2 mutein comprises a T57A mutation that corresponds to SEQ ID NO: 16. In some embodiments, the IL-2 mutein comprises a K68E mutation that corresponds to SEQ ID NO: 16. In some embodiments, the IL-2 mutein comprises a V89A mutation that corresponds to SEQ ID NO: 16. In some embodiments, the IL-2 mutein comprises a N91R mutation that corresponds to SEQ ID NO: 16. In some embodiments, the IL-2 mutein comprises a Q94P mutation that corresponds to SEQ ID NO: 16. In some embodiments, the IL-2 mutein comprises a N108D or a N108R mutation that corresponds to SEQ ID NO: 16. In some embodiments, the IL-2 mutein comprises a C145A or C145S mutation that corresponds to SEQ ID NO: 16. These substitutions can be used alone or in combination with one another. In some embodiments, the mutein comprises each of these substitutions. In some embodiments, the mutein comprises 1, 2, 3, 4, 5, 6, 7, or 8 of these mutations. In some embodiments, the mutein comprises a wild-type residue at one or more of positions 35, 36, 42, 104, 115, or 146 that correspond to SEQ ID NO: 16 or the equivalent positions at SEQ ID NO: 15 (i.e., positions 15, 16, 22, 84, 95, and 126).

In some embodiments, the IL-2 mutein comprises a N29S mutation that corresponds to SEQ ID NO: 15. In some embodiments, the IL-2 mutein comprises a Y31S or a Y31H mutation that corresponds to SEQ ID NO: 15. In some embodiments, the IL-2 mutein comprises a K35R mutation that corresponds to SEQ ID NO: 15. In some embodiments, the IL-2 mutein comprises a T37A mutation that corresponds to SEQ ID NO: 15. In some embodiments, the IL-2 mutein comprises a K48E mutation that corresponds to SEQ ID NO: 15. In some embodiments, the IL-2 mutein comprises a V69A mutation that corresponds to SEQ ID NO: 15. In some embodiments, the IL-2 mutein comprises a N71R mutation that corresponds to SEQ ID NO: 15. In some embodiments, the IL-2 mutein comprises a Q74P mutation that corresponds to SEQ ID NO: 15. In some embodiments, the IL-2 mutein comprises a N88D or a N88R mutation that corresponds to SEQ ID NO: 15. In some embodiments, the IL-2 mutein comprises a C125A or C125S mutation that corresponds to SEQ ID NO: 15. These substitutions can be used alone or in combination with one another. In some embodiments, the mutein comprises 1, 2, 3, 4, 5, 6, 7, or 8 of these mutations. In some embodiments, the mutein comprises each of these substitutions. In some embodiments, the mutein comprises a wild-type residue at one or more of positions 35, 36, 42, 104, 115, or 146 that correspond to SEQ ID NO: 16 or the equivalent positions of SEQ ID NO: 15 (e.g., positions 15, 16, 22, 84, 95, and 126).

For any of the IL-2 muteins described herein, in some embodiments, one or more of positions 35, 36, 42, 104, 115, or 146 that correspond to SEQ ID NO: 16 or the equivalent positions at SEQ ID NO: 15 (i.e., positions 15, 16, 22, 84, 95, or 126) are wild-type (i.e., are as shown in SEQ ID NOs: 15 or 16). In some embodiments, 2, 3, 4, 5, 6, or each of positions 35, 36, 42, 104, 115, or 146 that correspond to SEQ ID NO: 16 or the equivalent positions at SEQ ID NO: 15 (i.e., positions 15, 16, 22, 84, 95, and 126) are wild-type.

In some embodiments, the IL-2 mutein comprises or consists of a sequence of:

(SEQ ID NO: 17)
MYRMQLLSCIALSLALVTNSAPTSSSTKKTQLQLEHLLLDL
QMILNGISNHKNPRLARMLTFKFYMPEKATEIKHLQCLEEE
LKPLEEALRLAPSKNFHLRPRDLISDINVIVLELKGSETTFMC
EYADETATIVEFLNRWITFSQSIISTLT

In some embodiments, the IL-2 mutein comprises or consist of a sequence of:

(SEQ ID NO: 18)
MYRMQLLSCIALSLALVTNSAPTSSSTKKTQLQLEHLLLDL
QMILNGISNHKNPRLARMLTFKFYMPEKATELKHIQCLEEE
LKPLEEALRLAPSKNFHLRPRDLISDINVIVLELKGSETTFMC
EYADETATIVEFLNRWITFSQSIISTLT

In some embodiments, the IL-2 mutein comprises or consists of a sequence of:

(SEQ ID NO: 19)
MYRMQLLSCIALSLALVTNSAPTSSSTKKTQLQLEHLLLDL
QMILNGISNHKNPRLARMLTFKFYMPEKATELKHLQCLEEE
LKPLEEALRLAPSKNFHIRPRDLISDINVIVLELKGSETTFMC
EYADETATIVEFLNRWITFSQSIISTLT

In some embodiments, the IL-2 mutein comprises or consists of a sequence of:

(SEQ ID NO: 20)
MYRMQLLSCIALSLALVTNSAPTSSSTKKTQLQLEHLLLDL
QMILNGISNHKNPRLARMLTFKFYMPEKATELKHLQCLEEE
LKPLEEALRLAPSKNFHLRPRDLISDINVIVLELKGSETTFMC
EYADETATIVEFINRWITFSQSIISTLT

In some embodiments, the IL-2 mutein sequences described herein do not comprise the IL-2 leader sequence. The IL-2 leader sequence can be represented by the sequence of MYRMQLLSCIALSLALVTNS (SEQ ID NO: 21). Therefore, in some embodiments, the sequences illustrated above can also encompass peptides without the leader sequence. Although SEQ ID NOs; 17-21 are illustrated with mutations at one of positions 73, 76, 100, or 138 that correspond to SEQ ID NO: 16 or positions at one or more of positions 53, 56, 80, or 118 that correspond to SEQ ID NO: 15, the peptides can comprise one, two, three or four of the mutations at these positions. In some embodiments, the substitution at each position is isoleucine or other type of conservative amino acid substitution. In some embodiments, one or more leucines at the recited positions are substituted with, independently, isoleucine, valine, methionine, or phenylalanine.

In some embodiments, the IL-2 mutein molecule is fused to a Fc region or other linker region as described herein. Examples of such fusion proteins can be found in U.S. Pat. Nos. 9,580,486, 7,105,653, 9,616,105, 9,428,567, US2017/0051029, WO2016/164937, US2014/0286898A1, WO2014153111A2, WO2010/085495, WO2016014428A2, WO2016025385A1, US2017/0037102, and US2006/0269515, each of which are incorporated by reference in its entirety.

In some embodiments, the Fc region comprises what is known as a LALA mutation. Using the Kabat numbering of the Fc region, this would correspond to L247A, L248A, and G250A. In some embodiments, using the EU numbering of the Fc region, the Fc region comprises a L234A mutation, a L235A mutation, and/or a G237A mutation. Regardless of the numbering system used, in some embodiments, the Fc portion comprises mutations that correspond to these residues. In some embodiments, the Fc region comprises N297G or N297A (Kabat numbering) mutations. The Kabat numbering is based upon a full-length sequence, but would be used in a fragment based upon a traditional alignment used by one of skill in the art for the Fc region.

In some embodiments, the Fc region comprises or consists of a sequence of:

(SEQ ID NO: 51)
DKTHTCPPCPAPEAAGAPSVFLFPPKPKDTLMISRTPEVTCV
VVDVSHEDPEVKFNWYVDGVEVHNAKTKPREEQYNSTYR
VVSVLTVLHQDWLNGKEYKCKVSNKALPAPIEKTISKAKG
QPREPQVYTLPPSREEMTKNQVSLTCLVKGFYPSDIAVEWE
SNGQPENNYKTTPPVLDSDGSFFLYSKLTVDKSRWQQGNV
FSCSVMHEALHNHYTQKSLSLSPG
or
(SEQ ID NO: 23)
DKTHTCPPCPAPELLGGPSVFLFPPKPKDTLMISRTPEVTCV
VVDVSHEDPEVKFNWYVDGVEVHNAKTKPREEQYNSTYR
VVSVLTVLHQDWLNGKEYKCKVSNKALPAPIEKTISKAKG
QPREPQVYTLPPSREEMTKNQVSLTCLVKGFYPSDIAVEWE
SNGQPENNYKTTPPVLDSDGSFFLYSKLTVDKSRWQQGNV
FSCSVMHEALHNHYTQKSLSLSPG.

In some embodiments, the IL-2 mutein is linked to the Fc region. Non-limiting examples of linkers are glycine/serine linkers. For example, a glycine/serine linkers can be a sequence of GGGGSGGGGSGGGGSGGGGS (SEQ ID NO: 24) or GGGGSGGGGSGGGGS (SEQ ID NO: 25). This is simply a non-limiting example and the linker can have varying number of GGGGS (SEQ ID NO: 26) or GGGGA repeats (SEQ ID NO: 27). In some embodiments, the linker comprises 1, 2, 3, 4, 5, 6, 7, 8, 9, or 10 of the GGGGS (SEQ ID NO: 26) or GGGGA (SEQ ID NO: 27) repeats.

Thus, the IL-2/Fc fusion can be represented by the formula of Z_{IL-2M}-L_{gs}-Z_{Fc}, wherein Z_{IL-2M} is an IL-2 mutein as described herein, L_{gs} is a linker sequence as described herein (e.g., glycine/serine linker) and Z_{Fc} is a Fc region described herein or known to one of skill in the art. In some embodiments, the formula can be in the reverse orientation Z_{Fc}-L_{gs}-Z_{IL-2M}.

In some embodiments, the IL-2/Fc fusion comprises or consist of a sequence of

(SEQ ID NO: 28)
MYRMQLLSCIALSLALVTNSAPTSSSTKKTQLQLEHLLLDL
QMILNGISNHKNPRLARMLTFKFYMPEKATEIKHLQCLEEE
LKPLEEALRLAPSKNFHLRPRDLISDINVIVLELKGSETTFMC
EYADETATIVEFLNRWITFSQSIISTLTGGGGSGGGGSGGGG
SGGGGSDKTHTCPPCPAPEAAGAPSVFLFPPKPKDTLMISRT
PEVTCVVVDVSHEDPEVKFNWYVDGVEVHNAKTKPREEQ
YNSTYRVVSVLTVLHQDWLNGKEYKCKVSNKALPAPIEKT
ISKAKGQPREPQVYTLPPSREEMTKNQVSLTCLVKGFYPSDI
AVEWESNGQPENNYKTTPPVLDSDGSFFLYSKLTVDKSRW
QQGNVFSCSVMHEALHNHYTQKSLSLSPG
(SEQ ID NO: 29)
MYRMQLLSCIALSLALVTNSAPTSSSTKKTQLQLEHLLLDL
QMILNGISNHKNPRLARMLTFKFYMPEKATELKHIQCLEEE
LKPLEEALRLAPSKNFHLRPRDLISDINVIVLELKGSETTFMC
EYADETATIVEFLNRWITFSQSIISTLTGGGGSGGGGSGGGG
SGGGGSDKTHTCPPCPAPEAAGAPSVFLFPPKPKDTLMISRT
PEVTCVVVDVSHEDPEVKFNWYVDGVEVHNAKTKPREEQ
YNSTYRVVSVLTVLHQDWLNGKEYKCKVSNKALPAPIEKT
ISKAKGQPREPQVYTLPPSREEMTKNQVSLTCLVKGFYPSDI
AVEWESNGQPENNYKTTPPVLDSDGSFFLYSKLTVDKSRW
QQGNVFSCSVMHEALHNHYTQKSLSLSPG
(SEQ ID NO: 30)
MYRMQLLSCIALSLALVTNSAPTSSSTKKTQLQLEHLLLDL
QMILNGISNHKNPRLARMLTFKFYMPEKATELKHLQCLEEE
LKPLEEALRLAPSKNFHIRPRDLISDINVIVLELKGSETTFMC
EYADETATIVEFLNRWITFSQSIISTLTGGGGSGGGGSGGGG
SGGGGSDKTHTCPPCPAPEAAGAPSVFLFPPKPKDTLMISRT
PEVTCVVVDVSHEDPEVKFNWYVDGVEVHNAKTKPREEQ
YNSTYRVVSVLTVLHQDWLNGKEYKCKVSNKALPAPIEKT
ISKAKGQPREPQVYTLPPSREEMTKNQVSLTCLVKGFYPSDI
AVEWESNGQPENNYKTTPPVLDSDGSFFLYSKLTVDKSRW
QQGNVFSCSVMHEALHNHYTQKSLSLSPG
(SEQ ID NO: 31)
MYRMQLLSCIALSLALVTNSAPTSSSTKKTQLQLEHLLLDL
QMILNGISNHKNPRLARMLTFKFYMPEKATELKHLQCLEEE
LKPLEEALRLAPSKNFHLRPRDLISDINVIVLELKGSETTFMC
EYADETATIVEFINRWITFSQSIISTLTGGGGSGGGGSGGGGS
GGGGSDKTHTCPPCPAPEAAGAPSVFLFPPKPKDTLMISRTP
EVTCVVVDVSHEDPEVKFNWYVDGVEVHNAKTKPREEQY
NSTYRVVSVLTVLHQDWLNGKEYKCKVSNKALPAPIEKTIS
KAKGQPREPQVYTLPPSREEMTKNQVSLTCLVKGFYPSDIA
VEWESNGQPENNYKTTPPVLDSDGSFFLYSKLTVDKSRWQ
QGNVFSCSVMHEALHNHYTQKSLSLSPG.

In some embodiments, the IL-2/Fc fusion comprises or consists of a sequence selected from the Table 2 below.

TABLE 2
IL-2/Fc Fusion Protein Amino Acid Sequences

| Sequence | |
|---|---|
| Identification | Sequence |
| SEQ ID NO: 32 | APTSSSTKKTQLQLEHLLLDLQMILNGINNYKNPKLTRMLTFKFYMPKKATELKHLQCLEE |
| | ELKPLEEVLNLAQSKNFHLRPRDLISRINVIVLELKGSETTFMCEYADETATIVEFLNRWI |
| | TFSQSIISTLTGGGGAGGGGDKTHTCPPCPAPELLGGPSVFLFPPKPKDTLMISRTPEVTC |
| | VVVDVSHEDPEVKFNWYVDGVEVHNAKTKPREEQYASTYRVVSVLTVLHQDWLNGKEYKCK |
| | VSNKALPAPIEKTISKAKGQPREPQVYTLPPSRDELTKNQVSLTCLVKGFYPSDIAVEWES |
| | NGQPENNYKTTPPVLDSDGSFFLYSKLTVDKSRWQQGNVFSCSVMHEALHNHYTOKSLSLS |
| | PGK |
| SEQ ID NO: 33 | APTSSSTKKTQLQLEHLLLHLQMILNGINNYKNPKLTRMLTFKFYMPKKATELKHLQCLEE |
| | ELKPLEEVLNLAQSKNFHLRPRDLISNINVIVLELKGSETTFMCEYADETATIVEFLNRWI |
| | TFSQSIISTLTVECPPCPAPPVAGPSVFLFPPKPKDTLMISRTPEVTCVVVDVSHEDPEVQ |
| | FNWYVDGVEVHNAKTKPREEQFNSTFRVVSVLTVVHQDWLNGKEYKCKVSNKGLPAPIEKT |
| | ISKTKGQPREPQVYTLPPSREEMTKNQVSLTCLVKGFYPSDIAVEWESNGQPENNYKTTPP |
| | MLDSDGSFFLYSKLTVDKSRWQQGNVFSCSVMHEALHNHYTQKSLSLSPGK |
| SEQ ID NO: 34 | APTSSSTKKTQLQLEHLLLDLQMILNGINNYKNPKLTRMLTFKFYMPKKATELKHLQCLEE |
| | ELKPLEEVLNLAQSKNFHLRPRDLISRINVIVLELKGSETTFMCEYADETATIVEFLNRWI |
| | TFSQSIISTLTDKTHTCPPCPAPELLGGPSVFLFPPKPKDTLMISRTPEVTCVVVDVSHED |
| | PEVKFNWYVDGVEVHNAKTKPREEQYASTYRVVSVLTVLHQDWLNGKEYKCKVSNKALPAP |
| | IEKTISKAKGQPREPQVYTLPPSRDELTKNQVSLTCLVKGFYPSDIAVEWESNGQPENNYK |
| | TTPPVLDSDGSFFLYSKLTVDKSRWQQGNVFSCSVMHEALHNHYTQKSLSLSPG |
| SEQ ID NO: 35 | APTSSSTKKTQLQLEHLLLDLQMILNGINNYKNPKLTRMLTFKFYMPKKATELKHLQCLEE |
| | ELKPLEEVLNLAQSKNFHLRPRDLISRINVIVLELKGSETTFMCEYADETATIVEFLNRWI |
| | TFSQSIISTLTGGGGSDKTHTCPPCPAPELLGGPSVFLFPPKPKDTLMISRTPEVTCVVVD |
| | VSHEDPEVKFNWYVDGVEVHNAKTKPREEQYASTYRVVSVLTVLHQDWLNGKEYKCKVSNK |
| | ALPAPIEKTISKAKGQPREPQVYTLPPSRDELTKNQVSLTCLVKGFYPSDIAVEWESNGQP |
| | ENNYKTTPPVLDSDGSFFLYSKLTVDKSRWQQGNVFSCSVMHEALHNHYTQKSLSLSPG |
| SEQ ID NO: 36 | APTSSSTKKTQLQLEHLLLDLQMILNGINNYKNPKLTRMLTFKFYMPKKATELKHLQCLEE |
| | ELKPLEEVLNLAQSKNFHLRPRDLISRINVIVLELKGSETTFMCEYADETATIVEFLNRWI |
| | TFSQSIISTLTGGGGSGGGGSDKTHTCPPCPAPELLGGPSVFLFPPKPKDTLMISRTPEVT |
| | CVVVDVSHEDPEVKFNWYVDGVEVHNAKTKPREEQYASTYRVVSVLTVLHQDWLNGKEYKC |
| | KVSNKALPAPIEKTISKAKGQPREPQVYTLPPSRDELTKNQVSLTCLVKGFYPSDIAVEWE |
| | SNGQPENNYKTTPPVLDSDGSFFLYSKLTVDKSRWQQGNVFSCSVMHEALHNHYTOKSLSL |
| | SPG |
| SEQ ID NO: 37 | APTSSSTKKTQLQLEHLLLDLQMILNGINNYKNPKLTRMLTFKFYMPKKATELKHLQCLEE |
| | ELKPLEEVLNLAQSKNFHLRPRDLISRINVIVLELKGSETTFMCEYADETATIVEFLNRWI |
| | TFSQSIISTLTGGGGSGGGGSGGGGSDKTHTCPPCPAPELLGGPSVFLFPPKPKDTLMISR |
| | TPEVTCVVVDVSHEDPEVKFNWYVDGVEVHNAKTKPREEQYASTYRVVSVLTVLHQDWLNG |
| | KEYKCKVSNKALPAPIEKTISKAKGQPREPQVYTLPPSRDELTKNQVSLTCLVKGFYPSDI |
| | AVEWESNGQPENNYKTTPPVLDSDGSFFLYSKLTVDKSRWQQGNVFSCSVMHEALHNYHTQ |
| | KSLSLSPG |
| SEQ ID NO: 38 | APTSSSTKKTQLQLEHLLLDLQMILNGINNYKNPKLTRMLTFKFYMPKKATELKHLQCLEE |
| | ELKPLEEVLNLAQSKNFHLRPRDLISRINVIVLELKGSETTFMCEYADETATIVEFLNRWI |
| | TFSQSIISTLTGGGGSGGGGSGGGGSGGGGSDKTHTCPPCPAPELLGGPSVFLFPPKPKDT |
| | LMISRTPEVTCVVVDVSHEDPEVKFNWYVDGVEVHNAKTKPREEQYASTYRVVSVLTVLHQ |
| | DWLNGKEYKCKVSNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQGNVFSCSVMHEALH |
| | NHYTQKSLSLSPG |
| SEQ ID NO: 39 | APTSSSTKKTQLQLEHLLLHLQMILNGINNYKNPKLTRMLTFKFYMPKKATELKHLQCLEE |
| | ELKPLEEVLNLAQSKNFHLRPRDLISNINVIVLELKGSETTFMCEYADETATIVEFLNRWI |
| | TFSQSIISTLTGGGGSGGGGSGGGGSDKTHTCPPCPAPELLGGPSVFLFPPKPKDTLMISR |
| | TPEVTCVVVDVSHEDPEVKFNWYVDGVEVHNAKTKPREEQYASTYPVVSVLTVLHQDWING |
| | KEYKCKVSNKALPAPIEKTISKAKGQPREPQVYTLPPSRDELTKNQVSLTCLVKGFYPSDI |
| | AVEWESNGQPENNYKTTPPVLDSDGSFFLYSKLTVDKSRWQQGNVFSCSVMHEALHNHYTQ |
| | KSLSLSPG |

In some embodiments, the IL-2 muteins comprises one or more of the sequences provided in the following table, which, in some embodiments, shows the IL-2 mutein fused with other proteins or linkers. The table also provides sequences for a variety of Fc domains or variants that the IL-2 can be fused with:

| SEQ ID | Brief | |
|---|---|---|
| NO: | Description | Amino Acid Sequence |
| 40 | Human IL-2 | APTSSSTKKTQLQLEHLLLDLQMILNGINNYKNPKLTRMLTFKFYMPKKA |
| | with C125S | TELKHLQCLEEELKPLEEVLNLAQSKNFHLRPRDLISNINVIVLELKGSE |
| | mutation | TTFMCEYADETATIVEFLNRWITFSQSIISTLT |
| 41 | Human IL-2 | APASSSTKKTQLQLEHLLLDLQMILNGINNYKNPKLTRMLTFKFYMPKKA |
| | with C125S | TELKHLQCLEEELKPLEEVLNLAQSKNFHLRPRDLISNINVIVLELKGSE |
| | and T3A | TTFMCEYADETATIVEFLNRWITFSQSIISTLT |
| | mutations | |
| 42 | Human IL-2 | APTSSSTKKTQLQLEHLLLDLQMILNGINNYKNPKLTRMLTFKFYMPKKA |
| | with N88R and | TELKHLQCLEEELKPLEEVLNLAQSKNFHLRPRDLISRINVIVLELKGSE |
| | C125S | TTFMCEYADETATIVEFLNRWITFSQSIISTLT |
| 43 | Human IL-2 | APTSSSTKKTQLQLEHLLLDLQMILNGINNYKNPKLTRMLTFKFYMPKKA |
| | with V69A, | TELKHLQCLEEELKPLEEALNLAPSKNFHLRPRDLISNINVIVLELKGSE |
| | Q74P and | TTFMCEYADETATIVEFLNRWITFSQSIISTLT |
| | C125S | |
| | mutations | |
| 44 | Human IL-2 | APTSSSTKKTQLQLEHLLLDLQMILNGINNYKNPKLTRMLTFKFYMPKKA |
| | with V69A, | TELKHLQCLEEELKPLEEALNLAPSKNFHLRPRDLISDINVIVLELKGSE |
| | Q74P, N88D | TTFMCEYADETATIVEFLNRWITFSQSIISTLT |
| | and C125S | |
| | mutations | |
| 45 | Human IL-2 | APTSSSTKKTQLQLEHLLLDLQMILNGINNYKNPKLTRMLTFKFYMPKKA |
| | with V69A, | TELKHLQCLEEELKPLEEALNLAPSKNFHLRPRDLISRINVIVLELKGSE |
| | Q74P, N88R | TTFMCEYADETATIVEFLNRWITFSQSIISTLT |
| | and C125S | |
| | mutations | |
| 46 | Human IL-2 | APTSSSTKKTQLQLEHLLLDLQMILNGINNYKNPKLTRMLTFKFYMPKKA |
| | with N88D and | TELKHLQCLEEELKPLEEVLNLAQSKNFHLRPRDLISDINVIVLELKGSE |
| | C125S | TTFMCEYADETATIVEFLNRWITFSQSIISTLT |
| 47 | Human IL-2 | APTSSSTKKTQLQLEHLLLDLQMILNGINNYKNPKLTRMLTFKFYMPKKA |
| | with L53I, | TEIKHLQCLEEELKPLEEALNLAPSKNFHLRPRDLISDINVIVLELKGSE |
| | V69A, Q74P, | TTFMCEYADETATIVEFLNRWITFSQSIISTLT |
| | N88D and | |
| | C125S | |
| | mutations | |
| 48 | Human IL-2 | APTSSSTKKTQLQLEHLLLDLQMILNGINNYKNPKLTRMLTFKFYMPKKA |
| | with L56I, | TELKHIQCLEEELKPLEEALNLAPSKNFHLRPRDLISDINVIVLELKGSE |
| | V69A, Q74P, | TTFMCEYADETATIVEFLNRWITFSQSIISTLT |
| | N88D and | |
| | C125S | |
| | mutations | |
| 49 | Human IL-2 | APTSSSTKKTQLQLEHLLLDLQMILNGINNYKNPKLTRMLTFKFYMPKKA |
| | with V69A, | TELKHLQCLEEELKPLEEALNLAPSKNFHIRPRDLISDINVIVLELKGSE |
| | Q74P, L80I, | TTFMCEYADETATIVEFLNRWITFSQSIISTLT |
| | N88D and | |
| | C125S | |
| | mutations | |
| 50 | Human IL-2 | APTSSSTKKTQLQLEHLLLDLQMILNGINNYKNPKLTRMLTFKFYMPKKA |
| | with V69A, | TELKHLQCLEEELKPLEEALNLAPSKNFHLRPRDLISDINVIVLELKGSE |
| | Q74P, N88D, | TTFMCEYADETATIVEFINRWITFSQSIISTLT |
| | L118I, and | |
| | C125S | |
| | mutations | |
| 51 | Human IgG1 Fc | DKTHTCPPCPAPEAAGAPSVFLFPPKPKDTLMISRTPEVTCVVVDVSHED |
| | (N-terminal | PEVKFNWYVDGVEVHNAKTKPREEQYNSTYRVVSVLTVLHQDWLNGKEYK |
| | fusions) with | CKVSNKALPAPIEKTISKAKGQPREPQVYTLPPSREEMTKNQVSLTCLVK |
| | L234A, L235A, | GFYPSDIAVEWESNGQPENNYKTTPPVLDSDGSFFLYSKLTVDKSRWQQG |
| | and G237A | NVFSCSVMHEALHNHYTQKSLSLSPG |
| | mutations | |
| 25 | GGGGSGGGGSGGG | GGGGGGGGSGGGGS |
| | GS linker (15 | |
| | amino acids) | |
| 24 | GGGGSGGGGSGGG | GGGGSGGGGSGGGGSGGGGS |
| | GSGGGGS | |
| | linker (20 | |
| | amino acids) | |
| 26 | GGGGS linker | GGGGS |
| | (5 amino | |
| | acids ) | |
| 52 | Human IgG1 Fc | DKTHTCPPCPAPELLGGPSVFLFPPKPKDTLMISRTPEVTCVVVDVSHED |
| | (truncated) | PEVKFNWYVDGVEVHNAKTKPREEQYGSTYRVVSVLTVLHQDWLNGKEYK |
| | with N297G | CKVSNKALPAPIEKTISKAKGQPREPQVYTLPPSREEMTKNQVSLTCLVK |
| | mutation | GFYPSDIAVEWESNGQPENNYKTTPPVLDSDGSFFLYSKLTVDKSRWOOG |
| | | NVFSCSVMHEALHNHYTQKSLSLSPG |
| 53 | Antibody | ASTKGPSVFPLAPSSKSTSGGTAALGCLVKDYFPEPVTVSWNSGALTSGV |
| | Heavy Chain | HTFPAVLOSSGLYSLSSVVTVPSSSLGTQTYICNVNHKPSNTKVDKKVEP |
| | CH1-CH2-CH3 | KSCDKTHTCPPCPAPEAAGAPSVELFPPKPKDTLMISRTPEVTCVVVDVS |
| | domains | HEDPEVKFNWYVDGVEVHNAKTKPREEQYNSTYRVVSVLTVLHQDWLNGK |
| | (human IgG1 | EYKCKVSNKALPAPIEKTISKAKGQPREPQVYTLPPSREEMTKNQVSLTC |
| | with L234A, | LVKGFYPSDIAVEWESNGQPENNYKTTPPVLDSDGSFFLYSKLTVDKSRW |
| | L235A, and | QQGNVFSCSVMHEALHNHYTQKSLSLSPG |
| | G237A) | |
| 54 | Antibody | RTVAAPSVFIFPPSDEQLKSGTASVVCLLNNFYPREAKVQWKVDNALQSG |
| | Kappa | NSQESVTEQDSKDSTYSLSSTLTLSKADYEKHKVYACEVTHQGLSSPVTK |
| | Constant | SFNRGEC |
| | Domain | |
| | (human) | |
| 55 | IL-2-G4Sx3-Fc | APTSSSTKKTQLQLEHLLLDLQMILNGINNYKNPKLTRMLTFKFYMPKKA |
| | | TELKHLQCLEEELKPLEEVLNLAQSKNFHLRPRDLISNINVIVLELKGSE |
| | | TTFMCEYADETATIVEFLNRWITFSQSIISTLTGGGGSGGGGSGGGGSDK |
| | | THTCPPCPAPEAAGAPSVFLFPPKPKDTLMISRTPEVTCVVVDVSHEDPE |
| | | VKFNWYVDGVEVHNAKTKPREEQYNSTYRVVSVLTVLHQDWLNGKEYKCK |
| | | VSNKALPAPIEKTISKAKGQPREPQVYTLPPSREEMTKNQVSLTCLVKGF |
| | | YPSDIAVEWESNGQPENNYKTTPPVLDSDGSFFLYSKLTVDKSRWQQGNV |
| | | FSCSVMHEALHNHYTQKSLSLSPG |
| 56 | IL-2 T3A- | APASSSTKKTQLQLEHLLLDLQMILNGINNYKNPKLTRMLTFKFYMPKKA |
| | G4Sx3-Fc | TELKHLQCLEEELKPLEEVLNLAQSKNFHLRPRDLISNINVIVLELKGSE |
| | | TTFMCEYADETATIVEFLNRWITFSQSIISTLTGGGGSGGGGSGGGGSDK |
| | | THTCPPCPAPEAAGAPSVELFPPKPKDTLMISRTPEVTCVVVDVSHEDPE |
| | | VKFNWYVDGVEVHNAKTKPREEQYNSTYRVVSVLTVLHQDWLNGKEYKCK |
| | | VSNKALPAPIEKTISKAKGQPREPQVYTLPPSREEMTKNQVSLTCLVKGF |
| | | YPSDIAVEWESNGQPENNYKTTPPVLDSDGSFFLYSKLTVDKSRWQQGNV |
| | | FSCSVMHEALHNHYTQKSLSLSPG |
| 57 | IL-2 N88R- | APTSSSTKKTQLQLEHLLLDLQMILNGINNYKNPKLTRMLTFKFYMPKKA |
| | G4Sx3-Fc | TELKHLQCLEEELKPLEEVLNLAQSKNFHLRPRDLISRINVIVLELKGSE |
| | | TTFMCEYADETATIVEFLNRWITFSQSIISTLTGGGGSGGGGSGGGGSDK |
| | | THTCPPCPAPEAAGAPSVFLFPPKPKDTLMISRTPEVTCVVVDVSHEDPE |
| | | VKFNWYVDGVEVHNAKTKPREEQYNSTYRVVSVLTVLHQDWLNGKEYKCK |
| | | VSNKALPAPIEKTISKAKGQPREPQVYTLPPSREEMTKNQVSLTCLVKGF |
| | | YPSDIAVEWESNGQPENNYKTTPPVLDSDGSFFLYSKLTVDKSRWQQGNV |
| | | FSCSVMHEALHNHYTQKSLSLSPG |
| 58 | IL-2 V69A, | APTSSSTKKTQLQLEHLLLDLQMILNGINNYKNPKLTRMLTFKFYMPKKA |
| | Q74P,-G4Sx3- | TELKHLQCLEEELKPLEEALNLAPSKNFHLRPRDLISNINVIVLELKGSE |
| | Fc | TTFMCEYADETATIVEFLNRWITFSQSIISTLTGGGGSGGGGSGGGGSDK |
| | | THTCPPCPAPEAAGAPSVFLFPPKPKDTLMISRTPEVTCVVVDVSHEDPE |
| | | VKFNWYVDGVEVHNAKTKPREEQYNSTYRVVSVLTVLHQDWLNGKEYKCK |
| | | VSNKALPAPIEKTISKAKGQPREPQVYTLPPSREEMTKNQVSLTCLVKGF |
| | | YPSDIAVEWESNGQPENNYKTTPPVLDSDGSFFLYSKLTVDKSRWQQGNV |
| | | FSCSVMHEALHNHYTQKSLSLSPG |
| 59 | IL-2 N88D | APTSSSTKKTQLQLEHLLLDLQMILNGINNYKNPKLTRMLTFKFYMPKKA |
| | V69A, Q74P- | TELKHLQCLEEELKPLEEALNLAPSKNFHLRPRDLISDINVIVLELKGSE |
| | G4Sx3-Fc | TTFMCEYADETATIVEFLNRWITFSQSIISTLTGGGGSGGGGSGGGGSDK |
| | | THTCPPCPAPEAAGAPSVELFPPKPKDTLMISRTPEVTCVVVDVSHEDPE |
| | | VKFNWYVDGVEVHNAKTKPREEQYNSTYRVVSVLTVLHQDWLNGKEYKCK |
| | | VSNKALPAPIEKTISKAKGQPREPQVYTLPPSREEMTKNQVSLTCLVKGF |
| | | YPSDIAVEWESNGQPENNYKTTPPVLDSDGSFFLYSKLTVDKSRWQQGNV |
| | | FSCSVMHEALHNHYTQKSLSLSPG |
| 60 | IL-2 N88R | APTSSSTKKTQLQLEHLLLDLQMILNGINNYKNPKLTRMLTFKFYMPKKA |
| | V69A, Q74P- | TELKHLQCLEEELKPLEEALNLAPSKNFHLRPRDLISRINVIVLELKGSE |
| | G4Sx3-Fc | TTFMCEYADETATIVEFLNRWITFSQSIISTLTGGGGSGGGGSGGGGSDK |
| | | THTCPPCPAPEAAGAPSVELFPPKPKDTLMISRTPEVTCVVVDVSHEDPE |
| | | VKFNWYVDGVEVHNAKTKPREEQYNSTYRVVSVLTVLHQDWLNGKEYKCK |
| | | VSNKALPAPIEKTISKAKGQPREPQVYTLPPSREEMTKNQVSLTCLVKGF |
| | | YPSDIAVEWESNGQPENNYKTTPPVLDSDGSFFLYSKLTVDKSRWQQGNV |
| | | FSCSVMHEALHNHYTQKSLSLSPG |
| 61 | IL-2 N88D- | APTSSSTKKTQLQLEHLLLDLQMILNGINNYKNPKLTRMLTFKFYMPKKA |
| | G4Sx3-Fc | TELKHLQCLEEELKPLEEVLNLAQSKNFHLRPRDLISDINVIVLELKGSE |
| | | TTFMCEYADETATIVEFLNRWITFSQSIISTLTGGGGSGGGGSGGGGSDK |
| | | THTCPPCPAPEAAGAPSVFLFPPKPKDTLMISRTPEVTCVVVDVSHEDPE |
| | | VKFNWYVDGVEVHNAKTKPREEQYNSTYRVVSVLTVLHQDWLNGKEYKCK |
| | | VSNKALPAPIEKTISKAKGQPREPQVYTLPPSREEMTKNQVSLTCLVKGF |
| | | YPSDIAVEWESNGQPENNYKTTPPVLDSDGSFFLYSKLTVDKSRWQQGNV |
| | | FSCSVMHEALHNHYTQKSLSLSPG |
| 62 | IL-2 L53I | APTSSSTKKTQLQLEHLLLDLQMILNGINNYKNPKLTRMLTFKFYMPKKA |
| | N88D V69A, | TEIKHLQCLEEELKPLEEALNLAPSKNFHLRPRDLISDINVIVLELKGSE |
| | Q74P,C125S- | TTFMCEYADETATIVEFLNRWITFSQSIISTLTGGGGSGGGGSGGGGSGG |
| | G4Sx4-Fc | GGSDKTHTCPPCPAPEAAGAPSVFLFPPKPKDTLMISRTPEVTCVVVDVS |
| | | HEDPEVKFNWYVDGVEVHNAKTKPREEQYNSTYRVVSVLTVLHQDWLNGK |
| | | EYKCKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKSRW |
| | | QQGNVFSCSVMHEALHNHYTQKSLSLSPG |
| 63 | IL-2 L56I | APTSSSTKKTQLQLEHLLLDLQMILNGINNYKNPKLTRMLTFKFYMPKKA |
| | N88D V69A, | TELKHIQCLEEELKPLEEALNLAPSKNFHLRPRDLISDINVIVLELKGSE |
| | Q74P, C125S | TTFMCEYADETATIVEFLNRWITFSQSIISTLTGGGGSGGGGSGGGGSGG |
| | G4Sx4-Fc | GGSDKTHTCPPCPAPEAAGAPSVFLFPPKPKDTLMISRTPEVTCVVVDVS |
| | | HEDPEVKFNWYVDGVEVHNAKTKPREEQYNSTYRVVSVLTVLHQDWLNGK |
| | | EYKCKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKSRW |
| | | QQGNVFSCSVMHEALHNHYTQKSLSLSPG |
| 64 | IL-2 L80I | APTSSSTKKTQLQLEHLLLDLQMILNGINNYKNPKLTRMLTFKFYMPKKA |
| | N88D V69A, | TELKHLQCLEEELKPLEEALNLAPSKNFHIRPRDLISDINVIVLELKGSE |
| | C125S Q74P- | TTFMCEYADETATIVEFLNRWITFSQSIISTLTGGGGSGGGGSGGGGSGG |
| | G4Sx4-Fc | GGSDKTHTCPPCPAPEAAGAPSVFLFPPKPKDTLMISRTPEVTCVVVDVS |
| | | HEDPEVKFNWYVDGVEVHNAKTKPREEQYNSTYRVVSVLTVLHQDWLNGK |
| | | EYKCKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKSRW |
| | | QQGNVFSCSVMHEALHNHYTQKSLSLSPG |
| 65 | IL-2 L118I | APTSSSTKKTQLQLEHLLLDLQMILNGINNYKNPKLTRMLTFKFYMPKKA |
| | N88D V69A, | TELKHLQCLEEELKPLEEALNLAPSKNFHLRPRDLISDINVIVLELKGSE |
| | Q74P, C125S- | TTFMCEYADETATIVEFINRWITFSQSIISTLTGGGGSGGGGSGGGGSGG |
| | G4Sx4-Fc | GGSDKTHTCPPCPAPEAAGAPSVFLFPPKPKDTLMISRTPEVTCVVVDVS |
| | | HEDPEVKFNWYVDGVEVHNAKTKPREEQYNSTYRVVSVLTVLHQDWLNGK |
| | | EYKCKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKSRW |
| | | QQGNVFSCSVMHEALHNHYTQKSLSLSPG |
| 66 | IL-2 N88D | APTSSSTKKTQLQLEHLLLDLQMILNGINNYKNPKLTRMLTFKFYMPKKA |
| | V69A, Q74P- | TELKHLQCLEEELKPLEEALNLAPSKNFHLRPRDLISDINVIVLELKGSE |
| | G4Sx4-Fc | TTFMCEYADETATIVEFLNRWITFSQSIISTLTGGGGSGGGGSGGGGSGG |
| | | GGSDKTHTCPPCPAPEAAGAPSVFLFPPKPKDTLMISRTPEVTCVVVDVS |
| | | HEDPEVKFNWYVDGVEVHNAKTKPREEQYNSTYRVVSVLTVLHQDWLNGK |
| | | EYKCKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKSRW |
| | | QQGNVFSCSVMHEALHNHYTQKSLSLSPG |
| 67 | Fc-G4S-IL-2 | DKTHTCPPCPAPELLGGPSVFLFPPKPKDTLMISRTPEVTCVVVDVSHED |
| | N88D V69A, | PEVKFNWYVDGVEVHNAKTKPREEQYGSTYRVVSVLTVLHQDWLNGKEYK |
| | Q74P | CKVSNKALPAPIEKTISKAKGQPREPQVYTLPPSREEMTKNQVSLTCLVK |
| | | GFYPSDIAVEWESNGQPENNYKTTPPVLDSDGSFFLYSKLTVDKSRWOOG |
| | | NVFSCSVMHEALHNHYTQKSLSLSPGGGGGSAPTSSSTKKTQLQLEHLLL |
| | | DLQMILNGINNYKNPKLTRMLTFKFYMPKKATELKHLQCLEEELKPLEEA |
| | | LNLAPSKNFHLRPRDLISDINVIVLELKGSETTFMCEYADETATIVEFLN |
| | | RWITFAQSIISTLT |
| 68 | IL-2 N88D | APTSSSTKKTQLQLEHLLLDLQMILNGINNYKNPKLTRMLTFKFYMPKKA |
| | V69A, Q74P, | TEX₁KHX₂QCLEEELKPLEEALNLAPSKNFHX₃RPRDLISDINVIVLELKG |
| | C125S-G4Sx4- | SETTFMCEYADETATIVEFX₄NRWITFSQSIISTLTGGGGSGGGGSGGGGS |
| | Fc, wherein | GGGGSDKTHTCPPCPAPEAAGAPSVFLFPPKPKDTLMISRTPEVTCVVVD |
| | at least one | VSHEDPEVKFNWYVDGVEVHNAKTKPREEQYNSTYRVVSVLTVLHQDWLN |
| | of X₁, X₂, X₃, | GKEYKCKVSNKALPAPIEKTISKAKGQPREPQVYTLPPSREEMTKNQVSL |
| | and X₄ is I | TCLVKGFYPSDIAVEWESNGQPENNYKTTPPVLDSDGSFFLYSKLTVDKS |
| | and the | RWQQGNVFSCSVMHEALHNHYTQKSLSLSPG |
| | remainder are | |
| | L or I. | |
| 69 | IL-2 N88D | APTSSSTKKTQLQLEHLLLDLQMILNGINNYKNPKLTRMLTFKFYMPKKA |
| | V69A, Q74P, | TEX₁KHX₂QCLEEELKPLEEALNLAPSKNFHX₃RPRDLISDINVIVLELKG |
| | C125S, | SETTFMCEYADETATIVEFX₄NRWITFSQSIISTLT |
| | wherein at | |
| | least one of | |
| | X₁, X₂, X₃, | |
| | and X₄ is I | |
| | and the | |
| | remainder are | |
| | L or I. | |

In some embodiments, the sequences shown in the table or throughout comprise one or more mutations that correspond to positions L53, L56, L80, and L118. In some embodiments, the sequences shown in the table or throughout do not comprise one or more mutations that correspond to positions L53, L56, L80, and L118. In some embodiments, the sequences shown in the table or throughout the present application comprise one or more mutations that correspond to positions L591, L631, 124L, L941, L961 or L1321 or other substitutions at the same positions. In some embodiments, the sequences shown in the table or throughout the present application do not comprise one or more mutations that correspond to positions L591, L631, 124L, L941, L961 or L1321 or other substitutions at the same positions. In some embodiments, the mutation is a leucine to isoleucine substitution. In some embodiments, the mutein does not comprise other mutations than as shown or described herein. In some embodiments, the peptide comprises or consists of a sequence of SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, or SEQ ID NO: 65, SEQ ID NO: 66, SEQ ID NO: 67, SEQ ID NO: 68, or SEQ ID NO: 69.

In some embodiments, the protein comprises an IL-2 mutein as provided for herein. In some embodiments, a polypeptide is provided comprising or consisting of SEQ ID NO: 68 or SEQ ID NO: 69, wherein at least one of X₁, X₂, X₃, and X₄ is I and the remainder are L or I. In some embodiments, X₁, X₂, and X₃ are L and X₄ is I. In some embodiments, X₁, X₂, and X₄ are L and X₃ is I. In some embodiments, X₂, X₃, and X₄ are L and X₁ is I. In some embodiments, X₁, X₃, and X₄ are L and X₂ is I. In some embodiments, X₁ and X₂ are L and X₃ and X₄ are I. In some embodiments, X₁ and X₃ are L and X₂ and X₄ are I. In some embodiments, X₁ and X₄ are L and X₂ and X₃ are I. In some embodiments, X₂ and X₃ are L and X₁ and X₄ are I. In some embodiments, X₂ and X₄ are L and X₁ and X₃ are I. In some embodiments, X₃ and X₄ are L and X₁ and X₂ are I. In some embodiments, X₁, X₂, and X₃ are L and X₄ is I. In some embodiments, X₂, X₃, and X₄ are L and X₁ is I. In some embodiments, X₁, X₃, and X₄ are L and X₂ is I. In some embodiments, X₁, X₂, and X₄ are L and X₃ is I.

In some embodiments, the Fc portion of the fusion is not included. In some embodiments, the peptide consists essentially of an IL-2 mutein provided for herein. In some embodiments, the protein is free of an Fc portion.

The sequences are for illustrative purposes only and are not intended to be limiting. In some embodiments, the compound comprises or consist of an amino acid sequence of SEQ ID NO: 62, 63, 64, or 65. In some embodiments, the compound comprises or consist of an amino acid sequence of SEQ ID NO: 62, 63, 64, or 65 with or without a C125A or C125S mutation. In some embodiments, the residue at position 125 is C, S, or A. In some embodiments, the compound comprises or consist of an amino acid sequence of SEQ ID NO: 68 or SEQ ID NO: 69, wherein at least one of X₁, X₂, X₃, and X₄ is I and the remainder are L or I. In some embodiments, a polypeptide is provided comprising or consisting of SEQ ID NO: 68 or SEQ ID NO: 69, wherein at least one of X₁, X₂, X₃, and X₄ is I and the remainder are L or I. In some embodiments, X₁, X₂, and X₃ are L and X₄ is I. In some embodiments, X₁, X₂, and X₄ are L and X₃ is I. In some embodiments, X₂, X₃, and X₄ are L and X₁ is I. In some embodiments, X₁, X₃, and X₄ are L and X₂ is I. In some embodiments, X₁ and X₂ are L and X₃ and X₄ are I. In some embodiments, X₁ and X₃ are L and X₂ and X₄ are I. In some embodiments, X₁ and X₄ are L and X₂ and X₃ are I. In some embodiments, X₂ and X₃ are L and X₁ and X₄ are I. In some embodiments, X₂ and X₄ are L and X₁ and X₃ are I. In some embodiments, X₃ and X₄ are L and X₁ and X₂ are I. In some embodiments, X₁, X₂, and X₃ are L and X₄ is I. In some embodiments, X₂, X₃, and X₄ are L and X₁ is I. In some embodiments, X₁, X₃, and X₄ are L and X₂ is I. In some embodiments, X₁, X₂, and X₄ are L and X₃ is I.

In some embodiments, each of the proteins may also have the C125S and the LALA and/or G237A mutations as provided for herein. The C125 substitution can also be C125A as described throughout the present application.

In some embodiments, an IL-2 mutein molecule comprises at least 60, 70, 80, 85, 90, 95, 96, 97, 98, or 99% sequence identity or homology with a naturally occurring human IL-2 molecule, e.g., a naturally occurring IL-2 sequence disclosed herein or those that incorporated by reference.

As described herein, the IL-2 muteins can be part of a bispecific molecule with a tethering moiety, such as an anti-Robo2, anti-COL4A3, anti-COL4A4, or anti-COL4A5 antibody that will target the IL-2 mutein to a Robo2, COL4A3, COL4A4, or COL4A5 expressing cell. In some embodiments, the IL-2 muteins can be part of a bispecific molecule with a tethering moiety, such as an anti-Robo2, anti-COL4A3, anti-COL4A4, or anti-COL4A5 antibody that will target the IL-2 mutein to a Robo2, COL4A3, COL4A4, or COL4A5 expressing membrane. As described herein, the bispecific molecule can be produced from two polypeptide chains.

In some embodiments, an anti-Robo2 antibody, anti-COL4A3 antibody, anti-COL4A4 antibody, or anti-COL4A5 antibody, or any antigen binding fragment thereof, is linked to a complement modulator effector domain. In some embodiments, the anti-Robo2 antibody, anti-COL4A3 antibody, anti-COL4A4 antibody, or anti-COL4A5 antibody, or any antigen binding fragment thereof, is linked to a CD55 effector domain. In some embodiments, the effector domain has a CD55 sequence as provided herein. In some embodiments, the anti-Robo2 antibody, anti-COL4A3 antibody, anti-COL4A4 antibody, or anti-COL4A5 antibody, or any antigen binding fragment thereof, is linked to a CD59 effector domain. In some embodiments, the effector domain has a CD59 sequence as provided herein. In some embodiments, the anti-Robo2 antibody, anti-COL4A3 antibody, anti-COL4A4 antibody, or anti-COL4A5 antibody, or any antigen binding fragment thereof, is linked to a CR1 effector domain. In some embodiments, the effector domain has a CR1 sequence as provided herein. In some embodiments, the anti-Robo2 antibody, anti-COL4A3 antibody, anti-COL4A4 antibody, or anti-COL4A5 antibody, or any antigen binding fragment thereof, is linked to a DCP effector domain. In some embodiments, the effector domain has a DCP sequence as provided herein.

### Auto-Immune Disorders

As provided herein, therapeutic compounds (proteins) and methods described herein can be used to treat a subject having, or at risk for having, an unwanted autoimmune response, that affects the kidney. Examples of such conditions are provided herein and include, but are not limited to Goodpasture's Syndrome (anti-GBM disease), inflammatory renal disease, glomerulonephritis, nephritis, lupus, lupus nephritis, IgA nephritis, membranous nephropathy, membranoproliferative glomerulonephritis, acute kidney injury, and chronic kidney disease as well as any other autoimmune or inflammation disorders that can affect the kidneys. Other examples include, but are not limited to, focal segmented glomerular sclerosis (FSGS) and other diseases that can affect kidney, for example lupus nephritis, systemic scleroderma, membranous glomerular nephropathy (MGN), membranous nephropathy (MN), minimal change disease (MCD), IgA nephropathy, ANCA-associated vasculitis (AAV), Sjögren's syndrome, and Scleroderma, systemic sclerosis (SSc).

Other examples of autoimmune disorders and diseases that can be treated with the compounds, compositions, and proteins provided herein include, but are not limited to, anti-glomerular basement membrane nephritis, lupus nephritis, membranous glomerulonephropathy, or chronic kidney disease (“CKD”).

In some embodiments, the treatment minimizes rejection of, minimizes immune effector cell mediated damage to, prolongs the survival of subject tissue undergoing, or a risk for, autoimmune attack.

### Glomerular Disorders

As provided herein, therapeutic compounds (proteins) and methods described herein can be used to treat a subject having, or at risk for having, an unwanted complement-mediated disorder, that effect the kidney. Examples of such disorders are provided herein and include, but are not limited to atypical hemolytic uremic syndrome (aHUS), anti-neutrophil cytoplasmic antibody mediated vasculitis (ANCA), C3 glomerulopathy, IgA nephropathy, immune complex membranoproliferative glomerulonephritis, ischemic reperfusion injury, lupus nephritis, membranous nephropathy, and chronic transplant mediated glomerulopathy.

Also provided herein are methods of treating e.g., therapeutically treating or prophylactically treating (or preventing), or ameliorating symptoms of glomerulonephropathies and glomerulonephritides. Examples of glomerulonephropathies that can be treated with the proteins of the invention include, but are not limited to, immune-complex glomerulonephritis (GN), pauci-immune GN, anti-glomerular basement membrane GN, monoclonal immunoglobulin GN, C3 glomerulopathy, nephrotic syndrome (NS), primary congenital NS (CNS), renal tubular acidosis (RTA), inherited renal tubulopathies, Faconi syndrome, primary nephrogenic diabetes insipidus, Goodpasture syndrome, Alport syndrome. In some embodiments, the glomerulonephritis is a primary glomerulonephritis. In some embodiments, the primary glomerulonephritis can be, but is not limited to, minimal change disease, focal segmental glomerular sclerosis, membranous nephropathy, immunoglobulin A nephropathy, C3 glomerulopathy (DDD, C3 GN), idiopathic immune complex membranoproliferative GN, C4 glomerulopathy, infection-related GN, renal-limited GN, renal limited vasculitis, collagenofibrotic glomerulopathy, Thin basement membranes nephropathy, lipoprotein glomerulopathy, ‘Pure’ mesangial proliferative GN, IgM nephropathy, C1q nephropathy, and Idiopathic nodular glomerulosclerosis (diabetic nephropathy without diabetes).

In some embodiments, the proteins of the invention minimize immune effector cell mediated damage to, prolong the survival of subject tissue undergoing, or a risk for, a complement-mediated disorder.

### Therapeutic Compounds

A therapeutic compound, which can be a protein, comprises a specific targeting moiety functionally associated with an effector binding/modulating moiety. In some embodiments, the specific targeting moiety (e.g. anti-Robo2 antibody, anti-COL4A3 antibody, anti-COL4A4 antibody, or anti-COL4A5 antibody,) and effector binding/modulating moiety are linked to one another by a covalent or noncovalent bond, e.g., a covalent or non-covalent bond directly linking the one to the other. In other embodiments, a specific targeting moiety and effector binding/modulating moiety are linked, e.g., covalently or noncovalently, through a linker moiety. For example, in the case of a fusion protein, a polypeptide sequence comprising the specific targeting moiety and a polypeptide sequence comprising the specific effector moiety can be directly linked to one another or linked through one or more linker sequences. In some embodiments, the linker moiety comprises a polypeptide. Linkers are not, however, limited to polypeptides. In some embodiments, a linker moiety comprises other backbones, e.g., a non-peptide polymer, e.g., a PEG polymer. In some embodiments, a linker moiety can comprise a particle, e.g., a nanoparticle, e.g., a polymeric nanoparticle. In some embodiments, a linker moiety can comprise a branched molecule, or a dendrimer.

In some embodiments, a therapeutic compound comprises a protein comprising a specific targeting moiety covalently or non-covalently conjugated to an effector binding/modulating moiety. In some embodiments, a therapeutic molecule comprises a fusion protein having comprising a specific targeting moiety fused, e.g., directly or through a linking moiety comprising one or more amino acid residues, to an effector binding/modulating moiety. In some embodiments, a therapeutic molecule comprises a protein comprising a specific targeting moiety linked by a non-covalent bond or a covalent bond, e.g., a covalent bond other than a peptide bond, e.g., a sulfhydryl bond, to an effector binding/modulating moiety.

In some embodiments, a therapeutic compound comprises protein, e.g., a fusion protein, comprising:
1.a) a specific targeting moiety comprising a target specific binding polypeptide;
1.b) a specific targeting moiety comprising a target ligand binding molecule;
1.c) a specific targeting moiety comprising an antibody molecule;
1.d) a specific targeting moiety comprising a single chain antibody molecule, e.g., a scFv domain; or
1.e) a specific targeting moiety comprising a first of the light or heavy chain variable region of an antibody molecule, and wherein the other variable region is covalently or non-covalently associated with the first;
and
2.a) an effector binding/modulating moiety comprising an effector specific binding polypeptide;
2.b) an effector binding/modulating moiety comprising an effector ligand binding molecule;
2.c) an effector binding/modulating moiety comprising an antibody molecule;
2.d) an effector binding/modulating moiety comprising a single chain antibody molecule, e.g., a scFv domain; or
2.e) an effector binding/modulating moiety comprising a first of the light or heavy chain variable region of an antibody molecule, and wherein the other variable region is covalently or non-covalently associated with the first.

In some embodiments, a therapeutic compound comprises 1.a and 2.a.

In some embodiments, a therapeutic compound comprises 1.a and 2.b.

In some embodiments, a therapeutic compound comprises 1.a and 2.c.

In some embodiments, a therapeutic compound comprises 1.a and 2.d.

In some embodiments, a therapeutic compound comprises 1.a and 2.e.

In some embodiments, a therapeutic compound comprises 1.b and 2.a.

In some embodiments, a therapeutic compound comprises 1.b and 2.b.

In some embodiments, a therapeutic compound comprises 1.b and 2.c.

In some embodiments, a therapeutic compound comprises 1.b and 2.d.

In some embodiments, a therapeutic compound comprises 1.b and 2.e.

In some embodiments, a therapeutic compound comprises 1.c and 2.a.

In some embodiments, a therapeutic compound comprises 1.c and 2.b.

In some embodiments, a therapeutic compound comprises 1.c and 2.c.

In some embodiments, a therapeutic compound comprises 1.c and 2.d.

In some embodiments, a therapeutic compound comprises 1.c and 2.e.

In some embodiments, a therapeutic compound comprises 1.d and 2.a.

In some embodiments, a therapeutic compound comprises 1.d and 2.b.

In some embodiments, a therapeutic compound comprises 1.d and 2.c.

In some embodiments, a therapeutic compound comprises 1.d and 2.d.

In some embodiments, a therapeutic compound comprises 1.d and 2.e.

In some embodiments, a therapeutic compound comprises 1.e and 2.a.

In some embodiments, a therapeutic compound comprises 1.e and 2.b.

In some embodiments, a therapeutic compound comprises 1.e and 2.c.

In some embodiments, a therapeutic compound comprises 1.e and 2.d.

In some embodiments, a therapeutic compound comprises 1.e and 2.e.

In these non-limiting examples, the targeting moiety can be the anti-Robo2 antibody, anti-COL4A3 antibody, anti-COL4A4 antibody, or anti-COL4A5 antibody and the effector binding/modulating moiety can be the complement modulator, the PD-1 agonist, or the IL-2 mutein effector domain. In some embodiments, the complement modulator is a CD55 protein. In some embodiments, the complement modulator is a CD59 protein. In some embodiments, the complement modulator is a CR1 protein. In some embodiments, the complement modulator is a DCP protein. Non-limiting examples of each are provided herein. Therapeutic compounds disclosed herein can, for example, comprise a plurality of effector binding/modulating and specific targeting moieties. Any suitable linker or platform can be used to present the plurality of moieties. The linker is typically coupled or fused to one or more effector binding/modulating and targeting moieties.

In some embodiments, two (or more) linkers associate, either covalently or non-covalently, e.g., to form a hetero- or homodimeric therapeutic compound. For example, the linker can comprise an Fc region and two Fc regions associate with one another. In some embodiments of a therapeutic compound comprising two linker regions, the linker regions can self-associate, e.g., as two identical Fc regions. In some embodiments of a therapeutic compound comprising two linker regions, the linker regions are not capable of, or not capable of substantial, self-association, e.g., the two Fc regions can be members of a knob and hole pair.

In some embodiments, non-limiting exemplary configurations of therapeutic compounds comprise the following (e.g., in N to C terminal order):

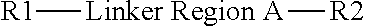

wherein,
R1, and R2, each independently comprise an effector binding/modulating moiety, e.g., complement modulator, anti-PD-1 molecule (antibody), IL-2 mutein, or is absent;
Linker Region A comprises moieties that can associate with one another, e.g., Linker A comprises an Fc moiety provided that an effector binding/modulating moiety and a specific targeting moiety are present.

In some embodiments, the complement modulator comprises, but is not limited to, a CD55 protein, a CD59 protein, a CR1 protein, and a DCP protein.

In some embodiments:
R1 comprises an effector binding/modulating moiety, e.g., complement modulator, anti-PD-1 molecule, IL-2 mutein, or is absent, provided that R2 is not absent;
R2 comprises a specific targeting moiety, or is absent;
Linker Region A comprises moieties that can associate with one another, e.g., Linker A comprises an Fc moiety, provided that one of R1 is present and R2 is present.

In some embodiments:
R1 comprises a specific targeting moiety, or is absent;
R2 comprises an effector binding/modulating moiety, e.g., complement modulator, anti-PD-1 molecule, IL-2 mutein, or is absent;
Linker Region A comprises moieties that can associate with one another, e.g., Linker A comprises an Fc moiety, provided that one of R1 is present and R2 is present.

In some embodiments, the complement modulator comprises, but is not limited to, CD55, CD59, CR1, and DCP.

In some embodiments:
R1, and R2, each independently comprise: an effector binding modulating moiety that modulates the complement system, e.g., CD55, CD59, CR1, or DCP; and a specific targeting moiety, or is absent; provided that an effector binding moiety and a specific targeting moiety are present.

In some embodiments, the specific targeting moiety is an anti-Robo2, anti-COL4A3, anti-COL4A4, or anti-COL4A5 antibody.

In some embodiments, Linker A and Linker B comprise Fc moieties (e.g., self pairing Fc moieties).

In some embodiments:
R1 comprises an effector binding modulating moiety that modulates the complement system, e.g., CD55, CD59, CR1, or DCP; and
R2 comprises specific targeting moieties, e.g., scFv molecules against a tissue antigen.

In some embodiments, the specific targeting moiety is an anti-Robo2, anti-COL4A3, anti-COL4A4, or anti-COL4A5 antibody.

In some embodiments, Linker A and Linker B comprise Fc moieties (e.g., self pairing Fc moieties).

In some embodiments:
R1 comprises CD55;
R1 comprises CD59;
R1 comprises CR1; or
R1 comprises DCP; and
R2 comprises specific targeting moieties, e.g., scFv molecules against a tissue antigen.

In some embodiments, the specific targeting moiety is an anti-Robo2, anti-COL4A3, anti-COL4A4, or anti-COL4A5 antibody.

In some embodiments, Linker A comprises Fc moieties (e.g., self pairing Fc moieties).

In some embodiments:
R1 comprises a functional complement modulator molecule (CD55, CD59, CR1, or DCP); and
R2 comprises specific targeting moieties, e.g., scFv molecules against a tissue antigen.

In some embodiments, the specific targeting moiety is an anti-Robo2, anti-COL4A3, anti-COL4A4, or anti-COL4A5 antibody.

In some embodiments:
R1 comprises specific targeting moieties, e.g., an anti-tissue antigen antibody; and
R2 comprises a functional complement modulator molecule (CD55, CD59, CR1, or DCP), e.g., an scFv molecule.

In some embodiments, the specific targeting moiety is an anti-Robo2, anti-COL4A3, anti-COL4A4, or anti-COL4A5 antibody.

In some embodiments, Linker A comprises Fc moieties (e.g., self pairing Fc moieties).

In some embodiments:
R1 comprises specific targeting moieties, e.g., an anti-tissue antigen antibody; and
R2 comprises CD55;
R2 comprises CD59;
R2 comprises CR1; or
R2 comprises DCP, e.g., an scFv molecule.

In some embodiments, the specific targeting moiety is an anti-Robo2, anti-COL4A3, anti-COL4A4, or anti-COL4A5 antibody.

In some embodiments, Linker A comprises Fc moieties (e.g., self pairing Fc moieties).

In some embodiments:
R1, and R2, each independently comprise: an effector binding modulating moiety that activates an inhibitory receptor on an immune cell, e.g., a T cell or a B cell, e.g., a PD-L1 molecule or a functional anti-PD-1 antibody molecule (an agonist of PD-1), a specific targeting moiety, or is absent; provided that an effector binding moiety and a specific targeting moiety are present.

In some embodiments, Linker A comprises Fc moieties (e.g., self pairing Fc moieties).

In some embodiments:
R1 comprises an effector binding modulating moiety that activates an inhibitory receptor on an immune cell, e.g., a T cell or a B cell, e.g., a PD-L1 molecule or an functional anti-PD-1 antibody molecule (an agonist of PD-1); and
R2 comprises specific targeting moieties, e.g., scFv molecules against a tissue antigen.

In some embodiments, Linker A comprises Fc moieties (e.g., self pairing Fc moieties).

In some embodiments:
R1 comprises a functional anti-PD-1 antibody molecule (an agonist of PD-1); and
R2 comprises specific targeting moieties, e.g., scFv molecules against a tissue antigen. In some embodiments, Linker A comprises Fc moieties (e.g., self pairing Fc moieties).

In some embodiments:
R1 comprises specific targeting moieties, e.g., an anti-tissue antigen antibody; and
R2 comprises a functional anti-PD-1 antibody molecule (an agonist of PD-1), e.g., an scFv molecule.
   In some embodiments, Linker A comprises Fc moieties (e.g., self pairing Fc moieties).

In some embodiments:
R1 comprises a PD-L1 molecule (an agonist of PD-1); and
R2 comprises specific targeting moieties, e.g., scFv molecules against a tissue antigen; and in some embodiments, Linker A comprises Fc moieties (e.g., self pairing Fc moieties).

In some embodiments:
R1 comprises specific targeting moieties, e.g., an anti-tissue antigen antibody; and
R2 comprises a PD-L1 molecule (an agonist of PD-1).
   In some embodiments, Linker A comprises Fc moieties (e.g., self pairing Fc moieties).

In some embodiments:
R1, and R2, each independently comprise: an IL-2 mutein molecule; a specific targeting moiety, or is absent;
provided that an IL-2 mutein molecule and a specific targeting moiety are present.

In an embodiment, Linker A comprises Fc moieties (e.g., self pairing Fc moieties or Fc moieties that do not, or do not substantially self pair).

In an embodiment:
R1 comprises an IL-2 mutein molecule; and
R2 comprises specific targeting moieties, e.g., scFv molecules against a tissue antigen.

In an embodiment Linker A and Linker B comprise Fc moieties (e.g., self pairing Fc moieties or Fc moieties that do not, or do not substantially self pair).

In some embodiments, one of R1, and R2, each comprises a complement modulator, e.g., a CD55, CD55, CR1, or DCP molecule. In some embodiments, one of R1, and R2, each comprises an entity that binds, activates, or maintains, a regulatory immune cell, e.g., a Treg cell or a Breg cell, for example, an IL-2 mutein molecule.

In some embodiments, one of R1, and R2, each comprises an agonistic anti-PD-1 antibody. In some embodiments, the PD-1 antibody is replaced with an IL-2 mutein molecule.

In some embodiments, the targeting moiety of the molecules provided for herein are a Robo2, COL4A3, COL4A4, or COL4A5 targeting moiety, such as an antibody.

In some embodiments, non-limiting exemplary configurations of therapeutic compounds comprise the following (e.g., in N to C terminal order):

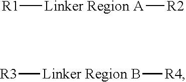

wherein,
R1-Linker Region A-R2 and R3-Linker Region B-R4 are associated with each other to produce a homodimer or a heterodimer;
R1, R2, R3 and R4, each independently comprise an effector binding/modulating moiety, e.g., complement modulator, anti-PD-1 molecule (antibody), IL-2 mutein, or is absent;
Linker Region A and Linker Region B comprise moieties that can associate with one another, e.g., Linker A and Linker B each comprises an Fc moiety provided that an effector binding/modulating moiety and a specific targeting moiety are present.

In some embodiments, the complement modulator comprises, but is not limited to, a CD55 protein, a CD59 protein, a CR1 protein, and a DCP protein.

In some embodiments:
R1 comprises an effector binding/modulating moiety, e.g., complement modulator, anti-PD-1 molecule, IL-2 mutein, or is absent, provided that at least one of R1 and R2 is not absent, and at least one of R3 and R4 is not absent;
R2 comprises a specific targeting moiety, or is absent;
R3 comprises an effector binding/modulating moiety, e.g., complement modulator, anti-PD-1 molecule, IL-2 mutein, or is absent;
R4 comprises a specific targeting moiety, or is absent;
Linker Region A and Linker Region B comprise moieties that can associate with one another, e.g., Linker A and Linker B each comprises an Fc moiety, provided that one of R1 or R3 is present and one of R2 or R4 is present.

In some embodiments:
R1 comprises a specific targeting moiety, or is absent;
R2 comprises an effector binding/modulating moiety, e.g., complement modulator, anti-PD-1 molecule, IL-2 mutein, or is absent;
R3 comprises a specific targeting moiety, or is absent;
R4 comprises an effector binding/modulating moiety, e.g., complement modulator, anti-PD-1 molecule, IL-2 mutein, or is absent;
Linker Region A and Linker Region B comprise moieties that can associate with one another, e.g., Linker A and Linker B each comprises an Fc moiety, provided that one of R1 or R3 is present and one of R2 or R4 is present.

In some embodiments, the complement modulator comprises, but is not limited to, CD55, CD59, CR1, and DCP.

Non-limiting examples include, but are not limited to:

| | Linker | | | Linker | | |
|---|---|---|---|---|---|---|
| R1 | Region A | R2 | R3 | Region B | R4 | Other |
| HCVR and | Fc Region | scFv | HCVR | Fc Region | scFv | Self Pairing |
| LCVR | | | and | | | Linker Regions |
| | | | LCVR | | | |
| HCVR and | Fc Region | scFv | HCVR | Fc Region | scFv | Non-Self |
| LCVR | | | and | | | Pairing linker |
| | | | LCVR | | | regions |
| HCVR and | Fc Region | scFv | HCVR | Fc Region | scFv | Self Pairing |
| LCVR (or | | | and | | | Linker Regions |
| absent) | | | LCVR | | | One of R1 or |
| | | | (or | | | R3 is absent. |
| | | | absent) | | | |
| HCVR and | Fc Region | scFv | HCVR | Fc Region | scFv | Non-Self |
| LCVR (or | | | and | | | Pairing Linker |
| absent) | | | LCVR | | | Regions |
| | | | (or | | | One of R1 or |
| | | | absent) | | | R3 is absent. |
| HCVR and | Fc Region | scFv (or | HCVR | Fc Region | scFv (or | Self Pairing |
| LCVR | | absent) | and | | absent) | linker regions |
| | | | LCVR | | | One of R2 or |
| | | | | | | R4 is absent. |
| HCVR and | Fc Region | scFv (or | HCVR | Fc Region | scFv (or | Non-Self |
| LCVR | | absent) | and | | absent) | Pairing linker |
| | | | LCVR | | | regions |
| | | | | | | One of R2 or |
| | | | | | | R4 is absent. |
| HCVR and | Fc Region | scFv | HCVR | Fc Region | scFv | Self Pairing |
| LCVR | | | and | | | Linker Regions |
| | | | LCVR | | | R1 and R3 are |
| | | | | | | the same |
| HCVR and | Fc Region | scFv | HCVR | Fc Region | scFv | Non-Self |
| LCVR | | | and | | | Pairing linker |
| | | | LCVR | | | regions |
| | | | | | | R1 and R3 are |
| | | | | | | different |
| HCVR and | Fc Region | scFv | HCVR | Fc Region | scFv | Self Pairing |
| LCVR | | | and | | | Linker Regions |
| | | | LCVR | | | R2 and R4 are |
| | | | | | | the same |
| HCVR and | Fc Region | scFv | HCVR | Fc Region | scFv | Non-Self |
| LCVR | | | and | | | Pairing linker |
| | | | LCVR | | | regions |
| | | | | | | R2 and R4 are |
| | | | | | | different |

HCVR and LCVR: refers to a moiety comprising an antigen binding portion of a heavy and light chain variable region, typically with the heavy chain fused to the Linker region.
Self pairing: wherein a liker region can pair with itself, e.g., an Fc region that can pair a copy of itself.
Non-self pairing: wherein a Linker Region does not pair with itself, or does not substantially pair with itself, e.g., an Fc region does not, or does not significantly pair with itself, e.g., wherein Linker Region A and Linker Region B are members of a knob and hole pair.

In some embodiments:
R1, R2, R3 and R4 each independently comprise: an effector binding modulating moiety that modulates the complement system, e.g., a CD55 protein, a CD59 protein, a CR1 protein, or a DCP protein; and a specific targeting moiety, or is absent; provided that an effector binding moiety and a specific targeting moiety are present.

In some embodiments, the specific targeting moiety is an anti-Robo2 antibody, an anti-COL4A3 antibody, an anti-COL4A4 antibody, or an anti-COL4A5 antibody.

In some embodiments, Linker A and Linker B comprise Fc moieties (e.g., self pairing Fc moieties).

In some embodiments:
R1 and R3 independently comprise an effector binding modulating moiety that modulates the complement system, e.g., a CD55 protein, a CD59 protein, a CR1 protein, or a DCP protein; and
R2 and R4 independently comprise specific targeting moieties, e.g., scFv molecules against a tissue antigen.

In some embodiments, the specific targeting moiety is an anti-Robo2 antibody, anti-COL4A3, an anti-COL4A4 antibody, or an anti-COL4A5 antibody.

In some embodiments, Linker A and Linker B comprise Fc moieties (e.g., self pairing Fc moieties).

In some embodiments:
R1 and R3 independently comprise a CD55 protein;
R1 and R3 independently comprise a CD59 protein;
R1 and R3 independently comprise a CR1 protein; or
R1 and R3 independently comprise a DCP protein; and
R2 and R4 independently comprise specific targeting moieties, e.g., scFv molecules against a tissue antigen.

In some embodiments, the specific targeting moiety is an anti-Robo2 antibody, an anti-COL4A3, an anti-COL4A4 antibody, or an anti-COL4A5 antibody.

In some embodiments, Linker A and Linker B comprise Fc moieties (e.g., self pairing Fc moieties).

In some embodiments:
R1 and R3 independently comprise a functional complement modulator molecule (a CD55 protein, a CD59 protein, a CR1 protein, or a DCP protein); and
R2 and R4 independently comprise specific targeting moieties, e.g., scFv molecules against a tissue antigen.

In some embodiments, the specific targeting moiety is an anti-Robo2 antibody, anti-COL4A3, an anti-COL4A4 antibody, or an anti-COL4A5 antibody.

In some embodiments:
R1 and R3 independently comprise specific targeting moieties, e.g., an anti-tissue antigen antibody; and
R2 and R4 independently comprise a functional complement modulator molecule (a CD55 protein, a CD59 protein, a CR1 protein, or a DCP protein), e.g., an scFv molecule.

In some embodiments, the specific targeting moiety is an anti-Robo2 antibody, anti-COL4A3, an anti-COL4A4 antibody, or an anti-COL4A5 antibody.

In some embodiments, Linker A and Linker B comprise Fc moieties (e.g., self pairing Fc moieties).

In some embodiments:
R1 and R3 independently comprise specific targeting moieties, e.g., an anti-tissue antigen antibody; and
R2 and R4 independently comprise a CD55 protein;
R2 and R4 independently comprise a CD59 protein;
R2 and R4 independently comprise a CR1 protein; or
R2 and R4 independently comprise a DCP protein, e.g., an scFv molecule.

In some embodiments, the specific targeting moiety is an anti-Robo2 antibody, anti-COL4A3, an anti-COL4A4 antibody, or an anti-COL4A5 antibody.

In some embodiments, Linker A and Linker B comprise Fc moieties (e.g., self pairing Fc moieties).

In some embodiments:
R1, R2, R3 and R4 each independently comprise: an effector binding modulating moiety that activates an inhibitory receptor on an immune cell, e.g., a T cell or a B cell, e.g., a PD-L1 molecule or a functional anti-PD-1 antibody molecule (an agonist of PD-1), a specific targeting moiety, or is absent; provided that an effector binding moiety and a specific targeting moiety are present.

In some embodiments, Linker A and Linker B comprise Fc moieties (e.g., self pairing Fc moieties).

In some embodiments:
R1 and R3 independently comprise an effector binding modulating moiety that activates an inhibitory receptor on an immune cell, e.g., a T cell or a B cell, e.g., a PD-L1 molecule or an functional anti-PD-1 antibody molecule (an agonist of PD-1); and
R2 and R4 independently comprise specific targeting moieties, e.g., scFv molecules against a tissue antigen.
   In some embodiments, Linker A and Linker B comprise Fc moieties (e.g., self pairing Fc moieties).

In some embodiments:
R1 and R3 independently comprise a functional anti-PD-1 antibody molecule (an agonist of PD-1); and
R2 and R4 independently comprise specific targeting moieties, e.g., scFv molecules against a tissue antigen.

In some embodiments, Linker A and Linker B comprise Fc moieties (e.g., self pairing Fc moieties).

In some embodiments:
R1 and R3 independently comprise specific targeting moieties, e.g., an anti-tissue antigen antibody; and
R2 and R4 independently comprise a functional anti-PD-1 antibody molecule (an agonist of PD-1), e.g., an scFv molecule.
   In some embodiments, Linker A and Linker B comprise Fc moieties (e.g., self pairing Fc moieties).

In some embodiments:
R1 and R3 independently comprise a PD-L1 molecule (an agonist of PD-1); and
R2 and R4 independently comprise specific targeting moieties, e.g., scFv molecules against a tissue antigen; and in some embodiments, Linker A and Linker B comprise Fc moieties (e.g., self pairing Fc moieties).

In some embodiments:
R1 and R3 independently comprise specific targeting moieties, e.g., an anti-tissue antigen antibody; and
R2 and R4 independently comprise a PD-L1 molecule (an agonist of PD-1).
   In some embodiments, Linker A and Linker B comprise Fc moieties (e.g., self pairing Fc moieties).

In some embodiments:
In an embodiment:
R1, R2, R3 and R4 each independently comprise: an IL-2 mutein molecule; a specific targeting moiety, or is absent;
provided that an IL-2 mutein molecule and a specific targeting moiety are present.

In an embodiment, Linker A and Linker B comprise Fc moieties (e.g., self pairing Fc moieties or Fc moieties that do not, or do not substantially self pair).

In an embodiment:
R1 and R3 each comprise an IL-2 mutein molecule; and
R2 and R4 independently comprise specific targeting moieties, e.g., scFv molecules against a tissue antigen.

In an embodiment Linker A and Linker B comprise Fc moieties (e.g., self pairing Fc moieties or Fc moieties that do not, or do not substantially self pair).

In some embodiments, one of R1, R2, R3, and R4 comprises a complement modulator, e.g., a CD55 protein, a CD59 protein, a CR1 protein, or a DCP protein. In some embodiments, one of R1, R2, R3, and R4 comprises an entity that binds, activates, or maintains, a regulatory immune cell, e.g., a Treg cell or a Breg cell, for example, an IL-2 mutein molecule.

In some embodiments, one of R1, R2, R3, and R4 comprises an agonistic anti-PD-1 antibody. In some embodiments, the PD-1 antibody is replaced with an IL-2 mutein molecule.

In some embodiments, the targeting moiety of the molecules provided for herein are a Robo2, COL4A3, COL4A4, or COL4A5 targeting moiety, such as an antibody.

Non-limiting exemplary configurations of therapeutic compounds comprise the following (e.g., in N to C terminal order):

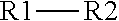

wherein,
R1, and R2, each independently comprises an effector binding/modulating moiety, e.g., complement modulator, anti-PD-1 molecule (antibody), IL-2 mutein; and a targeting moiety, e.g. anti-Robo2 antibody, anti-COL4A3 antibody, anti-COL4A4 antibody, or anti-COL4A5 antibody.

In some embodiments, the complement modulator comprises, but is not limited to, a CD55 protein, a CD59 protein, a CR1 protein, or a DCP protein.

In some embodiments:
R1 comprises an effector binding/modulating moiety, e.g., complement modulator, anti-PD-1 molecule, IL-2 mutein, or is absent;
R2 comprises a specific targeting moiety.

In some embodiments:
R1 comprises a specific targeting moiety, or is absent;
R2 comprises an effector binding/modulating moiety, e.g., complement modulator, anti-PD-1 molecule, IL-2 mutein, or is absent;

In some embodiments, the complement modulator comprises, but is not limited to, a CD55 protein, a CD59 protein, a CR1 protein, or a DCP protein.

In some embodiments:
R1, and R2, each independently comprise: an effector moiety that modulates the complement system, e.g., a CD55 protein, a CD59 protein, a CR1 protein, or a DCP protein; and a specific targeting moiety, or is absent; provided that an effector binding moiety and a specific targeting moiety are present.

In some embodiments, the specific targeting moiety is an anti-Robo2 antibody, anti-COL4A3, an anti-COL4A4 antibody, or an anti-COL4A5 antibody.

In some embodiments:
R1 comprises an effector moiety that modulates the complement system, e.g., a CD55 protein, a CD59 protein, a CR1 protein, or a DCP protein; and
R2 comprises specific targeting moiety, e.g., scFv molecules against a tissue antigen.

In some embodiments, the specific targeting moiety is an anti-Robo2 antibody, anti-COL4A3, an anti-COL4A4 antibody, or an anti-COL4A5 antibody.

In some embodiments:
R1 comprises a CD55 protein;
R1 comprises a CD59 protein;
R1 comprises a CR1 protein; or
R1 comprises a DCP protein; and
R2 comprises specific targeting moiety, e.g., scFv molecules against a tissue antigen.

In some embodiments, the specific targeting moiety is an anti-Robo2 antibody, anti-COL4A3, an anti-COL4A4 antibody, or an anti-COL4A5 antibody.

In some embodiments:
R1 comprises a functional complement modulator molecule (a CD55 protein, a CD59 protein, a CR1 protein, or a DCP protein); and
R2 comprises comprise specific targeting moiety, e.g., scFv molecules against a tissue antigen.

In some embodiments, the specific targeting moiety is an anti-Robo2 antibody, anti-COL4A3, an anti-COL4A4 antibody, or an anti-COL4A5 antibody.

In some embodiments:
R1 comprises specific targeting moieties, e.g., an anti-tissue antigen antibody; and
R2 comprises a functional complement modulator molecule (a CD55 protein, a CD59 protein, a CR1 protein, or a DCP protein), e.g., an scFv molecule.

In some embodiments, the specific targeting moiety is an anti-Robo2 antibody, anti-COL4A3, an anti-COL4A4 antibody, or an anti-COL4A5 antibody.

In some embodiments:
R1 comprises specific targeting moieties, e.g., an anti-tissue antigen antibody; and
R2 comprises a CD55 protein;
R2 comprises a CD59 protein;
R2 comprises a CR1 protein; or
R2 comprises a DCP protein, e.g., an scFv molecule.

In some embodiments, the specific targeting moiety is an anti-Robo2 antibody, anti-COL4A3, an anti-COL4A4 antibody, or an anti-COL4A5 antibody.

In some embodiments:
R1, and R2, each independently comprise: an effector binding modulating moiety that activates an inhibitory receptor on an immune cell, e.g., a T cell or a B cell, e.g., a PD-L1 molecule or a functional anti-PD-1 antibody molecule (an agonist of PD-1), a specific targeting moiety, or is absent; provided that an effector binding moiety and a specific targeting moiety are present.

In some embodiments, Linker A and Linker B comprise Fc moieties (e.g., self pairing Fc moieties).

In some embodiments:
R1 comprises an effector binding modulating moiety that activates an inhibitory receptor on an immune cell, e.g., a T cell or a B cell, e.g., a PD-L1 molecule or an functional anti-PD-1 antibody molecule (an agonist of PD-1); and
R2 comprises specific targeting moieties, e.g., scFv molecules against a tissue antigen.

In some embodiments:
R1 comprises a functional anti-PD-1 antibody molecule (an agonist of PD-1); and
R2 comprises specific targeting moieties, e.g., scFv molecules against a tissue antigen.

In some embodiments:
R1 comprises specific targeting moieties, e.g., an anti-tissue antigen antibody; and
R2 comprises a functional anti-PD-1 antibody molecule (an agonist of PD-1), e.g., an scFv molecule.

In some embodiments:
R1 comprises a PD-L1 molecule (an agonist of PD-1); and
R2 comprises specific targeting moieties, e.g., scFv molecules against a tissue antigen.

In some embodiments:
R1 comprises specific targeting moieties, e.g., an anti-tissue antigen antibody; and
R2 comprises a PD-L1 molecule (an agonist of PD-1).

In some embodiments:
In an embodiment:
R1, and R2, each independently comprise: an IL-2 mutein molecule; a specific targeting moiety, or is absent;
provided that an IL-2 mutein molecule and a specific targeting moiety are present.
In an embodiment:
R1 comprises an IL-2 mutein molecule; and
R2 comprises specific targeting moieties, e.g., scFv molecules against a tissue antigen.

In some embodiments, one of R1, and R2 each independently comprises a complement modulator, e.g., a a CD55 protein, a CD59 protein, a CR1 protein, or a DCP protein. In some embodiments, one of R1, and R2, each comprises an entity that binds, activates, or maintains, a regulatory immune cell, e.g., a Treg cell or a Breg cell, for example, an IL-2 mutein molecule.

### Linker Regions

As discussed herein, specific targeting and effector binding/modulating moieties can be linked by linker regions. Any linker region described herein can be used as a linker. For example, Linker Regions A and B can comprise Fc regions. In some embodiments, a therapeutic compound comprises a Linker Region that can self-associate. In some embodiments, a therapeutic compound comprises a Linker Region that has a moiety that minimizes self association, and typically Linker Region A and Linker Region B are heterodimers. Linkers also include glycine/serine linkers. In some embodiments, the linker can comprise one or more repeats of GGGGS (SEQ ID NO: 26). In some embodiments, the linker comprises 1, 2, 3, 4, or 5 repeats of SEQ ID NO: 26. In some embodiments, the linker comprises the amino acid sequence: GGGGS (SEQ ID NO: 26), GGGGSGGGGSGGGGSGGGGS (SEQ ID NO: 24), or GGGGSGGGGSGGGGS (SEQ ID NO: 25). These linkers can be used in any of the therapeutic compounds or compositions provided herein.

The linker region can comprise an Fc region that has been modified (e.g., mutated) to produce a heterodimer. In some embodiments, the CH3 domain of the Fc region can be mutated. Examples of such Fc regions can be found in, for example, U.S. Pat. No. 9,574,010, which is hereby incorporated by reference in its entirety. The Fc region as defined herein comprises a CH3 domain or fragment thereof, and may additionally comprise one or more addition constant region domains, or fragments thereof, including hinge, CH1, or CH2. It will be understood that the numbering of the Fc amino acid residues is that of the EU index as in Kabat et al 1991, NIH Publication 91-3242, National Technical Information Service, Springfield, Va. The “EU index as set forth in Kabat” refers to the EU index numbering of the human IgG1 Kabat antibody. For convenience, Table B of U.S. Pat. No. 9,574,010 provides the amino acids numbered according to the EU index as set forth in Kabat of the CH2 and CH3 domain from human IgG1, which is hereby incorporated by reference. Table 1.1 of U.S. Pat. No. 9,574,010 provides mutations of variant Fc heterodimers that can be used as linker regions. Table 1.1 of U.S. Pat. No. 9,574,010 is hereby incorporated by reference.

In some embodiments, the Linker Region A comprises a first CH3 domain polypeptide and a the Linker Region B comprises a second CH3 domain polypeptide, the first and second CH3 domain polypeptides independently comprising amino acid modifications as compared to a wild-type CH3 domain polypeptide, wherein the first CH3 domain polypeptide comprises amino acid modifications at positions T350, L351, F405, and Y407, and the second CH3 domain polypeptide comprises amino acid modifications at positions T350, T366, K392 and T394, wherein the amino acid modification at position T350 is T350V, T3501, T350L or T350M; the amino acid modification at position L351 is L351Y; the amino acid modification at position F405 is F405A, F405V, F405T or F405S; the amino acid modification at position Y407 is Y407V, Y407A or Y407I; the amino acid modification at position T366 is T366L, T366I, T366V, or T366M; the amino acid modification at position K392 is K392F, K392L or K392M; and the amino acid modification at position T394 is T394W, and wherein the numbering of amino acid residues is according to the EU index as set forth in Kabat.

In some embodiments, the amino acid modification at position K392 is K392M or K392L. In some embodiments, the amino acid modification at position T350 is T350V. In some embodiments, the first CH3 domain polypeptide further comprises one or more amino acid modifications selected from Q347R and one of S400R or S400E. In some embodiments, the second CH3 domain polypeptide further comprises one or more amino acid modifications selected from L351Y, K360E, and one of N390R, N390D or N390E. In some embodiments, the first CH3 domain polypeptide further comprises one or more amino acid modifications selected from Q347R and one of S400R or S400E, and the second CH3 domain polypeptide further comprises one or more amino acid modifications selected from L351Y, K360E, and one of N390R, N390D or N390E. In some embodiments, the amino acid modification at position T350 is T350V. In some embodiments, the amino acid modification at position F405 is F405A. In some embodiments, the amino acid modification at position Y407 is Y407V. In some embodiments, the amino acid modification at position T366 is T366L or T3661. In some embodiments, the amino acid modification at position F405 is F405A, the amino acid modification at position Y407 is and Y407V, the amino acid modification at position T366 is T366L or T366I, and the amino acid modification at position K392 is K392M or K392L. In some embodiments, the first CH3 domain polypeptide comprises the amino acid modifications T350V, L351Y, S400E, F405V and Y407V, and the second CH3 domain polypeptide comprises the amino acid modifications T350V, T366L, N390R, K392M and T394W. In some embodiments, the first CH3 domain polypeptide comprises the amino acid modifications T350V, L351Y, S400E, F405T and Y407V, and the second CH3 domain polypeptide comprises the amino acid modifications T350V, T366L, N390R, K392M and T394W. In some embodiments, the first CH3 domain polypeptide comprises the amino acid modifications T350V, L351Y, S400E, F405S and Y407V, and the second CH3 domain polypeptide comprises the amino acid modifications T350V, T366L, N390R, K392M and T394W. In some embodiments, the first CH3 domain polypeptide comprises the amino acid modifications T350V, L351Y, S400E, F405A and Y407V, and the second CH3 domain polypeptide comprises the amino acid modifications T350V, L351Y, T366L, N390R, K392M and T394W. In some embodiments, the first CH3 domain polypeptide comprises the amino acid modifications Q347R, T350V, L351Y, S400E, F405A and Y407V, and the second CH3 domain polypeptide comprises the amino acid modifications T350V, K360E, T366L, N390R, K392M and T394W. In some embodiments, the first CH3 domain polypeptide comprises the amino acid modifications T350V, L351Y, S400R, F405A and Y407V, and the second CH3 domain polypeptide comprises the amino acid modifications T350V, T366L, N390D, K392M and T394W. In some embodiments, the first CH3 domain polypeptide comprises the amino acid modifications T350V, L351Y, S400R, F405A and Y407V, and the second CH3 domain polypeptide comprises the amino acid modifications T350V, T366L, N390E, K392M and T394W. In some embodiments, the first CH3 domain polypeptide comprises the amino acid modifications T350V, L351Y, S400E, F405A and Y407V, and the second CH3 domain polypeptide comprises the amino acid modifications T350V, T366L, N390R, K392L and T394W. In some embodiments, the first CH3 domain polypeptide comprises the amino acid modifications T350V, L351Y, S400E, F405A and Y407V, and the second CH3 domain polypeptide comprises the amino acid modifications T350V, T366L, N390R, K392F and T394W.

In some embodiments, an isolated heteromultimer comprising a heterodimeric CH3 domain comprising a first CH3 domain polypeptide and a second CH3 domain polypeptide, the first CH3 domain polypeptide comprising amino acid modifications at positions F405 and Y407, and the second CH3 domain polypeptide comprising amino acid modifications at positions T366 and T394, wherein: (i) the first CH3 domain polypeptide further comprises an amino acid modification at position L351, and (ii) the second CH3 domain polypeptide further comprises an amino acid modification at position K392, wherein the amino acid modification at position F405 is F405A, F405T, F405S or F405V; and the amino acid modification at position Y407 is Y407V, Y407A, Y407L or Y407I; the amino acid modification at position T394 is T394W; the amino acid modification at position L351 is L351Y; the amino acid modification at position K392 is K392L, K392M, K392V or K392F, and the amino acid modification at position T366 is T366I, T366L, T366M or T366V, wherein the heterodimeric CH3 domain has a melting temperature (Tm) of about 70° C. or greater and a purity greater than about 90%, and wherein the numbering of amino acid residues is according to the EU index as set forth in Kabat.

In some embodiments, the Linker Region A comprises a first CH3 domain polypeptide and the Linker Region B comprises a second CH3 domain polypeptide, wherein the first CH3 domain polypeptide comprising amino acid modifications at positions F405 and Y407, and the second CH3 domain polypeptide comprising amino acid modifications at positions T366 and T394, wherein: (i) the first CH3 domain polypeptide further comprises an amino acid modification at position L351, and (ii) the second CH3 domain polypeptide further comprises an amino acid modification at position K392, wherein the amino acid modification at position F405 is F405A, F405T, F405S or F405V; and the amino acid modification at position Y407 is Y407V, Y407A, Y407L or Y407I; the amino acid modification at position T394 is T394W; the amino acid modification at position L351 is L351Y; the amino acid modification at position K392 is K392L, K392M, K392V or K392F, and the amino acid modification at position T366 is T366I, T366L, T366M or T366V, wherein the heterodimeric CH3 domain has a melting temperature (Tm) of about 70 C. or greater and a purity greater than about 90%, and wherein the numbering of amino acid residues is according to the EU index as set forth in Kabat. In some embodiments, the amino acid modification at position F405 is F405A. In some embodiments, the amino acid modification at position T366 is T366I or T366L. In some embodiments, the amino acid modification at position Y407 is Y407V. In some embodiments, the amino acid modification at position F405 is F405A, the amino acid modification at position Y407 is Y407V, the amino acid modification at position T366 is T366I or T366L, and the amino acid modification at position K392 is K392L or K392M. In some embodiments, the amino acid modification at position F405 is F405A, the amino acid modification at position Y407 is Y407V, the amino acid modification at position T366 is T366L, and the amino acid modification at position K392 is K392M. In some embodiments, the amino acid modification at position F405 is F405A, the amino acid modification at position Y407 is Y407V, the amino acid modification at position T366 is T366L, and the amino acid modification at position K392 is K392L. In some embodiments, the amino acid modification at position F405 is F405A, the amino acid modification at position Y407 is Y407V, the amino acid modification at position T366 is T366I, and the amino acid modification at position K392 is K392M. In some embodiments, the amino acid modification at position F405 is F405A, the amino acid modification at position Y407 is Y407V, the amino acid modification at position T366 is T366I, and the amino acid modification at position K392 is K392L. In some embodiments, the first CH3 domain polypeptide further comprises an amino acid modification at position 5400 selected from S400D and S400E, and the second CH3 domain polypeptide further comprises the amino acid modification N390R. In some embodiments, the amino acid modification at position F405 is F405A, the amino acid modification at position Y407 is Y405V, the amino acid modification at position 5400 is S400E, the amino acid modification at position T366 is T366L, and the amino acid modification at position K392 is K392M.

In some embodiments, the modified first and second CH3 domains are comprised by an Fc construct based on a type G immunoglobulin (IgG). The IgG can be an IgG1, IgG2, IgG3, or IgG4.

Other Linker Region A and Linger Region B comprising variant CH3 domains are described in U.S. Pat. Nos. 9,499,634 and 9,562,109, each of which is incorporated by reference in its entirety.

Linker Region A and Linker Region B can be complementary fragments of a protein, e.g., a naturally occurring protein such as human serum albumin. In some embodiments, one of Linker Region A and Linker Region B comprises a first, e.g., an N-terminal fragment of the protein, e.g., hSA, and the other comprises a second, e.g., a C-terminal fragment of the protein.

In an embodiment the fragments comprise an N-terminal and a C-terminal fragment. In an embodiment the fragments comprise two internal fragments. Typically the fragments do not overlap. In an embodiment the first and second fragment, together, provide the entire sequence of the original protein, e.g., hSA. The first fragment provides a N-terminus and a C-terminus for linking, e.g., fusing, to other sequences, e.g., sequences of R1, R2, R3, or R4 (as defined herein).

Linker Region A and the Linker Region B can be derived from albumin polypeptide. In some embodiments, the albumin polypeptide is selected from native human serum albumin polypeptide and human alloalbumin polypeptide. The albumin polypeptide can be modified such that the Linker Region A and Linker Region B interact with one another to form heterodimers. Examples of modified albumin polypeptides are described in U.S. Pat. Nos. 9,388,231 and 9,499,605, each of which is hereby incorporated by reference in its entirety.

Accordingly, provided herein are multifunctional heteromultimer proteins of the formula R1-Linker Region A-R2 and R3-Linker Region B-R4, wherein the Linker Region A and Linker Region B form a heteromultimer. In some embodiments, the Linker Region A comprises a first polypeptide and the Linker Region B comprises a second polypeptide; wherein each of said first and second polypeptides comprises an amino acid sequence comprising a segment of an albumin polypeptide selected from native human serum albumin polypeptide and human alloalbumin polypeptide; wherein said first and second polypeptides are obtained by segmentation of said albumin polypeptide at a segmentation site, such that the segmentation results in a deletion of zero to 3 amino acid residues at the segmentation site; wherein said first polypeptide comprises at least one mutation selected from A194C, L198C, W214C, A217C, L331C and A335C, and said second polypeptide comprises at least one mutation selected from L331C, A335C, V343C, L346C, A350C, V455C, and N458C; and wherein said first and second polypeptides self-assemble to form a quasi-native structure of the monomeric form of the albumin polypeptide.

In some embodiments, the segmentation site resides on a loop of the albumin polypeptide that has a high solvent accessible surface area (SASA) and limited contact with the rest of the albumin structure. In some embodiments, the segmentation results in a complementary interface between the transporter polypeptides. These segmentation sites are described, for example, in U.S. Pat. No. 9,388,231, which is hereby incorporated by reference in its entirety.

In some embodiments, the first polypeptide comprises residues 1-337 or residues 1-293 of the albumin polypeptide with one or more of the mutations described herein. In some embodiments, the second polypeptide comprises residues of 342-585 or 304-585 of the albumin polypeptide with one or more of the mutations described herein. In some embodiments, the first polypeptide comprises residues 1-339, 1-300, 1-364, 1-441, 1-83, 1-171, 1-281, 1-293, 1-114, 1-337, or 1-336 of the albumin protein. In some embodiments, the second polypeptide comprises residues 301-585, 365-585, 442-585, 85-585, 172-585, 282-585, or 115-585, 304-585, 340-585, or 342-585 of the albumin protein.

In some embodiments, the first and second polypeptide comprise the residues of the albumin protein as shown in the table below. The sequence of the albumin protein is described below.

| First Polypeptide Residues | Second Polypeptide Residues |
|---|---|
| 1-300 | 301-585 |
| 1-364 | 365-585 |
| 1-441 | 442-585 |
| 1-83 | 85-585 |
| 1-171 | 172-585 |
| 1-281 | 282-585 |
| 1-114 | 115-585 |
| 1-339 | 340-585 |
| 1-337 | 342-585 |
| 1-293 | 304-585 |
| 1-336 | 342-585 |

In some embodiments, the first and second polypeptides comprise a linker that can form a covalent bond with one another, such as a disulfide bond. A non-limiting example of the linker is a peptide linker. In some embodiments, the peptide linker comprises GGGGS (SEQ ID NO: 26). The linker can be fused to the C-terminus of the first polypeptide and the N-terminus of the second polypeptide. The linker can also be used to attach the moieties described herein without abrogating the ability of the linkers to form a disulfide bond. In some embodiments, the first and second polypeptides do not comprise a linker that can form a covalent bond. In some embodiments, the first and second polypeptides have the following substitutions.

| First Polypeptide Substitution | Second Polypeptide Substitution |
|---|---|
| A217C | V343C |
| L331C | A350C |
| A217C | L346C |
| W214C | V343C |
| A335C | L346C |
| L198C | V455C |
| A217C | A335C |
| A217C | L331C |
| L198C | N458C |
| A194C | V455C |

The sequence of the albumin polypeptide can be the sequence of human albumin as shown, in the post-protein form with the N-terminal signaling residues removed

(MKWVTFISLLFLFSSAYSRGVFRR, SEQ ID NO: 112)
(human albumin, SEQ ID NO: 113)
DAHKSEVAHRFKDLGEENFKALVLIAFAQYLQQCPFEDHV
KLVNEVTEFAKTCVADESAENCDKSLHTLFGDKLCTVATL
RETYGEMADCCAKQEPERNECFLQHKDDNPNLPRLVRPEV
DVMCTAFHDNEETFLKKYLYEIARRHPYFYAPELLFFAKRY
KAAFTECCQAADKAACLLPKLDELRDEGKASSAKQRLKCA
SLQKFGERAFKAWAVARLSQRFPKAEFAEVSKLVTDLTKV
HTECCHGDLLECADDRADLAKYICENQDSISSKLKECCEKP
LLEKSHCIAEVENDEMPADLPSLAADFVESKDVCKNYAEA
KDVFLGMFLYEYARRHPDYSVVLLLRLAKTYETTLEKCCA
AADPHECYAKVFDEFKPLVEEPQNLIKQNCELFEQLGEYKF
QNALLVRYTKKVPQVSTPTLVEVSRNLGKVGSKCCKHPEA
KRMPCAEDYLSVVLNQLCVLHEKTPVSDRVTKCCTESLVN
RRPCFSALEVDETYVPKEFNAETFTFHADICTLSEKERQIKK
QTALVELVKHKPKATKEQLKAVMDDFAAFVEKCCKADDK
ETCFAEEGKKLVAASQAALGL

In some embodiments, the Linker Region A and the Linker Region B form a heterodimer as described herein.

In some embodiments, the protein comprises at the N-terminus an antibody comprised of F(ab′)2 on an IgG1 Fc backbone fused with scFvs on the C-terminus of the IgG Fc backbone. In some embodiments, the IgG Fc backbone is a IgG1 Fc backbone. In some embodiments, the IgG1 backbone is replaced with a IgG4 backbone, IgG2 backbone, or other similar IgG backbone. The IgG backbones described in this paragraph can be used throughout this application where a Fc region is referred to as part of the therapeutic compound. Thus, in some embodiments, the antibody comprised of F(ab′)2 on an IgG1 Fc backbone can be an anti-Robo2 antibody, an anti-COL4A3 antibody, an anti-COL4A4 antibody, or an anti-COL4A5 antibody on an IgG1 Fc or any other targeting moiety or effector binding/modulating moiety provided herein.

In some embodiments, the scFV segments fused to the C-terminus is be an anti-PD-1 antibody, if the N-terminus region is an anti-Robo2 antibody, an anti-COL4A3 antibody, an anti-COL4A4 antibody, or an anti-COL4A5 antibody if the N-terminus region is an anti-PD-1 antibody. In this non-limiting example, the N-terminus is the targeting moiety, such as any one of the ones provided for herein, and the C-terminus is the effector binding/modulating moiety, such as any of the ones provided for herein. Alternatively, in some embodiments, the N-terminus is the effector binding/modulating moiety, such as any one of the ones provided for herein, and the C-terminus is the targeting moiety, such as any of the ones provided for herein.

In some embodiments, the N-terminus is the targeting moiety, such as any one of the ones provided for herein, and the C-terminus is the effector binding/modulating moiety, such as any of the ones provided for herein.

In some embodiments, the therapeutic compound comprises two polypeptides that homodimerize. In some embodiments, the N-terminus of the protein comprises an effector binding/modulating moiety that is fused to a human IgG1 Fc domain (e.g., CH2 and/or CH3 domains). In some embodiments, the C-terminus of the Fc domain is another linker that is fused to the targeting moiety. Thus, in some embodiments, the molecule could be represented using the formula of R1-Linker A-Fc Region-Linker B-R2, wherein R1 is an effector binding/modulating moiety, R2 is a targeting moiety, Linker A and Linker B are independently linkers as provided for herein. In some embodiments, Linker 1 and Linker 2 are different.

In some embodiments, the molecule could be represented using the formula of R1-Linker A-Fc Region-Linker B-R2, wherein R1 is a targeting moiety, R2 is an effector binding/modulating moiety, Linker A and Linker B are independently linkers as provided for herein. In some embodiments, Linker A and Linker B are different. The linkers can be chosen from the non-limiting examples provided for herein. In some embodiments, R1 and R2 are independently selected from F(ab′)2 and scFV antibody domains. In some embodiments, R1 and R2 are different antibody domains. In some embodiments, the scFV is in the V_{L}-V_{H} domain orientation.

In some embodiments, the therapeutic compound is a bispecific antibody. In some embodiments, the bispecific antibodies are comprised of four polypeptide chains comprising the following:
Chain 1: nt-V_{H}1-CH1-CH2-CH3-Linker A-scFv[V_{L}2-Linker B-V_{H}2]-ct
Chain 2: nt-V_{H}1-CH1-CH2-CH3-Linker A-scFv[V_{L}2-Linker B-V_{H}2]-ct
Chain 3: nt-V_{L}1-CL-ct
Chain 4: nt-V_{L}1-CL-ct,
wherein chains 1 and 2 are identical to each other, and chains 3 and 4 are identical to each other,
wherein chain 1 forms a homodimer with chain 2; and chain 3 and 4 associate with chain 1 and chain 2. That is, when each light chain associates with each heavy chain, V_{L}1 associates with V_{H}1 and CL associates with CH1 to form two functional Fab units. Without being bound to any particular theory, each scFv unit is intrinsically functional since V_{L}2 and V_{H}2 are covalently linked in tandem with a linker as provided herein (e.g., GGGGS (SEQ ID NO: 26), GGGGSGGGGSGGGGSGGGGS (SEQ ID NO: 24), or GGGGSGGGGSGGGGS (SEQ ID NO: 25). The sequences of Linker A and Linker B, which are independent of one another can be the same or different and as otherwise described throughout the application. Thus, in some embodiments, Linker A comprises GGGGS (SEQ ID NO: 26), or two repeats thereof, GGGGSGGGGSGGGGS (SEQ ID NO: 25), or GGGGSGGGGSGGGGSGGGGS (SEQ ID NO: 24). In some embodiments, Linker B comprises GGGGS (SEQ ID NO: 26), or two repeats thereof, GGGGSGGGGSGGGGS (SEQ ID NO: 25), or GGGGSGGGGSGGGGSGGGGS (SEQ ID NO: 24). The scFv may be arranged in the NT-V_{H}2-V_{L}2-CT or NT-V_{L}2-V_{H}2-CT orientation. V_{L} refers to the variable light chain, and V_{H} refers to the variable heavy chain. Fab refers to the fragment antigen-binding region of an antibody, and scFv refers to the single chain fragment variable antibody. NT or nt refers to N-terminus and CT or ct refers to C-terminus of the protein. CH1, CH2, and CH3 are the domains from the IgG Fc region, and CL refers to Constant Light chain, which can be either kappa or lambda family light chains.

In some embodiments, the V_{H}1 and V_{L}1 domains are derived from the effector molecule and the V_{H}2 and V_{L}2 domains are derived from the targeting moiety. In some embodiments the V_{H}1 and V_{L}1 domains are derived from a targeting moiety and the V_{H}2 and V_{L}2 domains are derived from an effector binding/modulating moiety.

In some embodiments, the V_{H}1 and V_{L}1 domains are derived from an anti-PD-1 antibody, and the V_{H}2 and V_{L}2 domains are derived from an anti-Robo2 antibody. In some embodiments the V_{H}1 and V_{L}1 domains are derived from an anti-Robo2 antibody and the V_{H}2 and V_{L}2 domains are derived from an anti-PD-1 antibody.

In some embodiments, Linker A comprises 1, 2, 3, 4, or 5 GGGGS (SEQ ID NO: 26) repeats. In some embodiments, Linker B comprises 1, 2, 3, 4, or 5 GGGGS (SEQ ID NO: 26) repeats. For the avoidance of doubt, the sequences of Linker A and Linker B, which are used throughout this application, are independent of one another. Therefore, in some embodiments, Linker A and Linker B can be the same or different. In some embodiments, Linker A comprises GGGGS (SEQ ID NO: 26), or two repeats thereof, GGGGSGGGGSGGGGS (SEQ ID NO: 25), or GGGGSGGGGSGGGGSGGGGS (SEQ ID NO: 24). In some embodiments, Linker B comprises GGGGS (SEQ ID NO: 26), or two repeats thereof, GGGGSGGGGSGGGGS (SEQ ID NO: 25), or GGGGSGGGGSGGGGSGGGGS (SEQ ID NO: 24).

In some embodiments, the therapeutic compound comprises a light chain and a heavy chain. In some embodiments, the light and heavy chain begin at the N-terminus with the V_{H} domain of a targeting moiety followed by the CH1 domain of a human IgG1, which is fused to a Fc region (e.g., CH2-CH3) of human IgG1. In some embodiments, at the C-terminus of the Fc region is fused to a linker as provided herein, such as but not limited to, GGGGS (SEQ ID NO: 26), or two or three repeats thereof, or GGGGSGGGGSGGGGS (SEQ ID NO: 25). The linker can then be fused to an effector binding/modulating moiety, such as any one of the effector moieties provided for herein. The polypeptides can homodimerize through the heavy chain homodimerization, which results in a therapeutic compound having two effector moieties, such as two anti-PD-1 antibodies. In this orientation, the targeting moiety is an IgG format, there are two Fab arms that each recognize binding partner of the targeting moiety, for example, Robo2 being bound by the Robo2 targeting moiety.

In some embodiments, the targeting moiety is a Robo2 antibody. In some embodiments, the targeting moiety is a COL4A3 antibody. In some embodiments, the targeting moiety is a COL4A4 antibody. In some embodiments, the targeting moiety is a COL4A5 antibody.

In some embodiments, the antibody is linked to another antibody or therapeutic. In some embodiments, the anti-Robo2 antibody is linked to a complement modulator, PD-1 antibody, an IL-2 mutein as provided herein or that is incorporated by reference. In some embodiments, the complement modulator is selected from, a CD55 protein, a CD59 protein, a CR1 protein, and a DCP protein. In some embodiments, the anti-COL4A3 antibody is linked to a complement modulator, PD-1 antibody, an IL-2 mutein as provided herein or that is incorporated by reference. In some embodiments, the complement modulator is selected from, a CD55 protein, a CD59 protein, a CR1 protein, and a DCP protein. In some embodiments, the anti-COL4A4 antibody is linked to a complement modulator, PD-1 antibody, an IL-2 mutein as provided herein or that is incorporated by reference. In some embodiments, the complement modulator is selected from, but not limited to, a CD55 protein, a CD59 protein, a CR1 protein, and a DCP protein. In some embodiments, the anti-COL4A5 antibody is linked to a complement modulator, PD-1 antibody, an IL-2 mutein as provided herein or that is incorporated by reference. In some embodiments, the complement modulator is selected from, a CD55 protein, a CD59 protein, a CR1 protein, and a DCP protein.

In some embodiments, the anti-Robo2 antibody, the anti-COL4A3 antibody, the anti-COL4A4 antibody, or the anti-COL4A5 antibody, as provided herein, is linked to an IL-2 mutein comprising L118I, N88D, V69A, Q74P, and C125S mutations, and having the following sequence:

(SEQ ID NO: 50)
APTSSSTKKTQLQLEHLLLDLQMILNGINNYKNPKLTRMLTFKFYMPKKA
TELKHLQCLEEELKPLEEALNLAPSKNFHLRPRDLISDINVIVLELKGSE
TTEMCEYADETATIVEFINRWITFSQSIISTLT

Other examples of IL-2 muteins are described in U.S. Pat. Nos. 10,174,091 and 10,676,516, each of which is incorporated by reference in its entirety. Other examples of IL-2 muteins include, but are not limited to, those described in WO2010085495, WO2016/164937, US2014/0286898A1, WO2014153111A2, WO2010/085495, cytotoxic WO2016014428A2, WO2016025385A1, and US20060269515, each of which is hereby incorporated by reference in its entirety.

In some embodiments, as provided for herein, the anti-Robo2 antibody, the anti-COL4A3 antibody, the anti-COL4A4 antibody, or the anti-COL4A5 antibody is linked directly or indirectly to a PD-1 antibody or binding fragment thereof. Examples of PD-1 antibodies are described in U.S. application Ser. No. 16/99,7238, International Application Nos. PCT/US2020/046920, PCT/US2022/070791, and PCT/US2021/046656, each of which is hereby incorporated by reference in its entirety.

In some embodiments, the PD-1 antibody comprises a sequence as shown in PD-1 Antibody Tables as provided for in U.S. application Ser. No. 16/99,7238, International Application Nos. PCT/US2020/046920, PCT/US2022/070791, and PCT/US2021/046656, each of which is hereby incorporated by reference in its entirety. In some embodiments, the antibody is in a scFV format as illustrated in the PD-1 Antibody Tables referenced therein. In some embodiments, the antibody comprises a CDR1 from any one of clones of the PD-1 Antibody Table, a CDR2 from any one of clones of the PD-1 Antibody Table, and a CDR3 from any one of clones of the PD-1 Antibody Table as referenced therein. In some embodiments, the antibody comprises a LCDR1 from any one of clones of the PD-1 Antibody Table, a LCDR2 from any one of clones of the PD-1 Antibody Table, and a LCDR3 from any one of clones of the PD-1 Antibody Table as referenced therein. In some embodiments, the amino acid residues of the CDRs shown above contain mutations. In some embodiments, the CDRs contain 1, 2, 3, 4, 5, 6, 7, 8, 9, or 10 substitutions or mutations. In some embodiments, the substitution is a conservative substitution.

In some embodiments, the PD-1 antibody has a V_{H} region selected from any one of clones of the PD-1 Antibody Table and a V_{L} region selected from any one of clones as set forth in the PD-1 Antibody Table as referenced therein.

In some embodiments, as provided for herein, the PD-1 antibody, or binding fragment thereof, is linked directly or indirectly to an anti-Robo2 antibody. In some embodiments, as provided for herein, the PD-1 antibody, or binding fragment thereof, is linked directly or indirectly to an anti-COL4A3 antibody. In some embodiments, as provided for herein, the PD-1 antibody, or binding fragment thereof, is linked directly or indirectly to an anti-COL4A4 antibody. In some embodiments, as provided for herein, the PD-1 antibody, or binding fragment thereof, is linked directly or indirectly to an anti-COL4A5 antibody.

In some embodiments, as provided for herein, the anti-Robo2 antibody, or binding fragment thereof, is linked directly or indirectly to an IL-2 mutein or binding fragment thereof. In some embodiments, as provided for herein, the anti-COL4A3 antibody, or binding fragment thereof, is linked directly or indirectly to an IL-2 mutein or binding fragment thereof. In some embodiments, as provided for herein, the anti-COL4A4 antibody, or binding fragment thereof, is linked directly or indirectly to an IL-2 mutein or binding fragment thereof. In some embodiments, as provided for herein, the anti-COL4A5 antibody, or binding fragment thereof, is linked directly or indirectly to an IL-2 mutein or binding fragment thereof. The IL-2 mutein can be any mutein as provided for herein or other IL-2 muteins known to one of skill in the art.

In some embodiments, as provided for herein, the anti-Robo2 antibody, or binding fragment thereof, is linked directly or indirectly to a complement modulator molecule. In some embodiments, as provided for herein, the anti-COL4A3 antibody, or binding fragment thereof, is linked directly or indirectly to a complement modulator molecule. In some embodiments, as provided for herein, the anti-COL4A4 antibody, or binding fragment thereof, is linked directly or indirectly to a complement modulator molecule. In some embodiments, as provided for herein, the anti-COL4A5 antibody, or binding fragment thereof, is linked directly or indirectly to a complement modulator molecule. In some embodiments, the complement modulator is selected from a CD55 protein, a CD59 protein, a CR1 protein, and a DCP protein.

In some embodiments, the complement modulator molecule is linked to the anti-Robo2 antibody in the N- to C-terminus direction. In some embodiments, the C-terminus of an anti-Robo2 antibody scFv is linked via a G/S linked to the N-terminus of a complement modulator molecule. In some embodiments, the complement modulator molecule is linked to the anti-COL4A3 antibody in the N- to C-terminus direction. In some embodiments, the C-terminus of an anti-COL4A3 antibody scFv is linked via a G/S linked to the N-terminus of a complement modulator molecule. In some embodiments, the complement modulator molecule is linked to the anti-COL4A4 antibody in the N- to C-terminus direction. In some embodiments, the C-terminus of an anti-COL4A4 antibody scFv is linked via a G/S linked to the N-terminus of a complement modulator molecule. In some embodiments, the complement modulator molecule is linked to the anti-COL4A5 antibody in the N- to C-terminus direction. In some embodiments, the C-terminus of an anti-COL4A5 antibody scFv is linked via a G/S linked to the N-terminus of a complement modulator molecule. In some embodiments, the complement modulator is selected from CD55, CD59, CR1, and DCP.

In some embodiments, the complement modulator molecule is linked to the anti-Robo2 antibody in the C- to N-terminus direction. In some embodiments, the C-terminus of complement modulator molecule is linked via a G/S linker to the N-terminus of an Fc molecule further linked at the N-terminus of the Fc molecule via a G/S linker to the N-terminus of an anti-Robo2 antibody scFv. In some embodiments, the complement modulator molecule is linked to the anti-COL4A3 antibody in the C- to N-terminus direction. In some embodiments, the C-terminus of complement modulator molecule is linked via a G/S linker to the N-terminus of an Fc molecule further linked at the N-terminus of the Fc molecule via a G/S linker to the N-terminus of an anti-COL4A3 antibody scFv. In some embodiments, the complement modulator molecule is linked to the anti-COL4A4 antibody in the C- to N-terminus direction. In some embodiments, the C-terminus of complement modulator molecule is linked via a G/S linker to the N-terminus of an Fc molecule further linked at the N-terminus of the Fc molecule via a G/S linker to the N-terminus of an anti-COL4A4 antibody scFv. In some embodiments, the complement modulator molecule is linked to the anti-COL4A5 antibody in the C- to N-terminus direction. In some embodiments, the C-terminus of complement modulator molecule is linked via a G/S linker to the N-terminus of an Fc molecule further linked at the N-terminus of the Fc molecule via a G/S linker to the N-terminus of an anti-COL4A5 antibody scFv. In some embodiments, the complement modulator is selected from CD55, CD59, CR1, and DCP.

In some embodiments, the anti-Robo2 antibody linked to the complement modulator molecule comprises a heterodimeric molecule, further comprising a bivalent anti-Robo2 antibody and monovalent human complement modulator molecules. In some embodiments, the anti-COL4A3 antibody linked to the complement modulator molecule comprises a heterodimeric molecule, further comprising a bivalent anti-COL4A3 antibody and monovalent human complement modulator molecules. In some embodiments, the anti-COL4A4 antibody linked to the complement modulator molecule comprises a heterodimeric molecule, further comprising a bivalent anti-COL4A4 antibody and monovalent human complement modulator molecules. In some embodiments, the anti-COL4A5 antibody linked to the complement modulator molecule comprises a heterodimeric molecule, further comprising a bivalent anti-COL4A5 antibody and monovalent human complement modulator molecules. In some embodiments, the complement modulator is selected from CD55, CD59, CR1, and DCP.

In some embodiments, the anti-Robo2 antibody linked to the complement modulator molecule comprises a heterodimeric molecule, further comprising a monovalent anti-Robo2 antibody, an anti-COL4A3 antibody, an anti-COL4A4 antibody, or an anti-COL4A5 antibody and monovalent human complement modulator molecules. In some embodiments, the anti-COL4A3 antibody linked to the complement modulator molecule comprises a heterodimeric molecule, further comprising a monovalent anti-COL4A3 antibody, anti-COL4A4 antibody, anti-COL4A5 antibody, or an anti-Robo2 antibody and monovalent human complement modulator molecules. In some embodiments, the anti-COL4A4 antibody linked to the complement modulator molecule comprises a heterodimeric molecule, further comprising a monovalent anti-COL4A4 antibody, anti-COL4A3 antibody, anti-COL4A5 antibody, or an anti-Robo2 antibody and monovalent human complement modulator molecules. In some embodiments, the anti-COL4A5 antibody linked to the complement modulator molecule comprises a heterodimeric molecule, further comprising a monovalent anti-COL4A5 antibody, anti-COL4A3 antibody, anti-COL4A4 antibody, or an anti-Robo2 antibody and monovalent human complement modulator molecules. In some embodiments, the complement modulator is selected from CD55, CD59, CR1, and DCP.

In some embodiments, the C-terminus of the complement modulator is linked via a G/S or A/E linker to the N-terminus of the targeting moiety, e.g., anti-Robo2 antibody, anti-COL4A3 antibody, anti-COL4A4 antibody, or anti-COL4A5 antibody.

The molecules comprising an anti-Robo2 antibody and a complement modulator, a PD-1 antibody, or an IL-2 mutein, can be various formats as described herein. In some embodiments, the complement modulator is selected from a CD55 protein, a CD59 protein, a CR1 protein, and a DCP protein. For example, the molecules comprising an anti-Robo2 antibody and a complement modulator, a PD-1 antibody, or an IL-2 mutein, can be in the following formats:

### CD55 ML-N Format:

Heavy Chain: NT-[VH_anti-Robo2 antibody]-[CH1-CH2-CH3]-[LinkerA/B/C]-[CD55]-CT
Light Chain: NT-[VK_anti-Robo2 antibody]-[CL]-CT

### CD55 ML-N(2) Format:

Heavy Chain: NT-[VH_anti-COL4A3 antibody]-[CH1-CH2-CH3]-[LinkerA/B/C]-[CD55]-CT
Light Chain: NT-[VK_anti-COL4A3 antibody]-[CL]-CT

### CD55 ML-N(3) Format:

Heavy Chain: NT-[CD55]-[LinkerA/B/C]-[VH_anti-Robo2 antibody]-[CH1-CH2-CH3]-CT Light Chain: NT-[VK_anti-Robo2 antibody]-[CL]-CT CD55 ML-N(4) Format:
Heavy Chain: NT-[CD55]-[LinkerA/B/C]-[VH_anti-COL4A3 antibody]-[CH1-CH2-CH3]-CT
Light Chain: NT-[VK_anti-COL4A3 antibody]-[CL]-CT

### CD55 ML-N(5) Format:

Heavy Chain 1: NT-[VH_anti-Robo2 antibody]-[CH1-CH2-CH3]-[LinkerA/B/C/D]-[CD55]-CT
Heavy Chain 2: NT-[VH_anti-Robo2 antibody]-[CH1-CH2-CH3]-CT
Light Chain: NT-[VK_anti-Robo2 antibody]-[CL]-CT

### CD55 ML-N(6) Format:

Heavy Chain 1: NT-[VH_anti-COL4A3 antibody]-[CH1-CH2-CH3]-[LinkerA/B/C/D]-[CD55]-CT
Heavy Chain 2: NT-[VH_anti-COL4A3 antibody]-[CH1-CH2-CH3]-CT
Light Chain: NT-[VK_anti-COL4A3 antibody]-[CL]-CT

### CD55 ML-N(7) Format:

Heavy Chain 1: NT-[VH_anti-Robo2 antibody]-[CH1-CH2-CH3]-CT
Heavy Chain 2: NT-[IgG1Fc]-[LinkerA/B/C/D]-[CD55]-CT
Light Chain: NT-[VK_anti-Robo2 antibody]-[CL]-CT

### CD55 ML-N(8) Format:

Heavy Chain 1: NT-[VH_anti-COL4A3 antibody]-[CH1-CH2-CH3]-CT
Heavy Chain 2: NT-[IgG1Fc]-[LinkerA/B/C/D]-[CD55]-CT
Light Chain: NT-[VK_anti-COL4A3 antibody]-[CL]-CT

### CD55 ML-N(9) Format:

Heavy Chain 1: NT-[VH_anti-Robo2 antibody]-[CH1]-[IgG2H]-[CD55]-CT
Light Chain: NT-[VK_anti-Robo2 antibody]-[CL]-CT

### CD55 ML-N(10) Format:

Heavy Chain 1: NT-[VH_anti-COL4A3 antibody]-[CH1]-[IgG2H]-[CD55]-CT
Light Chain: NT-[VK_anti-COL4A3 antibody]-[CL]-CT

### CD55 CL-N Format:

Light Chain: NT-[VK_anti-Robo2 antibody]-[CL]-[LinkerA/B/C]-[CD55]-CT
Heavy Chain: NT-[VH_anti-Robo2 antibody]-[CH1-CH2-CH3]-CT

### CD55 CL-N(2) Format:

Light Chain: NT-[VK_anti-COL4A3 antibody]-[CL]-[LinkerA/B/C]-[CD55]-CT
Heavy Chain: NT-[VH_anti-COL4A3 antibody]-[CH1-CH2-CH3]-CT
   CD55 CL-N(3) Format: 15 Light Chain: NT-[CD55]-[LinkerA/B/C/D]-[VK_anti-Robo2 antibody]-[CL]-CT
Heavy Chain: NT-[VH_anti-Robo2 antibody]-[CH1-CH2-CH3]-CT

### CD55 CL-N(4) Format:

Light Chain: NT-[CD55]-[LinkerA/B/C/D]-[VK_anti-COL4A3 antibody]-[CL]-CT
Heavy Chain: NT-[VH_anti-COL4A3 antibody]-[CH1-CH2-CH3]-CT

### CD59 ML-N Format:

Heavy Chain: NT-[VH_anti-Robo2 antibody]-[CH1-CH2-CH3]-[LinkerA/B/C]-[CD59]-CT
Light Chain: NT-[VK_anti-Robo2 antibody]-[CL]-CT

### CD59 ML-N(2) Format:

Heavy Chain: NT-[VH_anti-COL4A3 antibody]-[CH1-CH2-CH3]-[LinkerA/B/C]-[CD59]-CT
Light Chain: NT-[VK_anti-COL4A3 antibody]-[CL]-CT

### CD59 ML-N(3) Format:

Heavy Chain: NT-[CD59]-[LinkerA/B/C]-[VH_anti-Robo2 antibody]-[CH1-CH2-CH3]-CT
Light Chain: NT-[VK_anti-Robo2 antibody]-[CL]-CT

### CD59 ML-N(4) Format:

Heavy Chain: NT-[CD59]-[LinkerA/B/C]-[VH_anti-COL4A3 antibody]-[CH1-CH2-CH3]-CT
Light Chain: NT-[VK_anti-COL4A3 antibody]-[CL]-CT

### CD59 ML-N(5) Format:

Heavy Chain 1: NT-[VH_anti-Robo2 antibody]-[CH1-CH2-CH3]-[LinkerA/B/C/D]-[CD59]-CT
Heavy Chain 2: NT-[VH_anti-Robo2 antibody]-[CH1-CH2-CH3]-CT
Light Chain: NT-[VK_anti-Robo2 antibody]-[CL]-CT

### CD59 ML-N(6) Format:

Heavy Chain 1: NT-[VH_anti-COL4A3 antibody]-[CH1-CH2-CH3]-[LinkerA/B/C/D]-[CD59]-CT
Heavy Chain 2: NT-[VH_anti-COL4A3 antibody]-[CH1-CH2-CH3]-CT
Light Chain: NT-[VK_anti-COL4A3 antibody]-[CL]-CT

### CD59 ML-N(7) Format:

Heavy Chain 1: NT-[VH_anti-Robo2 antibody]-[CH1-CH2-CH3]-CT
Heavy Chain 2: NT-[IgG1Fc]-[LinkerA/B/C/D]-[CD59]-CT
Light Chain: NT-[VK_anti-Robo2 antibody]-[CL]-CT

### CD59 ML-N(8) Format:

Heavy Chain 1: NT-[VH_anti-COL4A3 antibody]-[CH1-CH2-CH3]-CT
Heavy Chain 2: NT-[IgG1Fc]-[LinkerA/B/C/D]-[CD59]-CT
Light Chain: NT-[VK_anti-COL4A3 antibody]-[CL]-CT

### CD59 ML-N(9) Format:

Heavy Chain 1: NT-[VH_anti-Robo2 antibody]-[CH1]-[IgG2H]-[CD59]-CT
Light Chain: NT-[VK_anti-Robo2 antibody]-[CL]-CT

### CD59 ML-N(10) Format:

Heavy Chain 1: NT-[VH_anti-COL4A3 antibody]-[CH1]-[IgG2H]-[CD59]-CT
Light Chain: NT-[VK_anti-COL4A3 antibody]-[CL]-CT

### CD59 CL-N Format:

Light Chain: NT-[VK_anti-Robo2 antibody]-[CL]-[LinkerA/B/C]-[CD59]-CT
Heavy Chain: NT-[VH_anti-Robo2 antibody]-[CH1-CH2-CH3]-CT

### CD59 CL-N(2) Format:

Light Chain: NT-[VK_anti-COL4A3 antibody]-[CL]-[LinkerA/B/C]-[CD59]-CT
Heavy Chain: NT-[VH_anti-COL4A3 antibody]-[CH1-CH2-CH3]-CT
   CD59 CL-N(3) Format: 30 Light Chain: NT-[CD59]-[LinkerA/B/C/D]-[VK_anti-Robo2 antibody]-[CL]-CT
Heavy Chain: NT-[VH_anti-Robo2 antibody]-[CH1-CH2-CH3]-CT

### CD59 CL-N(4) Format:

Light Chain: NT-[CD59]-[LinkerA/B/C/D]-[VK_anti-COL4A3 antibody]-[CL]-CT
Heavy Chain: NT-[VH_anti-COL4A3 antibody]-[CH1-CH2-CH3]-CT

### CR1 ML-N Format:

Heavy Chain: NT-[VH_anti-Robo2 antibody]-[CH1-CH2-CH3]-[LinkerA/B/C]-[CR1]-CT
Light Chain: NT-[VK_anti-Robo2 antibody]-[CL]-CT

### CR1 ML-N(2) Format:

Heavy Chain: NT-[VH_anti-COL4A3 antibody]-[CH1-CH2-CH3]-[LinkerA/B/C]-[CR1]-CT
Light Chain: NT-[VK_anti-COL4A3 antibody]-[CL]-CT

### CR1 ML-N(3) Format:

Heavy Chain: NT-[CR1]-[LinkerA/B/C]-[VH_anti-Robo2 antibody]-[CH1-CH2-CH3]-CT
Light Chain: NT-[VK_anti-Robo2 antibody]-[CL]-CT

### CR1 ML-N(4) Format:

Heavy Chain: NT-[CR1]-[LinkerA/B/C]-[VH_anti-COL4A3 antibody]-[CH1-CH2-CH3]-CT
Light Chain: NT-[VK_anti-COL4A3 antibody]-[CL]-CT

### CR1 ML-N(5) Format:

Heavy Chain 1: NT-[VH_anti-Robo2 antibody]-[CH1-CH2-CH3]-[LinkerA/B/C/D]-[CR1]-CT
Heavy Chain 2: NT-[VH_anti-Robo2 antibody]-[CH1-CH2-CH3]-CT
Light Chain: NT-[VK_anti-Robo2 antibody]-[CL]-CT

### CR1 ML-N(6) Format:

Heavy Chain 1: NT-[VH_anti-COL4A3 antibody]-[CH1-CH2-CH3]-[LinkerA/B/C/D]-[CR1]-CT
Heavy Chain 2: NT-[VH_anti-COL4A3 antibody]-[CH1-CH2-CH3]-CT
Light Chain: NT-[VK_anti-COL4A3 antibody]-[CL]-CT

### CR1 ML-N(7) Format:

Heavy Chain 1: NT-[VH_anti-Robo2 antibody]-[CH1-CH2-CH3]-CT
Heavy Chain 2: NT-[IgG1Fc]-[LinkerA/B/C/D]-[CR1]-CT
Light Chain: NT-[VK_anti-Robo2 antibody]-[CL]-CT

### CR1 ML-N(8) Format:

Heavy Chain 1: NT-[VH_anti-COL4A3 antibody]-[CH1-CH2-CH3]-CT
Heavy Chain 2: NT-[IgG1Fc]-[LinkerA/B/C/D]-[CR1]-CT
Light Chain: NT-[VK_anti-COL4A3 antibody]-[CL]-CT

### CR1 ML-N(9) Format:

Heavy Chain 1: NT-[VH_anti-Robo2 antibody]-[CH1]-[IgG2H]-[CR1]-CT
Light Chain: NT-[VK_anti-Robo2 antibody]-[CL]-CT

### CR1 ML-N(10) Format:

Heavy Chain 1: NT-[VH_anti-COL4A3 antibody]-[CH1]-[IgG2H]-[CR1]-CT
Light Chain: NT-[VK_anti-COL4A3 antibody]-[CL]-CT

### CR1 CL-N Format:

Light Chain: NT-[VK_anti-Robo2 antibody]-[CL]-[LinkerA/B/C]-[CR1]-CT
Heavy Chain: NT-[VH_anti-Robo2 antibody]-[CH1-CH2-CH3]-CT

### CR1 CL-N(2) Format:

Light Chain: NT-[VK_anti-COL4A3 antibody]-[CL]-[LinkerA/B/C]-[CR1]-CT
Heavy Chain: NT-[VH_anti-COL4A3 antibody]-[CH1-CH2-CH3]-CT

### CR1 CL-N(3) Format:

Light Chain: NT-[CR1]-[LinkerA/B/C/D]-[VK_anti-Robo2 antibody]-[CL]-CT
Heavy Chain: NT-[VH_anti-Robo2 antibody]-[CH1-CH2-CH3]-CT

### CR1 CL-N(4) Format:

Light Chain: NT-[CR1]-[LinkerA/B/C/D]-[VK_anti-COL4A3 antibody]-[CL]-CT
Heavy Chain: NT-[VH_anti-COL4A3 antibody]-[CH1-CH2-CH3]-CT

### DCP ML-N Format:

Heavy Chain: NT-[VH_anti-Robo2 antibody]-[CH1-CH2-CH3]-[LinkerA/B/C]-[DCP]-CT
Light Chain: NT-[VK_anti-Robo2 antibody]-[CL]-CT

### DCP ML-N(2) Format:

Heavy Chain: NT-[VH_anti-COL4A3 antibody]-[CH1-CH2-CH3]-[LinkerA/B/C]-[DCP]-CT
Light Chain: NT-[VK_anti-COL4A3 antibody]-[CL]-CT

### DCP ML-N(3) Format:

Heavy Chain: NT-[DCP]-[LinkerA/B/C]-[VH_anti-Robo2 antibody]-[CH1-CH2-CH3]-CT
Light Chain: NT-[VK_anti-Robo2 antibody]-[CL]-CT

### DCP ML-N(4) Format:

Heavy Chain: NT-[DCP]-[LinkerA/B/C]-[VH_anti-COL4A3 antibody]-[CH1-CH2-CH3]-CT
Light Chain: NT-[VK_anti-COL4A3 antibody]-[CL]-CT

### DCP ML-N(5) Format:

Heavy Chain 1: NT-[VH_anti-Robo2 antibody]-[CH1-CH2-CH3]-[LinkerA/B/C/D]-[DCP]-CT
Heavy Chain 2: NT-[VH_anti-Robo2 antibody]-[CH1-CH2-CH3]-CT
Light Chain: NT-[VK_anti-Robo2 antibody]-[CL]-CT

### DCP ML-N(6) Format:

Heavy Chain 1: NT-[VH_anti-COL4A3 antibody]-[CH1-CH2-CH3]-[LinkerA/B/C/D]-[DCP]-CT
Heavy Chain 2: NT-[VH_anti-COL4A3 antibody]-[CH1-CH2-CH3]-CT
Light Chain: NT-[VK_anti-COL4A3 antibody]-[CL]-CT

### DCP ML-N(7) Format:

Heavy Chain 1: NT-[VH_anti-Robo2 antibody]-[CH1-CH2-CH3]-CT
Heavy Chain 2: NT-[IgG1Fc]-[LinkerA/B/C/D]-[DCP]-CT
Light Chain: NT-[VK_anti-Robo2 antibody]-[CL]-CT

### DCP ML-N(8) Format:

Heavy Chain 1: NT-[VH_anti-COL4A3 antibody]-[CH1-CH2-CH3]-CT
Heavy Chain 2: NT-[IgG1Fc]-[LinkerA/B/C/D]-[DCP]-CT
Light Chain: NT-[VK_anti-COL4A3 antibody]-[CL]-CT

### DCP ML-N(9) Format:

Heavy Chain 1: NT-[VH_anti-Robo2 antibody]-[CH1]-[IgG2H]-[DCP]-CT
Light Chain: NT-[VK_anti-Robo2 antibody]-[CL]-CT

### DCP ML-N(10) Format:

Heavy Chain 1: NT-[VH_anti-COL4A3 antibody]-[CH1]-[IgG2H]-[DCP]-CT
Light Chain: NT-[VK_anti-COL4A3 antibody]-[CL]-CT

### DCP CL-N Format:

Light Chain: NT-[VK_anti-Robo2 antibody]-[CL]-[LinkerA/B/C]-[DCP]-CT
Heavy Chain: NT-[VH_anti-Robo2 antibody]-[CH1-CH2-CH3]-CT

### DCP CL-N(2) Format:

Light Chain: NT-[VK_anti-COL4A3 antibody]-[CL]-[LinkerA/B/C]-[DCP]-CT
Heavy Chain: NT-[VH_anti-COL4A3 antibody]-[CH1-CH2-CH3]-CT

### DCP CL-N(3) Format:

Light Chain: NT-[DCP]-[LinkerA/B/C/D]-[VK_anti-Robo2 antibody]-[CL]-CT
Heavy Chain: NT-[VH_anti-Robo2 antibody]-[CH1-CH2-CH3]-CT

### DCP CL-N(4) Format:

Light Chain: NT-[DCP]-[LinkerA/B/C/D]-[VK_anti-COL4A3 antibody]-[CL]-CT
Heavy Chain: NT-[VH_anti-COL4A3 antibody]-[CH1-CH2-CH3]-CT

### PD-1 ML-N Format:

Heavy Chain: NT-[VH_PD-1]-[CH1-CH2-CH3]-[LinkerA]-[anti-Robo2 antibody scFv]-CT
Light Chain: NT-[VK_PD-1]-[CK]-CT

### PD-1 ML-N(2) Format:

Heavy Chain: NT-[VH_PD-1]-[CH1-CH2-CH3]-[LinkerC]-[anti-Robo2 antibody scFab]-CT
Light Chain: NT-[VK_PD-1]-[CK]-CT

### PD-1 ML-C Format:

Heavy Chain: NT-[VH_anti-Robo2 antibody]-[CH1-CH2-CH3]-[LinkerA]-[PD-1scFv]-CT
Light Chain: NT-[VK_anti-Robo2 antibody]-[CK]-CT

### PD-1 CL-N Format:

Light Chain: NT-[VH_PD-1]-[CK]-[LinkerD]-[anti-Robo2 antibody scFab]-CT
Heavy Chain: NT-[VK_PD-1]-[CH1-CH2-CH3]-CT

### PD-1 IgG Format:

Heavy Chain: NT-[VH_PD-1]-[CH1-CH2-CH3]
Light Chain: NT-[VK_PD-1]-[CK]-CT

### IL-2 ML-N Format:

Heavy Chain: NT-[VH_anti-Robo2 antibody]-[CH1-CH2-CH3]-[LinkerA]-[IL-2 mutein]-CT
Light Chain: NT-[VK_anti-Robo2 antibody]-[CL]-CT

### IL-2 CL-N Format:

Light Chain: NT-[VK_anti-Robo2 antibody]-[CL]-[LinkerC/D]-[IL-2 mutein]-CT
Heavy Chain: NT-[VH_anti-Robo2 antibody]-[CH1-CH2-CH3]-CT
   The abbreviations used above are as follows:

| Component | Description |
|---|---|
| NT | N-terminus |
| CT | C-terminus |
| VH_PD-1 | VH domain of PD-1 antibody |
| | as provided herein. |
| VK_PD-1 | VK domain of PD-1 antibody |
| | as provided herein. |
| PD-1scFv | PD-1 antibody in scFv |
| | comprising the VH and VK |
| | domain. |
| VH_ anti-Robo2 antibody | VH domain of anti-Robo2 |
| | antibody Ab as provided |
| | herein. |
| VH_ anti-COL4A3 antibody | VH domain of anti-COL4A3 |
| | antibody Ab as provided |
| | herein. |
| VK_ anti-Robo2 antibody | VK domain of- anti-Robo2 |
| | antibody Ab as provided |
| | herein. This can also be |
| | substituted with a VL |
| | sequences as provided herein. |
| VK_ anti-COL4A3 antibody | VK domain of anti-COL4A3 |
| | antibody Ab as provided |
| | herein. This can also be |
| | substituted with a VL |
| | sequences as provided herein. |
| anti-Robo2 antibody scFv | anti-Robo2 antibody scFV Ab |
| | as provided herein. |
| anti-COL4A3 antibody scFv | anti-COL4A3 antibody scFV |
| | Ab as provided herein. |
| anti-Robo2 antibody scFab | anti-Robo2 antibody scFab Ab |
| | as provided herein. |
| anti-COL4A3 antibody scFab | anti-COL4A3 antibody scFab |
| | Ab as provided herein. |
| VH_ anti-Robo2 antibody_BM1 | Rat anti-mouse anti-Robo2 |
| | antibody placeholder VH |
| | domain |
| VH_ anti-COL4A3 | Rat anti-mouse anti-COL4A3 |
| antibody_BM1 | antibody placeholder VH |
| | domain |
| VK_ anti-Robo2 antibody_BM1 | Rat anti-mouse anti-Robo2 |
| | antibody placeholder VK |
| | domain |
| VK_ anti-COL4A3 | Rat anti-mouse anti-COL4A3 |
| antibody_BM1 | antibody placeholder VK |
| | domain |
| Robo2antibody scFv_BM1 | Rat anti-mouse anti-Robo2 |
| | antibody placeholder scFv |
| COL4A3antibody scFv_BM1 | Rat anti-mouse anti-COL4A3 |
| | antibody placeholder scFv |
| VH_PD-1_BM1 | Anti-human PD-1 agonist |
| | placeholder VH domain |
| VK_PD-1_BM1 | Anti-human PD-1 agonist |
| | placeholder VK domain |
| CH1-CH2-CH3 | Human IgG1 Constant Heavy |
| | 1 (CH1), Constant Heavy 2 |
| | (CH2), and Constant Heavy 3 |
| | (CH3) domains |
| IgG1 Fc | Human IgG1 Fc |
| IgG2H | Human IgG2 Hinger region |
| CK | Human constant kappa |
| | domain |
| CL | Constant lambda domain |
| IL-2_Mutein | IL-2 moiety such as those |
| | provided herein. |
| CD55 | CD55 effector domains such |
| | as those provided herein |
| CD59 | CD59 effector domains such |
| | as those provided herein |
| CR1 | CR1 effector domains such as |
| | those provided herein |
| DCP | DCP effector domains such as |
| | those provided herein |
| Linker_A | G/S, or G/S/E, or A/E/K |
| | linker (5 amino acid length) |
| Linker_B | G/S, or G/S/E, or A/E/K |
| | linker (10 amino acid length) |
| Linker_C | G/S, or G/S/E, or A/E/K |
| | linker (15 amino acid length) |
| Linker_D | G/S, or G/S/E, or A/E/K |
| | linker (20 amino acid length) |

The sequence of CH1-CH2-CH3 can be for example,

(SEQ ID NO: 53)
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGAPSVFLFPPKP
KDTLMISRTPEVTCVVVDVSHEDPEVKFNWYVDGVEVHNAKTKPREEQYNSTYRVVSVLTVLHQD
WLNGKEYKCKVSNKALPAPIEKTISKAKGOPREPQVYTLPPSREEMTKNQVSLTCLVKGFYPSDI
AVEWESNGQPENNYKTTPPVLDSDGSFFLYSKLTVDKSRWQQGNVFSCSVMHEALHNHYTOKSLS
LSPG;
or
(KIH mutations; SEQ ID NO: 115)
ASTKGPSVFPLAPSSKSTSGGTAALGCLVKDYFPEPVTVSWNSGALTSGVHTFPAVLOSSGLYSL
SSVVTVPSSSLGTQTYICNVNHKPSNTKVDKKVEPKSCDKTHTCPPCPAPEAAGAPSVELFPPKP
KDTLMISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVYTLPPCREEMTKNQVSLSCAVKGFYPSDI
AVEWESNGQPENNYKTTPPVLDSDGSFFLVSKLTVDKSRWQQGNVFSCSVMHEALHNHYTOKSLS
LSPG;
or
(KIH mutations; SEQ ID NO: 116)
ASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPEAAGAPSVELFPPKP
KDTLMISRTPEVTCVVVDVSHEDPEVKENWYVDGVEVHNAKTKPREEQYNSTYRVVSVLTVLHQD
WLNGKEYKCKVSNKALPAPIEKTISKAKGQPREPQVCTLPPSREEMTKNQVSLWCLVKGFYPSDI
AVEWESNGQPENNYKTTPPVLDSDGSFFLYSKLTVDKSRWQQGNVFSCSVMHEALHNHYTOKSLS
LSPG;
or
(KIH mutations; SEQ ID NO: 117)
ASTKGPSVFPLAPSSKSTSGGTAALGCLVKDYFPEPVTVSWNSGALTSGVHTFPAVLOSSGLYSL
SSVVTVPSSSLGTQTYICNVNHKPSNTKVDKKVEPKSCDKTHTCPPCPAPEAAGAPSVELFPPKP
KDTLMISRTPEVTCVVVDVSHEDPEVKFNWYVDGVEVHNAKTKPREEQYNSTYRVVSVLTVLHQD
WLNGKEYKCKVSNKALPAPIEKTISKAKGQPREPQVCTLPPSREEMTKNQVSLSCAVKGFYPSDI
AVEWESNGQPENNYKTTPPVLDSDGSFFLVSKLTVDKSRWQQGNVFSCSVMHEALHNHYTOKSLS
LSPG;
or
(KIH mutations; SEQ ID NO: 118)
ASTKGPSVFPLAPSSKSTSGGTAALGCLVKDYFPEPVTVSWNSGALTSGVHTFPAVLOSSGLYSL
SSVVTVPSSSLGTQTYICNVNHKPSNTKVDKKVEPKSCDKTHTCPPCPAPEAAGAPSVFLFPPKP
KDTLMISRTPEVTCVVVDVSHEDPEVKENWYVDGVEVHNAKTKPREEQYNSTYRVVSVLTVLHQD
WLNGKEYKCKVSNKALPAPIEKTISKAKGQPREPQVYTLPPCREEMTKNQVSLWCLVKGFYPSDI
AVEWESNGQPENNYKTTPPVLDSDGSFFLYSKLTVDKSRWQQGNVFSCSVMHEALHNHYTQKSLS
LSPG
The sequence of CK/CL can be, for example,
(SEQ ID NO: 54)
RTVAAPSVFIFPPSDEQLKSGTASVVCLLNNFYPREAKVQWKVDNALQSGNSQESVTEQDSKDST
YSLSSTLTLSKADYEKHKVYACEVTHQGLSSPVTKSENRGEC.

The sequence of IgG2 hinge can be, for example, EPKSCCVECPPCPAPPAAAGA (SEQ ID NO: 119).

In some embodiments, if the therapeutic compound comprises an Fc portion, the Fc domain, (portion) bears mutations to render the Fc region “effectorless,” meaning unable to bind FcRs. The mutations that render Fc regions effectorless are known. In some embodiments, the mutations in the Fc region, which is according to the known numbering system, are selected from the group consisting of: K322A, L234A, L235A, G237A, L234F, L235E, N297, and P331S, or any combination thereof. In some embodiments, the Fc mutations comprises a mutation at L234 and/or L235 and/or G237. In some embodiments, the Fc mutations comprise L234A and/or L235A mutations, which can be referred to as LALA mutations. In some embodiments, the Fc mutations comprise L234A, L235A, and G237A mutations.

In some embodiments, the Fc portion has a sequence of:

(SEQ ID NO: 120)
DKTHTCPPCPAPEAAGAPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQVYT
LPPCREEMTKNQVSLSCAVKGFYPSDIAVEWESNGQPENNYKTTPPVLDSDGSFFLVSKLTVDKS
RWQQGNVFSCSVMHEALHNHYTOKSLSLSPG;
or
(SEQ ID NO: 121)
DKTHTCPPCPAPEAAGAPSVELFPPKPKDTLMISRTPEVTCVVVDVSHEDPEVKENWYVDGVEVH
NAKTKPREEQYNSTYRVVSVLTVLHQDWLNGKEYKCKVSNKALPAPIEKTISKAKGQPREPQVCT
LPPSREEMTKNQVSLWCLVKGFYPSDIAVEWESNGQPENNYKTTPPVLDSDGSFFLYSKLTVDKS
RWQQGNVFSCSVMHEALHNHYTOKSLSLSPG;
or
(SEQ ID NO: 122)
DKTHTCPPCPAPEAAGAPSVFLFPPKPKDTLMISRTPEVTCVVVDVSHEDPEVKENWYVDGVEVH
NAKTKPREEQYNSTYRVVSVLTVLHQDWLNGKEYKCKVSNKALPAPIEKTISKAKGQPREPQVYT
LPPCREEMTKNQVSLWCLVKGFYPSDIAVEWESNGQPENNYKTTPPVLDSDGSFFLYSKLTVDKS
RWQQGNVFSCSVMHEALHNHYTOKSLSLSPG;
or
(SEQ ID NO: 123)
DKTHTCPPCPAPEAAGAPSVELFPPKPKDTLMISRTPEVTCVVVDVSHEDPEVKENWYVDGVEVH
NAKTKPREEQYNSTYRVVSVLTVLHQDWLNGKEYKCKVSNKALPAPIEKTISKAKGQPREPQVCT
LPPSREEMTKNQVSLSCAVKGFYPSDIAVEWESNGQPENNYKTTPPVLDSDGSFFLVSKLTVDKS
RWQQGNVFSCSVMHEALHNHYTQKSLSLSPG.

Disclosed herein are Linker Region polypeptides, therapeutic peptides, and nucleic acids encoding the polypeptides (e.g., therapeutic compounds), vectors comprising the nucleic acid sequences, and cells comprising the nucleic acids or vectors.

Therapeutic compounds can comprise a plurality of specific targeting moieties. In some embodiments, the therapeutic compound comprises a plurality one specific targeting moiety, a plurality of copies of a donor specific targeting moiety or a plurality of tissue specific targeting moieties. In some embodiments, a therapeutic compound comprises a first and a second donor specific targeting moiety, e.g., a first donor specific targeting moiety specific for a first donor target and a second donor specific targeting moiety specific for a second donor target, e.g., wherein the first and second target are found on the same donor tissue. In some embodiments, the therapeutic compound comprises e.g., a first specific targeting moiety for a tissue specific target and a second specific targeting moiety for a second target, e.g., wherein the first and second target are found on the same or different target tissue.

Polypeptides Derived from Reference, E.G., Human Polypeptides

In some embodiments, a component of a therapeutic molecule is derived from or based on a reference molecule, e.g., in the case of a therapeutic molecule for use in humans, from a naturally occurring human polypeptide. For example, in some embodiments, all or a part of a CD55, CD59, CR1, or DCP molecule, a specific targeting moiety, a target ligand binding molecule, or a tissue specific targeting moiety, is based on or derived from a naturally occurring human polypeptide. A PD-L1 molecule can be based on or derived from a human PD-L1 sequence.

In some embodiments, a therapeutic compound component, e.g., a PD-L1 molecule:
a) comprises all or a portion of, e.g., an active portion of, a naturally occurring form of the human polypeptide;
b) comprises all or a portion of, e.g., an active portion of, a human polypeptide having a sequence appearing in a database, e.g., GenBank database, as of Jan. 11, 2017, a naturally occurring form of the human polypeptide that is not associated with a disease state;
c) comprises a human polypeptide having a sequence that differs by no more than 1, 2, 3, 4, 5, 10, 20, or 30 amino acid residues from a sequence of a) or b);
d) comprises a human polypeptide having a sequence that differs by no more than 1, 2, 3, 4, 5 10, 20, or 30% its amino acids residues from a sequence of a) or b);
e) comprises a human polypeptide having a sequence that does not differ substantially from a sequence of a) or b); or
f) comprises a human polypeptide having a sequence of c), d), or e) that does not differ substantially in biological activity, e.g., ability to enhance or inhibit an immune response, from a human polypeptide having the sequence of a) or b).

### Pharmaceutical Compositions and Kits

In another aspect, the present embodiments provide compositions, e.g., pharmaceutically acceptable compositions, which include a therapeutic compound, polypeptide, protein, or molecule described herein, formulated together with a pharmaceutically acceptable carrier. As used herein, “pharmaceutically acceptable carrier” includes any and all solvents, dispersion media, isotonic and absorption delaying agents, and the like that are physiologically compatible.

The carrier can be suitable for intravenous, intramuscular, subcutaneous, parenteral, rectal, local, ophthalmic, topical, spinal or epidermal administration (e.g., by injection or infusion). As used herein, the term “carrier” means a diluent, adjuvant, or excipient with which a compound is administered. In some embodiments, pharmaceutical carriers can also be liquids, such as water and oils, including those of petroleum, animal, vegetable or synthetic origin, such as peanut oil, soybean oil, mineral oil, sesame oil, and the like. The pharmaceutical carriers can also be saline, gum acacia, gelatin, starch paste, talc, keratin, colloidal silica, urea, and the like. In addition, auxiliary, stabilizing, thickening, lubricating, and coloring agents can be used. The carriers can be used in pharmaceutical compositions comprising the therapeutic compounds provided for herein.

The compositions and compounds of the embodiments provided herein may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Typical compositions are in the form of injectable or infusible solutions. In some embodiments, the mode of administration is parenteral (e.g., intravenous, subcutaneous, intraperitoneal, intramuscular). In some embodiments, the therapeutic molecule is administered by intravenous infusion or injection. In another embodiment, the therapeutic molecule is administered by intramuscular or subcutaneous injection. In another embodiment, the therapeutic molecule is administered locally, e.g., by injection, or topical application, to a target site. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection, and infusion.

Therapeutic compositions typically are sterile and stable under the conditions of manufacture and storage. The composition can be formulated as a solution, microemulsion, dispersion, liposome, or other ordered structure suitable to high therapeutic molecule concentration. Sterile injectable solutions can be prepared by incorporating the active compound (i.e., therapeutic molecule) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, a therapeutic compound is orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound by other than parenteral administration, it may be necessary to coat the compound with, or co-administer the compound with, a material to prevent its inactivation. Therapeutic compositions can also be administered with medical devices known in the art.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms are dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for a therapeutically or prophylactically effective amount of a therapeutic compound is 0.1-30 mg/kg, more preferably 1-25 mg/kg. Dosages and therapeutic regimens of the therapeutic compound can be determined by a skilled artisan. In certain embodiments, the therapeutic compound is administered by injection (e.g., subcutaneously or intravenously) at a dose of about 1 to 40 mg/kg, e.g., 1 to 30 mg/kg, e.g., about 5 to 25 mg/kg, about 10 to 20 mg/kg, about 1 to 5 mg/kg, 1 to 10 mg/kg, 5 to 15 mg/kg, 10 to 20 mg/kg, 15 to 25 mg/kg, or about 3 mg/kg. The dosing schedule can vary from e.g., once a week to once every 2, 3, or 4 weeks. In one embodiment, the therapeutic compound is administered at a dose from about 10 to 20 mg/kg every other week. The therapeutic compound can be administered by intravenous infusion at a rate of more than 20 mg/min, e.g., 20-40 mg/min, and typically greater than or equal to 40 mg/min to reach a dose of about 35 to 440 mg/m2, typically about 70 to 310 mg/m2, and more typically, about 110 to 130 mg/m2. In embodiments, the infusion rate of about 110 to 130 mg/m2 achieves a level of about 3 mg/kg. In other embodiments, the therapeutic compound is administered by intravenous infusion at a rate of less than 10 mg/min, e.g., less than or equal to 5 mg/min to reach a dose of about 1 to 100 mg/m2, e.g., about 5 to 50 mg/m2, about 7 to 25 mg/m2, or, about 10 mg/m2. In some embodiments, the therapeutic compound is infused over a period of about 30 min. It is to be noted that dosage values may vary with the type and severity of the condition to be alleviated. It is to be further understood that for any particular subject, specific dosage regimens can be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions may include a “therapeutically effective amount” or a “prophylactically effective amount” of a therapeutic molecule. A “therapeutically effective amount” refers to an amount effective, at dosages and for periods of time necessary, to achieve the desired therapeutic result. A therapeutically effective amount of a therapeutic molecule may vary according to factors such as the disease state, age, sex, and weight of the individual, and the ability of the therapeutic compound to elicit a desired response in the individual. A therapeutically effective amount is also one in which any toxic or detrimental effects of a therapeutic molecule t is outweighed by the therapeutically beneficial effects. A “therapeutically effective dosage” preferably inhibits a measurable parameter, e.g., immune attack at least about 20%, more preferably by at least about 40%, even more preferably by at least about 60%, and still more preferably by at least about 80% relative to untreated subjects. The ability of a compound to inhibit a measurable parameter, e.g., immune attack, can be evaluated in an animal model system predictive of efficacy in glomerular disorders or autoimmune disorders. Alternatively, this property of a composition can be evaluated by examining the ability of the compound to inhibit, such inhibition in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Also within the scope of the embodiments is a kit comprising a therapeutic compound described herein. The kit can include one or more other elements including: instructions for use; other reagents, e.g., a label, a therapeutic agent, or an agent useful for chelating, or otherwise coupling, a therapeutic molecule to a label or other therapeutic agent, or a radioprotective composition; devices or other materials for preparing the a therapeutic molecule for administration; pharmaceutically acceptable carriers; and devices or other materials for administration to a subject.

In some embodiments, embodiments provided herein also include, but are not limited to:

1. A protein comprising a glomerular targeting moiety and an effector moiety, wherein
the glomerular targeting moiety is an antibody that binds to a Robo2 protein, an antibody that binds to a COL4A3 protein, an antibody that binds to a COL4A4 protein, or an antibody that binds to a COL4A5 protein; and
the effector moiety is a complement modulator selected from the group consisting of a CD55 protein, a CD59 protein, a CR1 protein, and a DCP protein.
   2. The protein of embodiment 1, wherein the protein binds to a glomerular cell.
   3. The protein of embodiment 1 or embodiment 2, wherein the complement modulator inhibits a complement system.
   4. The protein of embodiment 1, wherein the glomerular targeting moiety is an anti-Robo2 antibody that blocks Robo2 binding to SLIT1, SLIT2, SLIT3, and/or SLIT4.
   5. The protein of embodiment 1, wherein the glomerular targeting moiety is an anti-Robo2 antibody that does not block Robo2 binding to SLIT1, SLIT2, SLIT3, or SLIT4.
   6. The protein of any one of embodiments 1-5, wherein the protein has the formula from N-terminus to C-terminus:
R1-Linker Region A-R2
   wherein, R1 and R2, each independently comprises the effector moiety or the glomerular targeting moiety, and said Linker Region may be absent.
   7. The protein of embodiment 6, wherein Linker Region A comprises
an Fc region;
a glycine/serine linker; or
is absent.
   8. The protein of embodiment 1, wherein the glomerular targeting moiety is in a Fab or scFv configuration.
   9. The protein of embodiment 6, wherein
one of R1 and R2 is the complement modulator and one of R1 and R2 is an anti-Robo2 antibody;
one of R1 and R2 is the CD55 protein and one of R1 and R2 is an anti-Robo2 antibody;
one of R1 and R2 is the CD59 protein and one of R1 and R2 is an anti-Robo2 antibody;
one of R1 and R2 is the CR1 protein and one of R1 and R2 is an anti-Robo2 antibody;
one of R1 and R2 is the DCP protein and one of R1 and R2 is an anti-Robo2 antibody;
one of R1 and R2 is the complement modulator and one of R1 and R2 is an anti-COL4A3 antibody, an anti-COL4A4 antibody, or an anti-COL4A5 antibody;
one of R1 and R2 is the CD55 protein and one of R1 and R2 is an anti-COL4A3 antibody, an anti-COL4A4 antibody, or an anti-COL4A5 antibody;
one of R1 and R2 is the CD59 protein and one of R1 and R2 is an anti-COL4A3 antibody, an anti-COL4A4 antibody, or an anti-COL4A5 antibody;
one of R1 and R2 is the CR1 protein and one of R1 and R2 is an anti-COL4A3 antibody, an anti-COL4A4 antibody, or an anti-COL4A5 antibody;
one of R1 and R2 is the DCP protein and one of R1 and R2 is an anti-COL4A3 antibody, an anti-COL4A4 antibody, or an anti-COL4A5 antibody;
one of R1 and R2 is the TL-2 mutein and one of R1 and R2 is an anti-COL4A3 antibody, an anti-COL4A4 antibody, or an anti-COL4A5 antibody;
one of R1 and R2 is the PD-1 agonist and one of R1 and R2 is an anti-COL4A3 antibody, an anti-COL4A4 antibody, or an anti-COL4A5 antibody.
   10. The protein of embodiment 6, wherein two R1-Linker Region A-R2 proteins form a dimer, wherein each of the proteins can be the same or different.
   11. The protein of embodiment 1, wherein the glomerular targeting moiety comprises:
an amino acid sequence as set forth in SEQ ID NO: 70 or an amino acid sequence having at least 90% identity to the amino acid sequence of SEQ ID NO: 70;
an amino acid sequence as set forth in SEQ ID NO: 71 or an amino acid sequence having at least 90% identity to the amino acid sequence of SEQ ID NO: 71;
an amino acid sequence as set forth in SEQ ID NO: 72 or an amino acid sequence having at least 90% identity to the amino acid sequence of SEQ ID NO: 72;
an amino acid sequence as set forth in SEQ ID NO: 73 or an amino acid sequence having at least 90% identity to the amino acid sequence of SEQ ID NO: 73;
an amino acid sequence as set forth in SEQ ID NO: 74 or an amino acid sequence having at least 90% identity to the amino acid sequence of SEQ ID NO: 74;
an amino acid sequence as set forth in SEQ ID NO: 75 or an amino acid sequence having at least 90% identity to the amino acid sequence of SEQ ID NO: 75;
an amino acid sequence as set forth in SEQ ID NO: 76 or an amino acid sequence having at least 90% identity to the amino acid sequence of SEQ ID NO: 76;
an amino acid sequence as set forth in SEQ ID NO: 77 or an amino acid sequence having at least 90% identity to the amino acid sequence of SEQ ID NO: 77;
an amino acid sequence as set forth in SEQ ID NO: 78 or an amino acid sequence having at least 90% identity to the amino acid sequence of SEQ ID NO: 78;
an amino acid sequence as set forth in SEQ ID NO: 79 or an amino acid sequence having at least 90% identity to the amino acid sequence of SEQ ID NO: 79; or
an amino acid sequence as set forth in SEQ ID NO: 80 or an amino acid sequence having at least 90% identity to the amino acid sequence of SEQ ID NO: 80.
   12. The protein of embodiment 1, wherein the glomerular targeting moiety comprises:
an amino acid sequence as set forth in SEQ ID NO: 70;
an amino acid sequence as set forth in SEQ ID NO: 71;
an amino acid sequence as set forth in SEQ ID NO: 72;
an amino acid sequence as set forth in SEQ ID NO: 73;
an amino acid sequence as set forth in SEQ ID NO: 74;
an amino acid sequence as set forth in SEQ ID NO: 75;
an amino acid sequence as set forth in SEQ ID NO: 76;
an amino acid sequence as set forth in SEQ ID NO: 77;
an amino acid sequence as set forth in SEQ ID NO: 78;
an amino acid sequence as set forth in SEQ ID NO: 79; or
an amino acid sequence as set forth in SEQ ID NO: 80.
   13. The protein of embodiment 1, wherein
the glomerular targeting moiety comprises
   an amino acid sequence as set forth in SEQ ID NO: 70;
   an amino acid sequence as set forth in SEQ ID NO: 71;
   an amino acid sequence as set forth in SEQ ID NO: 72;
   an amino acid sequence as set forth in SEQ ID NO: 73;
   an amino acid sequence as set forth in SEQ ID NO: 74;
   an amino acid sequence as set forth in SEQ ID NO: 75;
   an amino acid sequence as set forth in SEQ ID NO: 76;
   an amino acid sequence as set forth in SEQ ID NO: 77;
   an amino acid sequence as set forth in SEQ ID NO: 78;
   an amino acid sequence as set forth in SEQ ID NO: 79; or
   an amino acid sequence as set forth in SEQ ID NO: 80; and
the effector moiety comprises
   an amino acid sequence as set forth in SEQ ID NO: 1;
   an amino acid sequence as set forth in SEQ ID NO: 2;
   an amino acid sequence as set forth in SEQ ID NO: 3;
   an amino acid sequence as set forth in SEQ ID NO: 4;
   an amino acid sequence as set forth in SEQ ID NO: 5;
   an amino acid sequence as set forth in SEQ ID NO: 6;
   an amino acid sequence as set forth in SEQ ID NO: 7;
   an amino acid sequence as set forth in SEQ ID NO: 8;
   an amino acid sequence as set forth in SEQ ID NO: 9;
   an amino acid sequence as set forth in SEQ ID NO: 10;
   an amino acid sequence as set forth in SEQ ID NO: 11; or
   an amino acid sequence as set forth in SEQ ID NO: 12.
      14. An antibody or antigen binding fragment thereof comprising:
a light chain comprising an amino acid sequence as set forth in SEQ ID NO:83 or an amino acid sequence having at least 90% identity to SEQ ID NO: 83, and a heavy chain comprising an amino acid sequence as set forth in SEQ ID NO:81 or an amino acid sequence having at least 90% identity to SEQ ID NO: 81;
a light chain comprising an amino acid sequence as set forth in SEQ ID NO:87 or an amino acid sequence having at least 90% identity to SEQ ID NO: 87, and a heavy chain comprising an amino acid sequence as set forth in SEQ ID NO:85 or an amino acid sequence having at least 90% identity to SEQ ID NO: 85;
a light chain comprising an amino acid sequence as set forth in SEQ ID NO:102 or an amino acid sequence having at least 90% identity to SEQ ID NO: 102, and a heavy chain comprising an amino acid sequence as set forth in SEQ ID NO:100 or an amino acid sequence having at least 90% identity to SEQ ID NO: 100; or
a light chain comprising an amino acid sequence as set forth in SEQ ID NO:108 or an amino acid sequence having at least 90% identity to SEQ ID NO:108, and a heavy chain comprising an amino acid sequence as set forth in SEQ ID NO:110 or an amino acid sequence having at least 90% identity SEQ ID NO: 110.
   15. An antibody or antigen binding fragment thereof comprising:
a light chain comprising an amino acid sequence as set forth in SEQ ID NO: 83, and a heavy chain comprising an amino acid sequence as set forth in SEQ ID NO: 81;
a light chain comprising an amino acid sequence as set forth in SEQ ID NO: 87, and a heavy chain comprising an amino acid sequence as set forth in SEQ ID NO: 85;
a light chain comprising an amino acid sequence as set forth in SEQ ID NO: 102, and a heavy chain comprising an amino acid sequence as set forth in SEQ ID NO: 100; or
a light chain comprising an amino acid sequence as set forth in SEQ ID NO: 108, and a heavy chain comprising an amino acid sequence as set forth in SEQ ID NO: 110.
   16. A protein comprising:
an amino acid sequence as set forth in SEQ ID NO: 81 or an amino acid sequence having at least 90% identity to SEQ ID NO: 81;
an amino acid sequence as set forth in SEQ ID NO: 83 or an amino acid sequence having at least 90% identity to SEQ ID NO: 83;
an amino acid sequence as set forth in SEQ ID NO: 85 or an amino acid sequence having at least 90% identity to SEQ ID NO: 85;
an amino acid sequence as set forth in SEQ ID NO: 87 or an amino acid sequence having at least 90% identity to SEQ ID NO: 87;
an amino acid sequence as set forth in SEQ ID NO: 89 or an amino acid sequence having at least 90% identity to SEQ ID NO: 89;
an amino acid sequence as set forth in SEQ ID NO: 90 or an amino acid sequence having at least 90% identity to SEQ ID NO: 90;
an amino acid sequence as set forth in SEQ ID NO:91 or an amino acid sequence having at least 90% identity to SEQ ID NO: 91;
an amino acid sequence as set forth in SEQ ID NO: 92 or an amino acid sequence having at least 90% identity to SEQ ID NO: 92;
an amino acid sequence as set forth in SEQ ID NO: 93 or an amino acid sequence having at least 90% identity to SEQ ID NO: 93;
an amino acid sequence as set forth in SEQ ID NO: 94 or an amino acid sequence having at least 90% identity to SEQ ID NO: 94;
an amino acid sequence as set forth in SEQ ID NO: 95 or an amino acid sequence having at least 90% identity to SEQ ID NO: 95;
an amino acid sequence as set forth in SEQ ID NO: 96 or an amino acid sequence having at least 90% identity to SEQ ID NO: 96;
an amino acid sequence as set forth in SEQ ID NO: 97 or an amino acid sequence having at least 90% identity to SEQ ID NO: 97;
an amino acid sequence as set forth in SEQ ID NO: 99 or an amino acid sequence having at least 90% identity to SEQ ID NO: 99;
an amino acid sequence as set forth in SEQ ID NO: 100 or an amino acid sequence having at least 90% identity to SEQ ID NO: 100;
an amino acid sequence as set forth in SEQ ID NO: 102 or an amino acid sequence having at least 90% identity to SEQ ID NO: 102;
an amino acid sequence as set forth in SEQ ID NO: 104 or an amino acid sequence having at least 90% identity to SEQ ID NO: 104;
an amino acid sequence as set forth in SEQ ID NO: 105 or an amino acid sequence having at least 90% identity to SEQ ID NO: 105;
an amino acid sequence as set forth in SEQ ID NO: 106 or an amino acid sequence having at least 90% identity to SEQ ID NO: 106;
an amino acid sequence as set forth in SEQ ID NO: 107 or an amino acid sequence having at least 90% identity to SEQ ID NO: 107;
an amino acid sequence as set forth in SEQ ID NO: 108 or an amino acid sequence having at least 90% identity to SEQ ID NO: 108; or
an amino acid sequence as set forth in SEQ ID NO: 110 or an amino acid sequence having at least 90% identity to SEQ ID NO: 110.
   17. A protein comprising:
an amino acid sequence as set forth in SEQ ID NO: 81;
an amino acid sequence as set forth in SEQ ID NO: 83;
an amino acid sequence as set forth in SEQ ID NO: 85;
an amino acid sequence as set forth in SEQ ID NO: 87;
an amino acid sequence as set forth in SEQ ID NO: 89;
an amino acid sequence as set forth in SEQ ID NO: 90;
an amino acid sequence as set forth in SEQ ID NO: 91;
an amino acid sequence as set forth in SEQ ID NO: 92;
an amino acid sequence as set forth in SEQ ID NO: 93;
an amino acid sequence as set forth in SEQ ID NO: 94;
an amino acid sequence as set forth in SEQ ID NO: 95;
an amino acid sequence as set forth in SEQ ID NO: 96;
an amino acid sequence as set forth in SEQ ID NO: 97;
an amino acid sequence as set forth in SEQ ID NO: 99;
an amino acid sequence as set forth in SEQ ID NO: 100;
an amino acid sequence as set forth in SEQ ID NO: 102;
an amino acid sequence as set forth in SEQ ID NO: 104;
an amino acid sequence as set forth in SEQ ID NO: 105;
an amino acid sequence as set forth in SEQ ID NO: 106;
an amino acid sequence as set forth in SEQ ID NO: 107;
an amino acid sequence as set forth in SEQ ID NO: 108; or
an amino acid sequence as set forth in SEQ ID NO: 110.
   18. A pharmaceutical composition comprising the protein of any of embodiments 1-13 or 16-17 or the antibody or antigen binding fragment of embodiment 14 or embodiment 15, and a pharmaceutically acceptable carrier.
   19. A method of inhibiting a complement system in the kidney glomerulus, the method comprising tethering of the protein of any of embodiments 1-13 or 16-17 to the glomerulus, wherein tethering allows localized inhibition of the complement system within the glomerulus.
   20. A method of inhibiting a complement system in the kidney glomerulus of a subject in need thereof, the method comprising tethering of the protein of any of embodiments 1-13 or 16-17 to the glomerulus of the subject in need thereof, wherein tethering allows localized inhibition of the complement system within the glomerulus of the subject.
   21. The method of embodiment 20, wherein the subject has a kidney disease, selected from the group consisting of: Goodpasture's Syndrome (anti-GBM disease), Alport syndrome, inflammatory renal disease, glomerulonephritis, nephritis, lupus, lupus nephritis, IgA nephritis, membranous nephropathy, membranoproliferative glomerulonephritis, acute kidney injury, and chronic kidney disease, focal segmented glomerular sclerosis (FSGS), lupus nephritis, systemic scleroderma, membranous glomerular nephropathy (MGN), membranous nephropathy (MN), minimal change disease (MCD), IgA nephropathy, ANCA-associated vasculitis (AAV), Sjögren's syndrome, Scleroderma, and systemic sclerosis (SSc), or any combination thereof.
   22. A method of treating a subject with a kidney inflammatory disorder selected from the group consisting of. Goodpasture's Syndrome (anti-GBM disease), Alport syndrome, inflammatory renal disease, glomerulonephritis, nephritis, lupus, lupus nephritis, IgA nephritis, membranous nephropathy, membranoproliferative glomerulonephritis, acute kidney injury, chronic kidney disease, focal segmented glomerular sclerosis (FSGS), lupus nephritis, systemic scleroderma, membranous glomerular nephropathy (MGN), membranous nephropathy (MN), minimal change disease (MCD), IgA nephropathy, ANCA-associated vasculitis (AAV), Sjögren's syndrome, Scleroderma, and systemic sclerosis (SSc), or any combination thereof, the method comprising administering the protein of any of embodiments 1-18, or the pharmaceutical compositions of embodiment 19, to the subject to treat the disorder.
   23. A method of treating glomerular disorder in a subject in need thereof, comprising administering the protein of any of embodiments 1-13 or 16-17, the antibody or antigen-binding fragment of embodiment 14 or 15, or the pharmaceutical compositions of embodiment 18, to the subject to treat the disorder, wherein the glomerular disorder is selected from the group consisting of: atypical hemolytic uremic syndrome (aHUS), anti-neutrophil cytoplasmic antibody mediated vasculitis (ANCA), C3 glomerulopathy, IgA nephropathy, immune complex membranoproliferative glomerulonephritis, ischemic reperfusion injury, lupus nephritis, membranous nephropathy, chronic transplant mediated glomerulopathy, immune-complex glomerulonephritis (GN), pauci-immune GN, anti-glomerular basement membrane GN, monoclonal immunoglobulin GN, C3 glomerulopathy, nephrotic syndrome (NS), primary congenital NS (CNS), renal tubular acidosis (RTA), inherited renal tubulopathies, Faconi syndrome, primary nephrogenic diabetes insipidus, minimal change disease, focal segmental glomerular sclerosis, membranous nephropathy, immunoglobulin A nephropathy, C3 glomerulopathy (DDD, C3 GN), idiopathic immune complex membranoproliferative GN, C4 glomerulopathy, infection-related GN, Renal-limited GN, renal limited vasculitis, collagenofibrotic glomerulopathy, thin basement membranes nephropathy, lipoprotein glomerulopathy, ‘Pure’ mesangial proliferative GN, IgM nephropathy, C1q nephropathy, and Idiopathic nodular glomerulosclerosis, or any combination thereof,
   24. An isolated nucleic acid molecule encoding the protein of any of embodiments 1-17.
   25. An expression vector comprising the isolated nucleic acid of embodiment 24.
   26. A host cell comprising the nucleic acid of embodiment 23 or the expression vector of embodiment 25.
   27. A method of producing a protein of any one of embodiments 1-17 comprising:
(a) culturing a host cell comprising one or more nucleic acids of embodiment 24 in a culture medium under conditions favorable for expression of the one or more nucleic acids; and
(b) optionally recovering the polypeptide from the culture medium.

The following examples are illustrative, but not limiting, of the compounds, compositions and methods described herein. Other suitable modifications and adaptations known to those skilled in the art are within the scope of the following embodiments.

### EXAMPLES

### Example 1: Bifunctional Polypeptides were Made in Different Orientations

Bifunctional polypeptides described herein were made with CD55, CD59, CR1, or DCP complement modulator at the C-terminus and an anti-Robo2 or COL4A3 tethers in IgG1 format. Bifunctional polypeptides were made with CD55, CD59, CR1, or DCP complement modulator at the N-terminus and anti-Robo2 or COL4A3 tethers in scFv format. Bifunctional polypeptides were also made as Fc-less molecules where CD55, CD59, CR1, or DCP complement modulator are at the C-terminus and an anti-Robo2 or COL4A3 tethers are in VHH format. Alternatively, bifunctional polypeptides were made as Fc-less molecules where CD55, CD59, CR1, or DCP complement modulator are at the N-terminus and an anti-Robo2 or COL4A3 tethers are in scFv format. These are represented in FIG. 12-FIG. 18.

### Example 2: Generation of Anti-ROBO2 and COL4A3 Tethered Complement Modulator Bifunctional Molecules

Test articles (TAs) with anti-Robo2 or COL4A3 tethers were fused to various complement modulator (CD55, CD59, CR1, decay-cofactor protein (DCP)) in various orientations, as provided in Example 1. TAs were generated in pTT5 vector and expressed using Expi293F cells following the manufacturer's protocol (Gibco, A14635). The expressed TAs were purified using Protein A or Ni-NTA agarose columns. The yield was determined from 50 mL expression. The data showed yields of: 19.6 mg/L for CDAB1; 22.4 mg/L for CDAB3; 8.2 mg/L for CDAB5; 21.0 mg/L for CDAB6; 3.8 mg/L for CDAB7; 21.2 mg/L for CDAB8; 68.4 mg/L for CDAB9; 24.4 mg/L for CDAB10; 18.8 mg/L for CDAB12; 47.2 mg/L for CDAB11; 90.0 mg/L for CDAB2; 16.0 mg/L for CDAB13; 12.0 mg/L for CDAB14; 7.2 mg/L for CDAB15; 14.2 mg/L for CDAB16; 2.2 mg/L for CDAB17; and 3.8 mg/L for CDAB18. Purified TAs were run in SDS-PAGE gel in reducing and non-reducing conditions with a protein ladder to determine molecular weights. The data showed molecule weights of: 216.1 kDa for CDAB1; 64.2 kDa for CDAB3; 35.7 kDa for CDAB5; 177.8 kDa for CDAB6; 123 kDa for CDAB7; 214.4 kDa for CDAB8; 200.1 kDa for CDAB9; 200.1 kDa for CDAB10; 200.4 kDa for CDAB12; 239.6 kDa for CDAB20; 49.5 kDa for CDAB 11; 217.8 kDa for CDAB2; 63.2 kDa for CDAB4; 218.0 kDa for CDAB13; 64.1 kDa for CDAB14; 36.7 kDa for CDAB15; 179.8 kDa for CDAB16; 125.0 kDa for CDAB17; and 218.9 kDa for CDAB18. TAs were also run in HPLC-SEC column (Agilent, PL1580-3301) to determine % peak of interest (% POI) in solution. The data showed POIs of: 100% for CDAB1; 65% for CDAB3; 64% for CDAB5; 59% for CDAB6; 57% for CDAB7; 100% for CDAB8; 100% for CDAB9; 100% for CDAB10; 100% for CDAB12; 96% for CDAB11; 100% for CDAB2; 200% for CDAB13; 49% for CDAB14; 58% for CDAB15; 86% for CDAB16; 58% for CDAB17; and 97% for CDAB18. Accordingly, the bifunctional molecules comprising anti-Robo2/COL4A3 tethered to CD55, CD59, CR1, or DCP complement modulator in various orientations produced good yields and showed expected molecular weights by SDS-PAGE. Most construct were monodisperse in solution without substantial aggregation.

### Example 3: COL4A3 Tethered Complement Modulator Bifunctionals Bind to their Targets

Anti-human Fc (for CDAB1, CDAB5, Fc-rat COL4A3) or anti-Streptavidin (for CDAB3) or HIS1K (COL4A3-His) biosensors were equilibrated in assay buffer (1% BSA in 1×PBS with 0.05% Tween-20) for 10 minutes before the experiment was setup. Immobilizing reagents were diluted to 4 μg/mL in assay buffer and 200 μL pipetted to 96 well plate. Mouse Robo2 or TAs were titrated down, two-fold dilutions (starting at 50 nM as the highest concentration, 7-point dilution). The experiment was conducted using data acquisition software version 10.0 for OCTET96 RED. Test articles were captured using anti-human Fc biosensors for 180 s. Biosensors loaded with test articles were then equilibrated in assay buffer for 120 s. Association was performed in wells with mRobo2 or TA for 180 seconds. Dissociation was performed in wells with assay buffer for 180 s. The data showed CDAB1 to have Kon(1/Ms) of 9.38E+05; Kdis(1/s) of 1.29E−03; and K_{D}(M) of 1.38E−09. The data showed CDAB6 to have Kon(1/Ms) of 4.12E+05; Kdis(1/s) of 2.94E−03; and K_{D}(M) of 7.13E−09. The data showed CDAB3 to have Kon(1/Ms) of 2.21E+05; Kdis(1/s) of 2.91E−03; K_{D}(M) of 1.32E−08. The data showed CDAB2 to have Kon(1/Ms) of 8.79E+04; Kdis(1/s) of less than 1.0E−07; K_{D}(M) of less than 1.0E−12; and EC50(M) of 3.38E−10. The data showed CDAB13 to have K_{D}(M) of 4.80E−06. The data showed CDAB14 to have K_{D}(M) of 2.30E−05.

An immunosorbent plate was coated with monoFc-conjugated COL4A3, COL4A5, and monoFc alone at a concentration of 2 μg/mL in PBS pH 7.4, 100 μl/well, and incubated overnight at 4° C. Wells were washed with PBS pH 7.4 containing 0.05% Tween-20 (wash buffer) three times, and then blocked with 300 ul/well Superblock™ (blocking buffer) for two hours at room temperature. After three washes with wash buffer, TAs were serially diluted 1:5 from 50 nM in blocking buffer. The diluted material was added to the COL4 coated plate at 100 μl/well for 1 hour at room temperature. After three washes with wash buffer, a goat anti-human Kappa HRP conjugated polyclonal antibody, diluted to 1:5000 in assay buffer, was added to the plate at 100 μl/well for 1 hr at room temperature. After three washes with wash buffer, the assay was developed with TMB, and stopped with 1N HCL. OD 450 nm was measured. The experiment included appropriate controls for non-specific binding of test articles (e.g. monoFc-COL4A5, monoFc alone) to the plate/block in the absence of antigen. MonoFc-Human COL4A3, monoFc-Mouse COL4A3, and human COL4A3-Flag showed binding in the ELISA assay.

Accordingly, anti-Robo2 and COL4A3 tethered complement modulator bifunctionals show binding specificity to their targets.

### Example 4: Bifunctional muCD55 and huCD55 Constructs Inhibit Terminal Complement Complex (C5b-9a) Formation In Vitro

The Hycult Classical Complement Mouse Assay (Cat #HIT420) measuring C5b-9a deposition was used to determine inhibitory activity for all CD55 constructs. All kit components were prepared according to the manufacturer's protocol. Complement-preserved normal mouse serum was diluted 1:4 in cold sample dilution buffer. Test articles were diluted to 2000 or 4000 nM, depending on the experiment, with cold sample dilution buffer followed by 1:3, 5-point serial dilution. 50 μl diluted TA and 50 μl diluted serum were pipetted into the kit plate on ice with 50 μl sample dilution buffer plus 50 μl diluted serum as a 100% activation control and 100 μl sample dilution buffer as a negative control. The plate was sealed and incubated for 1 hr at 37° C., then washed 4× with kit wash buffer. 100 μl diluted kit tracer solution was pipetted into each well and the plate was sealed and incubated for 1 hr at 37° C. After washing 4× with wash buffer 100 μl diluted kit streptavidin was pipetted into each well and the plate was sealed and incubated for 1 hr at 37° C. The plate was washed 4× with wash buffer, 100 μl diluted kit TMB substrate was pipetted into each well and the plate was incubated for 10 min at room temperature in the dark. 100 μl kit stop solution was added to each well and OD was read at 450 nM. Results were calculated as percent inhibition compared to serum-only control. The tethered activity assay was developed by Hycult and followed the same protocol, with the following modifications: (1) plates were coated with a mixture of muIgM to activate the complement pathway and recROBO2 for bifunctional tethering, (2) TA were added to plates, incubated, and washed to remove unbound TA prior to the addition of serum. The data showed soluble IC50(nM) of 193.20 and tethered IC50(nM) of 1.5 for CDAB1; soluble IC50 of 285.40 for CDAB13; soluble IC50 of 342.50 for CDAB18; soluble IC50 of 733.60 for CDAB8; soluble IC50 of 77.65 for CDAB9; soluble IC50 of 55.05 for CDAB10; soluble IC50 of 538.30 for CDAB21; soluble IC50 of 14.24 for CDAB11; and soluble IC50 of 155.60 for CDAB2. Additionally, the data showed maximum inhibition at 200 nM of 59.53% for CDAB1; 83.82% for CDAB3; 94.58% for CDAB4; 44.60% for CDAB6; 81.39% for CDAB22; 101.05% for CDAB23; and 46.66% for isotype control. MuCD55 and huCD55 constructs inhibited C5b-9a deposition in a dose-dependent manner compared to mouse serum-only control.

### Example 5: Bifunctionals Specifically Stain Kidney Glomeruli Ex Vivo and Localize to the Glomerulus In Vivo

Tissue sections of 5 μm thickness were prepared from OCT-embedded kidney tissues collected from uninjured 129X1/SVJ mice (all test articles [TA] were anti-ROBO2 bifunctional molecules) or the same strain with nephrotoxic serum (NTS)-induced glomerulopathy (GBM6 TA). Slides were acetone fixed for 20 mins, washed with PBS (3×), blocked with BlockAid buffer (Thermofisher Scientific, cat #10710) for 10 mins and stained with TA (100 nM) and anti-nephrin (Abcam, cat #ab216341; glomerular marker) overnight at 4° C. Sections were then washed with PBS (3×) followed by staining at room temperature for 2 hrs with fluorochrome conjugated anti-human kappa (Southern Biotech, cat #2060-31) to detect TA and fluorochrome conjugated anti rabbit IgG (Biolegend, cat #406421) as a secondary for the anti-nephrin co-stain. Sections were washed with PBS and stained with DAPI for 5 mins to identify cell nuclei (Thermo fisher, cat #R37606). Finally, slides were mounted with gold antifade reagent (Thermo fisher, cat #P36934). 20× images were acquired on Olympus FV3000 utilizing Fluoview (FV31S-SW). Kidneys from vehicle-only treated mice served as a negative control for anti-kappa and anti-rabbit IgG staining. Only TA specific for glomerular target proteins ROBO2 and COL4A3NC1 stained kidney glomeruli and colocalized with nephrin, indicating antibody specificity. CDAB1 specifically colocalized with nephrin staining in uninjured mouse tissues while CDAB24 only stained tissues and colocalized with nephrin in tissue sections taken from NTS injury mice. Isotype control molecule did not stain kidney tissue.

In a separate experiment, seven week-old female 129X1/SVJ mice were dosed subcutaneously with 10 mpk TA (CDAB1, isotype control, CDAB8, CDAB10) or vehicle control. Four mice per group were necropsied at day 2 and day 5 following dosing. Right and left kidneys were collected, embedded in OCT and frozen. Tissue sections of 5 μm thickness were prepared, acetone fixed for 20 mins, washed with PBS (3×), blocked with BlockAid buffer (Thermofisher Scientific, cat #10710) for 10 mins and stained with anti-nephrin (Abcam, cat #ab216341; glomerular marker) overnight at 4° C. Sections were then washed with PBS (3×) followed by staining at room temperature for 2 hrs with fluorochrome conjugated anti-human kappa (Southern Biotech, cat #2060-31) to detect TA and fluorochrome conjugated anti rabbit IgG (Biolegend, cat #406421) as a secondary for the anti-nephrin co-stain. Sections were washed with PBS and stained with DAPI for 5 mins to identify cell nuclei (Thermo fisher, cat #R37606). Finally, slides were mounted with gold antifade reagent (Thermo fisher, cat #P36934). 20× images were acquired on Olympus FV3000 utilizing Fluoview (FV31S-SW). Kidneys from vehicle-only treated mice served as a negative control for anti-kappa staining.

Anti-kappa staining indicated localization of anti-ROBO2:CD55 constructs, but not the isotype control molecule, to the glomerulus in mice dosed with the bifunctional molecules.

### Example 6: Bifunctionals Specifically Stain Kidney Ex Vivo and Localize to the Kidney In Vivo

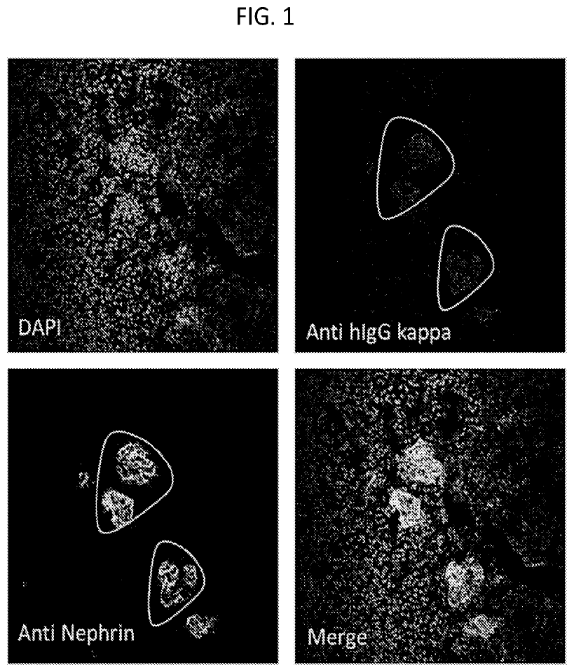

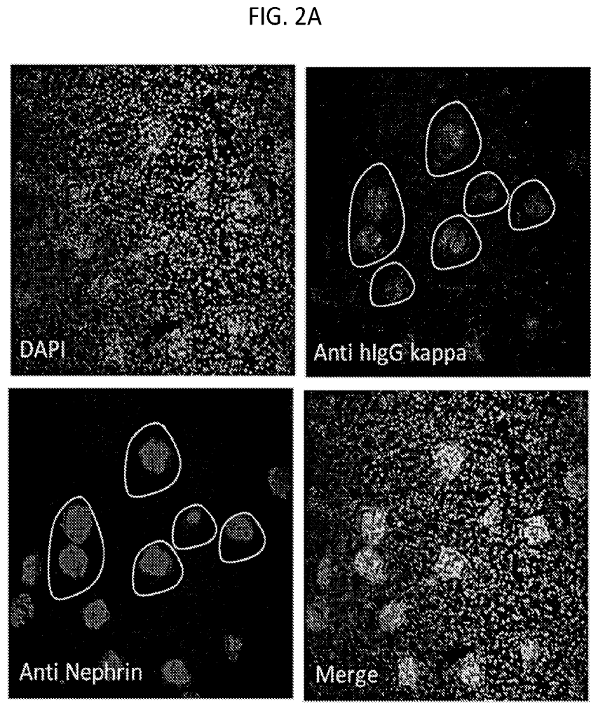

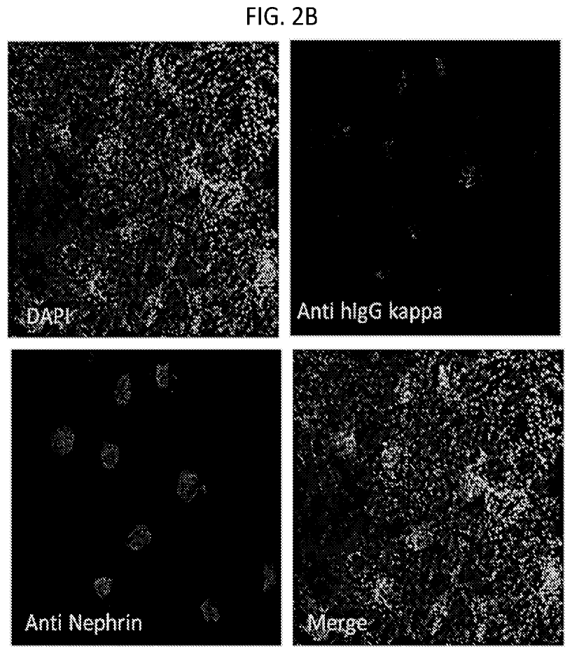

Tissue sections of 5 μm thickness were prepared from OCT-embedded kidney tissues collected from 129X1/SVJ mice previously subcutaneously injected with 10 mg/kg CDAB2. Tissue sections were prepared and stained as above. Kidneys from CDAB2 treated mice stained positive for anti-hIgG kappa and co-localized with anti-nephrin. Accordingly, CDAB2 shows ex vivo and in vivo localization to kidney (FIG. 1, FIG. 2A, and FIG. 2B).

The Examples provided herein demonstrate that molecules provided herein can be used to specifically localize therapeutics at the kidney glomerulus, such as CD55, CD59, CR1, DCP, IL-2 mutein, or PD-1 agonist, and also other therapeutic molecules, such as those described herein. The Example provided herein also demonstrate the potential of the kidney glomerulus-targeting complement modulator bispecific molecules to substantially improve complement-mediated disorder of the kidney glomerulus, or decrease inflammation in the kidney that is associated with autoimmune disease, while reducing adverse effects and improving patient quality of life.

The disclosures of each and every patent, patent application, and publication cited herein are hereby incorporated herein by reference in their entirety. While various embodiments have been disclosed with reference to specific aspects, it is apparent that other aspects and variations of these embodiments may be devised by others skilled in the art without departing from the true spirit and scope of the embodiments. The appended claims are intended to be construed to include all such aspects and equivalent variations.

What is claimed is:

1. A protein comprising a glomerular targeting moiety and an effector moiety, wherein
the glomerular targeting moiety is an antibody that binds to a Robo2 protein, an antibody that binds to a COL4A3 protein, an antibody that binds to a COL4A4 protein, or an antibody that binds to a COL4A5 protein; and
the effector moiety is a complement modulator selected from the group consisting of a CD55 protein, a CD59 protein, a CR1 protein, and a DCP protein.

2. The protein of claim 1, wherein the protein binds to a glomerular cell.

3. The protein of claim 1, wherein the complement modulator inhibits a complement system.

4. The protein of claim 1, wherein the glomerular targeting moiety is an anti-Robo2 antibody that blocks Robo2 binding to SLIT1, SLIT2, SLIT3, and/or SLIT4.

5. The protein of claim 1, wherein the glomerular targeting moiety is an anti-Robo2 antibody that does not block Robo2 binding to SLIT1, SLIT2, SLIT3, or SLIT4.

6-10. (canceled)

11. The protein of claim 1, wherein the glomerular targeting moiety comprises:
an amino acid sequence as set forth in SEQ ID NO: 70 or an amino acid sequence having at least 90% identity to the amino acid sequence of SEQ ID NO: 70;
an amino acid sequence as set forth in SEQ ID NO: 71 or an amino acid sequence having at least 90% identity to the amino acid sequence of SEQ ID NO: 71;
an amino acid sequence as set forth in SEQ ID NO: 72 or an amino acid sequence having at least 90% identity to the amino acid sequence of SEQ ID NO: 72;
an amino acid sequence as set forth in SEQ ID NO: 73 or an amino acid sequence having at least 90% identity to the amino acid sequence of SEQ ID NO: 73;
an amino acid sequence as set forth in SEQ ID NO: 74 or an amino acid sequence having at least 90% identity to the amino acid sequence of SEQ ID NO: 74;
an amino acid sequence as set forth in SEQ ID NO: 75 or an amino acid sequence having at least 90% identity to the amino acid sequence of SEQ ID NO: 75;
an amino acid sequence as set forth in SEQ ID NO: 76 or an amino acid sequence having at least 90% identity to the amino acid sequence of SEQ ID NO: 76;
an amino acid sequence as set forth in SEQ ID NO: 77 or an amino acid sequence having at least 90% identity to the amino acid sequence of SEQ ID NO: 77;
an amino acid sequence as set forth in SEQ ID NO: 78 or an amino acid sequence having at least 90% identity to the amino acid sequence of SEQ ID NO: 78;
an amino acid sequence as set forth in SEQ ID NO: 79 or an amino acid sequence having at least 90% identity to the amino acid sequence of SEQ ID NO: 79; or
an amino acid sequence as set forth in SEQ ID NO: 80 or an amino acid sequence having at least 90% identity to the amino acid sequence of SEQ ID NO: 80.

12. The protein of claim 1, wherein the glomerular targeting moiety comprises:
an amino acid sequence as set forth in SEQ ID NO: 70;
an amino acid sequence as set forth in SEQ ID NO: 71;
an amino acid sequence as set forth in SEQ ID NO: 72;
an amino acid sequence as set forth in SEQ ID NO: 73;
an amino acid sequence as set forth in SEQ ID NO: 74;
an amino acid sequence as set forth in SEQ ID NO: 75;
an amino acid sequence as set forth in SEQ ID NO: 76;
an amino acid sequence as set forth in SEQ ID NO: 77;
an amino acid sequence as set forth in SEQ ID NO: 78;
an amino acid sequence as set forth in SEQ ID NO: 79; or
an amino acid sequence as set forth in SEQ ID NO: 80.

13. The protein of claim 1, wherein the glomerular targeting moiety comprises
an amino acid sequence as set forth in SEQ ID NO: 70;
an amino acid sequence as set forth in SEQ ID NO: 71;
an amino acid sequence as set forth in SEQ ID NO: 72;
an amino acid sequence as set forth in SEQ ID NO: 73;
an amino acid sequence as set forth in SEQ ID NO: 74;
an amino acid sequence as set forth in SEQ ID NO: 75;
an amino acid sequence as set forth in SEQ ID NO: 76;
an amino acid sequence as set forth in SEQ ID NO: 77;
an amino acid sequence as set forth in SEQ ID NO: 78;
an amino acid sequence as set forth in SEQ ID NO: 79; or
an amino acid sequence as set forth in SEQ ID NO: 80; and
wherein the effector moiety comprises
an amino acid sequence as set forth in SEQ ID NO: 1;
an amino acid sequence as set forth in SEQ ID NO: 2;
an amino acid sequence as set forth in SEQ ID NO: 3;
an amino acid sequence as set forth in SEQ ID NO: 4;
an amino acid sequence as set forth in SEQ ID NO: 5;
an amino acid sequence as set forth in SEQ ID NO: 6;
an amino acid sequence as set forth in SEQ ID NO: 7;
an amino acid sequence as set forth in SEQ ID NO: 8;
an amino acid sequence as set forth in SEQ ID NO: 9;
an amino acid sequence as set forth in SEQ ID NO: 10;
an amino acid sequence as set forth in SEQ ID NO: 11; or
an amino acid sequence as set forth in SEQ ID NO: 12.

14. An antibody or antigen binding fragment thereof comprising:
a light chain comprising an amino acid sequence as set forth in SEQ ID NO:83 or an amino acid sequence having at least 90% identity to SEQ ID NO: 83, and a heavy chain comprising an amino acid sequence as set forth in SEQ ID NO:81 or an amino acid sequence having at least 90% identity to SEQ ID NO: 81;
a light chain comprising an amino acid sequence as set forth in SEQ ID NO:87 or an amino acid sequence having at least 90% identity to SEQ ID NO: 87, and a heavy chain comprising an amino acid sequence as set forth in SEQ ID NO:85 or an amino acid sequence having at least 90% identity to SEQ ID NO: 85;
a light chain comprising an amino acid sequence as set forth in SEQ ID NO:102 or an amino acid sequence having at least 90% identity to SEQ ID NO: 102, and a heavy chain comprising an amino acid sequence as set forth in SEQ ID NO:100 or an amino acid sequence having at least 90% identity to SEQ ID NO: 100; or
a light chain comprising an amino acid sequence as set forth in SEQ ID NO:108 or an amino acid sequence having at least 90% identity to SEQ ID NO:108, and a heavy chain comprising an amino acid sequence as set forth in SEQ ID NO:110 or an amino acid sequence having at least 90% identity SEQ ID NO: 110.

15. The antibody or antigen binding fragment thereof, of claim 14 comprising:
a light chain comprising an amino acid sequence as set forth in SEQ ID NO: 83, and a heavy chain comprising an amino acid sequence as set forth in SEQ ID NO: 81;
a light chain comprising an amino acid sequence as set forth in SEQ ID NO: 87, and a heavy chain comprising an amino acid sequence as set forth in SEQ ID NO: 85;
a light chain comprising an amino acid sequence as set forth in SEQ ID NO: 102, and a heavy chain comprising an amino acid sequence as set forth in SEQ ID NO: 100; or
a light chain comprising an amino acid sequence as set forth in SEQ ID NO: 108, and a heavy chain comprising an amino acid sequence as set forth in SEQ ID NO: 110.

16. A protein comprising:
an amino acid sequence as set forth in SEQ ID NO: 81 or an amino acid sequence having at least 90% identity to SEQ ID NO: 81;
an amino acid sequence as set forth in SEQ ID NO: 83 or an amino acid sequence having at least 90% identity to SEQ ID NO: 83;
an amino acid sequence as set forth in SEQ ID NO: 85 or an amino acid sequence having at least 90% identity to SEQ ID NO: 85;
an amino acid sequence as set forth in SEQ ID NO: 87 or an amino acid sequence having at least 90% identity to SEQ ID NO: 87;
an amino acid sequence as set forth in SEQ ID NO: 89 or an amino acid sequence having at least 90% identity to SEQ ID NO: 89;
an amino acid sequence as set forth in SEQ ID NO: 90 or an amino acid sequence having at least 90% identity to SEQ ID NO: 90;
an amino acid sequence as set forth in SEQ ID NO:91 or an amino acid sequence having at least 90% identity to SEQ ID NO: 91;
an amino acid sequence as set forth in SEQ ID NO: 92 or an amino acid sequence having at least 90% identity to SEQ ID NO: 92;
an amino acid sequence as set forth in SEQ ID NO: 93 or an amino acid sequence having at least 90% identity to SEQ ID NO: 93;
an amino acid sequence as set forth in SEQ ID NO: 94 or an amino acid sequence having at least 90% identity to SEQ ID NO: 94;
an amino acid sequence as set forth in SEQ ID NO: 95 or an amino acid sequence having at least 90% identity to SEQ ID NO: 95;
an amino acid sequence as set forth in SEQ ID NO: 96 or an amino acid sequence having at least 90% identity to SEQ ID NO: 96;
an amino acid sequence as set forth in SEQ ID NO: 97 or an amino acid sequence having at least 90% identity to SEQ ID NO: 97;
an amino acid sequence as set forth in SEQ ID NO: 99 or an amino acid sequence having at least 90% identity to SEQ ID NO: 99;
an amino acid sequence as set forth in SEQ ID NO: 100 or an amino acid sequence having at least 90% identity to SEQ ID NO: 100;
an amino acid sequence as set forth in SEQ ID NO: 102 or an amino acid sequence having at least 90% identity to SEQ ID NO: 102;
an amino acid sequence as set forth in SEQ ID NO: 104 or an amino acid sequence having at least 90% identity to SEQ ID NO: 104;
an amino acid sequence as set forth in SEQ ID NO: 105 or an amino acid sequence having at least 90% identity to SEQ ID NO: 105;
an amino acid sequence as set forth in SEQ ID NO: 106 or an amino acid sequence having at least 90% identity to SEQ ID NO: 106;
an amino acid sequence as set forth in SEQ ID NO: 107 or an amino acid sequence having at least 90% identity to SEQ ID NO: 107;
an amino acid sequence as set forth in SEQ ID NO: 108 or an amino acid sequence having at least 90% identity to SEQ ID NO: 108; or
an amino acid sequence as set forth in SEQ ID NO: 110 or an amino acid sequence having at least 90% identity to SEQ ID NO: 110.

17. The protein of claim 16 comprising:
an amino acid sequence as set forth in SEQ ID NO: 81;
an amino acid sequence as set forth in SEQ ID NO: 83;
an amino acid sequence as set forth in SEQ ID NO: 85;
an amino acid sequence as set forth in SEQ ID NO: 87;
an amino acid sequence as set forth in SEQ ID NO: 89;
an amino acid sequence as set forth in SEQ ID NO: 90;
an amino acid sequence as set forth in SEQ ID NO: 91;
an amino acid sequence as set forth in SEQ ID NO: 92;
an amino acid sequence as set forth in SEQ ID NO: 93;
an amino acid sequence as set forth in SEQ ID NO: 94;
an amino acid sequence as set forth in SEQ ID NO: 95;
an amino acid sequence as set forth in SEQ ID NO: 96;
an amino acid sequence as set forth in SEQ ID NO: 97;
an amino acid sequence as set forth in SEQ ID NO: 99;
an amino acid sequence as set forth in SEQ ID NO: 100;
an amino acid sequence as set forth in SEQ ID NO: 102;
an amino acid sequence as set forth in SEQ ID NO: 104;
an amino acid sequence as set forth in SEQ ID NO: 105;
an amino acid sequence as set forth in SEQ ID NO: 106;
an amino acid sequence as set forth in SEQ ID NO: 107;
an amino acid sequence as set forth in SEQ ID NO: 108; or
an amino acid sequence as set forth in SEQ ID NO: 110.

18. A pharmaceutical composition comprising the protein of claim 1, and a pharmaceutically acceptable carrier.

19. (canceled)

20. A method of inhibiting a complement system in the kidney glomerulus of a subject in need thereof, the method comprising tethering of the protein of claim 1 to the glomerulus of the subject in need thereof, wherein tethering allows localized inhibition of the complement system within the glomerulus of the subject.

21. The method of claim 20, wherein the subject has a kidney disease, selected from the group consisting of: Goodpasture's Syndrome (anti-GBM disease), Alport syndrome, inflammatory renal disease, glomerulonephritis, nephritis, lupus, lupus nephritis, IgA nephritis, membranous nephropathy, membranoproliferative glomerulonephritis, acute kidney injury, and chronic kidney disease, focal segmented glomerular sclerosis (FSGS), lupus nephritis, systemic scleroderma, membranous glomerular nephropathy (MGN), membranous nephropathy (MN), minimal change disease (MCD), IgA nephropathy, ANCA-associated vasculitis (AAV), Sjögren's syndrome, Scleroderma, and systemic sclerosis (SSc), or any combination thereof.

22. A method of treating a subject with a kidney inflammatory disorder selected from the group consisting of: Goodpasture's Syndrome (anti-GBM disease), Alport syndrome, inflammatory renal disease, glomerulonephritis, nephritis, lupus, lupus nephritis, IgA nephritis, membranous nephropathy, membranoproliferative glomerulonephritis, acute kidney injury, chronic kidney disease, focal segmented glomerular sclerosis (FSGS), lupus nephritis, systemic scleroderma, membranous glomerular nephropathy (MGN), membranous nephropathy (MN), minimal change disease (MCD), IgA nephropathy, ANCA-associated vasculitis (AAV), Sjögren's syndrome, Scleroderma, and systemic sclerosis (SSc), or any combination thereof, the method comprising administering the protein of claim 1, to the subject to treat the disorder.

23. A method of treating glomerular disorder in a subject in need thereof, comprising administering the pharmaceutical compositions of claim 18, to the subject to treat the disorder, wherein the glomerular disorder is selected from the group consisting of: atypical hemolytic uremic syndrome (aHUS), anti-neutrophil cytoplasmic antibody mediated vasculitis (ANCA), C3 glomerulopathy, IgA nephropathy, immune complex membranoproliferative glomerulonephritis, ischemic reperfusion injury, lupus nephritis, membranous nephropathy, chronic transplant mediated glomerulopathy, immune-complex glomerulonephritis (GN), pauci-immune GN, anti-glomerular basement membrane GN, monoclonal immunoglobulin GN, C3 glomerulopathy, nephrotic syndrome (NS), primary congenital NS (CNS), renal tubular acidosis (RTA), inherited renal tubulopathies, Faconi syndrome, primary nephrogenic diabetes insipidus, minimal change disease, focal segmental glomerular sclerosis, membranous nephropathy, immunoglobulin A nephropathy, C3 glomerulopathy (DDD, C3 GN), idiopathic immune complex membranoproliferative GN, C4 glomerulopathy, infection-related GN, Renal-limited GN, renal limited vasculitis, collagenofibrotic glomerulopathy, thin basement membranes nephropathy, lipoprotein glomerulopathy, ‘Pure’ mesangial proliferative GN, IgM nephropathy, C1q nephropathy, and Idiopathic nodular glomerulosclerosis, or any combination thereof.

24. An isolated nucleic acid molecule encoding the protein of claim 1.

25. An expression vector comprising the isolated nucleic acid of claim 24.

26. A host cell comprising the nucleic acid of claim 23.

27. A method of producing a protein comprising:
(a) culturing a host cell comprising one or more nucleic acids of claim 24 in a culture medium under conditions favorable for expression of the one or more nucleic acids; and
(b) optionally recovering the polypeptide from the culture medium.

28. A host cell comprising the expression vector of claim 25.
